(19) United States
(12) Patent Application Publication
(10) Pub. No.: US 2016/0145222 A1
(43) Pub. Date: May 26, 2016

(54) Methods for Controlling Pests in Soybean

(71) Applicant: BASF SE, Ludwigshafen (DE)

(72) Inventors: Pascal BINDSCHAEDLER, Roemerberg (DE); Wolfgang VON DEYN, Neustadt (DE); Juergen LANGEWALD, Mannheim (DE); Franz-Josef BRAUN, Durham, NC (US); Anna CRISTADORO, Raleigh, NC (US); W. David ROGERS, Durham, NC (US)

(21) Appl. No.: 14/897,957

(22) PCT Filed: Jun. 20, 2014
(86) PCT No.: PCT/EP2014/063020

§ 371 Date: Dec. 11, 2015

Related U.S. Application Data
(60) Provisional application No. 61/837,670, filed on Jun. 21, 2013.

(51) Int. Cl. C07D 275/02; A01N 43/80; C07D 417/10; A01N 47/30

(52) U.S. Cl. CPC C07D 275/02; A01N 47/30; A01N 43/80; C07D 417/10

(57) ABSTRACT

The present invention relates to agricultural methods for controlling pests on soybean plants and soybean crops and the general use of isothiazoline compounds of formula (I)

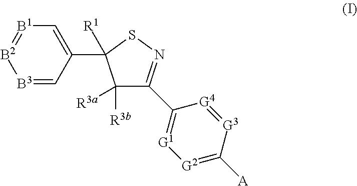

wherein the variables are as defined in the description for pest control.

The compounds of the formula (I) are highly suitable for controlling animal pests on soybean, especially of from the family of Pentatomidae (stink bugs).

The present invention relates to methods of pest control in soybean crops by using isothiazoline compounds of formula (I)

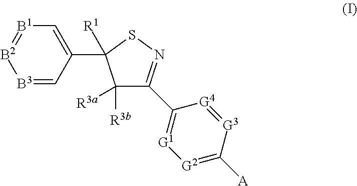

wherein the variables are as defined further below.

Soybeans are considered to be a source of complete protein (Henkel, J., 2000, “Soy:Health Claims for Soy Protein, Question About Other Components”. FDA Consumer (Food and Drug Administration 34 (3): 18-20). For this reason, soy is a good source of protein. According to the US Food and Drug Administration, soy protein products can be good substitutes for animal products because soy offers a ‘complete’ protein profile. Soy protein products can replace animal-based foods—which also have complete proteins but tend to contain more fat, especially saturated fat—without requiring major adjustments elsewhere in the diet.

Soybean protein isolate is highly valuable as it has a biological value of 74 (Protein Quality Evaluation: Report of the Joint FAO/WHO Expert Consultation. Bethesda, Md. (USA): Food and Agriculture Organization of the United Nations (Food and Nutrition Paper No. 51). December 1989).

In agriculture soybeans can produce at least twice as much protein per acre than some other major vegetable or grain crop, e.g. five to 10 times more protein per acre than land set aside for grazing animals to make milk, and up to 15 times more protein per acre than land set aside for meat production (“Soy Benefits”, National Soybean Research Laboratory, February 2012).

Thus, soybeans can be regarded as an importantly global crop providing oil and protein.

Nevertheless, soybean plants are vulnerable to a wide range of bacterial diseases, fungal diseases, viral diseases and parasites. Soybeans are considered to be e.g. the second-most valuable agricultural export in the United States behind corn.

Consequently, in view of the importance of soybean in agriculture, proper pest management is required in order not to jeopardize yield and quality of the soybean crops.

Stink bugs (order of Hemiptera, family of Pentatomidae) are animal pests and true bugs which are probably one of the most common pest problems in soybean (Stewart et al., Soybean Insects—Stink bugs, University of Tennessee Institute of Agriculture, W200 09-0098).

Stink bugs feed on over 52 plants, including native and ornamental trees, shrubs, vines, weeds, and many cultivated crops such as corn and cotton, as well as numerous uncultivated plants, and their preferred hosts are nearly all wild plants. They build up on these hosts and move to soybeans late in the season as their preferred foods mature.

Stink bugs may feed on many parts of the plant, however they typically target developing seed including the pods, meaning that injury to soybean seed is the primary problem associated with stink bug infestations.

Brown or blackish spots may occur where their mouthparts penetrate the plant tissue, but little external signs of feeding injury may be present. Feeding may cause deformation, shriveling or abortion of small seed. Larger seed may only be partly discolored by feeding injury, but this can affect seed quality. High levels of seed abortion may cause the “green bean effect” where foliage is retained and plant maturity is delayed (Stewart et al., Soybean Insects—Stink bugs, University of Tennessee Institute of Agriculture, W200 09-0098).

Stink bugs inflict mechanical injury to the seed as well as transmit the yeast-spot disease organism. The degree of damage caused by this pest depends to some extent on the developmental stage of the seed when it is pierced by the stink bug's needlelike mouthparts. The younger the seed when damaged, the greater the yield reduction. Although late season infestations may not affect yield, bean oil content and germination will be reduced.

The green stink bug (Acrosternum hilare) is in certain regions e.g. one of the most common species that feeds on soybean, the brown stink bug (Euschistus servus) is another common component of the stink bug complex.

Of the complex of sucking bugs that occur in cultivation, the brown stinkbug Euschistus heros is currently considered to be the most abundant species in northern Parana to Central Brazil (Correa-Ferreira & Panizzi, 1999), and is a significant problem in soybean (Schmidt et al., 2003). The bugs occur in soybeans from the vegetative stage and are harmful from the beginning of pod formation until grain maturity. They cause damage to the seed (Galileo & Heinrichs 1978, Panizzi & Slansky Jr., 15, 1985) and can also open the way to fungal diseases and cause physiological disorders, such as soybean leaf retention (Galileo & Heinrichs 1978, Todd & Herzog, 1980).

Other plant feeding species that may be present include the red-shouldered stink bug (Thyanta custator) and the dusky-brown stink bug (Euschistus tristigmus). Another species, the southern green stink bug (Nezara viridula) is often confined to the southernmost counties of the US. Predatory (beneficial) stink bugs such as the spined soldier bug (Podisus maculaventris) may also be found in soybean and are sometimes mistaken for brown or dusky-brown stink bugs.

Control of stinkbugs in soybean is often vital to prevent significant economic damage.

Insecticides commonly used to control stinkbugs include pyrethroids, neonicotinoids and organophosphates, although pyrethroid insecticides are usually the method of choice for controlling stink bugs in soybean. However, there are increasing problems with insecticide resistance, particularly in brown stink bug populations and particularly to pyrethroids. Euschistus heros can also be difficult to manage using organophosphates or endosulfan (Sosa-Gomez et al., 2009). There is therefore a need for effective alternative methods of controlling stinkbugs in soybean.

Particular insecticides from the class of gamma-aminobutyric acid (GABA)-gated chloride channel antagonists (disclosed in e.g. WO 2005/085216 (EP1731512), WO2009/002809 and WO2009/080250) seem to be effective at controlling stinkbugs, especially in soybean such as described in WO2012/104331.

Regarding the compounds applied in the methods of the present invention, related insecticidal aryl azoline compounds are described in WO 2011/092287, WO 2011/073444, WO 2010/090344, WO 2009/112275 and WO 97/23212.

Related insecticidal aryl isothiazoline compounds are described in WO 2013/037626. However, these documents do not describe compounds having the characteristic substituents and substituents' arrangement as claimed in the present invention.

Compounds applied in methods of the present inventions have been encompassed in WO2013/092943. However, their excellent suitability in methods for the protection and treatment of soybean, as well as their extraordinary activity and efficiency against pests of the family of pentatomidae, stink bugs, in particular against Euschistus spp and more particularly against Euschistus heros, have not been described therein.

Surprisingly it has been found that the compounds of formula (I) provide an efficient control against pests on soybean, especially against pests of the family of pentatomidae, stink bugs. These compounds therefore represent an important new solution for controlling pests of the family of pentatomidae, stink bugs, and thereby safeguarding soybean plants, crops and propagation material from the infestion by such stinkbugs, particularly where stink bugs are resistant to current methods.

In a first aspect the invention provides a method comprising applying to soybean plants, to crops of soybean plants, soybean propagation material or to the locus thereof, a compound of formula I

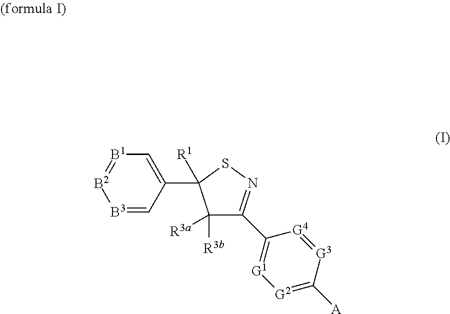

wherein
A is a group A¹, A² or A³;
   wherein
   A¹ is selected from the group consisting of —C(═NR⁶)R⁸, —S(O)ₙR⁹, and —N(R⁵)R⁶;
   A² is a group of following formula:

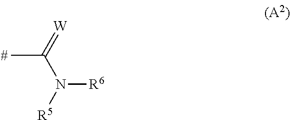

wherein
# denotes the bond to the remainder of the molecule;
W is selected from O and S;
A³ is a group of following formula:

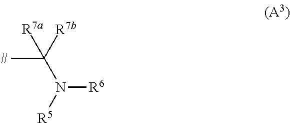

wherein
denotes the bond to the aromatic ring of formula (I);
B¹, B² and B³ are each independently selected from the group consisting of N and CR², with the proviso that at most two of B¹, B² and B³ are N;
G¹, G², G³ and G⁴ are each independently selected from the group consisting of N and CR⁴, with the proviso that at most two of G¹, G², G³ and G⁴ are N;
R¹ is selected from the group consisting of C₁-C₄-alkyl, C₁-C₄-haloalkyl, C₁-C₄-alkoxy-C₁-C₄-alkyl, C₁-C₄-haloalkoxy-C₁-C₄-alkyl-, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl and —C(═O)OR¹⁵;
each R² is independently selected from the group consisting of hydrogen, halogen, cyano, azido, nitro, —SCN, —SF₅, C₁-C₆-alkyl, C₃-C₈-cycloalkyl, C₂-C₆-alkenyl, C₂-C₆-alkynyl, wherein the four last mentioned aliphatic and cycloaliphatic radicals may be partially or fully halogenated and/or may be substituted by one or more radicals R⁸,
   —Si(R¹²)₃, —OR⁹, —S(O)ₙR⁹, and —NR^{10a}R^{10b};
R^{3a}, R^{3b} are each independently selected from the group consisting of hydrogen, halogen, hydroxyl, —CO₂R^{3d}, C₁-C₃-alkyl, C₁-C₃-haloalkyl, C₂-C₃-alkenyl, C₂-C₃-alkynyl, C₁-C₃-alkoxy, C₁-C₃-haloalkoxy, C₁-C₃-alkylthio, C₁-C₃-haloalkylthio, C₁-C₃-alkylsulfonyl and C₁-C₃-haloalkylsulfonyl; or
   R^{3a} and R^{3b} together form a group ═O, ═C(R^{3c})₂, ═NOH or ═NOCH₃;
each R^{3c} is independently selected from the group consisting of hydrogen, halogen, CH₃ and CF₃;
R^{3d} is selected from the group consisting of hydrogen, C₁-C₆-alkyl and C₁-C₃-alkyloxy-C₁-C₃-alkyl-;
each R⁴ is independently selected from the group consisting of hydrogen, halogen, cyano, azido, nitro, —SCN, —SF₅, C₁-C₆-alkyl which may be partially or fully halogenated and/or may be substituted by one or more radicals R⁸, C₃-C₈-cycloalkyl which may be partially or fully halogenated and/or may be substituted by one or more radicals R⁸, C₂-C₆-alkenyl which may be partially or fully halogenated and/or may be substituted by one or more radicals R⁸, C₂-C₆-alkynyl which may be partially or fully halogenated and/or may be substituted by one or more radicals R⁸,
   —Si(R¹²)₃, —OR⁹, —NR^{10a}R^{10b},
   phenyl which may be substituted by 1, 2, 3, 4 or 5 radicals R¹¹, and a 3-, 4-, 5-, 6-7-, 8-, 9- or 10-membered saturated, partially unsaturated or maximally unsaturated heteromonocyclic or heterobicyclic ring containing 1, 2, 3 or 4 heteroatoms or heteroatom groups selected from N, O, S, NO, SO and SO₂, as ring members, where the heteromonocyclic or heterobicyclic ring may be substituted by one or more radicals R¹¹;
each R⁵ is independently selected from the group consisting of hydrogen, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted with one or more substituents R⁸,
R⁶ in A² is selected from hydrogen, C₁-C₆-alkyl, C₁-C₆-haloalkyl, C₁-C₄-alkyl which carries one radical R⁸, C₂-C₆-alkenyl, C₂-C₆-haloalkenyl, C₂-C₆-alkynyl, C₃-C₆-cycloalkyl which may be substituted by 1 to 4 substituents selected from halogen and cyano;
   —N(R^{10a})R^{10b}, —CH═NOR⁹;
   phenyl which may be substituted with 1, 2, 3, 4, or 5 substituents R¹¹; and a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from N, O, S, NO, SO and SO₂, as ring members, where the heteromonocyclic or heterobicyclic ring may be substituted with one or more substituents R¹¹; or
   R⁵ and R⁶ form together a group ═S(O)ₘ(R⁹)₂;
each R⁶ in all other cases is independently selected from the group consisting of hydrogen, cyano, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted by one or more substituents R⁸,
   —OR⁹, —NR^{10a}R^{10b}, —S(O)ₙR⁹, —C(═O)NR^{10a}N(R^{10a})R^{10b}), —Si(R¹²)₃, —C(═O)R⁸,
   phenyl which may be substituted with 1, 2, 3, 4, or 5 substituents R¹¹, and
      a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from N, O, S, NO, SO and SO₂, as ring members, where the heteromonocyclic or heterobicyclic ring may be substituted with one or more substituents R¹¹;
   or
   R⁵ and R⁶ or R⁵ and R^{6a}, together with the nitrogen atom to which they are bound, form a 3-, 4-, 5-, 6-, 7- or 8-membered saturated, partially unsaturated or maximally unsaturated heterocyclic ring, where the ring may further contain 1, 2, 3 or 4 heteroatoms or heteroatom-containing groups selected from O, S, N, SO, SO₂, C═O and C═S as ring members, wherein the heterocyclic ring may be substituted with 1, 2, 3, 4 or 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic or cycloaliphatic moieties in the twelve last-mentioned radicals may be substituted by one or more radicals R⁸, and phenyl which may be substituted with 1, 2, 3, 4 or 5 substituents R¹¹;
R^{7a}, R^{7b} are each independently selected from the group consisting of hydrogen, halogen, cyano, C₁-C₆-alkyl, C₃-C₈-cycloalkyl, C₂-C₆-alkenyl and C₂-C₆-alkynyl, wherein the four last-mentioned aliphatic and cycloaliphatic radicals may be partially or fully halogenated and/or may be substituted by one or more radicals R⁸;
each R⁸ is independently selected from the group consisting of cyano, azido, nitro, —SCN, —SF₅, C₃-C₈-cycloalkyl, C₃-C₈-halocycloalkyl, C₃-C₈-cycloalkenyl, C₃-C₈-halocycloalkenyl, where the cycloaliphatic moieties in the four last-mentioned radicals may be substituted by one or more radicals R¹³;
   —Si(R¹²)₃, —OR⁹, —OSO₂R⁹, —S(O)ₙR⁹, —N(R^{10a})R^{10b}, —C(═O)N(R^{10a})R^{10b}, —C(═S)N(R^{10a})R^{10b}, —C(═O)OR⁹, —CH═NOR⁹;
   phenyl, optionally substituted with 1, 2, 3, 4 or 5 substituents R¹⁶, and a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more substituents R¹⁶,
   or
   two R⁸ present on the same carbon atom of an alkyl, alkenyl, alkynyl or cycloalkyl group together form a group ═O, ═C(R¹³)₂; ═S; ═S(O)ₘ(R¹⁵)₂, ═S(O)ₘR¹⁵N(R^{14a})R^{14b}, ═NR^{10a}, ═NOR⁹; or ═NN(R^{10a})R^{10b};
   or
   two radicals R⁸, together with the carbon atoms of an alkyl, alkenyl, alkynyl or cycloalkyl group which they are bonded to, form a 3-, 4-, 5-, 6-, 7- or 8-membered saturated or partially unsaturated carbocyclic or heterocyclic ring, where the heterocyclic ring comprises 1, 2, 3 or 4 heteroatoms or heteroatom groups independently selected from N, O, S, NO, SO and SO₂, as ring members, and where the carbocyclic or heterocyclic ring is optionally substituted with one or more substituents R¹⁶; and
   R⁸ as a substituent on a cycloalkyl ring is additionally selected from the group consisting of C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in these six radicals may be substituted by one or more radicals R¹³; and
   R⁸ in the groups —C(═NR⁶)R⁸, and —C(═O)R⁸ is additionally selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in the six last-mentioned radicals may be substituted by one or more radicals R¹³;
   each R⁹ is independently selected from the group consisting of hydrogen, cyano, C₁-C₆-alkyl, C₁-C₆-haloalkyl, C₃-C₈-cycloalkyl, C₃-C₈-cycloalkyl-C₁-C₄-alkyl-, C₃-C₈-halocycloalkyl, C₂-C₆-alkenyl, C₂-C₆-haloalkenyl, C₂-C₆-alkynyl, C₂-C₆-haloalkynyl, where the aliphatic and cycloaliphatic moieties in the nine last-mentioned radicals may be substituted by one or more radicals R¹³,
   —C₁-C₆-alkyl-C(═O)OR¹⁵, —C₁-C₆-alkyl-C(═O)N(R^{14a})R^{14b},
   —C₁-C₆-alkyl-C(═S)N(R^{14a})R^{14b}, —C₁-C₆-alkyl-C(═NR¹⁴)N(R^{14a})R^{14b},
   —Si(R¹²)₃, —S(O)ₙR¹⁵, —S(O)ₙN(R^{14a})R^{14b}, —N(R^{10a})R^{10b}, —N═C(R¹³)₂, —C(═O)R¹³, —C(═O)N(R^{14a})R^{14b}, —C(═S)N(R^{14a})R^{14b}, —C(═O)OR¹⁵,
   phenyl, optionally substituted with one or more substituents R¹⁶; and
      a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more substituents R¹⁶; and
      R⁹ in the groups —S(O)ₙR⁹ and —OSO₂R⁹ is additionally selected from the group consisting of C₁-C₆-alkoxy and C₁-C₆-haloalkoxy;
R^{10a}, R^{10b} independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, where the aliphatic and cycloaliphatic moieties in the eight last-mentioned radicals may be substituted by one or more radicals R¹³;
   —C₁-C₆-alkyl-C(═O)OR¹⁵, —C₁-C₆-alkyl-C(═O)N(R^{14a})R^{14b}, —C₁-C₆-alkyl-C(═S) N(R^{14a})R^{14b}, —C₁-C₆-alkyl-C(═NR¹⁴)N(R^{14a})R^{14b}, C₁-C₆-alkoxy, C₁-C₆-haloalkoxy, C₁-C₆-alkylthio, C₁-C₆-haloalkylthio,
   —S(O)ₙR¹⁵, —S(O)ₙN(R^{14a})R^{14b}, —C(═O)R¹³, —C(═O)OR¹⁵, —C(═O)N(R^{14a})R^{14b},
   —C(═S)R¹³, —C(═S)SR¹⁵, —C(═S)N(R^{14a})R^{14b}, —C(═NR¹⁴)R¹³;
   phenyl, optionally substituted with 1, 2, 3 or 4, substituents R¹⁶; and a 3-, 4-, 5-, 6- or 7-membered saturated, partially unsaturated or maximally unsaturated heterocyclic ring comprising 1, 2, 3 or 4 heteroatoms or heteroatom groups selected from N, O, S, NO, SO and SO₂, as ring members, where the heterocyclic ring is optionally substituted with one or more substituents R¹⁶;
   or
   R^{10a} and R^{10b} form together with the nitrogen atom they are bonded to a 3-, 4-, 5-, 6-, 7- or 8-membered saturated, partially unsaturated or maximally unsaturated heterocyclic ring, wherein the heterocyclic ring may additionally contain one or two heteroatoms or heteroatom groups selected from N, O, S, NO, SO and SO₂, as ring members, where the heterocyclic ring optionally carries one or more substituents selected from halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl, optionally substituted with 1, 2, 3, 4 or 5 substituents R¹⁶, and a 3-, 4-, 5-, 6-, or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring optionally carries one or more substituents R¹⁶;
   or R^{10a} and R^{10b} together form a group ═C(R¹³)₂, ═S(O)ₘ(R¹⁵)₂, ═S(O)ₘR¹⁵N(R^{14a})R^{14b}, ═NR¹⁴ or ═NOR¹⁵;
R¹¹ is independently selected from the group consisting of halogen, cyano, azido, nitro, —SCN, —SF₅, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted with one or more radicals R⁸,
   —OR⁹, —NR^{10a}R^{10b}, —S(O)ₙR⁹, —Si(R¹²)₃;
   phenyl, optionally substituted with 1, 2, 3, 4, or 5 substituents selected independently from R¹⁶; and
   a 3-, 4-, 5-, 6- or 7-membered saturated, partially unsaturated or maximally unsaturated aromatic heterocyclic ring comprising 1, 2, 3 or 4 heteroatoms or heteroatom groups selected from N, O, S, NO, SO and SO₂, as ring members, where the heterocyclic ring is optionally substituted with one or more substituents selected independently from R¹⁶;
   or two R¹¹ present on the same ring carbon atom of an unsaturated or partially unsaturated heterocyclic ring may together form a group ═O, ═C(R¹³)₂; ═S; ═S(O)ₘ(R¹⁵)₂; ═S(O)ₘR¹⁵N(R^{14a})R^{14b}, ═NR¹⁴, ═NOR¹⁵, or ═NN(R^{14a})R^{14b};
   or two R¹¹ bound on adjacent ring atoms form together with the ring atoms to which they are bound a saturated 3-, 4-, 5-, 6-, 7-, 8- or 9-membered ring, wherein the ring may contain 1 or 2 heteroatoms or heteroatom groups selected from O, S, N, NR¹⁴, NO, SO and SO₂ and/or 1 or 2 groups selected from C═O, C═S and C═NR¹⁴ as ring members, and wherein the ring may be substituted by one or more radicals selected from the group consisting of halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁶, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹⁶;
each R¹² is independently selected from the group consisting of hydrogen, halogen, C₁-C₆-alkyl, C₁-C₆-haloalkyl, C₁-C₆-alkoxy, C₁-C₆-haloalkoxy, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, and
   phenyl, optionally substituted with 1, 2, 3, 4, or 5 substituents R¹⁶;
each R¹³ is independently selected from the group consisting of cyano, nitro, —OH, —SH, —SCN, —SF₅, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —NR^{14a}R^{14b}, —C(═O)NR^{14a}R^{14b}, trimethylsilyl, triethylsilyl, tert-butyldimethylsilyl,
   C₃-C₈-cycloalkyl which may be unsubstituted, partially or fully halogenated and/or may carry 1 or 2 radicals selected from cyano, C₁-C₄-alkyl, C₃-C₄-cycloalkyl, C₁-C₄-alkoxy, C₁-C₄-haloalkoxy and oxo; phenyl, benzyl, phenoxy, where the phenyl moiety in the three last-mentioned radicals may be unsubstituted or carry 1, 2, 3, 4 or 5 substituents R¹⁶; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by 1, 2 or 3 substituents R¹⁶;
   or
   two R¹³ present on the same carbon atom of an alkyl, alkenyl, alkynyl or cycloalkyl group may together be ═O, ═CH(C₁-C₄-alkyl), ═C(C₁-C₄-alkyl)C₁-C₄-alkyl, ═NR¹⁷ or ═NOR¹⁷;
   and
   R¹³ as a substituent on a cycloalkyl ring is additionally selected from the group consisting of C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 substituents selected from CN, C₃-C₄-cycloalkyl, C₁-C₄-alkoxy, C₁-C₄-haloalkoxy and oxo;
   and
   R¹³ in the groups ═C(R¹³)₂, —N═C(R¹³)₂, —C(═O)R¹³, —C(═S)R¹³ and
   —C(═NR¹⁴)R¹³ is additionally selected from the group consisting of hydrogen, halogen, C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 radicals selected from CN, C₃-C₄-cycloalkyl, C₁-C₄-alkoxy, C₁-C₄-haloalkoxy and oxo;
each R¹⁴ is independently selected from the group consisting of hydrogen, cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —C(═O)NR^{18a}R^{18b}, trimethylsilyl, triethylsilyl, tert-butyldimethylsilyl,
   C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from CN, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl which may be substituted by 1 or 2 substituents selected from halogen and cyano; and oxo;
   C₃-C₈-cycloalkyl which may be unsubstituted, partially or fully halogenated and/or may carry 1 or 2 radicals selected from cyano, C₁-C₄-alkyl, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl, C₃-C₄-cycloalkyl-C₁-C₄-alkyl-, where the cycloalkyl moiety in the two last-mentioned radicals may be substituted by 1 or 2 substituents selected from halogen and cyano; and oxo;
   phenyl, benzyl, pyridyl, phenoxy, wherein the cyclic moieties in the four last-mentioned radicals may be unsubstituted and/or carry 1, 2 or 3 substituents selected from halogen, C₁-C₆-alkyl, C₁-C₆-haloalkyl, C₁-C₆-alkoxy, C₁-C₆-haloalkoxy and (C₁-C₆-alkoxy)carbonyl; and a 3-, 4-, 5- or 6-membered saturated, partially unsaturated or maximally unsaturated heterocyclic ring comprising 1 or 2 heteroatoms or heteroatom groups selected from N, O, S, NO, SO and SO₂, as ring members, where the heterocyclic ring is optionally substituted with one or more substituents R¹⁶;
R^{14a} and R^{14b}, independently of each other, have one of the meanings given for R¹⁴; or
   R^{14a} and R^{14b}, together with the nitrogen atom to which they are bound, form a 3-, 4-, 5-, 6- or 7-membered saturated, partially unsaturated or maximally unsaturated heterocyclic ring, wherein the heterocyclic ring may additionally contain 1 or 2 heteroatoms or heteroatom groups selected from N, O, S, NO, SO and SO₂, as ring members, where the heterocyclic ring optionally carries one or more substituents selected from halogen, C₁-C₄-alkyl, C₁-C₄-haloalkyl, C₁-C₄-alkoxy and C₁-C₄-haloalkoxy;
   or
   R^{14a} and R¹⁴ or R^{14b} and R¹⁴, together with the nitrogen atoms to which they are bound in the group —C(═NR¹⁴)N(R^{14a})R^{14b}, form a 3-, 4-, 5-, 6- or 7-membered partially unsaturated or maximally unsaturated heterocyclic ring, wherein the heterocyclic ring may additionally contain 1 or 2 heteroatoms or heteroatom groups selected from N, O, S, NO, SO and SO₂, as ring members, where the heterocyclic ring optionally carries one or more substituents selected from halogen, C₁-C₄-haloalkyl, C₁-C₄-alkoxy and C₁-C₄-haloalkoxy;
each R¹⁵ is independently selected from the group consisting of hydrogen, cyano, trimethylsilyl, triethylsilyl, tert-butyldimethylsilyl,
   C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo;
   C₃-C₈-cycloalkyl which may be unsubstituted, partially or fully halogenated and/or may carry 1 or 2 radicals selected from C₁-C₄-alkyl, C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo; phenyl, benzyl, pyridyl and phenoxy, wherein the four last-mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl;
each R¹⁶ is independently selected from the group consisting of halogen, nitro, cyano, —OH, —SH, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl;
   C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from C₃-C₄-cycloalkyl, C₁-C₄-alkoxy, C₁-C₄-haloalkoxy and oxo;
   C₃-C₈-cycloalkyl which may be unsubstituted, partially or fully halogenated and/or may carry 1 or 2 radicals selected from C₁-C₄-alkyl, C₃-C₄-cycloalkyl, C₁-C₄-alkoxy, C₁-C₄-haloalkoxy and oxo;
   phenyl, benzyl, pyridyl and phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl;
   or
   two R¹⁶ present together on the same atom of an unsaturated or partially unsaturated ring may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
   or
   two R¹⁶ on two adjacent carbon atoms form together with the carbon atoms they are bonded to a 4-, 5-, 6-, 7- or 8-membered saturated, partially unsaturated or maximally unsaturated ring, wherein the ring may contain 1 or 2 heteroatoms or heteroatom groups selected from N, O, S, NO, SO and SO₂, as ring members, and wherein the ring optionally carries one or more substituents selected from halogen, C₁-C₄-haloalkyl, C₁-C₄-alkoxy and C₁-C₄-haloalkoxy;
R¹⁷, R^{18a} and R^{18b}, independently of each other and independently of each occurrence, are selected from the group consisting of hydrogen, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from CN, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl which may be substituted by 1 or 2 substituents selected from halogen and cyano; and oxo;
   C₃-C₈-cycloalkyl which may be unsubstituted, partially or fully halogenated and/or may carry 1 or 2 radicals selected from cyano, C₁-C₄-alkyl and C₁-C₄-haloalkyl; phenyl and benzyl;
each n is independently 0, 1 or 2; and
each m is independently 0 or 1;
   and/or the stereoisomers, enantiomers, diastereomers and/or N-oxides, agriculturally or veterinary acceptable salts thereof;
   and wherein the method is for controlling and/or preventing pest infestation of the soybean plants and/or soybean crops.

In particular, the invention provides a method comprising applying to soybean plants, to crops of soybean plants, soybean propagation material or to the locus thereof, a compound of formula I

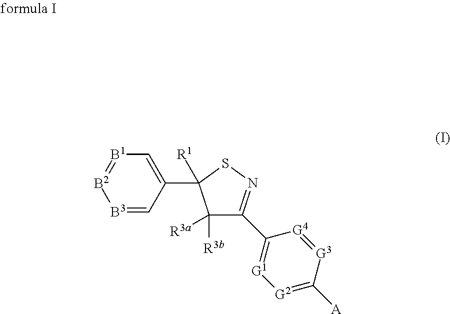

wherein
A is a group A¹, A² or A³;
   wherein
      A¹ is selected from the group consisting of —C(═NR⁶)R⁸, —S(O)ₙR⁹, and —N(R⁵)R⁶;
      A² is a group of following formula:

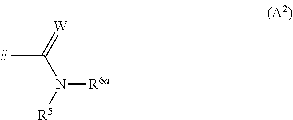

wherein
   # denotes the bond to the remainder of the molecule;
   W is selected from O and S;
A³ is a group of following formula:

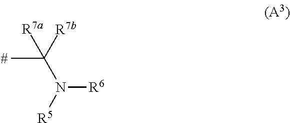

wherein
   # denotes the bond to the aromatic ring of formula (I);
B¹, B² and B³ are each independently selected from the group consisting of N and CR², with the proviso that at most two of B¹, B² and B³ are N;
G¹, G², G³ and G⁴ are each independently selected from the group consisting of N and CR⁴, with the proviso that at most two of G¹, G², G³ and G⁴ are N;
R¹ is selected from the group consisting of C₁-C₄-alkyl, C₁-C₄-haloalkyl, C₁-C₄-alkoxy-C₁-C₄-alkyl, C₁-C₄-haloalkoxy-C₁-C₄-alkyl-, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl and —C(═O)OR¹⁵;
each R² is independently selected from the group consisting of hydrogen, halogen, cyano, azido, nitro, —SCN, —SF₅, C₁-C₆-alkyl, C₃-C₈-cycloalkyl, C₂-C₆-alkenyl, C₂-C₆-alkynyl, wherein the four last mentioned aliphatic and cycloaliphatic radicals may be partially or fully halogenated and/or may be substituted by one or more radicals R⁸, —Si(R¹²)₃, —OR⁹, —S(O)ₙR⁹, and —NR^{10a}R^{10b};
   R^{3a}, R^{3b} are each independently selected from the group consisting of hydrogen, halogen, hydroxyl, —CO₂R^{3d}, C₁-C₃-alkyl, C₁-C₃-haloalkyl, C₂-C₃-alkenyl, C₂-C₃-alkynyl, C₁-C₃-alkoxy, C₁-C₃-haloalkoxy, C₁-C₃-alkylthio, C₁-C₃-haloalkylthio, C₁-C₃-alkylsulfonyl and C₁-C₃-haloalkylsulfonyl; or
   R^{3a} and R^{3b} together form a group ═O, ═C(R^{3c})₂, ═NOH or ═NOCH₃;
   wherein
   each R^{3c} is independently selected from the group consisting of hydrogen, halogen, CH₃ and CF₃; and
   R^{3d} is selected from the group consisting of hydrogen, C₁-C₆-alkyl and C₁-C₃-alkyloxy-C₁-C₃-alkyl-;
each R⁴ is independently selected from the group consisting of hydrogen, halogen, cyano, azido, nitro, —SCN, —SF₅, C₁-C₆-alkyl which may be partially or fully halogenated and/or may be substituted by one or more radicals R⁸, C₃-C₈-cycloalkyl which may be partially or fully halogenated and/or may be substituted by one or more radicals R⁸, C₂-C₆-alkenyl which may be partially or fully halogenated and/or may be substituted by one or more radicals R⁸, C₂-C₆-alkynyl which may be partially or fully halogenated and/or may be substituted by one or more radicals R⁸,
   —Si(R¹²)₃, —OR⁹, —S(O)ₙR⁹, —NR^{10a}R^{10b},
   phenyl which may be substituted by 1, 2, 3, 4 or 5 radicals R¹¹, and a 3-, 4-, 5-, 6-7-, 8-, 9- or 10-membered saturated, partially unsaturated or maximally unsaturated heteromonocyclic or heterobicyclic ring containing 1, 2, 3 or 4 heteroatoms or heteroatom groups selected from N, O, S, NO, SO and SO₂, as ring members, where the heteromonocyclic or heterobicyclic ring may be substituted by one or more radicals R¹¹;
each R⁵ is independently selected from the group consisting of hydrogen, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted with one or more substituents R⁸,
R^{6a} is selected from —X—R^{6b} and —N(R^{5a})R^{6c}; wherein
   X is selected from —C(R^{a})₂—, —C(R^{a})₂—C(R^{a})₂—, —C(R^{a})₂—C(═O)—NR^{10a}—C(R^{a})₂—, —C(R^{a})₂—C(R^{a})₂—C(═O)—NR^{10a}—C(R^{a})₂—, —C(R^{a})₂S(O)ₙ—C(R^{a})₂—, —C(R^{a})₂—C(R^{a})₂—S(O)ₙ—C(R^{a})₂—, —C(R^{a})₂—O—C(R^{a})₂—, and
      —C(R^{a})₂—C(R^{a})₂—O—C(R^{a})₂—, wherein
      each R^{a} is independently selected from the group consisting of hydrogen, halogen, C₁-C₃-alkyl, and C₁-C₃-haloalkyl;
   R^{5a} has independently one of the meanings given for R⁵;
   R^{6b} is selected from the group consisting of C₃-C₈-cycloalkyl, C₂-C₈-alkenyl, C₂-C₈-alkynyl, wherein the three last-mentioned radicals may be partially or fully halogenated and/or may be substituted by one or more substituents R⁸; and
   R^{6c} is selected from the group consisting of hydrogen, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, where the aliphatic and cycloaliphatic moieties in the eight last-mentioned radicals may be substituted by one or more radicals R¹³;
      —C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, C₁-C₆-alkoxy, C₁-C₆-haloalkoxy, C₁-C₆-alkylthio, C₁-C₆-haloalkylthio,
      —S(O)ₙR¹⁵, —S(O)ₙN(R^{14a})R^{14b}, —C(═O)R¹³, —C(═O)OR¹⁵, —C(═O)N(R^{14a})R^{14b}, —C(═S)R¹³, —C(═S)SR¹⁵, —C(═S)N(R^{14a})R^{14b}, and —C(═NR¹⁴)R¹³;
   where in case that R⁵ is hydrogen, R^{6a} is further selected from hydrogen, 1-cyanocyclopropyl, 1-cyanocyclobutyl and 1-cyanopentyl; or R⁵ and R^{6a} form together a group ═S(O)ₘ(R⁹)₂;
each R⁶ is independently selected from the group consisting of hydrogen, cyano, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted by one or more substituents R⁸,
   —OR⁹, —NR^{10a}R^{10b}, —S(O)ₙR⁹, —C(═O)NR^{10a}N(R^{10a})R^{10b}), —Si(R¹²)₃, —C(═O)R⁸, phenyl which may be substituted with 1, 2, 3, 4, or 5 substituents R¹¹, and a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from N, O, S, NO, SO and SO₂, as ring members, where the heteromonocyclic or heterobicyclic ring may be substituted with one or more substituents R¹¹;
   or
   R⁵ and R⁶ or R⁵ and R^{6a}, together with the nitrogen atom to which they are bound, form a 3-, 4-, 5-, 6-, 7- or 8-membered saturated, partially unsaturated or maximally unsaturated heterocyclic ring, where the ring may further contain 1, 2, 3 or 4 heteroatoms or heteroatom-containing groups selected from O, S, N, SO, SO₂, C═O and C═S as ring members, wherein the heterocyclic ring may be substituted with 1, 2, 3, 4 or 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic or cycloaliphatic moieties in the twelve last-mentioned radicals may be substituted by one or more radicals R⁸, and phenyl which may be substituted with 1, 2, 3, 4 or 5 substituents R¹¹;
R^{7a}, R^{7b} are each independently selected from the group consisting of hydrogen, halogen, cyano, C₁-C₆-alkyl, C₃-C₈-cycloalkyl, C₂-C₆-alkenyl and C₂-C₆-alkynyl, wherein the four last-mentioned aliphatic and cycloaliphatic radicals may be partially or fully halogenated and/or may be substituted by one or more radicals R⁸;
each R⁸ is independently selected from the group consisting of cyano, azido, nitro, —SCN, —SF₅, C₃-C₈-cycloalkyl, C₃-C₈-halocycloalkyl, C₃-C₈-cycloalkenyl, C₃-C₈-halocycloalkenyl, where the cycloaliphatic moieties in the four last-mentioned radicals may be substituted by one or more radicals R¹³;
   —Si(R¹²)₃, —OR⁹, —OSO₂R⁹, —S(O)ₙR⁹, —N(R^{10a})R^{10b}, —C(═O)N(R^{10a})R^{10b}, —C(═S)N(R^{10a})R^{10b}, —C(═O)OR⁹,
   phenyl, optionally substituted with 1, 2, 3, 4 or 5 substituents R¹⁶, and a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more substituents R¹⁶,
   or
   two R⁸ present on the same carbon atom of an alkyl, alkenyl, alkynyl or cycloalkyl group together form a group ═O, ═C(R¹³)₂; ═S; ═S(O)ₘ(R¹⁵)₂, ═S(O)ₘR¹⁵N(R^{14a})R^{14b}, ═NR^{10a}, ═NOR⁹; or ═NN(R^{10a})R^{10b};
   or
   two radicals R⁸, together with the carbon atoms of an alkyl, alkenyl, alkynyl or cycloalkyl group which they are bonded to, form a 3-, 4-, 5-, 6-, 7- or 8-membered saturated or partially unsaturated carbocyclic or heterocyclic ring, where the heterocyclic ring comprises 1, 2, 3 or 4 heteroatoms or heteroatom groups independently selected from N, O, S, NO, SO and SO₂, as ring members, and where the carbocyclic or heterocyclic ring is optionally substituted with one or more substituents R¹⁶; and R⁸ as a substituent on a cycloalkyl ring is additionally selected from the group consisting of C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in these six radicals may be substituted by one or more radicals R¹³; and
   R⁸ in the groups —C(═NR⁶)R⁸, and —C(═O)R⁸ is additionally selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in the six last-mentioned radicals may be substituted by one or more radicals R¹³;
each R⁹ is independently selected from the group consisting of hydrogen, cyano, C₁-C₆-alkyl, C₁-C₆-haloalkyl, C₃-C₈-cycloalkyl, C₃-C₈-cycloalkyl-C₁-C₄-alkyl-, C₃-C₈-halocycloalkyl, C₂-C₆-alkenyl, C₂-C₆-haloalkenyl, C₂-C₆-alkynyl, C₂-C₆-haloalkynyl, where the aliphatic and cycloaliphatic moieties in the nine last-mentioned radicals may be substituted by one or more radicals R¹³,
   —C₁-C₆-alkyl-C(═O)O R¹⁵, —C₁-C₆-alkyl-C(═O)N(R^{14a}) R^{14b}, —C₁-C₆-alkyl-C(═S)N(R^{14a})R^{14b}, —C₁-C₆-alkyl-C(═NR¹⁴)N(R^{14a})R^{14b},
   —Si(R¹²)₃, —S(O)ₙR¹⁵, —S(O)ₙN(R^{14a})R^{14b}, —N(R^{10a})R^{10b}, —N═C(R¹³)₂, —C(═O)R¹³, —C(═O)N(R^{14a})R^{14b}, —C(═S)N(R^{14a})R^{14b}, —C(═O)OR¹⁵,
   phenyl, optionally substituted with one or more substituents R¹⁶; and a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more substituents R¹⁶; and
   R⁹ in the groups —S(O)ₙR⁹ and —OSO₂R⁹ is additionally selected from the group consisting of C₁-C₆-alkoxy and C₁-C₆-haloalkoxy;
R^{10a}, R^{10b} independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, where the aliphatic and cycloaliphatic moieties in the eight last-mentioned radicals may be substituted by one or more radicals R¹³; —C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, C₁-C₆-alkoxy, C₁-C₆-haloalkoxy, C₁-C₆-alkylthio, C₁-C₆-haloalkylthio,
   —S(O)ₙR¹⁵, —S(O)ₙN(R^{14a})R^{14b}, —C(═O)R¹³, —C(═O)OR¹⁵, —C(═O)N(R^{14a})R^{14b}, —C(═S)R¹³, —C(═S)SR¹⁵, —C(═S)N(R^{14a})R^{14b}, —C(═NR¹⁴)R¹³;
   phenyl, optionally substituted with 1, 2, 3 or 4, substituents R¹⁶; and a 3-, 4-, 5-, 6- or 7-membered saturated, partially unsaturated or maximally unsaturated heterocyclic ring comprising 1, 2, 3 or 4 heteroatoms or heteroatom groups selected from N, O, S, NO, SO and SO₂, as ring members, where the heterocyclic ring is optionally substituted with one or more substituents R¹⁶;
   or
   R^{10a} and R^{10b} form together with the nitrogen atom they are bonded to a 3-, 4-, 5-, 6-, 7- or 8-membered saturated, partially unsaturated or maximally unsaturated heterocyclic ring, wherein the heterocyclic ring may additionally contain one or two heteroatoms or heteroatom groups selected from N, O, S, NO, SO and SO₂, as ring members, where the heterocyclic ring optionally carries one or more substituents selected from halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl, optionally substituted with 1, 2, 3, 4 or 5 substituents R¹⁶, and a 3-, 4-, 5-, 6-, or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring optionally carries one or more substituents R¹⁶;
   or R^{10a} and R^{10b} together form a group ═C(R¹³)₂, ═S(O)ₘ(R¹⁵)₂, ═S(O)ₘR¹⁵N(R^{14a})R^{14b}, ═NR¹⁴ or ═NOR¹⁵;
R¹¹ is independently selected from the group consisting of halogen, cyano, azido, nitro, —SCN, —SF₅, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted with one or more radicals R⁸,
   —OR⁹, —NR^{10a}R^{10b}, —S(O)ₙR⁹, —Si(R¹²)₃;
   phenyl, optionally substituted with 1, 2, 3, 4, or 5 substituents selected independently from R¹⁶; and
   a 3-, 4-, 5-, 6- or 7-membered saturated, partially unsaturated or maximally unsaturated aromatic heterocyclic ring comprising 1, 2, 3 or 4 heteroatoms or heteroatom groups selected from N, O, S, NO, SO and SO₂, as ring members, where the heterocyclic ring is optionally substituted with one or more substituents selected independently from R¹⁶;
   or two R¹¹ present on the same ring carbon atom of an unsaturated or partially unsaturated heterocyclic ring may together form a group ═O, ═C(R¹³)₂; ═S; ═S(O)ₘ(R¹⁵)₂; ═S(O)ₘR¹⁵N(R^{14a})R^{14b}, ═NR¹⁴, ═NOR¹⁵, or ═NN(R^{14a})R^{14b};
   or two R¹¹ bound on adjacent ring atoms form together with the ring atoms to which they are bound a saturated 3-, 4-, 5-, 6-, 7-, 8- or 9-membered ring, wherein the ring may contain 1 or 2 heteroatoms or heteroatom groups selected from O, S, N, NR¹⁴, NO, SO and SO₂ and/or 1 or 2 groups selected from C═O, C═S and C═NR¹⁴ as ring members, and wherein the ring may be substituted by one or more radicals selected from the group consisting of halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁶, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹⁶;
each R¹² is independently selected from the group consisting of hydrogen, halogen, C₁-C₆-alkyl, C₁-C₆-haloalkyl, C₁-C₆-alkoxy, C₁-C₆-haloalkoxy, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, and phenyl, optionally substituted with 1, 2, 3, 4, or 5 substituents R¹⁶;
each R¹³ is independently selected from the group consisting of cyano, nitro, —OH, —SH, —SCN, —SF₅, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —NR^{14a}R^{14b}, —C(═O)NR^{14a}R^{14b}, trimethylsilyl, triethylsilyl, tert-butyldimethylsilyl,
   C₃-C₈-cycloalkyl which may be unsubstituted, partially or fully halogenated and/or may carry 1 or 2 radicals selected from cyano, C₁-C₄-alkyl, C₃-C₄-cycloalkyl, C₁-C₄-alkoxy, C₁-C₄-haloalkoxy and oxo; phenyl, benzyl, phenoxy, where the phenyl moiety in the three last-mentioned radicals may be unsubstituted or carry 1, 2, 3, 4 or 5 substituents R¹⁶; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by 1, 2 or 3 substituents R¹⁶;
   or
   two R¹³ present on the same carbon atom of an alkyl, alkenyl, alkynyl or cycloalkyl group may together be ═O, ═CH(C₁-C₄-alkyl), ═C(C₁-C₄-alkyl)C₁-C₄-alkyl, ═NR¹⁷ or ═NOR¹⁷;
   and
   R¹³ as a substituent on a cycloalkyl ring is additionally selected from the group consisting of C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 substituents selected from CN, C₃-C₄-cycloalkyl, C₁-C₄-alkoxy, C₁-C₄-haloalkoxy and oxo;
   and
   R¹³ in the groups ═C(R¹³)₂, —N═C(R¹³)₂, —C(═O)R¹³, —C(═S)R¹³ and
   —C(═NR¹⁴)R¹³ is additionally selected from the group consisting of hydrogen, halogen, C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 radicals selected from CN, C₃-C₄-cycloalkyl, C₁-C₄-alkoxy, C₁-C₄-haloalkoxy and oxo;
each R¹⁴ is independently selected from the group consisting of hydrogen, cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —C(═O)NR^{18a}R^{18b}, trimethylsilyl, triethylsilyl, tert-butyldimethylsilyl,
   C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from CN, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl which may be substituted by 1 or 2 substituents selected from halogen and cyano; and oxo;
   C₃-C₈-cycloalkyl which may be unsubstituted, partially or fully halogenated and/or may carry 1 or 2 radicals selected from cyano, C₁-C₄-alkyl, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl, C₃-C₄-cycloalkyl-C₁-C₄-alkyl-, where the cycloalkyl moiety in the two last-mentioned radicals may be substituted by 1 or 2 substituents selected from halogen and cyano; and oxo;
   phenyl, benzyl, pyridyl, phenoxy, wherein the cyclic moieties in the four last-mentioned radicals may be unsubstituted and/or carry 1, 2 or 3 substituents selected from halogen, C₁-C₆-alkyl, C₁-C₆-haloalkyl, C₁-C₆-alkoxy, C₁-C₆-haloalkoxy and (C₁-C₆-alkoxy)carbonyl; and a 3-, 4-, 5- or 6-membered saturated, partially unsaturated or maximally unsaturated heterocyclic ring comprising 1 or 2 heteroatoms or heteroatom groups selected from N, O, S, NO, SO and SO₂, as ring members, where the heterocyclic ring is optionally substituted with one or more substituents R¹⁶;
R^{14a} and R^{14b}, independently of each other, have one of the meanings given for R¹⁴; or R^{14a} and R^{14b}, together with the nitrogen atom to which they are bound, form a 3-, 4-, 5-, 6- or 7-membered saturated, partially unsaturated or maximally unsaturated heterocyclic ring, wherein the heterocyclic ring may additionally contain 1 or 2 heteroatoms or heteroatom groups selected from N, O, S, NO, SO and SO₂, as ring members, where the heterocyclic ring optionally carries one or more substituents selected from halogen, C₁-C₄-alkyl, C₁-C₄-haloalkyl, C₁-C₄-alkoxy and C₁-C₄-haloalkoxy;
   or
   R^{14a} and R¹⁴ or R^{14b} and R¹⁴, together with the nitrogen atoms to which they are bound in the group —C(═NR¹⁴)N(R^{14a})R^{14b}, form a 3-, 4-, 5-, 6- or 7-membered partially unsaturated or maximally unsaturated heterocyclic ring, wherein the heterocyclic ring may additionally contain 1 or 2 heteroatoms or heteroatom groups selected from N, O, S, NO, SO and SO₂, as ring members, where the heterocyclic ring optionally carries one or more substituents selected from halogen, C₁-C₄-haloalkyl, C₁-C₄-alkoxy and C₁-C₄-haloalkoxy;
each R¹⁵ is independently selected from the group consisting of hydrogen, cyano, trimethylsilyl, triethylsilyl, tert-butyldimethylsilyl,
   C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo;
   C₃-C₈-cycloalkyl which may be unsubstituted, partially or fully halogenated and/or may carry 1 or 2 radicals selected from C₁-C₄-alkyl, C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo; phenyl, benzyl, pyridyl and phenoxy, wherein the four last-mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl;
each R¹⁶ is independently selected from the group consisting of halogen, nitro, cyano, —OH, —SH, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl;
   C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from C₃-C₄-cycloalkyl, C₁-C₄-alkoxy, C₁-C₄-haloalkoxy and oxo; C₃-C₈-cycloalkyl which may be unsubstituted, partially or fully halogenated and/or may carry 1 or 2 radicals selected from C₁-C₄-alkyl, C₃-C₄-cycloalkyl, C₁-C₄-alkoxy, C₁-C₄-haloalkoxy and oxo;
   phenyl, benzyl, pyridyl and phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl;
   or
   two R¹⁶ present together on the same atom of an unsaturated or partially unsaturated ring may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
   or
   two R¹⁶ on two adjacent carbon atoms form together with the carbon atoms they are bonded to a 4-, 5-, 6-, 7- or 8-membered saturated, partially unsaturated or maximally unsaturated ring, wherein the ring may contain 1 or 2 heteroatoms or heteroatom groups selected from N, O, S, NO, SO and SO₂, as ring members, and wherein the ring optionally carries one or more substituents selected from halogen, C₁-C₄-haloalkyl, C₁-C₄-alkoxy and C₁-C₄-haloalkoxy;
R¹⁷, R^{18a} and R^{18b}, independently of each other and independently of each occurrence, are selected from the group consisting of hydrogen, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from CN, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl which may be substituted by 1 or 2 substituents selected from halogen and cyano; and oxo;
   C₃-C₈-cycloalkyl which may be unsubstituted, partially or fully halogenated and/or may carry 1 or 2 radicals selected from cyano, C₁-C₄-alkyl and C₁-C₄-haloalkyl; phenyl and benzyl;
each n is independently 0, 1 or 2; and
each m is independently 0 or 1;
and/or the stereoisomers, enantiomers, diastereomers and/or N-oxides, agriculturally or veterinary acceptable salts thereof;
   and wherein the method is for controlling and/or preventing pest infestation of the soybean plants and/or soybean crops.

The compounds of formula (I), and their individual embodiments defined herein below, used in the methods of the present invention may exist in different geometric or optical isomers or tautomeric forms. This invention covers all such isomers and tautomers and mixtures thereof in all proportions as well as isotopic forms such as deuterated compounds. The compounds of the invention may contain one or more asymmetric carbon atoms and may exist as enantiomers (or as pairs of diastereoisomers) or as mixtures of such.

Furthermore, reference to these compounds in the methods of the present invention also includes reference to their agriculturally or veterinary salts and their N-oxides.

The compounds of the invention can be made according to the methods described in the patent application WO2013/092943 cited herein above.

The methods and uses of the invention are for controlling and/or preventing infestation of the soybean plants, soybean crops and soybean propagation material by pests. Preferably the methods and uses of the present invention are applied against pests from the family of pentatomidae, stink bugs. More preferably against stink bugs that are resistant to other insecticides, e.g. pyrethroid insecticides. Stinkbugs that are “resistant” to a particular insecticide refers e.g. to strains of stinkbugs that are less sensitive to that insecticide compared to the expected sensitivity of the same species of stinkbug. The expected sensitivity can be measured using e.g. a strain that has not previously been exposed to the insecticide.

In the present invention the terms pentatomidae and stink bugs are used as synonyms.

In one aspect of the present invention, the method comprises applying to soybean plants, soybean crops and/or propagation material of soybean plants a compound of formula I, wherein the method is for controlling and/or preventing infestation by pests.

Especially the method is for controlling and/or preventing infestation by pests from the family of pentatomidae, stink bugs.

Preferably the method is for controlling and/or preventing infestation by Acrosternum spp., Euschistus spp., Nezara spp., Piezodrus spp., Dichelops spp., Halyomorpha halys, Thyanta accerra, Podisus maculiventris and/or Megacopta cribraria, in particular for controlling and/or preventing infestation by Acrosternum spp., Euschistus spp., Nezara spp and/or Piezodrus spp., more particularly by Acrosternum hilare, Euschistus heros, Nezara viridula and/or Piezodrus guildini and even more particularly by Euschistus heros and/or Nezara viridula. Especially by Euschistus heros. Alternatively or additionally, the method is in particular for controlling and/or preventing infestation by Halyomorpha halys.

In a further aspect the invention provides the use of a compound of formula I for the general control of pests from the family of pentatomidae, stink bugs.

Preferably the use is for the control of Acrosternum spp., Euschistus spp., Nezara spp., Piezodrus spp., Dichelops spp., Halyomorpha halys, Thyanta accerra, Podisus maculiventris and/or Megacopta cribraria, in particular for the control of Acrosternum spp., Euschistus spp., Nezara spp and/or Piezodrus spp., more particularly for the control of Acrosternum hilare, Euschistus heros, Nezara viridula and/or Piezodrus guildini, even more particularly for the control of Euschistus heros and/or Nezara viridula, and especially for the control of Euschistus heros. Alternatively or additionally, the use is in particular for the control of Halyomorpha halys.

In another aspect, the present invention provide the use of the compounds of formula I for controlling pests, that are resistant to one or more other insecticides, preferably pyrethroid, neonicotinoids and organophosphates, and more preferably pyrethroid insecticides.

Preferably the compounds of formula I are used for controlling pests from the family of pentatomidae, stinkbugs, that are resistant to one or more other insecticides, preferably pyrethroid, neonicotinoids and organophosphates, and more preferably pyrethroid insecticides.

Preferably the compounds of formula I are used for the control of Acrosternum spp., Euschistus spp., Nezara spp., Piezodrus spp., Dichelops spp., Halyomorpha halys, Thyanta accerra, Podisus maculiventris and/or Megacopta cribraria, in particular for the control of Acrosternum spp., Euschistus spp., Nezara spp and/or Piezodrus spp., more particularly for the control of Acrosternum hilare, Euschistus heros, Nezara viridula and/or Piezodrus guildini, even more particularly of Euschistus heros and/or Nezara viridula, and especially for the control of Euschistus heros, that are resistant to one or more other insecticides, preferably pyrethroid, neonicotinoids and organophosphates, and more preferably pyrethroid insecticides. Alternatively or additionally, the the compounds of formula I are used for the control of Halyomorpha halys that is resistant to one or more other insecticides, preferably pyrethroid, neonicotinoids and organophosphates, and more preferably pyrethroid insecticides.

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine, in particular fluorine, chlorine or bromine.

The term “alkyl” as used herein and in the alkyl moieties of alkoxy, alkylthio, alkylsulfinyl, alkylsulfonyl, alkylcarbonyl, alkoxycarbonyl and the like refers to saturated straight-chain or branched hydrocarbon radicals having 1 to 2 (“C₁-C₂-alkyl”), 1 to 3 (“C₁-C₃-alkyl”), 1 to 4 (“C₁-C₄-alkyl”), 1 to 6 (“C₁-C₆-alkyl”), 1 to 8 (“C₁-C₈-alkyl”) or 1 to 10 (“C₁-C₁₀-alkyl”) carbon atoms. C₁-C₂-Alkyl is methyl or ethyl. C₁-C₃-Alkyl is additionally propyl and isopropyl. C₁-C₄-Alkyl is additionally butyl, 1-methylpropyl (sec-butyl), 2-methylpropyl (isobutyl) or 1,1-dimethylethyl (tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl. C₁-C₈-Alkyl is additionally also, for example, heptyl, octyl, 2-ethylhexyl and positional isomers thereof. C₁-C₁₀-Alkyl is additionally also, for example, nonyl, decyl and positional isomers thereof.

The term “haloalkyl” as used herein, which is also expressed as “alkyl which is partially or fully halogenated”, refers to straight-chain or branched alkyl groups having 1 to 2 (“C₁-C₂-haloalkyl”), 1 to 3 (“C₁-C₃-haloalkyl”), 1 to 4 (“C₁-C₄-haloalkyl”), 1 to 6 (“C₁-C₆-haloalkyl”), 1 to 8 (“C₁-C₈-haloalkyl”) or 1 to 10 (“C₁-C₁₀-haloalkyl”) carbon atoms (as mentioned above), where some or all of the hydrogen atoms in these groups are replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl or pentafluoroethyl. C₁-C₃-haloalkyl is additionally, for example, 1-fluoropropyl, 2-fluoropropyl, 3-fluoropropyl, 1,1-difluoropropyl, 2,2-difluoropropyl, 1,2-difluoropropyl, 3,3-difluoropropyl, 3,3,3-trifluoropropyl, heptafluoropropyl, 1,1,1-trifluoroprop-2-yl, 3-chloropropyl and the like. Examples for C₁-C₄-haloalkyl are, apart those mentioned for C₁-C₃-haloalkyl, 4-chlorobutyl and the like.

“Halomethyl” is methyl in which 1, 2 or 3 of the hydrogen atoms are replaced by halogen atoms. Examples are bromomethyl, chloromethyl, fluoromethyl, dichloromethyl, trichloromethyl, difluoromethyl, trifluoromethyl, chlorofluoromethyl, dichlorofluoromethyl, chlorodifluoromethyl and the like.

The term “alkenyl” as used herein refers to monounsaturated straight-chain or branched hydrocarbon radicals having 2 to 3 (“C₂-C₃-alkenyl”), 2 to 4 (“C₂-C₄-alkenyl”), 2 to 6 (“C₂-C₆-alkenyl”), 2 to 8 (“C₂-C₈-alkenyl”) or 2 to 10 (“C₂-C₁₀-alkenyl”) carbon atoms and a double bond in any position, for example C₂-C₃-alkenyl, such as ethenyl, 1-propenyl, 2-propenyl or 1-methylethenyl; C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like, or C₂-C₁₀-alkenyl, such as the radicals mentioned for C₂-C₆-alkenyl and additionally 1-heptenyl, 2-heptenyl, 3-heptenyl, 1-octenyl, 2-octenyl, 3-octenyl, 4-octenyl, 1-nonenyl, 2-nonenyl, 3-nonenyl, 4-nonenyl, 1-decenyl, 2-decenyl, 3-decenyl, 4-decenyl, 5-decenyl and the positional isomers thereof.

The term “haloalkenyl” as used herein, which is also expressed as “alkenyl which is partially or fully halogenated”, refers to unsaturated straight-chain or branched hydrocarbon radicals having 2 to 4 (“C₂-C₄-haloalkenyl”), 2 to 6 (“C₂-C₆-haloalkenyl”), 2 to 8 (“C₂-C₆-haloalkenyl”) or 2 to 10 (“C₂-C₁₀-haloalkenyl”) carbon atoms and a double bond in any position (as mentioned above), where some or all of the hydrogen atoms in these groups are replaced by halogen atoms as mentioned above, in particular fluorine, chlorine and bromine, for example chlorovinyl, chloroallyl and the like.

The term “alkynyl” as used herein refers to straight-chain or branched hydrocarbon groups having 2 to 3 (“C₂-C₃-alkynyl”), 2 to 4 (“C₂-C₄-alkynyl”), 2 to 6 (“C₂-C₆-alkynyl”), 2 to 8 (“C₂-C₈- alkynyl”), or 2 to 10 (“C₂-C₁₀-alkynyl”) carbon atoms and one or two triple bonds in any position, for example C₂-C₃-alkynyl, such as ethynyl, 1-propynyl or 2-propynyl; C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;

The term “haloalkynyl” as used herein, which is also expressed as “alkynyl which is partially or fully halogenated”, refers to unsaturated straight-chain or branched hydrocarbon radicals having 2 to 4 (“C₂-C₄-haloalkynyl”), 3 to 4 (“C₃-C₄-haloalkynyl”), 2 to 6 (“C₂-C₆-haloalkynyl”), 2 to 8 (“C₂- C₆-haloalkynyl”) or 2 to 10 (“C₂-C₁₀-haloalkynyl”) carbon atoms and one or two triple bonds in any position (as mentioned above), where some or all of the hydrogen atoms in these groups are replaced by halogen atoms as mentioned above, in particular fluorine, chlorine and bromine; The term “cycloalkyl” as used herein refers to mono- or bi- or polycyclic saturated hydrocarbon radicals having 3 to 8 (“C₃-C₈-cycloalkyl”), in particular 3 to 6 carbon atoms (“C₃-C₆-cycloalkyl”). Examples of monocyclic radicals having 3 to 6 carbon atoms comprise cyclopropyl, cyclobutyl, cyclopentyl and cyclohexyl. Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.2.1]heptyl, bicyclo[3.1.1]heptyl, bicyclo[2.2.2]octyl and bicyclo[3.2.1]octyl. Preferably, the term cycloalkyl denotes a monocyclic saturated hydrocarbon radical.

The term “halocycloalkyl” as used herein, which is also expressed as “cycloalkyl which is partially or fully halogenated”, refers to mono- or bi- or polycyclic saturated hydrocarbon groups having 3 to 8 (“C₃-C₈-halocycloalkyl”) or preferably 3 to 6 (“C₃-C₆-halocycloalkyl”) carbon ring members (as mentioned above) in which some or all of the hydrogen atoms are replaced by halogen atoms as mentioned above, in particular fluorine, chlorine and bromine.

The term “cycloalkenyl” as used herein refers to monocyclic hydrocarbon radicals with at least one C—C double bond in the ring, which ring is however not aromatic, the hydrocarbon radicals having 3 to 8 (“C₃-C₈-cycloalkyl) carbon atoms. Examples are cyclopropenyl, such as cycloprop-1-enyl and cycloprop-2-yl, cyclobutenyl, such as cyclobut-1-enyl and cyclobut-2-enyl, cyclopentenyl, such as cyclopent-1-enyl, cyclopent-2-enyl and cyclopent-3-enyl, cyclopentadienyl, such as cyclopenta-1,3-dienyl, cyclpenta-1,4-dienyl and cyclpenta-2,4-dienyl, cyclohexenyl, such as cyclohex-1-enyl, cyclohex-2-enyl and cyclohex-3-enyl, cyclohexadienyl, such as cyclohexa-1,3-dienyl, cyclohexa-1,4-dienyl, cyclohexa-1,5-dienyl and cyclohexa-2,5-dienyl, cycloheptenyl, cycloheptadienyl, cycloheptatrienyl cyclooctenyl, cyclooctadieny, cyclooctatrienyl and cyclooctatetraenyl.

The term “halocycloalkenyl” as used herein refers to monocyclic hydrocarbon radicals with at least one C—C double bond in the ring, which ring is however not aromatic, the hydrocarbon radicals having 3 to 8 (“C₃-C₈-halocycloalkyl”) carbon atoms, and wherein some or all of the hydrogen atoms are replaced by halogen atoms as mentioned above, in particular fluorine, chlorine and bromine.

The term “cycloalkyl-C₁-C₄-alkyl” refers to a C₃-C₈-cycloalkyl group (“C₃-C₈-cycloalkyl-C₁-C₄-alkyl”), preferably a C₃-C₆-cycloalkyl group (“C₃-C₆-cycloalkyl-C₁-C₄-alkyl”), more preferably a C₃-C₄-cycloalkyl group (“C₃-C₄-cycloalkyl-C₁-C₄-alkyl”) as defined above (preferably a monocyclic cycloalkyl group) which is bound to the remainder of the molecule via a C₁-C₄-alkyl group, as defined above. Examples for C₃-C₄-cycloalkyl-C₁-C₄-alkyl are cyclopropylmethyl, cyclopropylethyl, cyclopropylpropyl, cyclobutylmethyl, cyclobutylethyl and cyclobutylpropyl, Examples for C₃-C₆-cycloalkyl-C₁-C₄-alkyl, apart those mentioned for C₃-C₄-cycloalkyl-C₁-C₄-alkyl, are cyclopentylmethyl, cyclopentylethyl, cyclopentylpropyl, cyclohexylmethyl, cyclohexylethyl and cyclohexylpropyl. Examples for C₃-C₈-cycloalkyl-C₁-C₄-alkyl, apart those mentioned for C₃-C₆-cycloalkyl-C₁-C₄-alkyl, are cycloheptylmethyl, cycloheptylethyl, cyclooctylmethyl and the like. The term “C₃-C₈-halocycloalkyl-C₁-C₄-alkyl” refers to a C₃-C₈-halocycloalkyl group as defined above which is bound to the remainder of the molecule via a C₁-C₄-alkyl group, as defined above.

The term “C₁-C₂-alkoxy” is a C₁-C₂-alkyl group, as defined above, attached via an oxygen atom. The term “C₁-C₃-alkoxy” is a C₁-C₃-alkyl group, as defined above, attached via an oxygen atom. The term “C₁-C₄-alkoxy” is a C₁-C₄-alkyl group, as defined above, attached via an oxygen atom. The term “C₁-C₆-alkoxy” is a C₁-C₆-alkyl group, as defined above, attached via an oxygen atom. The term “C₁-C₁₀-alkoxy” is a C₁-C₁₀-alkyl group, as defined above, attached via an oxygen atom. C₁-C₂-Alkoxy is methoxy or ethoxy. C₁-C₃-Alkoxy is additionally, for example, n-propoxy and 1-methylethoxy (isopropoxy). C₁-C₄-Alkoxy is additionally, for example, butoxy, 1-methylpropoxy (sec-butoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof.

The term “C₁-C₂-haloalkoxy” is a C₁-C₂-haloalkyl group, as defined above, attached via an oxygen atom. The term “C₁-C₃-haloalkoxy” is a C₁-C₃-haloalkyl group, as defined above, attached via an oxygen atom. The term “C₁-C₄-haloalkoxy” is a C₁-C₄-haloalkyl group, as defined above, attached via an oxygen atom. The term “C₁-C₆-haloalkoxy” is a C₁-C₆-haloalkyl group, as defined above, attached via an oxygen atom. The term “C₁-C₁₀-haloalkoxy” is a C₁-C₁₀-haloalkyl group, as defined above, attached via an oxygen atom. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₃-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy or 1-(CH₂Br)-2-bromoethoxy. C₁-C₄-Haloalkoxy is additionally, for example, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy.

The term “C₁-C₄-alkoxy-C₁-C₄-alkyl” as used herein, refers to a straight-chain or branched alkyl group having 1 to 4 carbon atoms, as defined above, where one hydrogen atom is replaced by a C₁-C₄-alkoxy group, as defined above. The term “C₁-C₆-alkoxy-C₁-C₆-alkyl” as used herein, refers to a straight-chain or branched alkyl group having 1 to 6 carbon atoms, as defined above, where one hydrogen atom is replaced by a C₁-C₆-alkoxy group, as defined above. Examples are methoxymethyl, ethoxymethyl, propoxymethyl, isopropoxymethyl, n-butoxymethyl, sec-butoxymethyl, isobutoxymethyl, tert-butoxymethyl, 1-methoxyethyl, 1-ethoxyethyl, 1-propoxyethyl, 1-isopropoxyethyl, 1-n-butoxyethyl, 1-sec-butoxyethyl, 1-isobutoxyethyl, 1-tert-butoxyethyl, 2-methoxyethyl, 2-ethoxyethyl, 2-propoxyethyl, 2-isopropoxyethyl, 2-n-butoxyethyl, 2-sec-butoxyethyl, 2-isobutoxyethyl, 2-tert-butoxyethyl, 1-methoxypropyl, 1-ethoxypropyl, 1-propoxypropyl, 1-isopropoxypropyl, 1-n-butoxypropyl, 1-sec-butoxypropyl, 1-isobutoxypropyl, 1-tert-butoxypropyl, 2-methoxypropyl, 2-ethoxypropyl, 2-propoxypropyl, 2-isopropoxypropyl, 2-n-butoxypropyl, 2-sec-butoxypropyl, 2-isobutoxypropyl, 2-tert-butoxypropyl, 3-methoxypropyl, 3-ethoxypropyl, 3-propoxypropyl, 3-isopropoxypropyl, 3-n-butoxypropyl, 3-sec-butoxypropyl, 3-isobutoxypropyl, 3-tert-butoxypropyl and the like.

The term “C₁-C₆-alkoxy-methyl” as used herein, refers to methyl in which one hydrogen atom is replaced by a C₁-C₆-alkoxy group, as defined above. Examples are methoxymethyl, ethoxymethyl, propoxymethyl, isopropoxymethyl, n-butoxymethyl, sec-butoxymethyl, isobutoxymethyl, tert-butoxymethyl, pentyloxymethyl, hexyloxymethyl and the like.

C₁-C₆-Haloalkoxy-C₁-C₆-alkyl is a straight-chain or branched alkyl group having from 1 to 6, especially 1 to 4 carbon atoms (═C₁-C₆-haloalkoxy-C₁-C₄-alkyl), wherein one of the hydrogen atoms is replaced by a C₁-C₆-alkoxy group and wherein at least one, e.g. 1, 2, 3, 4 or all of the remaining hydrogen atoms (either in the alkoxy moiety or in the alkyl moiety or in both) are replaced by halogen atoms. C₁-C₄-Haloalkoxy-C₁-C₄-alkyl is a straight-chain or branched alkyl group having from 1 to 4 carbon atoms, wherein one of the hydrogen atoms is replaced by a C₁-C₄-alkoxy group and wherein at least one, e.g. 1, 2, 3, 4 or all of the remaining hydrogen atoms (either in the alkoxy moiety or in the alkyl moiety or in both) are replaced by halogen atoms. Examples are difluoromethoxymethyl (CHF₂OCH₂), trifluoromethoxymethyl, 1-difluoromethoxyethyl, 1-trifluoromethoxyethyl, 2-difluoromethoxyethyl, 2-trifluoromethoxyethyl, difluoro-methoxy-methyl (CH₃OCF₂), 1,1-difluoro-2-methoxyethyl, 2,2-difluoro-2-methoxyethyl and the like.

The term “C₁-C₂-alkylthio” is a C₁-C₂-alkyl group, as defined above, attached via a sulfur atom. The term “C₁-C₃-alkylthio” is a C₁-C₃-alkyl group, as defined above, attached via a sulfur atom. The term “C₁-C₄-alkylthio” is a C₁-C₄-alkyl group, as defined above, attached via a sulfur atom. The term “C₁-C₆-alkylthio” is a C₁-C₆-alkyl group, as defined above, attached via a sulfur atom. The term “C₁-C₁₀-alkylthio” is a C₁-C₁₀-alkyl group, as defined above, attached via a sulfur atom. C₁-C₂-Alkylthio is methylthio or ethylthio. C₁-C₃-Alkylthio is additionally, for example, n-propylthio or 1-methylethylthio (isopropylthio). C₁-C₄-Alkylthio is additionally, for example, butylthio, 1-methylpropylthio (sec-butylthio), 2-methylpropylthio (isobutylthio) or 1,1-dimethylethylthio (tertbutylthio). C₁-C₆-Alkylthio is additionally, for example, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio. C₁-C₈-Alkylthio is additionally, for example, heptylthio, octylthio, 2-ethylhexylthio and positional isomers thereof. C₁-C₁₀-Alkylthio is additionally, for example, nonylthio, decylthio and positional isomers thereof.

The term “C₁-C₂-haloalkylthio” is a C₁-C₂-haloalkyl group, as defined above, attached via a sulfur atom. The term “C₁-C₃-haloalkylthio” is a C₁-C₃-haloalkyl group, as defined above, attached via a sulfur atom. The term “C₁-C₄-haloalkylthio” is a C₁-C₄-haloalkyl group, as defined above, attached via a sulfur atom. The term “C₁-C₆-haloalkylthio” is a C₁-C₆-haloalkyl group, as defined above, attached via a sulfur atom. The term “C₁-C₁₀-haloalkylthio” is a C₁-C₁₀-haloalkyl group, as defined above, attached via a sulfur atom. C₁-C₂-Haloalkylthio is, for example, SCH₂F, SCHF₂, SCF₃, SCH₂Cl, SCHCl₂, SCCl₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio or SC₂F₅. C₁-C₃-Haloalkylthio is additionally, for example, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂—C₂F₅, 1-(CH₂F)-2-fluoroethylthio, 1-(CH₂Cl)-2-chloroethylthio or 1-(CH₂Br)-2-bromoethylthio. C₁-C₄-Haloalkylthio is additionally, for example, 4-fluorobutylthio, 4-chlorobutylthio, 4-bromobutylthio or nonafluorobutylthio. C₁-C₆-Haloalkylthio is additionally, for example, 5-fluoropentylthio, 5-chloropentylthio, 5-brompentylthio, 5-iodopentylthio, undecafluoropentylthio, 6-fluorohexylthio, 6-chlorohexylthio, 6-bromohexylthio, 6-iodohexylthio or dodecafluorohexylthio.

The term “C₁-C₂-alkylsulfinyl” is a C₁-C₂-alkyl group, as defined above, attached via a sulfinyl [S(O)] group. The term “C₁-C₄-alkylsulfinyl” is a C₁-C₄-alkyl group, as defined above, attached via a sulfinyl [S(O)] group. The term “C₁-C₆-alkylsulfinyl” is a C₁-C₆-alkyl group, as defined above, attached via a sulfinyl [S(O)] group. The term “C₁-C₁₀-alkylsulfinyl” is a C₁-C₁₀-alkyl group, as defined above, attached via a sulfinyl [S(O)] group. C₁-C₂-Alkylsulfinyl is methylsulfinyl or ethylsulfinyl. C₁-C₄-Alkylsulfinyl is additionally, for example, n-propylsulfinyl, 1-methylethylsulfinyl (isopropylsulfinyl), butylsulfinyl, 1-methylpropylsulfinyl (sec-butylsulfinyl), 2-methylpropylsulfinyl (isobutylsulfinyl) or 1,1-dimethylethylsulfinyl (tert-butylsulfinyl). C₁-C₆-Alkylsulfinyl is additionally, for example, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsuIfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl. C₁-C₈-Alkylsulfinyl is additionally, for example, heptylsulfinyl, octylsulfinyl, 2-ethylhexylsulfinyl and positional isomers thereof. C₁-C₁₀-Alkylsulfinyl is additionally, for example, nonylsulfinyl, decylsulfinyl and positional isomers thereof.

The term “C₁-C₂-haloalkylsulfinyl” is a C₁-C₂-haloalkyl group, as defined above, attached via a sulfinyl [S(O)] group. The term “C₁-C₄-haloalkylsulfinyl” is a C₁-C₄-haloalkyl group, as defined above, attached via a sulfinyl [S(O)] group. The term “C₁-C₆-haloalkylsulfinyl” is a C₁-C₆-haloalkyl group, as defined above, attached via a sulfinyl [S(O)] group. The term “C₁-C₁₀-haloalkylsulfinyl” is a C₁-C₁₀-haloalkyl group, as defined above, attached via a sulfinyl [S(O)] group. C₁-C₂-Haloalkylsulfinyl is, for example, S(O)CH₂F, S(O)CHF₂, S(O)CF₃, S(O)CH₂Cl, S(O)CHCl₂, S(O)CCl₃, chlorofluoromethylsulfinyl, dichlorofluoromethylsulfinyl, chlorodifluoromethylsulfinyl, 2-fluoroethylsulfinyl, 2-chloroethylsulfinyl, 2-bromoethylsulfinyl, 2-iodoethylsulfinyl, 2,2-difluoroethylsulfinyl, 2,2,2-trifluoroethylsulfinyl, 2-chloro-2-fluoroethylsulfinyl, 2-chloro-2,2-difluoroethylsulfinyl, 2,2-dichloro-2-fluoroethylsulfinyl, 2,2,2-trichloroethylsulfinyl or S(O)C₂F₅. C₁-C₄-Haloalkylsulfinyl is additionally, for example, 2-fluoropropylsulfinyl, 3-fluoropropylsulfinyl, 2,2-difluoropropylsulfinyl, 2,3-difluoropropylsulfinyl, 2-chloropropylsulfinyl, 3-chloropropylsulfinyl, 2,3-dichloropropylsulfinyl, 2-bromopropylsulfinyl, 3-bromopropylsulfinyl, 3,3,3-trifluoropropylsulfinyl, 3,3,3-trichloropropylsulfinyl, S(O)CH₂—C₂F₅, S(O)CF₂—C₂F₅, 1-(CH₂F)-2-fluoroethylsulfinyl, 1-(CH₂Cl)-2-chloroethylsulfinyl, 1-(CH₂Br)-2-bromoethylsulfinyl, 4-fluorobutylsulfinyl, 4-chlorobutylsulfinyl, 4-bromobutylsulfinyl or nonafluorobutylsulfinyl. C₁-C₆-Haloalkylsulfinyl is additionally, for example, 5-fluoropentylsulfinyl, 5-chloropentylsulfinyl, 5-brompentylsulfinyl, 5-iodopentylsulfinyl, undecafluoropentylsulfinyl, 6-fluorohexylsulfinyl, 6-chlorohexylsulfinyl, 6-bromohexylsulfinyl, 6-iodohexylsulfinyl or dodecafluorohexylsulfinyl.

The term “C₁-C₂-alkylsulfonyl” is a C₁-C₂-alkyl group, as defined above, attached via a sulfonyl [S(O)₂] group. The term “C₁-C₃-alkylsulfonyl” is a C₁-C₃-alkyl group, as defined above, attached via a sulfonyl [S(O)₂] group. The term “C₁-C₄-alkylsulfonyl” is a C₁-C₄-alkyl group, as defined above, attached via a sulfonyl [S(O)₂] group. The term “C₁-C₆-alkylsulfonyl” is a C₁-C₆-alkyl group, as defined above, attached via a sulfonyl [S(O)₂] group. The term “C₁-C₁₀-alkylsulfonyl” is a C₁-C₁₀-alkyl group, as defined above, attached via a sulfonyl [S(O)₂] group. C₁-C₂-Alkylsulfonyl is methylsulfonyl or ethylsulfonyl. C₁-C₃-Alkylsulfonyl is additionally, for example, n-propylsulfonyl or 1-methylethylsulfonyl (isopropylsulfonyl). C₁-C₄-Alkylsulfonyl is additionally, for example, butylsulfonyl, 1-methylpropylsulfonyl (sec-butylsulfonyl), 2-methylpropylsulfonyl (isobutylsulfonyl) or 1,1-dimethylethylsulfonyl (tert-butylsulfonyl). C₁-C₆-Alkylsulfonyl is additionally, for example, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl. C₁-C₈-Alkylsulfonyl is additionally, for example, heptylsulfonyl, octylsulfonyl, 2-ethylhexylsulfonyl and positional isomers thereof. C₁-C₁₀-Alkylsulfonyl is additionally, for example, nonylsulfonyl, decylsulfonyl and positional isomers thereof.

The term “C₁-C₂-haloalkylsulfonyl” is a C₁-C₂-haloalkyl group, as defined above, attached via a sulfonyl [S(O)₂] group. The term “C₁-C₃-haloalkylsulfonyl” is a C₁-C₃-haloalkyl group, as defined above, attached via a sulfonyl [S(O)₂] group. The term “C₁-C₄-haloalkylsulfonyl” is a C₁-C₄-haloalkyl group, as defined above, attached via a sulfonyl [S(O)₂] group. The term “C₁-C₆-haloalkylsulfonyl” is a C₁-C₆-haloalkyl group, as defined above, attached via a sulfonyl [S(O)₂] group. The term “C₁-C₁₀-haloalkylsulfonyl” is a C₁-C₁₀-haloalkyl group, as defined above, attached via a sulfonyl [S(O)₂] group. C₁-C₂-Haloalkylsulfonyl is, for example, S(O)₂CH₂F, S(O)₂CHF₂, S(O)₂CF₃, S(O)₂CH₂Cl, S(O)₂CHCl₂, S(O)₂CCl₃, chlorofluoromethylsulfonyl, dichlorofluoromethylsulfonyl, chlorodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl or S(O)₂C₂F₅. C₁-C₃-Haloalkylsulfonyl is additionally, for example, 2-fluoropropylsulfonyl, 3-fluoropropylsulfonyl, 2,2-difluoropropylsulfonyl, 2,3-difluoropropylsulfonyl, 2-chloropropylsulfonyl, 3-chloropropylsulfonyl, 2,3-dichloropropylsulfonyl, 2-bromopropylsulfonyl, 3-bromopropylsulfonyl, 3,3,3-trifluoropropylsulfonyl, 3,3,3-trichloropropylsulfonyl, S(O)₂CH₂—C₂F₅, S(O)₂CF₂—C₂F₅, 1-(CH₂F)-2-fluoroethylsulfonyl, 1-(CH₂Cl)-2-chloroethylsulfonylor 1-(CH₂Br)-2-bromoethylsulfonyl. C₁-C₄-Haloalkylsulfonyl is additionally, for example, 4-fluorobutylsulfonyl, 4-chlorobutylsulfonyl, 4-bromobutylsulfonyl or nonafluorobutylsulfonyl. C₁-C₆-Haloalkylsulfonyl is additionally, for example, 5-fluoropentylsulfonyl, 5-chloropentylsulfonyl, 5-brompentylsulfonyl, 5-iodopentylsulfonyl, undecafluoropentylsulfonyl, 6-fluorohexylsulfonyl, 6-chlorohexylsulfonyl, 6-bromohexylsulfonyl, 6-iodohexylsulfonyl or dodecafluorohexylsulfonyl.

The substituent “oxo” replaces a CH₂ group by a C(═O) group.

The term “alkylcarbonyl” is a C₁-C₆-alkyl (“C₁-C₆-alkylcarbonyl”), preferably a C₁-C₄-alkyl (“C₁-C₄-alkylcarbonyl”) group, as defined above, attached via a carbonyl [C(═O)] group. Examples are acetyl (methylcarbonyl), propionyl (ethylcarbonyl), propylcarbonyl, isopropylcarbonyl, nbutylcarbonyl and the like.

The term “haloalkylcarbonyl” is a C₁-C₆-haloalkyl (“C₁-C₆-haloalkylcarbonyl”), preferably a C₁-C₄-haloalkyl (“C₁-C₄-haloalkylcarbonyl”) group, as defined above, attached via a carbonyl [C(═O)] group. Examples are trifluoromethylcarbonyl, 2,2,2-trifluoroethylcarbonyl and the like.

The term “alkoxycarbonyl” is a C₁-C₆-alkoxy (“C₁-C₆-alkoxycarbonyl”), preferably a C₁-C₄-alkoxy (“C₁-C₄-alkoxycarbonyl”) group, as defined above, attached via a carbonyl [C(═O)] group. Examples are methoxycarbonyl), ethoxycarbonyl, propoxycarbonyl, isopropoxycarbonyl, n-butoxycarbonyl and the like.

The term “haloalkoxycarbonyl” is a C₁-C₆-haloalkoxy (“C₁-C₆-haloalkoxycarbonyl”), preferably a C₁-C₄-haloalkoxy (“C₁-C₄-haloalkoxycarbonyl”) group, as defined above, attached via a carbonyl [C(═O)] group. Examples are trifluoromethoxycarbonyl, 2,2,2-trifluoroethoxycarbonyl and the like.

The term “C₁-C₆-alkylamino” is a group —N(H)C₁-C₆-alkyl. Examples are methylamino, ethylamino, propylamino, isopropylamino, butylamino and the like.

The term “di-(C₁-C₆-alkyl)amino” is a group —N(C₁-C₆-alkyl)₂. Examples are dimethylamino, diethylamino, ethylmethylamino, dipropylamino, diisopropylamino, methylpropylamino, methylisopropylamino, ethylpropylamino, ethylisopropylamino, dibutylamino and the like.

The term “3-, 4-, 5-, 6-, 7-, 8-, 9- or 10-membered saturated, partially unsaturated or maximally unsaturated heterocyclic ring containing 1, 2 or 3 (or 4) heteroatoms or heteroatom groups selected from N, O, S, NO, SO and SO₂, as ring members” denotes a 3-, 4-, 5-, 6- or 7-membered saturated, partially unsaturated or maximum unsaturated heteromonocyclic ring or a 8-, 9- or 10-membered saturated, partially unsaturated or maximally unsaturated heterobicyclic ring containing 1, 2 or 3 (or 4) heteroatoms or heteroatom groups selected from N, O, S, NO, SO and SO₂, as ring members.

Unsaturated rings contain at least one C—C and/or C—N and/or N—N double bond(s). Maximally unsaturated rings contain as many conjugated C—C and/or C—N and/or N—N double bonds as allowed by the ring size. Maximally unsaturated 5- or 6-membered heterocyclic rings are aromatic. The heterocyclic ring may be attached to the remainder of the molecule via a carbon ring member or via a nitrogen ring member. As a matter of course, the heterocyclic ring contains at least one carbon ring atom. If the ring contains more than one O ring atom, these are not adjacent.

The term “3-, 4-, 5-, 6- or 7-membered saturated, partially unsaturated or maximum unsaturated heterocyclic ring containing 1, 2 or 3 (or 4) heteroatoms or heteroatom groups selected from N, O, S, NO, SO and SO₂, as ring members” [wherein “maximum unsaturated” includes also “aromatic”] as used herein denotes monocyclic radicals, the monocyclic radicals being saturated, partially unsaturated or maximum unsaturated (including aromatic). The term “3-, 4-, 5-, 6-, 7- or 8-membered saturated, partially unsaturated or maximum unsaturated heterocyclic ring containing 1, 2 or 3 (or 4) heteroatoms or heteroatom groups selected from N, O, S, NO, SO and SO₂, as ring members” [wherein “maximum unsaturated” includes also “aromatic”] as used herein further also encompasses 8-membered heteromonocyclic radicals containing 1, 2 or 3 (or 4) heteroatoms or heteroatom groups selected from N, O, S, NO, SO and SO₂, as ring members, the monocyclic radicals being saturated, partially unsaturated or maximum unsaturated (including aromatic). Unsaturated rings contain at least one C—C and/or C—N and/or N—N double bond(s). Maximum unsaturated rings contain as many conjugated C—C and/or C—N and/or N—N double bonds as allowed by the ring size. Maximum unsaturated 5- or 6-membered heterocyclic rings are aromatic. 7- and 8-membered rings cannot be aromatic. They are homoaromatic (7-membered ring, 3 double bonds) or have 4 double bonds (8-membered ring). The heterocyclic ring may be attached to the remainder of the molecule via a carbon ring member or via a nitrogen ring member. As a matter of course, the heterocyclic ring contains at least one carbon ring atom. If the ring contains more than one O ring atom, these are not adjacent.

Examples of a 3-, 4-, 5-, 6- or 7-membered saturated heterocyclic ring include: Oxiranyl, thiiranyl, aziridinyl, oxetanyl, thietanyl, azetidinyl, tetrahydrofuran-2-yl, tetrahydrofuran-3-yl, tetrahydrothien-2-yl, tetrahydrothien-3-yl, pyrrolidin-1-yl, pyrrolidin-2-yl, pyrrolidin-3-yl, pyrazolidin-1-yl, pyrazolidin-3-yl, pyrazolidin-4-yl, pyrazolidin-5-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, isothiazolidin-2-yl, isothiazolidin-3-yl, isothiazolidin-4-yl, isothiazolidin-5-yl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-1-yl, 1,3,4-triazolidin-2-yl, 2-tetrahydropyranyl, 4-tetrahydropyranyl, 1,3-dioxan-5-yl, 1,4-dioxan-2-yl, piperidin-1-yl, piperidin-2-yl, piperidin-3-yl, piperidin-4-yl, hexahydropyridazin-3-yl, hexahydropyridazin-4-yl, hexahydropyrimidin-2-yl, hexahydropyrimidin-4-yl, hexahydropyrimidin-5-yl, piperazin-1-yl, piperazin-2-yl, 1,3,5-hexahydrotriazin-1-yl, 1,3,5-hexahydrotriazin-2-yl and 1,2,4-hexahydrotriazin-3-yl, morpholin-2-yl, morpholin-3-yl, morpholin-4-yl, thiomorpholin-2-yl, thiomorpholin-3-yl, thiomorpholin-4-yl, 1-oxothiomorpholin-2-yl, 1-oxothiomorpholin-3-yl, 1-oxothiomorpholin-4-yl, 1,1-dioxothiomorpholin-2-yl, 1,1-dioxothiomorpholin-3-yl, 1,1-dioxothiomorpholin-4-yl, azepan-1-, -2-, -3- or -4-yl, oxepan-2-, -3-, -4- or -5-yl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like.

Examples of a 3-, 4-, 5-, 6- or 7-membered partially unsaturated heterocyclic ring include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl.

Examples for a 3-, 4-, 5-, 6- or 7-membered maximally unsaturated (including aromatic) heterocyclic ring are 5- or 6-membered heteroaromatic rings, such as 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl, 1,3,4-triazol-1-yl, 1,3,4-triazol-2-yl, 2-pyridinyl, 3-pyridinyl, 4-pyridinyl, 1-oxopyridin-2-yl, 1-oxopyridin-3-yl, 1-oxopyridin-4-yl,3-pyridazinyl, 4-pyridazinyl, 2-pyrimidinyl, 4-pyrimidinyl, 5-pyrimidinyl and 2-pyrazinyl, and also homoaromatic radicals, such as 1H-azepine, 1H-[1,3]-diazepine and 1H-[1,4]-diazepine.

Examples for a 8-, 9- or 10-membered saturated heterobicyclic ring containing 1, 2 or 3 (or 4) heteroatoms or heteroatom groups selected from N, O, S, NO, SO and SO₂, as ring members are:

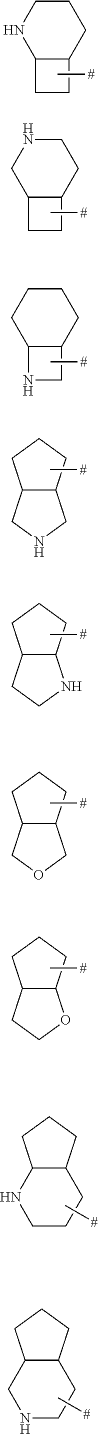

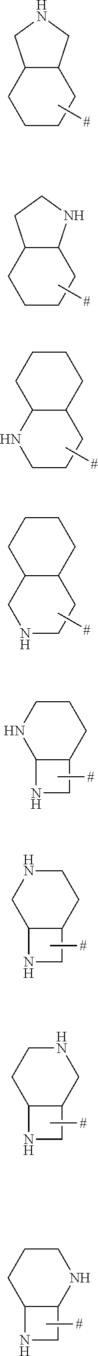

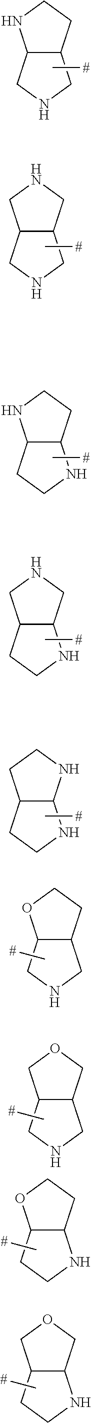

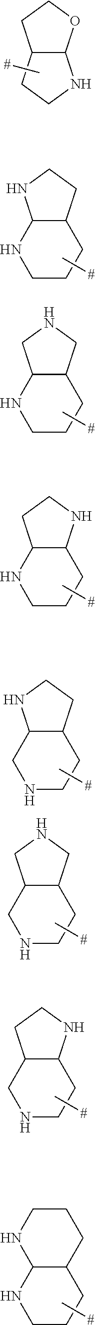

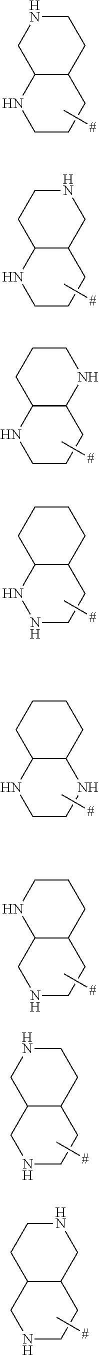

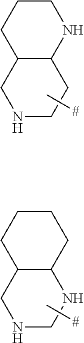

Examples for a 8-, 9- or 10-membered partially unsaturated heterobicyclic ring containing 1, 2 or 3 (or 4) heteroatoms or heteroatom groups selected from N, O, S, NO, SO and SO₂, as ring members are:

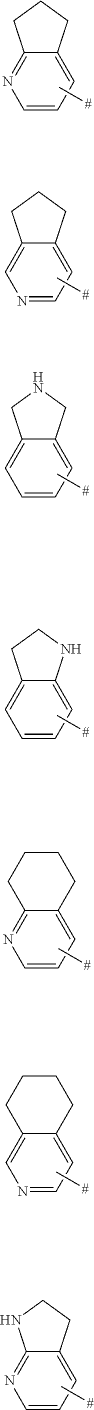

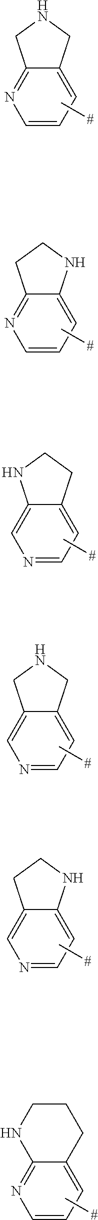

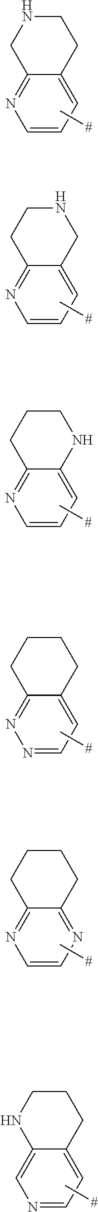

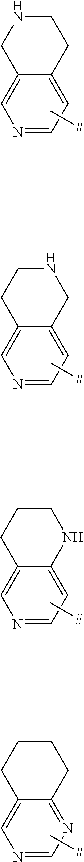

Examples for a 8-, 9- or 10-membered maximally unsaturated heterobicyclic ring containing 1, 2 or 3 (or 4) heteroatoms or heteroatom groups selected from N, O, S, NO, SO and SO₂, as ring members are:

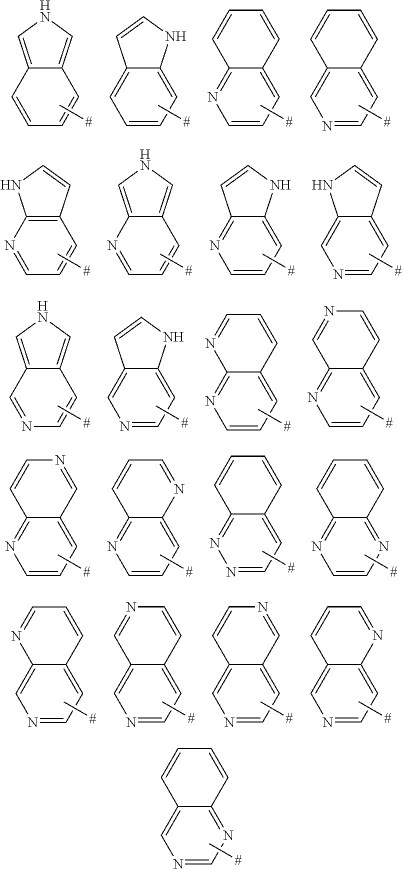

In the above structures # denotes the attachment point to the remainder of the molecule. The attachment point is not restricted to the ring on which is shown, but can be on either of the fused rings, and may be on a carbon or on a nitrogen ring atom. If the rings carry one or more substituents, these may be bound to carbon and/or to nitrogen ring atoms (if the latter are not part of a double bond).

A saturated 3-, 4-, 5-, 6-, 7-, 8- or 9-membered ring, wherein the ring may contain 1 or 2 heteroatoms or heteroatom groups selected from O, S, N, NR¹⁴, NO, SO and SO₂ and/or 1 or 2 groups selected from C═O, C═S and C═NR¹⁴ as ring members is either carbocyclic or heterocyclic. Examples are, in addition to the saturated heteromonocyclic rings mentioned above, carbocyclic rings, such as cyclopropyl, cyclopropanonyl, cyclobutyl, cyclobutanonyl, cyclopentyl, cyclopentanonyl, cyclohexyl, cyclohexanonyl, cyclohexadienonyl, cycloheptyl, cycloheptanonyl, cyclooctyl, cyclooctanonyl, furan-2-onyl, pyrrolidine-2-onyl, pyrrolidine-2,5-dionyl, piperidine-2-only, piperidine-2,6-dionyl and the like.

### EMBODIMENTS OF THE INVENTION

In one embodiment of the invention A in compounds of formula I is A¹.

In another embodiment of the invention A in compounds of formula I is A².

In another embodiment of the invention A in compounds of formula I is A³.

In one embodiment of the invention a method is provided, which method comprises applying to soybean plants, to a crop of soybean plants, to the locus thereof or to propagation material thereof, a compound of formula I.1:

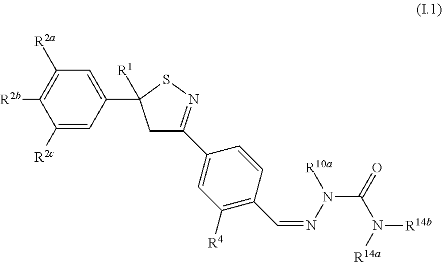

wherein
R¹ is C₁-C₄-haloalkyl and is in particular CF₃;
R^{2a}, R^{2b} and R^{2c} are selected independently from one another from hydrogen, halogen and C₁-C₂-haloalkyl, preferably from hydrogen, F, Cl, Br and CF₃;
R⁴ is selected from hydrogen, halogen, cyano, C₁-C₄-alkyl, C₁-C₄-haloalkyl, C₁-C₄-alkoxy, C₁-C₄-haloalkoxy, C₁-C₄-alkylthio and C₁-C₄-haloalkylthio; preferably from hydrogen, F, Cl, Br, CH₃, CF₃, OCH₃, OCF₂H, OCF₃, SCH₃, SCF₂H, and SCF₃;
R^{10a} and R^{14a} independently from each other are selected from hydrogen, C₁-C₆-alkyl, C₁-C₆-haloalkyl, preferably from hydrogen and CH₃;
R^{14b} is selected from hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkyl-C₁-C₄-alkyl-, where the cycloalkyl moieties in the three last-mentioned groups may be substituted by a cyano group; C₁-C₆-alkyl substituted with a cyano group, C₁-C₆-alkoxy, C₁-C₆-haloalkoxy, phenyl which is optionally substituted with 1, 2, 3 or 4, substituents each independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; and a heteromonocyclic ring selected from rings of formulae D-1 to D-181

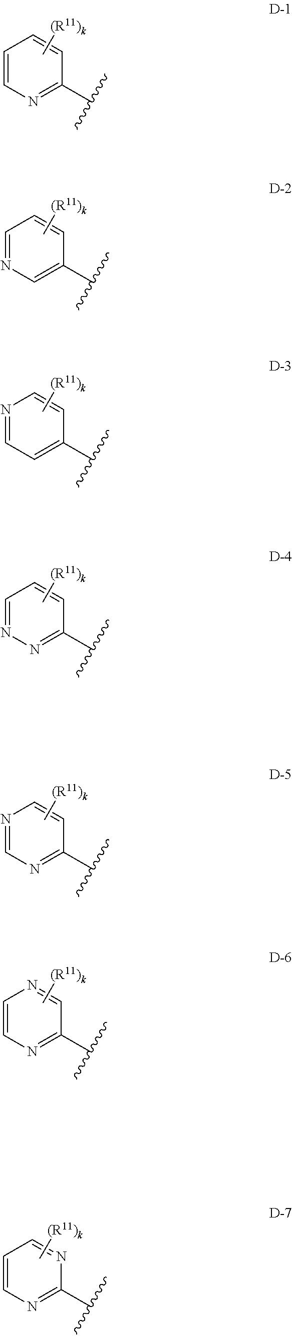

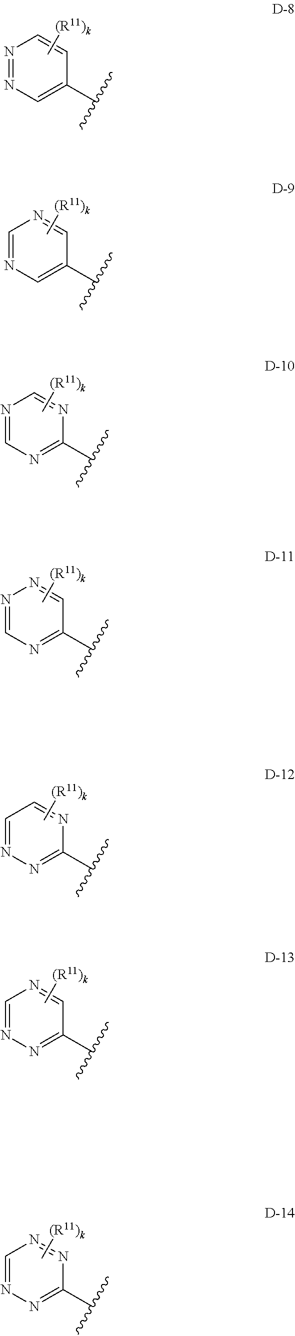

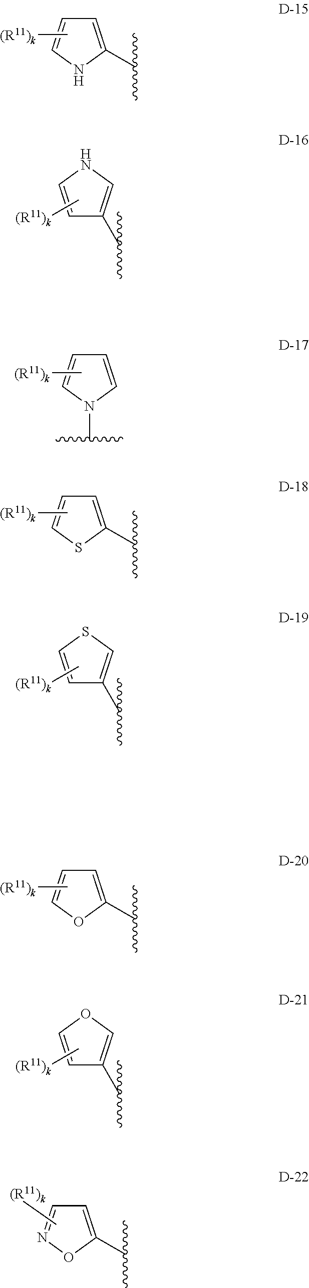

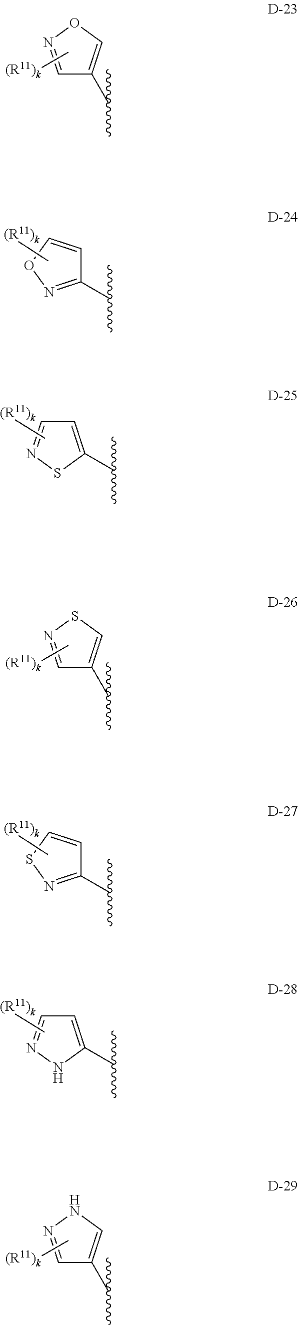

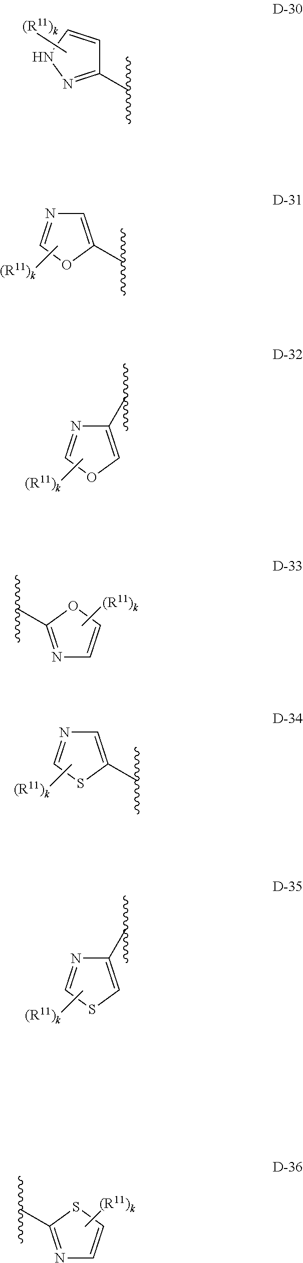

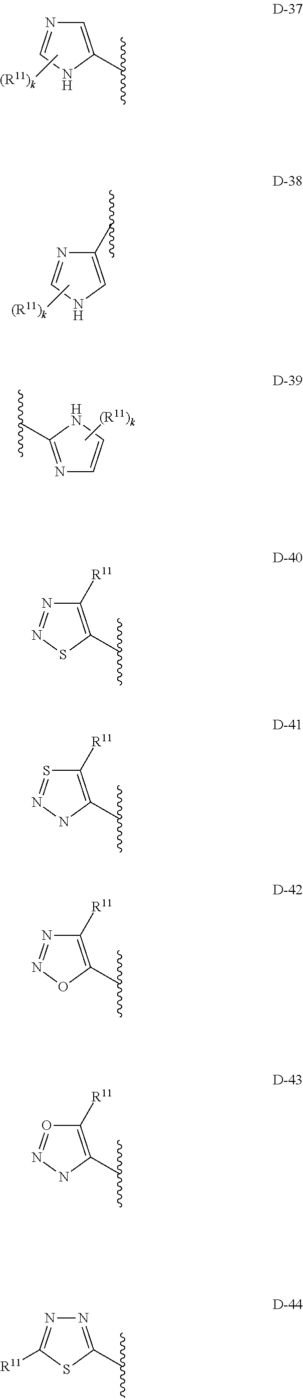

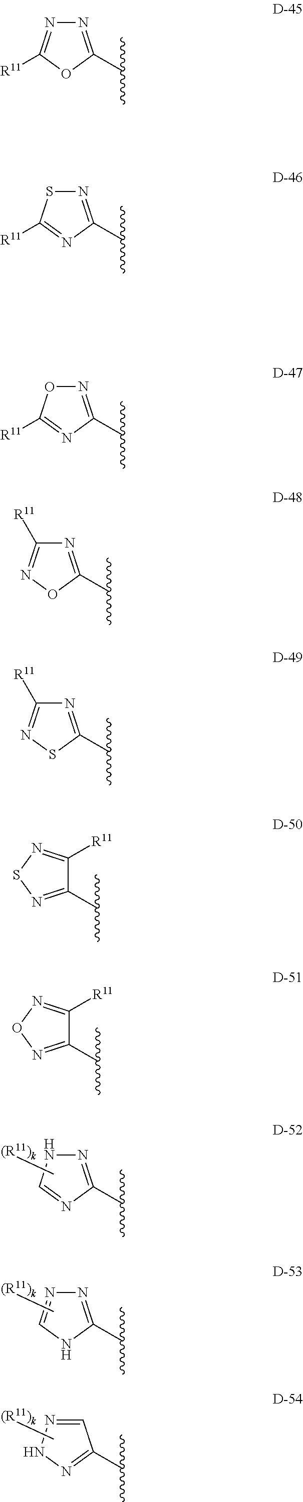

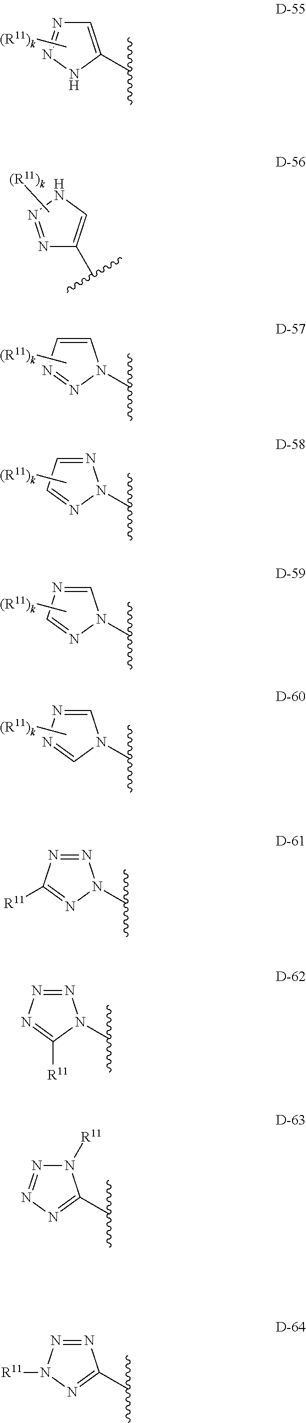

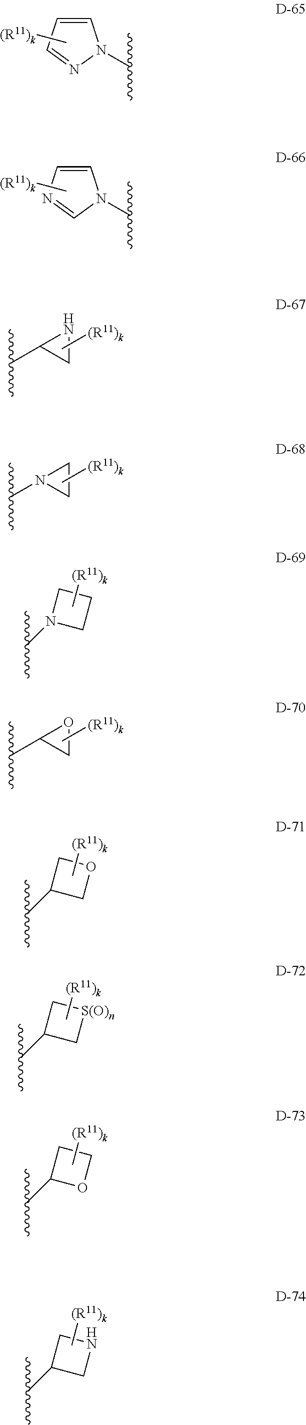

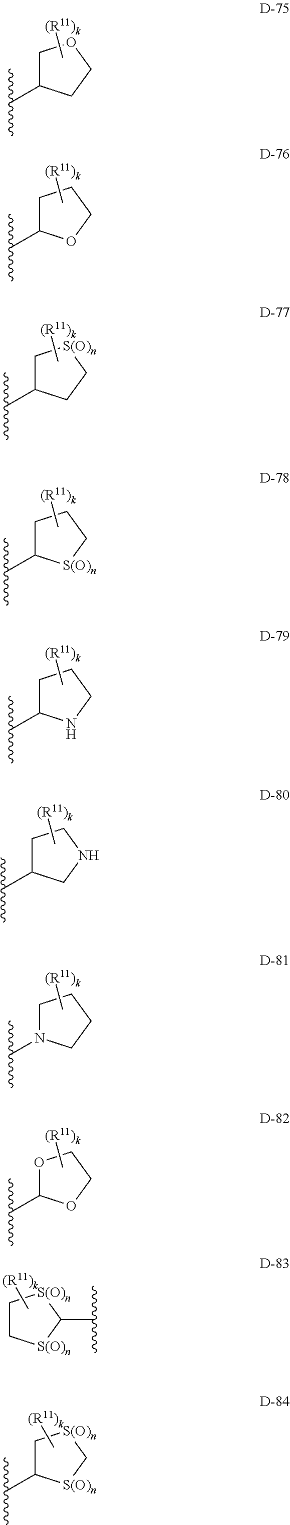

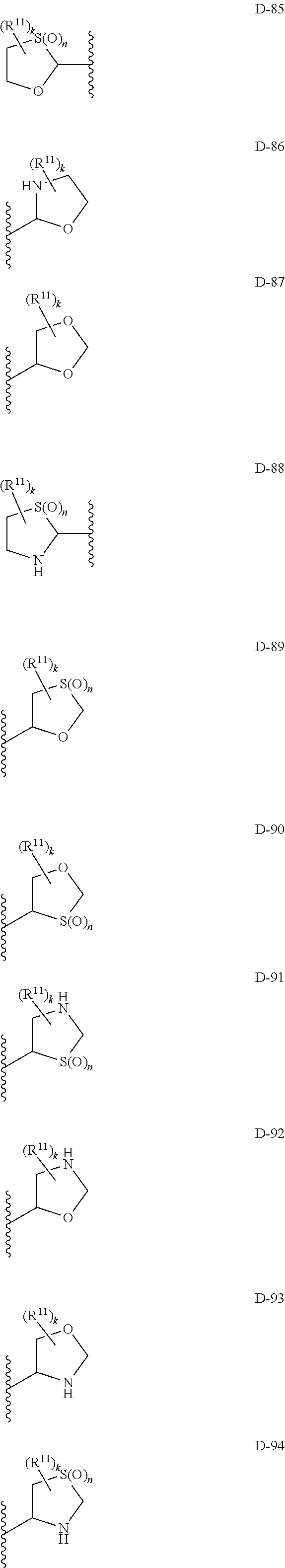

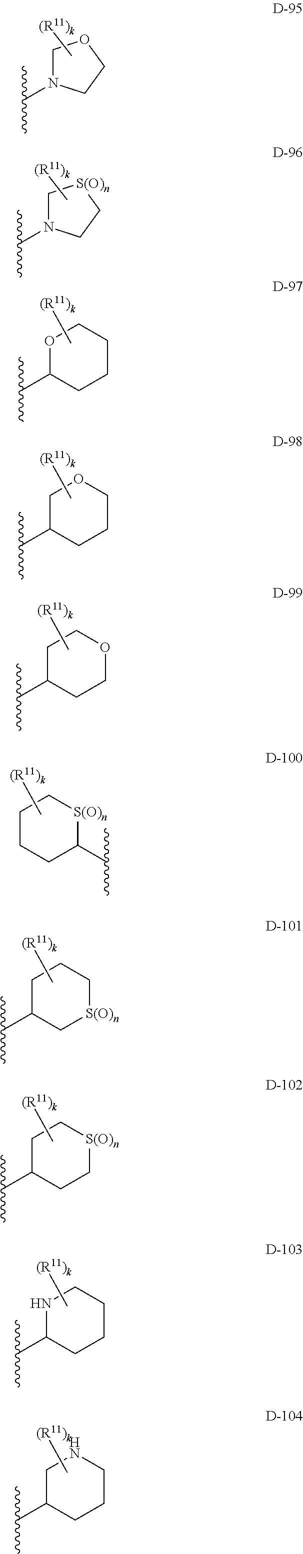

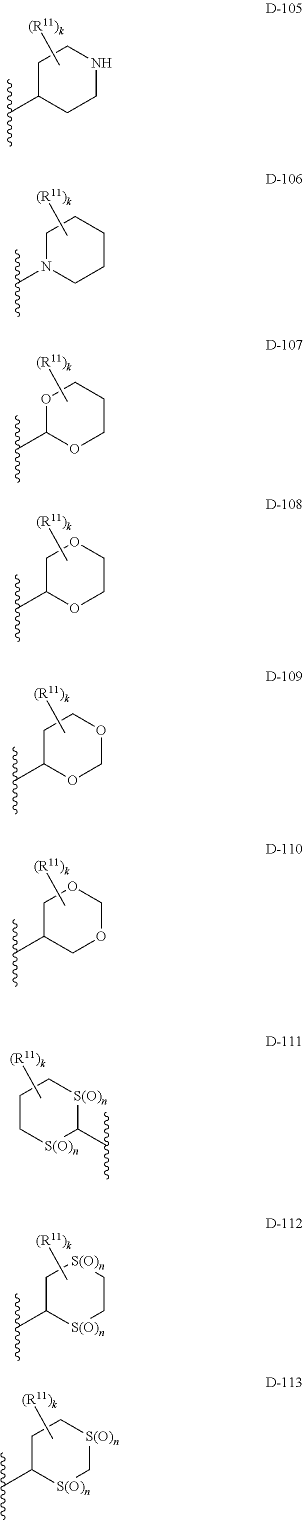

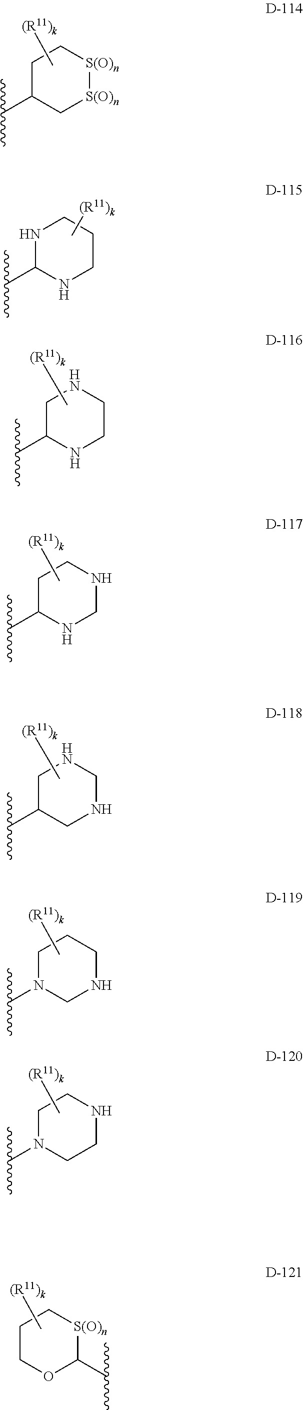

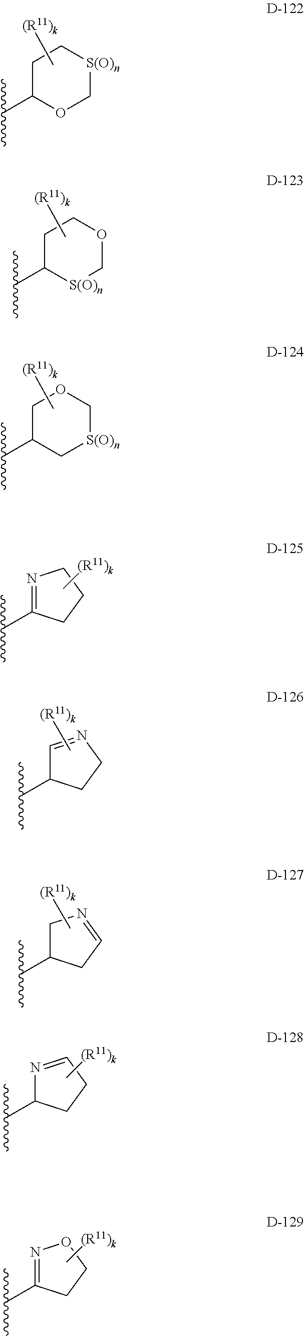

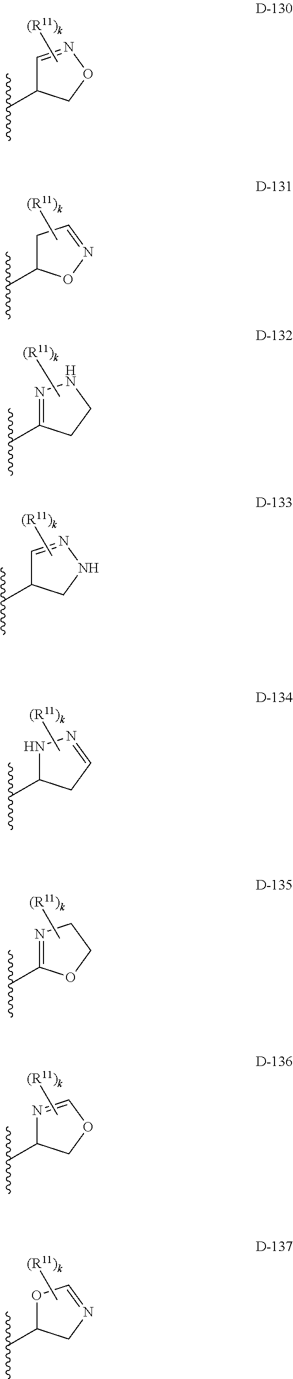

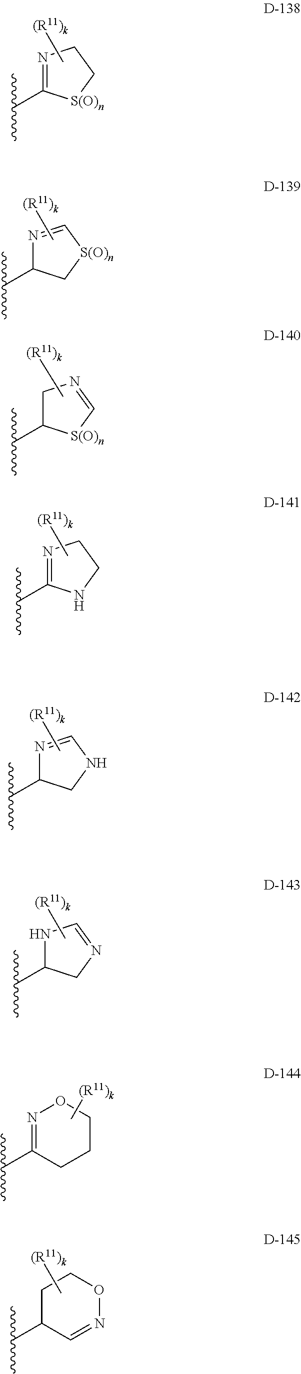

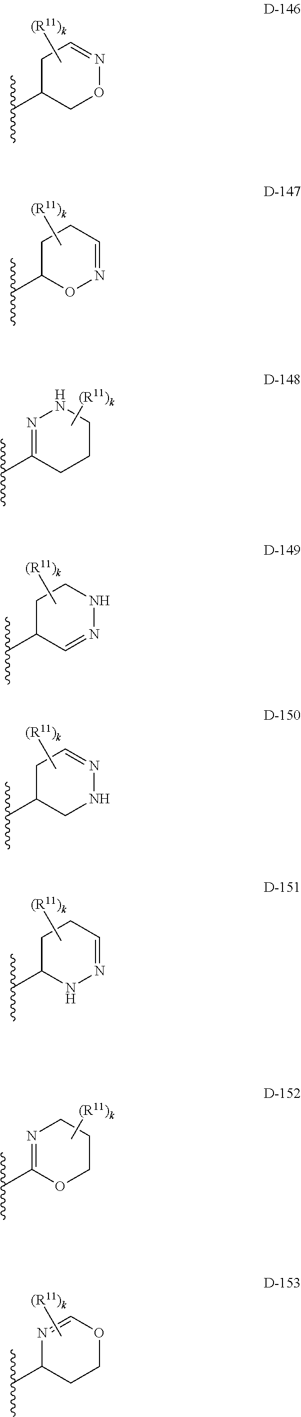

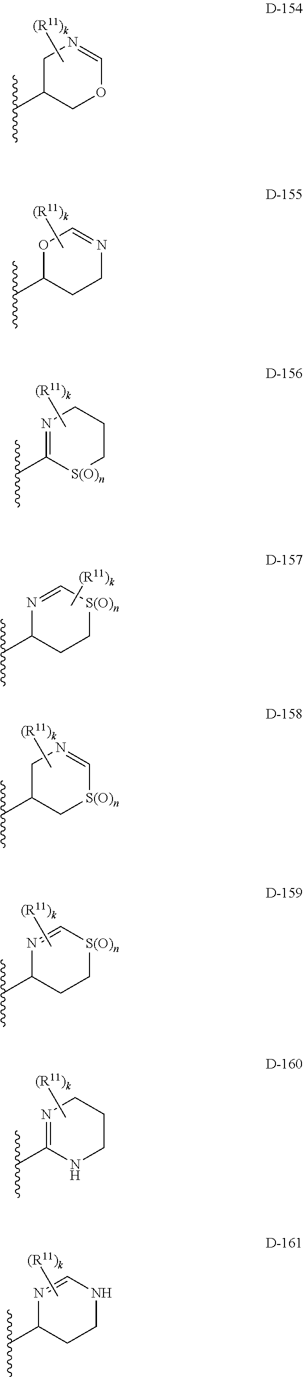

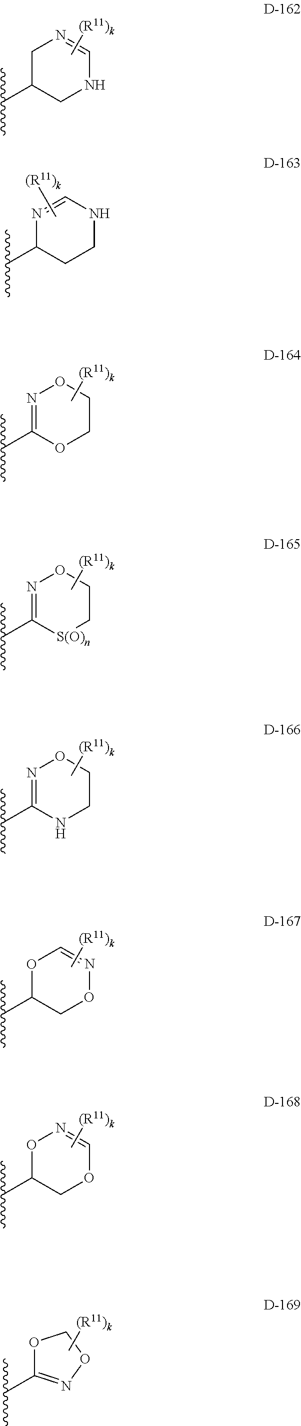

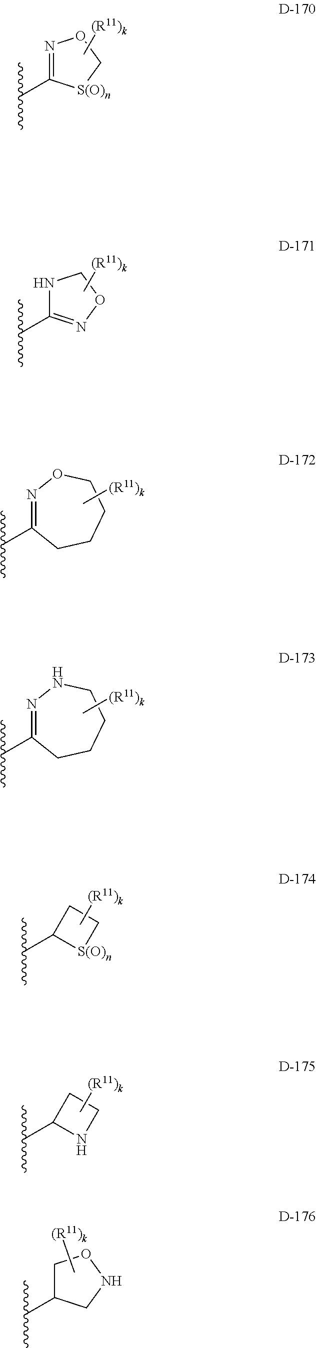

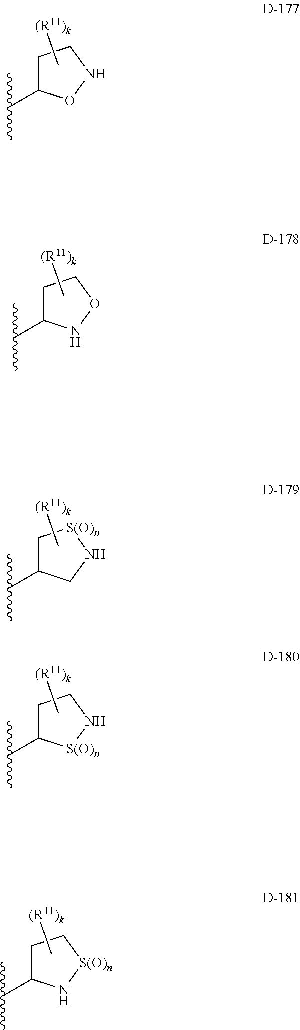

wherein
k is 0, 1, 2 or 3;
n is 0, 1 or 2; and
each R¹¹ is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl, C₁-C₄-alkylcarbonyl, C₁-C₄-alkylaminocarbonyl, or
   two R¹¹ present on the same carbon atom of a saturated heterocyclic ring may form together ═O or ═S;
      and wherein the method is for controlling and/or preventing pest infestation of the soybean plants and/or soybean crops.

In particular, in compounds I.1 R^{14b} is selected from C₁-C₆-alkyl, C₁-C₆-haloalkyl, C₂-C₆-alkynyl, C₂-C₆-haloalkynyl, C₃-C₆-cycloalkyl-C₁-C₄-alkyl-, where the cycloalkyl moiety may be substituted by a cyano group; C₁-C₆-alkoxy, C₁-C₆-haloalkoxy and phenyl.

In compounds I.1 R⁴ may further be selected from C₁-C₄-alkylsulfinyl and C₁-C₄-haloalkylsulfinyl.

In another aspect of the present invention, in the method comprising applying to soybean plants and/or crops of soybean plants a compound of formula I.1, the method is for controlling and/or preventing infestation by pests from the family of pentatomidae, stink bugs.

Preferably for controlling and/or preventing infestation by Acrosternum spp., Euschistus spp., Nezara spp., Piezodrus spp., Dichelops spp., Halyomorpha halys, Thyanta accerra, Podisus maculiventris and/or Megacopta cribraria, in particular for controlling and/or preventing infestation by Acrosternum spp., Euschistus spp., Nezara spp and/or Piezodrus spp., more particularly by Acrosternum hilare, Euschistus heros, Nezara viridula and/or Piezodrus guildini even more particularly by Euschistus heros and/or Nezara viridula, and especially by Euschistus heros. Alternatively or additionally, the method is in particular for controlling and/or preventing infestation by Halyomorpha halys.

In a further aspect the invention provides use of a compound of formula I.1 for control of pests from the family of pentatomidae, stink bugs. Preferably for the control of Acrosternum spp., Euschistus spp., Nezara spp., Piezodrus spp., Dichelops spp., Halyomorpha halys, Thyanta accerra, Podisus maculiventris and/or Megacopta cribraria, in particular for the control of Acrosternum spp., Euschistus spp., Nezara spp and/or Piezodrus spp., more particularly of Acrosternum hilare, Euschistus heros, Nezara viridula and/or Piezodrus guildini even more particularly for the control of Euschistus heros and/or Nezara viridula, and especially of Euschistus heros. Alternatively or additionally, the use is in particular for the control of Halyomorpha halys.

The use of compounds I.1 may be further for controlling pests, preferably pests from the family of pentatomidae, stinkbugs, preferably Acrosternum spp., Euschistus spp., Nezara spp., Piezodrus spp., Dichelops spp., Halyomorpha halys, Thyanta accerra, Podisus maculiventris and/or Megacopta cribraria, more preferably Acrosternum spp., Euschistus spp., Nezara spp and/or Piezodrus spp., in particular Acrosternum hilare, Euschistus heros, Nezara viridula and/or Piezodrus guildini more particularly Euschistus heros and/or Nezara viridula, and especially Euschistus heros, that are resistant to one or more other insecticides, preferably pyrethroid, neonicotinoids and organophosphates, and more preferably pyrethroid insecticides. Alternatively or additionally, the use of compounds I.1 may be in particular for the control of Halyomorpha halys that is resistant to one or more other insecticides, preferably pyrethroid, neonicotinoids and organophosphates, and more preferably pyrethroid insecticides.

In another embodiment of the invention a method is provided, which method comprises applying to soybean plants, to a crop of soybean plants, to the locus thereof or to propagation material thereof, a compound of formula I.2:

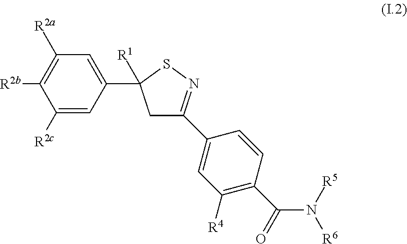

wherein
R¹ is C₁-C₄-haloalkyl, in particular CF₃;
R^{2a}, R^{2b} and R^{2c} are selected independently from one another from hydrogen, halogen and C₁-C₂-haloalkyl, preferably from hydrogen, F, Cl, Br and CF₃;
R⁴ is selected from hydrogen, halogen, cyano, C₁-C₄-alkyl, C₁-C₄-haloalkyl, C₁-C₄-alkoxy, C₁-C₄-haloalkoxy, C₁-C₄-alkylthio and C₁-C₄-haloalkylthio, preferably from hydrogen, F, Cl, Br, CH₃, and CF₃;
R⁵ is selected from hydrogen, C₁-C₆-alkyl, C₂-C₃-alkenyl, C₂-C₃-alkynyl, —CH₂—CN and C₁-C₆-alkoxy-methyl-, and preferably from hydrogen and C₁-C₄-alkyl; and
R⁶ is selected from hydrogen, C₁-C₆-alkyl, C₁-C₆-haloalkyl, C₁-C₄-alkyl which carries one radical R⁸, C₂-C₆-alkenyl, C₂-C₆-haloalkenyl, C₂-C₆-alkynyl, C₃-C₆-cycloalkyl which may be substituted by 1 to 4 substituents selected from halogen and cyano; N(R^{10a})R^{10b}, —CH═NOR⁹; phenyl which may be substituted with 1, 2, 3, 4, or 5 substituents R¹¹, and a heteromonocyclic ring selected from rings of formulae D-1 to D-181 with its respective substituents R¹¹ as defined herein above;
R⁸ is selected from hydrogen, OH, CN, C₃-C₈-cycloalkyl, C₂-C₆-alkenyl, C₂-C₆-alkynyl, wherein the three last-mentioned radicals may be partially or fully halogenated and/or may be substituted by one or more cyano group; C₁-C₆-alkoxy, C₁-C₆-alkylthio, C₁-C₆-alkylsulfinyl, C₁-C₆-alkylsulfonyl, wherein the four last-mentioned radicals may be partially or fully halogenated and/or may be substituted by one or more substituents selected from cyano, C₃-C₆-cycloalkyl, C₂-C₆-alkenyl, and C₂-C₆-alkynyl;
   —C(═O)N(R^{14a})R^{14b};
   phenyl, optionally substituted with 1, 2, 3, 4 or 5 substituents R¹¹; and a heteromonocyclic ring selected from rings of formulae D-1 to D-181 with its respective substituents R¹¹ as defined herein above;
R⁹ is selected from hydrogen, C₁-C₆-alkyl, C₃-C₆-cycloalkyl, C₃-C₆-cycloalkylmethyl, wherein the three last-mentioned radicals may be partially or fully halogenated and/or may be substituted by one or more CN;
R^{10a} is selected from the group consisting of hydrogen and C₁-C₆-alkyl;
R^{10b} is selected from the group consisting of hydrogen, C(═O)N(R^{14a})R^{14b}; phenyl, optionally substituted with 1, 2, 3, 4 or 5 substituents R¹¹; and a heteromonocyclic ring selected from rings of formulae D-1 to D-181 with its respective substituents R¹¹ as defined herein above;
each R¹¹ is independently selected from the group consisting of hydrogen, halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl, C₁-C₄-alkylcarbonyl, C₁-C₄-alkylaminocarbonyl; or
   two R¹¹ present on the same carbon atom of a saturated heterocyclic ring may form together ═O or ═S;
each R^{14a} is selected from the group consisting of hydrogen and C₁-C₆-alkyl;
each R^{14b} is selected from the group consisting of hydrogen, C₁-C₆-alkyl, C₃-C₆-cycloalkyl, C₃-C₆-cycloalkylmethyl, C₂-C₄-alkynyl, C₂-C₄-alkenyl, C₁-C₄-alkoxy, wherein the six last-mentioned radicals may be partially or fully halogenated and/or may be substituted with one cyano group;
   and wherein the method is for controlling and/or preventing pest infestation of the soybean plants and/or soybean crops.

In compounds I.2 R⁵ and R⁶ may further form together a group ═S(R⁹)₂, where R⁹ is as defined above and is preferably C₁-C₄-alkyl.

In compounds I.2 R⁴ is preferably selected from hydrogen, halogen and C₁-C₄-alkyl.

In compounds I.2 R⁶ is in particular selected from hydrogen, C₁-C₄-alkyl which carries one radical R⁸, C₃-C₆-alkenyl, C₃-C₆-haloalkenyl, C₃-C₆-alkynyl, C₃-C₆-cycloalkyl which may be substituted by 1 to 4 substituents selected from halogen and cyano; and —N(R^{10a})R^{10b}, where
R⁸ has one of the above definitions and is in particular selected from C₃-C₈-cycloalkyl which may be partially or fully halogenated and/or may be substituted by one or more cyano group;
   —O—CH₂—C₃-C₆-cycloalkyl, —O—C₃-C₆-alkenyl, —O—C₃-C₆-alkynyl, —S(O)ₙ—CH₂—C₃-C₆-cycloalkyl, —S(O)ₙ—C₃-C₆-alkenyl, —S(O)ₙ—C₃-C₆-alkynyl, where n is 0, 1 or 2; and —C(═O)N(R^{14a})R^{14b};
R^{10a} is hydrogen or C₁-C₄-alkyl;
R^{10b} is —C(═O)N(R^{14a})R^{14b};
R^{14a} is hydrogen or C₁-C₄-alkyl; and
R^{14b} is is selected from the group consisting of hydrogen, C₁-C₆-alkyl, C₃-C₆-cycloalkyl, C₃-C₆-cycloalkylmethyl, C₂-C₄-alkynyl and C₂-C₄-alkenyl, wherein the five last-mentioned radicals may be partially or fully halogenated and/or may be substituted with one cyano group.

In another aspect of the present invention, in the method comprising applying to soybean plants and/or crops of soybean plants a compound of formula I.2, the method is for controlling and/or preventing infestation by pests from the family of pentatomidae, stink bugs.

Preferably for controlling and/or preventing infestation by Acrosternum spp., Euschistus spp., Nezara spp., Piezodrus spp., Dichelops spp., Halyomorpha halys, Thyanta accerra, Podisus maculiventris and/or Megacopta cribraria, in particular for controlling and/or preventing infestation by Acrosternum spp., Euschistus spp., Nezara spp and/or Piezodrus spp, more particularly by Acrosternum hilare, Euschistus heros, Nezara viridula and/or Piezodrus guildini even more particularly Euschistus heros and/or Nezara viridula, and especially by Euschistus heros. Alternatively or additionally, the method is in particular for controlling and/or preventing infestation by Halyomorpha halys.

In a further aspect the invention provides use of a compound of formula I.2 for control of pests from the family of pentatomidae, stink bugs. Preferably for the control of Acrosternum spp., Euschistus spp., Nezara spp., Piezodrus spp., Dichelops spp., Halyomorpha halys, Thyanta accerra, Podisus maculiventris and/or Megacopta cribraria, in particular for the control of Acrosternum spp., Euschistus spp., Nezara spp and/or Piezodrus spp., more particularly of Acrosternum hilare, Euschistus heros, Nezara viridula and/or Piezodrus guildini even more particularly Euschistus heros and/or Nezara viridula, and especially of Euschistus heros. Alternatively or additionally, the use is in particular for the control of Halyomorpha halys.

The use of compounds I.2 may be further for controlling pests, preferably pests from the family of pentatomidae, stinkbugs, preferably of Acrosternum spp., Euschistus spp., Nezara spp., Piezodrus spp., Dichelops spp., Halyomorpha halys, Thyanta accerra, Podisus maculiventris and/or Megacopta cribraria, more preferably of Acrosternum spp., Euschistus spp., Nezara spp and/or Piezodrus spp., more particularly of Acrosternum hilare, Euschistus heros, Nezara viridula and/or Piezodrus guildini, even more particularly Euschistus heros and/or Nezara viridula, and especially of Euschistus spp. that are resistant to one or more other insecticides, preferably pyrethroid, neonicotinoids and organophosphates, and more preferably pyrethroid insecticides. Alternatively or additionally, the use of compounds I.2 may be in particular for the control of Halyomorpha halys that is resistant to one or more other insecticides, preferably pyrethroid, neonicotinoids and organophosphates, and more preferably pyrethroid insecticides.

As a particular embodiment of the invention a method is provided, which method comprises applying to soybean plants, to a crop of soybean plants, to the locus thereof or to propagation material thereof, a compound of formula I.2a:

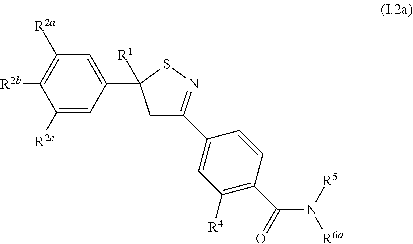

wherein
R¹ is C₁-C₄-haloalkyl and is in particular CF₃;
R^{2a}, R^{2b} and R^{2c} are selected independently from one another from hydrogen, halogen and C₁-C₂-haloalkyl, preferably from hydrogen, F, Cl, Br and CF₃;
R⁴ is selected from hydrogen, halogen, cyano, C₁-C₄-alkyl, C₁-C₄-haloalkyl, C₁-C₄-alkoxy, C₁-C₄-haloalkoxy, C₁-C₄-alkylthio and C₁-C₄-haloalkylthio, preferably from hydrogen, F, Cl, Br, CH₃, and CF₃;
R⁵ is selected from hydrogen, C₁-C₆-alkyl, C₂-C₃-alkenyl, C₂-C₃-alkynyl, CH₂—CN and C₁-C₆-alkoxy-methyl-, and preferably from hydrogen and C₁-C₄-alkyl; and
R^{6a} is selected from —X—R^{6b} and —N(R^{5a})R^{6c}; wherein
X is selected from —C(R^{a})₂—, —C(R^{a})₂—C(R^{a})₂—, —C(R^{a})₂—C(═O)—NR^{10a}—C(R^{a})₂—, —C(R^{a})₂S(O)ₙ—C(R^{a})₂—, —C(R^{a})₂—C(R^{a})₂—S(O)ₙ—C(R^{a})₂—, —C(R^{a})₂—O—C(R^{a})₂—, and —C(R^{a})₂—C(R^{a})₂—O—C(R^{a})₂—, and preferably from —C(R^{a})₂—, —C(R^{a})₂—C(═O)—NH—C(R^{a})₂—, —C(R^{a})₂—C(R^{a})₂—S(O)₂—C(R^{a})₂—, and —C(R^{a})₂—C(R^{a})₂—O—C(R^{a})₂—, wherein
   n is 0, 1 or 2, and
   each R^{a} is independently selected from the group consisting of hydrogen, halogen, C₁-C₃-alkyl, and C₁-C₃-haloalkyl and is preferably hydrogen;
R^{5a} is selected from hydrogen and C₁-C₆-alkyl, is preferably hydrogen;
R^{6b} is selected from the group consisting of C₃-C₆-cycloalkyl, C₂-C₆-alkenyl, C₂-C₆-alkynyl, wherein the three last-mentioned radicals may be partially or fully halogenated and/or may be substituted by one or more substituents R⁸;
R^{6c} is —C(═O)N(R^{14a})R^{14b}
R⁸ is selected from, cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —C(═O)N(R^{14a})R^{14b};
each R^{14a} is selected from the group consisting of hydrogen and C₁-C₆-alkyl;
each R^{14b} is selected from the group consisting of hydrogen, C₁-C₆-alkyl, C₃-C₆-cycloalkyl, C₃-C₆-cycloalkylmethyl, C₂-C₄-alkynyl, C₂-C₄-alkenyl, C₁-C₄-alkoxy, wherein the six last-mentioned radicals may be partially or fully halogenated and/or may be substituted with one cyano group;
where in case that R⁵ is hydrogen, R^{6a} is further selected from hydrogen, 1-cyanocyclopropyl, 1-cyanocyclobutyl and 1-cyanocyclopentyl;
   and wherein the method is for controlling and/or preventing pest infestation of the soybean plants and/or soybean crops.

In compounds I.2a R⁵ and R^{6a} may further form together a group ═S(R⁹)₂, where R⁹ is as defined above and is preferably C₁-C₄-alkyl.

In compounds I.2a R⁴ is preferably selected from hydrogen, halogen and C₁-C₄-alkyl.

In another aspect of the present invention, in the method comprising applying to soybean plants and/or crops of soybean plants a compound of formula I.2a, the method is for controlling and/or preventing infestation by pests from the family of pentatomidae, stink bugs.

Preferably for controlling and/or preventing infestation by Acrosternum spp., Euschistus spp., Nezara spp., Piezodrus spp., Dichelops spp., Halyomorpha halys, Thyanta accerra, Podisus maculiventris and/or Megacopta cribraria, in particular for controlling and/or preventing infestation by Acrosternum spp., Euschistus spp., Nezara spp and/or Piezodrus spp., more particularly by Acrosternum hilare, Euschistus heros, Nezara viridula and/or Piezodrus guildini, even more particularly by Euschistus heros and/or Nezara viridula, and especially by Euschistus heros. Alternatively or additionally, the method is in particular for controlling and/or preventing infestation by Halyomorpha halys.

In a further aspect the invention provides use of a compound of formula I.2a for control of pests from the family of pentatomidae, stink bugs. Preferably for the control of Acrosternum spp., Euschistus spp., Nezara spp., Piezodrus spp., Dichelops spp., Halyomorpha halys, Thyanta accerra, Podisus maculiventris and/or Megacopta cribraria, in particular for the control of Acrosternum spp., Euschistus spp., Nezara spp and/or Piezodrus spp., more particularly of Acrosternum hilare, Euschistus heros, Nezara viridula and/or Piezodrus guildini, even more particularly of Euschistus heros and/or Nezara viridula, and especially of Euschistus heros. Alternatively or additionally, the use is in particular for the control of Halyomorpha halys.

The use of compounds I.2a may be further for controlling pests, preferably pests from the family of pentatomidae, stinkbugs, preferably of Acrosternum spp., Euschistus spp., Nezara spp., Piezodrus spp., Dichelops spp., Halyomorpha halys, Thyanta accerra, Podisus maculiventris and/or Megacopta cribraria, more preferably of Acrosternum spp., Euschistus spp., Nezara spp and/or Piezodrus spp., in particular of Acrosternum hilare, Euschistus heros, Nezara viridula and/or Piezodrus guildini, more particularly of Euschistus heros and/or Nezara viridula, and especially of Euschistus spp. that are resistant to one or more other insecticides, preferably pyrethroid, neonicotinoids and organophosphates, and more preferably pyrethroid insecticides. Alternatively or additionally, the use of compounds I.2a may be in particular for the control of Halyomorpha halys that is resistant to one or more other insecticides, preferably pyrethroid, neonicotinoids and organophosphates, and more preferably pyrethroid insecticides.

In a further embodiment of the invention a method is provided, which method comprises applying to soybean plants, to a crop of soybean plants, to the locus thereof or to propagation material thereof, a compound of formula I.3:

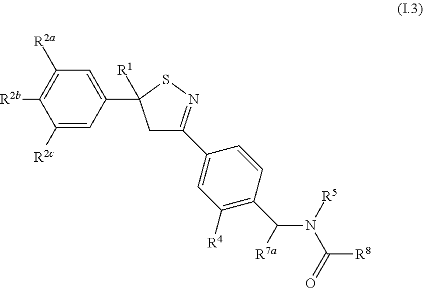

wherein
R¹ is C₁-C₄-haloalkyl and is in particular CF₃;
R^{2a}, R^{2b} and R^{2c} are selected independently from one another from hydrogen, halogen, C₁-C₂-haloalkyl and C₁-C₂-haloalkoxy, preferably from hydrogen, F, Cl, Br, I, CF₃ and OCF₃;
R⁴ is selected from hydrogen, halogen, cyano, nitro, C₁-C₄-alkyl, C₁-C₄-haloalkyl, C₁-C₄-alkoxy, C₁-C₄-haloalkoxy, C₁-C₄-alkylthio and C₁-C₄-haloalkylthio; preferably from hydrogen, nitro, F, Cl, Br, CH₃, CF₃, OCH₃, OCF₂H, OCF₃, SCH₃, SCF₂H, and SCF₃;
R⁵ is selected from hydrogen, C₁-C₄-alkyl, C₂-C₃-alkynyl, —CH₂—CN, C₁-C₆-alkoxy-methyl and C₃-C₆-cycloalkyl, and preferably from hydrogen and C₁-C₄-alkyl;
R^{7a} is hydrogen or methyl;
R⁸ is selected from the group consisting of hydrogen, C₁-C₆-alkyl, C₃-C₆-cycloalkyl, C₃-C₆-cycloalkenyl, C₂-C₆-alkenyl, wherein the three last-mentioned radicals may be partially or fully halogenated and/or may be substituted by one or more substituents R¹³; C₃-C₈-alkynyl, —N(R^{10a})R^{10b}, —C(═O)N(R^{14a})R^{14b}, —OR⁹, —CH═NOR⁹, phenyl, optionally substituted with 1, 2, 3, 4 or 5 substituents R¹¹; and a heterocyclic ring selected from rings of formulae D-1 to D-181 with its respective substituents R¹¹ as defined herein above;
R⁹ is selected from hydrogen, C₁-C₆-alkyl, C₃-C₆-cycloalkyl, C₃-C₆-cycloalkylmethyl, wherein the three last-mentioned radicals may be partially or fully halogenated and/or may be substituted by one or more cyano group;
R^{10a} is selected from the group consisting of hydrogen and C₁-C₆-alkyl;
R^{10b} is selected from the group consisting of hydrogen, C₁-C₆-alkyl, C₁-C₆-cycloalkyl, wherein the two last-mentioned radicals may be partially or fully halogenated and/or are optionally substituted by one cyano group; —C(═O)N(R^{14a})R^{14b}; phenyl, optionally substituted with 1, 2, 3, 4 or 5 substituents R¹¹; and a heteromonocyclic ring selected from rings of formulae D-1 to D-181 with its respective substituents R¹¹ as defined herein above;
each R¹¹ is independently selected from the group consisting of hydrogen, halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl, C₁-C₄-alkylcarbonyl, C₁-C₄-alkylaminocarbonyl; or
   two R¹¹ present on the same carbon atom of a saturated heterocyclic ring may form together ═O or ═S;
R¹³ is selected from cyano, C₃-C₆-cycloalkyl, C₃-C₆-halocycloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —N(R^{14a})R^{14b}, —C(═O)N(R^{14a})R^{14b}, phenyl, optionally substituted with 1, 2, 3, 4 or 5 substituents R¹¹, and a heterocyclic ring selected from rings of formulae D-1 to D-181 with its respective substituents R¹¹ as defined herein above, and is preferably selected from C₁-C₆-alkoxy, C₁-C₆-haloalkoxy, C₁-C₆-alkylthio, C₁-C₆-haloalkylthio, C₁-C₆-alkylsulfonyl and C₁-C₆-haloalkylsulfonyl;
   and as a substituent on a cycloalkyl or cycloalkenyl ring is additionally selected from C₁-C₄-alkyl and C₁-C₄-haloalkyl;
each R^{14a} is selected from the group consisting of hydrogen and C₁-C₆-alkyl; and
each R^{14b} is selected from the group consisting of hydrogen, C₁-C₆-alkyl, C₂-C₄-alkynyl, C₂-C₄-alkenyl, —CH₂—CN, C₁-C₆-haloalkyl, C₃-C₆-cycloalkyl, C₃-C₆-halocycloalkyl, C₁-C₄-alkoxy, C₁-C₄-haloalkoxy and C₃-C₆-cycloalkylmethyl;
   and wherein the method is for controlling and/or preventing pest infestation of the soybean plants and/or soybean crops.

In the above definitions, rings D-1 to D-181 are preferably selected from rings D-1, D-2, D-3, D-7, D-18, D-19, D-20, D-21, D-71, D-72, D-73, D-75, D-76, D-77, D-78, D-82, D-107, D-108, D-109 and D-110.

In compounds I.3
each R⁸ is independently preferably selected from the group consisting of hydrogen, C₁-C₆-alkyl, C₁-C₆-haloalkyl, C₁-C₆-alkyl substituted by one substituent R¹³; C₃-C₆-cycloalkyl, C₃-C₆-halocycloalkyl, C₃-C₆-cycloalkenyl, C₃-C₆-cycloalkyl substituted by one or more substituents R¹³; C₂-C₆-alkenyl, C₂-C₆-haloalkenyl, C₃-C₈-alkynyl, —C(═O)N(R^{14a})R^{14b}, —OR⁹, —CH═NOR⁹, phenyl, optionally substituted with 1, 2, 3, 4 or 5 substituents R¹¹; and a heterocyclic ring selected from rings of formulae D-1 to D-181 with its respective substituents R¹¹ as defined herein above, wherein R⁹, R¹³, R^{14a} and R^{14b} are as defined above; and where preferably:
   each R⁹ is independently selected from hydrogen, C₁-C₆-alkyl and C₁-C₆-haloalkyl;
   each R¹³ is independently selected from cyano, C₃-C₆-cycloalkyl, C₃-C₆-halocycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, —N(R^{14a})R^{14b}, —C(═O)N(R^{14a})R^{14b}, phenyl, optionally substituted with 1, 2, 3, 4 or 5 substituents R¹¹, and a heterocyclic ring selected from rings of formulae D-1 to D-181 with its respective substituents R¹¹ as defined herein above, and is preferably selected from C₁-C₆-alkoxy, C₁-C₆-haloalkoxy, C₁-C₆-alkylthio, C₁-C₆-haloalkylthio, C₁-C₆-alkylsulfonyl and C₁-C₆-haloalkylsulfonyl; and as a substituent on a cycloalkyl or cycloalkenyl ring is additionally selected from C₁-C₄-alkyl and C₁-C₄-haloalkyl;
   each R^{14a} is independently selected from hydrogen and C₁-C₄-alkyl; and
   each R^{14b} is independently selected from hydrogen, C₁-C₆-alkyl, C₁-C₆-haloalkyl, C₂-C₄-alkenyl, C₂-C₄-alkynyl, C₃-C₆-cycloalkyl, C₃-C₆-halocycloalkyl, and C₃-C₆-cycloalkylmethyl.

In a particular embodiment of the invention a method is provided, which method comprises applying to soybean plants, to a crop of soybean plants, to the locus thereof or to propagation material thereof, a compound of formula I.3:

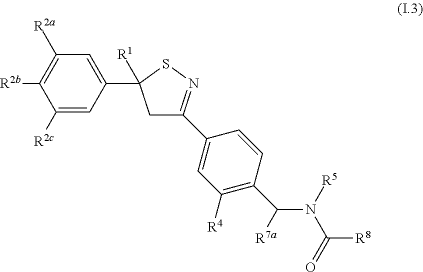

wherein
R¹ is C₁-C₄-haloalkyl and is in particular CF₃;
R^{2a}, R^{2b} and R^{2c} are selected independently from one another from hydrogen, halogen and C₁-C₂-haloalkyl, preferably from hydrogen, F, Cl, Br and CF₃;
R⁴ is selected from hydrogen, halogen, cyano, C₁-C₄-alkyl, C₁-C₄-haloalkyl, C₁-C₄-alkoxy, C₁-C₄-haloalkoxy, C₁-C₄-alkylthio and C₁-C₄-haloalkylthio; preferably from hydrogen, F, Cl, Br, CH₃, CF₃, OCH₃, OCF₂H, OCF₃, SCH₃, SCF₂H, and SCF₃;
R⁵ is selected from hydrogen, C₁-C₄-alkyl, C₂-C₃-alkynyl, —CH₂—CN and C₁-C₆-alkoxy-methyl-, and preferably from hydrogen and C₁-C₄-alkyl;
R^{7a} is hydrogen or methyl;
R⁸ is selected from the group consisting of hydrogen, C₁-C₆-alkyl, C₃-C₆-cycloalkyl, C₃-C₆-alkenyl, wherein the three last-mentioned radicals may be partially or fully halogenated and/or may be substituted by one or more substituents R¹³; C₃-C₈-alkynyl, —N(R^{10a})R^{10b}, —C(═O)N(R^{14a})R^{14b}, —CH═NOR⁹; phenyl, optionally substituted with 1, 2, 3, 4 or 5 substituents R¹¹; and a heterocyclic ring selected from rings of formulae D-1 to D-181 with its respective substituents R¹¹ as defined herein above;
R⁹ is selected from hydrogen, C₁-C₆-alkyl, C₃-C₆-cycloalkyl, C₃-C₆-cycloalkylmethyl, wherein the three last-mentioned radicals may be partially or fully halogenated and/or may be substituted by one or more cyano group;
R^{10a} is selected from the group consisting of hydrogen and C₁-C₆-alkyl;
R^{10b} is selected from the group consisting of hydrogen, C₁-C₆-alkyl, C₁-C₆-cycloalkyl, wherein the two last-mentioned radicals may be partially or fully halogenated and/or are optionally substituted by one cyano group; —C(═O)N(R^{14a})R^{14b}; phenyl, optionally substituted with 1, 2, 3, 4 or 5 substituents R¹¹; and a heteromonocyclic ring selected from rings of formulae D-1 to D-181 with its respective substituents R¹¹ as defined herein above;
each R¹¹ is independently selected from the group consisting of hydrogen, halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl, C₁-C₄-alkylcarbonyl, C₁-C₄-alkylaminocarbonyl; or
   two R¹¹ present on the same carbon atom of a saturated heterocyclic ring may form together ═O or ═S;
R¹³ is selected from cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —C(═O)N(R^{14a})R^{14b}, phenyl, optionally substituted with 1, 2, 3, 4 or 5 substituents R¹¹, and a heterocyclic ring selected from rings of formulae D-1 to D-181 with its respective substituents R¹¹ as defined herein above, and is preferably selected from C₁-C₆-alkoxy, C₁-C₆-haloalkoxy, C₁-C₆-alkylthio, C₁-C₆-haloalkylthio, C₁-C₆-alkylsulfonyl and C₁-C₆-haloalkylsulfonyl;
each R^{14a} is selected from the group consisting of hydrogen and C₁-C₆-alkyl; and
each R^{14b} is selected from the group consisting of hydrogen, C₁-C₆-alkyl, C₂-C₄-alkynyl, C₂-C₄-alkenyl, —CH₂—CN, C₁-C₆-haloalkyl, C₃-C₆-cycloalkyl, C₃-C₆-halocycloalkyl, C₁-C₄-alkoxy and C₁-C₄-haloalkoxy, C₃-C₆-cycloalkylmethyl;
   and wherein the method is for controlling and/or preventing pest infestation of the soybean plants and/or soybean crops.

In another aspect of the present invention, in the method comprising applying to soybean plants and/or crops of soybean plants a compound of formula I.3, the method is for controlling and/or preventing infestation by pests from the family of pentatomidae, stink bugs.

Preferably for controlling and/or preventing infestation by Acrosternum spp., Euschistus spp., Nezara spp., Piezodrus spp., Dichelops spp., Halyomorpha halys, Thyanta accerra, Podisus maculiventris and/or Megacopta cribraria, in particular for controlling and/or preventing infestation by Acrosternum spp., Euschistus spp., Nezara spp and/or Piezodrus spp., more particularly by Acrosternum hilare, Euschistus heros, Nezara viridula and/or Piezodrus guildini, even more particularly by Euschistus heros and/or Nezara viridula, and especially by Euschistus heros. Alternatively or additionally, the method is in particular for controlling and/or preventing infestation by Halyomorpha halys.

In a further aspect the invention provides use of a compound of formula I.3 for control of pests from the family of pentatomidae, stink bugs. Preferably for the control of Acrosternum spp., Euschistus spp., Nezara spp., Piezodrus spp., Dichelops spp., Halyomorpha halys, Thyanta accerra, Podisus maculiventris and/or Megacopta cribraria, in particular for the control of Acrosternum spp., Euschistus spp., Nezara spp and/or Piezodrus spp., more particularly of Acrosternum hilare, Euschistus heros, Nezara viridula and/or Piezodrus guildini even more particularly of Euschistus heros and/or Nezara viridula, and especially of Euschistus heros. Alternatively or additionally, the use is in particular for the control of Halyomorpha halys.

The use of compounds of formula I.3 may be further for controlling pests, preferably pests from the family of pentatomidae, stinkbugs, preferably of Acrosternum spp., Euschistus spp., Nezara spp., Piezodrus spp., Dichelops spp., Halyomorpha halys, Thyanta accerra, Podisus maculiventris and/or Megacopta cribraria, more preferably of Acrosternum spp., Euschistus spp., Nezara spp and/or Piezodrus spp., in particular of Acrosternum hilare, Euschistus heros, Nezara viridula and/or Piezodrus guildini, more particularly of Euschistus heros and/or Nezara viridula, and especially of Euschistus spp., that are resistant to one or more other insecticides, preferably pyrethroid, neonicotinoids and organophosphates, and more preferably pyrethroid insecticides. Alternatively or additionally, the use of compounds I.2a may be in particular for the control of Halyomorpha halys that is resistant to one or more other insecticides, preferably pyrethroid, neonicotinoids and organophosphates, and more preferably pyrethroid insecticides.

### Pests

The compounds applied in the methods of the present invention may be used on soybean to control, for example,

Elasmopalpus lignosellus, Diloboderus abderus, Diabrotica speciosa, Sternechus subsignatus, Formicidae, Agrotis ypsilon, Julus ssp., Anticarsia gemmatalis, Megacopta spp., Megascelis ssp., Procornitermes ssp., Gryllotalpidae, Nezara viridula, Piezodorus spp., Acrosternum spp., Neomegalotomus spp., Cerotoma trifurcata, Popillia japonica, Edessa spp., Liogenys fuscus, Euschistus heros, stem borer, Dectes spp, stalk borer, Scaptocoris castanea, phyllophaga spp., Pseudoplusia includens, Spodoptera spp., Bemisia tabaci, Agriotes spp., preferably Diloboderus abderus, Diabrotica speciosa, Nezara viridula, Piezodorus spp., Acrosternum spp., Cerotoma trifurcata, Popillia japonica, Euschistus heros, phyllophaga spp., Agriotes spp, Dectes texanus.

The compounds of the invention are preferably used on soybean to control stinkbugs, e.g. Nezara spp. (e.g. Nezara viridula, Nezara antennata, Nezara hilare), Piezodorus spp. (e.g. Piezodorus guildinii), Acrosternum spp. (e.g. Acrosternum hilare), Euschistus spp. {e.g. Euschistus heros, Euschistus servus), Halyomorpha halys, Megacopta cribaria, Plautia crossota, Riptortus clavatus, Rhopalus maculatus, Antestiopsis orbitalus, Dectes texanus, Dichelops spp. (e.g. Dichelops furcatus, Dichelops melacanthus), Eurygaster spp. (e.g. Eurygaster intergriceps, Eurygaster maurd), Oebalus spp. (e.g. Oebalus mexicana, Oebalus poecilus, Oebalus pugnase, Podisus sp., (e.g. Podisus maculiventris), Scotinophara spp. (e.g. Scotinophara lurida, Scotinophara coarctatd), Thyanta spp. (e.g. Thyanta accerra). Preferred targets include Acrosternum hilare, Antestiopsis orbitalus, Dichelops furcatus, Dichelops melacanthus, Euschistus heros, Euschistus servus, Megacopta cribaria, Nezara viridula, Nezara hilare, Piezodorus guildinii, Halyomorpha halys, Thyanta accerra, Podisus maculiventris, Megacopta cribaria. More preferred targets include Acrosternum hilare, Antestiopsis orbitalus, Dichelops furcatus, Dichelops melacanthus, Euschistus heros, Euschistus servus, Megacopta cribaria, Nezara viridula, Nezara hilare, Piezodorus guildinii, Halyomorpha halys. In one embodiment the stinkbug target is Nezara viridula, Piezodorus spp., especially Piezodorus guildinii, Acrosternum spp, Euschistus heros, and in particular Nezara viridula and/or Euschistus heros. The compounds of the invention are particularly effective against Euschistus and in particular Euschistus heros. Euschistus and in particular Euschistus heros are the preferred targets. The compounds of the invention are also particularly effective against Halyomorpha halys.

### Applications

Application is of the compounds of the invention is to a crop of soybean plants, the locus thereof or propagation material thereof. Preferably application is to a crop of soybean plants or the locus thereof, more preferably to a crop of soybean plants. Application may be before infestation or when the pest is present. Application of the compounds of the invention can be performed according to any of the usual modes of application, e.g. foliar, drench, soil, in furrow etc. However, control of stinkbugs is usually achieved by foliar application, which is the preferred mode of application according to the invention.

The pest, i.e. the stink bugs, the plant, soil or water in which the plant is growing can be contacted with the present compounds of formula I or composition(s) containing them by any further application method known in the art. As such, “contacting” includes both direct contact (applying the compounds/compositions directly on the animal pest or plant—typically to the foliage, stem or roots of the plant) and indirect contact (applying the compounds/compositions to the locus of the animal pest or plant).

The compounds of formula I and their individual embodiments or the pesticidal compositions comprising them may be used to protect growing plants and crops from attack or infestation by animal pests, especially from stink bugs, in particular from Euschistus, more particularly from E. heros, by contacting the plant/crop with a pesticidally effective amount of compounds of formula I. The term “crop” refers both to growing and harvested crops.

The compounds of the invention may be applied in combination with an attractant. An attractant is a chemical that causes the insect to migrate towards the location of application. For control of stinkbugs it can be advantageous to apply the compounds of the invention with an attractant, particularly when the application is foliar. Stinkbugs are often located near to the ground, and application of an attractant may encourage migration up the plant towards the active ingredient. Suitable attractants include glucose, sacchrose, salt, glutamate (e.g. Aji-No-Moto™), citric acid (e.g. Orobor™), soybean oil, peanut oil and soybean milk. Glutamate and citric acid are of particular interest, with citric acid being preferred.

An attractant may be premixed with the compound of the invention prior to application, e.g. as a readymix or tankmix, or by simultaneous application or sequential application to the plant. Suitable rates of attractants are for example 0.02 kg/ha-3 kg/ha.

The compounds of the invention are preferably used for pest control on soybean at 1:500 g/ha, preferably 10-70 g/ha.

The compounds of the invention are suitable for use on any soybean plant, including those that have been genetically modified to be resistant to active ingredients such as herbicides, or to produce biologically active compounds that control infestation by plant pests.

The compounds of the invention are preferably used for pest control on soybean at 1:500 g/ha, preferably 10-70 g/ha.

The compounds of the invention are suitable for use on any soybean plant, including those that have been genetically modified to be resistant to active ingredients such as herbicides, or to produce biologically active compounds that control infestation by plant pests.

In a further preferred embodiment, transgenic plants and plant cultivars obtained by genetic engineering methods, if appropriate in combination with conventional methods (Genetically Modified Organisms), and parts thereof, are treated. Particularly preferably, plants of the plant cultivars which are in each case commercially available or in use are treated according to the invention. Plant cultivars are understood as meaning plants having novel properties (“traits”) which have been obtained by conventional breeding, by mutagenesis or by recombinant DNA techniques.

These can be cultivars, bio- or genotypes. Depending on the plant species or plant cultivars, their location and growth conditions (soils, climate, vegetation period, diet), the treatment according to the invention may also result in superadditive “synergistic”) effects.

Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The preferred transgenic plants or plant cultivars (obtained by genetic engineering) which are to be treated according to the invention include all plants which, by virtue of the genetic modification, received genetic material which imparts particularly advantageous, useful traits to these plants.

Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products.

Further and particularly emphasized examples of such traits are a better defense of the plants against animal and microbial pests, such as against insects, mites, phytopathogenic fungi, bacteria and/or viruses, and also increased tolerance of the plants to certain herbicidally active compounds.

Traits that are emphasized in particular are the increased defense of the plants against insects, arachnids, nematodes and slugs and snails by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (referred to herein as “Bt plants”). Traits that are also particularly emphasized are the increased defense of the plants against fungi, bacteria and viruses by systemic acquired resistance (SAR), systemin, phytoalexins, elicitors and resistance genes and correspondingly expressed proteins and toxins.

Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants.

Examples of “Bt plants” are soya bean varieties which are sold under the trade names YIELD GARD(®)

Examples of herbicide-tolerant plants which may be mentioned are soya bean varieties which are sold under the trade names Roundup Ready(®) (tolerance to glyphosate), Liberty Link(®) (tolerance to phosphinotricin), IMI(®) (tolerance to imidazolinones) and STS(®) (tolerance to sulphonylureas).

Herbicide-resistant plants (plants bred in a conventional manner for herbicide tolerance) which may be mentioned include the varieties sold under the name Clearfield(®) (for example maize).

Of particular interest are soybean plants carrying trains conferring resistance to 2 AO (e.g. Enlist®), glyphosate (e.g. Roundup Ready®, Roundup Ready 2 Yield®), sulfonylurea (e.g. STS®), glufosinate (e.g. Liberty Link®, Ignite®), Dicamba (Monsanto) HPPD tolerance (e.g. isoxaflutole herbicide) (Bayer CropScience, Syngenta). Double or triple stack in soybean plants of any of the traits described here are also of interest, including glyphosate and sulfonyl-urea tolerance (e.g. Optimum GAT®, plants stacked with STS® and Roundup Ready® or Roundup Ready 2 Yield®), dicamba and glyphosate tolerance (Monsanto). Soybean Cyst Nematode resistance soybean (SCN®-Syngenta) and soybean with Aphid resistant trait (AMT®-Syngneta) are also of interest.

These statements also apply to plant cultivars having these genetic traits or genetic traits still to be developed, which plant cultivars will be developed and/or marketed in the future.

### Mixtures

The compounds of formula (I) may be applied in the methods of the present invention in mixtures with fertilizers (for example nitrogen-, potassium- or phosphorus-containing fertilizers). Suitable formulation types include granules of fertilizer. The mixtures preferably contain up to 25% by weight of the compound of the invention.

The compositions of this invention may contain other compounds having biological activity, for example micronutrients or compounds having fungicidal activity or which possess plant growth regulating, herbicidal, insecticidal, nematicidal or acaricidal activity.

The compounds applied in the methods of the present invention may be the sole active ingredient of the composition or it may be admixed with one or more additional active ingredients such as a pesticide, fungicide, synergist, herbicide or plant growth regulator where appropriate. An additional active ingredient may: provide a composition having a broader spectrum of activity or increased persistence at a locus; synergize the activity or complement the activity (for example by increasing the speed of effect or overcoming repellency) of the compound of the invention; or help to overcome or prevent the development of resistance to individual components. The particular additional active ingredient will depend upon the intended utility of the composition.

According to one embodiment of the present invention, individual components of the composition according to the invention such as parts of a kit or parts of a binary or ternary mixture may be mixed by the user himself in a spray tank and further auxiliaries may be added, if appropriate.

In a further embodiment, either individual components of the composition according to the invention or partially premixed components, e.g. components comprising compounds I and/or active substances from the groups M.1 to M.UN.X or F.I to F.XII, may be mixed by the user in a spray tank and further auxiliaries and additives may be added, if appropriate.

In a further embodiment, either individual components of the composition according to the invention or partially premixed components, e.g. components comprising compounds I and/or active substances from the groups M.1 to M.UN.X or F.I to F.XII, can be applied jointly (e.g. after tank mix) or consecutively.

The following list M of pesticides, grouped according the Mode of Action Classification of the Insecticide Resistance Action Committee (IRAC), together with which the compounds according to the invention can be used and with which potential synergistic effects might be produced, is intended to illustrate the possible combinations, but not to impose any limitation:
M.1 Acetylcholine esterase (AChE) inhibitors from the class of
M.1A carbamates, for example aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; or from the class of
M.1B organophosphates, for example acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion, parathionmethyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion;
M.2. GABA-gated chloride channel antagonists such as:
M.2A cyclodiene organochlorine compounds, as for example endosulfan or chlordane; or
M.2B fiproles (phenylpyrazoles), as for example ethiprole, fipronil, flufiprole, pyrafluprole and pyriprole;
M.3 Sodium channel modulators from the class of
M.3A pyrethroids, for example acrinathrin, allethrin, d-cis-trans allethrin, d-trans allethrin, bifenthrin, bioallethrin, bioallethrin S-cylclopentenyl, bioresmethrin, cycloprothrin, cyfluthrin, betacyfluthrin, cyhalothrin, lambda-cyhalothrin, gamma-cyhalothrin, cypermethrin, alphacypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, meperfluthrin,metofluthrin, momfluorothrin, permethrin, phenothrin, prallethrin, profluthrin, pyrethrin (pyrethrum), resmethrin, silafluofen, tefluthrin, tetramethylfluthrin, tetramethrin, tralomethrin and transfluthrin; or
M.3B sodium channel modulators such as DDT or methoxychlor;
M.4 Nicotinic acetylcholine receptor agonists (nAChR) from the class of
M.4A neonicotinoids, for example acteamiprid, chlothianidin, dinotefuran, imidacloprid, nitenpyram, thiacloprid and thiamethoxam; or the compounds
M.4A.1: 1-[(6-chloro-3-pyridinyl)methyl]-2,3,5,6,7,8-hexahydro-9-nitro-(5S,8R)-5,8-Epoxy-1H-imidazo[1,2-a]azepine; or
M.4A.2: 1-[(6-chloro-3-pyridyl)methyl]-2-nitro-1-[(E)-pentylideneamino]guanidine; or
M4.A.3: 1-[(6-chloro-3-pyridyl)methyl]-7-methyl-8-nitro-5-propoxy-3,5,6,7-tetrahydro-2H-imidazo[1,2-a]pyridine;
or M.4B nicotine.
M.5 Nicotinic acetylcholine receptor allosteric activators from the class of spinosyns, for example spinosad or spinetoram;
M.6 Chloride channel activators from the class of avermectins and milbemycins, for example abamectin, emamectin benzoate, ivermectin, lepimectin or milbemectin;
M.7 Juvenile hormone mimics, such as
M.7A juvenile hormone analogues as hydroprene, kinoprene and methoprene; or others as
M.7B fenoxycarb or M.7C pyriproxyfen;
M.8 miscellaneous non-specific (multi-site) inhibitors, for example
M.8A alkyl halides as methyl bromide and other alkyl halides, or
M.8B chloropicrin, or M.8C sulfuryl fluoride, or M.8D borax, or M.8E tartar emetic;
M.9 Selective homopteran feeding blockers, for example
M.9B pymetrozine, or M.9C flonicamid;
M.10 Mite growth inhibitors, for example
M.10A clofentezine, hexythiazox and diflovidazin, or M.10B etoxazole;
M.11 Microbial disruptors of insect midgut membranes, for example bacillus thuringiensis or bacillus sphaericus and the insecticdal proteins they produce such as bacillus thuringiensis subsp. israelensis, bacillus sphaericus, bacillus thuringiensis subsp. aizawai, bacillus thuringiensis subsp. kurstaki and bacillus thuringiensis subsp. tenebrionis, or the Bt crop proteins: Cry1Ab, Cry1Ac, Cry1Fa, Cry2Ab, mCry3A, Cry3Ab, Cry3Bb and Cry34/35Ab1;
M.12 Inhibitors of mitochondrial ATP synthase, for example
M.12A diafenthiuron, or
M.12B organotin miticides such as azocyclotin, cyhexatin or fenbutatin oxide, or M.12C propargite, or M.12D tetradifon;
M.13 Uncouplers of oxidative phosphorylation via disruption of the proton gradient, for example chlorfenapyr, DNOC or sulfluramid;
M.14 Nicotinic acetylcholine receptor (nAChR) channel blockers, for example nereistoxin analogues as bensultap, cartap hydrochloride, thiocyclam or thiosultap sodium;
M.15 Inhibitors of the chitin biosynthesis type 0, such as benzoylureas as for example bistrifluron, chlorfluazuron, diflubenzuron, flucycloxuron, flufenoxuron, hexaflumuron, lufenuron, novaluron, noviflumuron, teflubenzuron or triflumuron;
M.16 Inhibitors of the chitin biosynthesis type 1, as for example buprofezin;
M.17 Moulting disruptors, Dipteran, as for example cyromazine;
M.18 Ecdyson receptor agonists such as diacylhydrazines, for example methoxyfenozide, tebufenozide, halofenozide, fufenozide or chromafenozide;
M.19 Octopamin receptor agonists, as for example amitraz;
M.20 Mitochondrial complex III electron transport inhibitors, for example
M.20A hydramethylnon, or M.20B acequinocyl, or M.20C fluacrypyrim;
M.21 Mitochondrial complex I electron transport inhibitors, for example
M.21A METI acaricides and insecticides such as fenazaquin, fenpyroximate, pyrimidifen, pyridaben, tebufenpyrad or tolfenpyrad, or M.21B rotenone;
M.22 Voltage-dependent sodium channel blockers, for example
M.22A indoxacarb, or M.22B metaflumizone, or M.22C 1-[(E)-[2-(4-cyanophenyl)-1-[3-(trifluoromethyl)phenyl]ethylidene]amino]-3-[4-(difluoromethoxy)phenyl]urea;
M.23 Inhibitors of the of acetyl CoA carboxylase, such as Tetronic and Tetramic acid derivatives, for example spirodiclofen, spiromesifen or spirotetramat;
M.24 Mitochondrial complex IV electron transport inhibitors, for example
M.24A phosphine such as aluminium phosphide, calcium phosphide, phosphine or zinc phosphide, or M.24B cyanide.
M.25 Mitochondrial complex II electron transport inhibitors, such as beta-ketonitrile derivatives, for example cyenopyrafen or cyflumetofen;
M.28 Ryanodine receptor-modulators from the class of diamides, as for example flubendiamide, chlorantraniliprole (Rynaxypyr®), cyantraniliprole (Cyazypyr®), or the phthalamide compounds
M.28.1: (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and
M.28.2: (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, or the compound
M.28.3: 3-bromo-N-{2-bromo-4-chloro-6-[(1-cyclopropylethyl)carbamoyl]phenyl}-1-(3-chlorpyridin-2-yl)-1H-pyrazole-5-carboxamide (proposed ISO name: cyclaniliprole), or the compound
M.28.4: methyl-2-[3,5-dibromo-2-({[3-bromo-1-(3-chlorpyridin-2-yl)-1H-pyrazol-5-yl]carbonyl}amino)benzoyl]-1,2-dimethylhydrazinecarboxylate; or a compound selected from M.28.5a) to M.28.5l):
M.28.5a) N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide;
M.28.5b) N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide;
M.28.5c) N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide;
M.28.5d) N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide;
M.28.5e) N-[4,6-dichloro-2-[(diethyl-lambda-4-sulfanylidene)carbamoyl]-phenyl]-2-(3-chloro-2-pyridyl)-5-(difluoromethyl)pyrazole-3-carboxamide;
M.28.5f) N-[4,6-dibromo-2-[(di-2-propyl-lambda-4-sulfanylidene)carbamoyl]-phenyl]-2-(3-chloro-2-pyridyl)-5-(trifluoromethyl)pyrazole-3-carboxamide;
M.28.5g) N-[4-chloro-2-[(di-2-propyl-lambda-4-sulfanylidene)carbamoyl]-6-cyano-phenyl]-2-(3-chloro-2-pyridyl)-5-(trifluoromethyl)pyrazole-3-carboxamide;
M.28.5h) N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide;
M.28.5i) N-[2-(5-amino-1,3,4-thiadiazol-2-yl)-4-chloro-6-methyl-phenyl]-5-bromo-2-(3-chloro-2-pyridyl)pyrazole-3-carboxamide;
M.28.5j) 5-chloro-2-(3-chloro-2-pyridyl)-N-[2,4-dichloro-6-[(1-cyano-1-methyl-ethyl)carbamoyl]phenyl]pyrazole-3-carboxamide;
M.28.5k) 5-bromo-N-[2,4-dichloro-6-(methylcarbamoyl)phenyl]-2-(3,5-dichloro-2-pyridyl)pyrazole-3-carboxamide;
M.28.5l) N-[2-(tert-butylcarbamoyl)-4-chloro-6-methyl-phenyl]-2-(3-chloro-2-pyridyl)-5-(fluoromethoxy)pyrazole-3-carboxamide; or a compound selected from
M.28.6 N2-(1-cyano-1-methyl-ethyl)-N1-(2,4-dimethylphenyl)-3-iodo-phthalamide; or
M.28.7 3-chloro-N2-(1-cyano-1-methyl-ethyl)-N1-(2,4-dimethylphenyl)phthalamide;
M.UN.X insecticidal active compounds of unknown or uncertain mode of action, as for example azadirachtin, amidoflumet, benzoximate, bifenazate, bromopropylate, chinomethionat, cryolite, dicofol, flufenerim, flometoquin, fluensulfone, flupyradifurone, piperonyl butoxide, pyridalyl, pyrifluquinazon, sulfoxaflor, pyflubumide or the compounds
M.UN.X.1: 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide, or the compound
M.UN.X.2: cyclopropaneacetic acid, 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl]ester, or the compound
M.UN.X.3: 11-(4-chloro-2,6-dimethylphenyl)-12-hydroxy-1,4-dioxa-9-azadispiro[4.2.4.2]-tetradec-11-en-10-one, or the compound
M. UN.X.4: 3-(4′-fluoro-2,4-dimethylbiphenyl-3-yl)-4-hydroxy-8-oxa-1-azaspiro[4.5]dec-3-en-2-one, or the compound
M.UN.X.5: 1-[2-fluoro-4-methyl-5-[(2,2,2-trifluoroethyl)sulfinyl]phenyl]-3-(trifluoromethyl)-1H-1,2,4-triazole-5-amine, or actives on basis of bacillus firmus (Votivo, 1-1582); or M.UN.X.6; a compound selected from the group of
M.UN.X.6a) (E/Z)—N-[1-[(6-chloro-3-pyridyl)methyl]-2-pyridylidene]-2,2,2-trifluoro-acetamide;
M.UN.X.6b) (E/Z)—N-[1-[(6-chloro-5-fluoro-3-pyridyl)methyl]-2-pyridylidene]-2,2,2-trifluoro-acetamide;
M.UN.X.6c) (E/Z)-2,2,2-trifluoro-N-[1-[(6-fluoro-3-pyridyl)methyl]-2-pyridylidene]acetamide;
M.UN.X.6d) (E/Z)—N-[1-[(6-bromo-3-pyridyl)methyl]-2-pyridylidene]-2,2,2-trifluoro-acetamide;
M. UN.X.6e) (E/Z)—N-[1-[1-(6-chloro-3-pyridyl)ethyl]-2-pyridylidene]-2,2,2-trifluoro-acetamide;
M.UN.X.6f) (E/Z)—N-[1-[(6-chloro-3-pyridyl)methyl]-2-pyridylidene]-2,2-difluoro-acetamide;
M.UN.X.6g) (E/Z)-2-chloro-N-[1-[(6-chloro-3-pyridyl)methyl]-2-pyridylidene]-2,2-difluoro-acetamide;
M.UN.X.6h) (E/Z)—N-[1-[(2-chloropyrimidin-5-yl)methyl]-2-pyridylidene]-2,2,2-trifluoro-acetamide and
M.UN.X.6i) (E/Z)—N-[1-[(6-chloro-3-pyridyl)methyl]-2-pyridylidene]-2,2,3,3,3-pentafluoropropanamide); or the compounds
M.UN.X.7: 3-[3-chloro-5-(trifluoromethyl)phenyl]-4-oxo-1-(pyrimidin-5-ylmethyl)pyrido[1,2-a]pyrimidin-1-ium-2-olate; or
M.UN.X.8: 8-chloro-N-[2-chloro-5-methoxyphenyl)sulfonyl]-6-trifluoromethyl)-imidazo[1,2-a]pyridine-2-carboxamide; or
M.UN.X.9: 4-[5-(3,5-dichlorophenyl)-5-(trifluoromethyl)-4H-isoxazol-3-yl]-2-methyl-N-(1-oxothietan-3-yl)benzamide; or
M.UN.X.10: 5-[3-[2,6-dichloro-4-(3,3-dichloroallyloxy)phenoxy]propoxy]-1H-pyrazole; or
M. UN.X.11: 4-[5-(3,5-dichlorophenyl)-5-(trifluoromethyl)-4H-isoxazol-3-yl]-2-methyl-N-[2-oxo-2-(2,2,2-trifluoroethylamino)ethyl]benzamide; or
M.UN.X.12: 4-[5-[3-chloro-5-(trifluoromethyl)phenyl]-5-(trifluoromethyl)-4H-isoxazol-3-yl]-N-[2-oxo-2-(2,2,2-trifluoroethylamino)ethyl]naphthalene-1-carboxamide.

The commercially available compounds of the group M listed above may be found in The Pesticide Manual, 15th Edition, C. D. S. Tomlin, British Crop Protection Council (2011) among other publications.

The quinoline derivative flometoquin is shown in WO2006/013896. The aminofuranone compounds flupyradifurone is known from WO 2007/115644. The sulfoximine compound sulfoxaflor is known from WO2007/149134. The acaricide pyflubumide is known from WO2007/020986. The isoxazoline compounds have been described: M.UN.X.1 in WO2005/085216, M.UN.X.9 in WO2013/050317, M.UN.X.11 in WO2005/085216 and M.UN.X. in WO2009/002809 and in WO2011/149749. The pyripyropene derivative M.UN.X.2 has been described in WO 2006/129714. The spiroketal-substituted cyclic ketoenol derivative M.UN.X.3 is known from WO2006/089633 and the biphenyl-substituted spirocyclic ketoenol derivative M.UN.X.4 from WO2008/067911. Finally triazoylphenylsulfide like M.UN.X.5 have been described in WO2006/043635 and biological control agents on basis of bacillus firmus in WO2009/124707. The neonicotionids 4A.1 is known from WO20120/069266 and WO2011/06946, the M.4.A.2 from WO2013/003977, the M4.A.3.from WO2010/069266.

The Metaflumizone analogue M.22C is described in CN 10171577. The phthalamides M.28.1 and M.28.2 are both known from WO 2007/101540. The anthranilamide M.28.3 has been described in WO2005/077934. The hydrazide compound M.28.4 has been described in WO 2007/043677. The anthranilamides M.28.5a) to M.28.5h) can be prepared as described in WO 2007/006670, WO2013/024009 and WO2013/024010, the anthranilamide M.28.5i) is described in WO2011/085575, the M.28.5j) in WO2008/134969, the M.28.5k) in US2011/046186 and the M.28.5l) in WO2012/034403. The diamide compounds M.28.6 and M.28.7 can be found in CN102613183.

The compounds M.UN.X.6a) to M.UN.X.6i) listed in M.UN.X.6 have been described in WO2012/029672. The mesoionic antagonist compound M.UN.X.7 was described in WO2012/092115, the nematicide M.UN.X.8 in WO2013/055584 and the Pyridalyl-type analogue M.UN.X.10 in WO2010/060379.

Preferred additional pesticidally active ingredients are those selected from the IRAC group 1, the Acetylcholinesterase (AChE) inhibitors, herein from the group 1A (Carbamtes) Thiodicarb, Methomyl and Carbaryl, and from the group 1B(Organophosphates), especially Acephate, Chlorpyriphos and Dimethoate, from the group 2B, the fiproles, here especially ethiprole and fipronil, from the group 3, the pyrethroids, here especially lambda-cyhalothrin, alpha-cypermethrin or deltametrin, and from the group 4A, the neonicotinoids, here especially acetamiprid, clothianidin, dinotefuran, imidacloprid, nitenpyram, thiacloprid or thiomethoxam.

Especially combinations of compounds of the invention with fiproles, neonictinoids or pyrethroids may possibly exhibit synergistic control of stinkbugs (according to the Colby formula), in particular Euschistus, e.g. Euschistus heros.

The following list F of active substances, in conjunction with which the compounds according to the invention can be used, is intended to illustrate the possible combinations but does not limit them:

### F.I) Respiration Inhibitors

F.I-1) Inhibitors of complex III at Qo site:
strobilurins: azoxystrobin, coumethoxystrobin, coumoxystrobin, dimoxystrobin, enestroburin, fluoxastrobin, kresoxim-methyl, metominostrobin, orysastrobin, picoxystrobin, pyraclostrobin, pyrametostrobin, pyraoxystrobin, pyribencarb, triclopyricarb/chlorodincarb, trifloxystrobin, 2-[2-(2,5-dimethyl-phenoxymethyl)-phenyl]-3-methoxy-acrylic acid methyl ester and 2 (2-(3-(2,6-dichlorophenyl)-1-methyl-allylideneaminooxymethyl)-phenyl)-2-methoxyimino-N methylacetamide;
oxazolidinediones and imidazolinones: famoxadone, fenamidone;
F.I-2) Inhibitors of complex II (e.g. carboxamides):
carboxanilides: benodanil, benzovindiflupyr, bixafen, boscalid, carboxin, fenfuram, fenhexamid, fluopyram, flutolanil, furametpyr, isopyrazam, isotianil, mepronil, oxycarboxin, penflufen, penthiopyrad, sedaxane, tecloftalam, thifluzamide, tiadinil, 2-amino-4 methyl-thiazole-5-carboxanilide, N-(3′,4′,5′ trifluorobiphenyl-2 yl)-3-difluoromethyl-1-methyl-1H-pyrazole-4 carboxamide(fluxapyroxad), N-(4′-trifluoromethylthiobiphenyl-2-yl)-3 difluoromethyl-1-methyl-1H pyrazole-4-carboxamide, N-(2-(1,3,3-trimethyl-butyl)-phenyl)-1,3-dimethyl-5 fluoro-1H-pyrazole-4 carboxamide, 3-(difluoromethyl)-1-methyl-N-(1,1,3-trimethylindan-4-yl)pyrazole-4-carboxamide, 3-(trifluoromethyl)-1-methyl-N-(1,1,3-trimethylindan-4-yl)pyrazole-4-carboxamide, 1,3-dimethyl-N-(1,1,3-trimethylindan-4-yl)pyrazole-4-carboxamide, 3-(trifluoromethyl)-1,5-dimethyl-N-(1,1,3-trimethylindan-4-yl)pyrazole-4-carboxamide, 3-(difluoromethyl)-1,5-dimethyl-N-(1,1,3-trimethylindan-4-yl)pyrazole-4-carboxamide, 1,3,5-trimethyl-N-(1,1,3-trimethylindan-4-yl)pyrazole-4-carboxamide, 3-(difluoromethyl)-1-methyl-N-(1,1,3-trimethylindan-4-yl)pyrazole-4-carboxamide, 3-(trifluoromethyl)-1-methyl-N-(1,1,3-trimethylindan-4-yl)pyrazole-4-carboxamide, 1,3-dimethyl-N-(1,1,3-trimethylindan-4-yl)pyrazole-4-carboxamide, 3-(trifluoromethyl)-1,5-dimethyl-N-(1,1,3-trimethylindan-4-yl)pyrazole-4-carboxamide, 3-(difluoromethyl)-1,5-dimethyl-N-(1,1,3-trimethylindan-4-yl)pyrazole-4-carboxamide, 1,3,5-trimethyl-N-(1,1,3-trimethylindan-4-yl)pyrazole-4-carboxamide;
F.I-3) Inhibitors of complex III at Qi site: cyazofamid, amisulbrom, [(3S,6S,7R,8R)-8-benzyl-3-[(3-acetoxy-4-methoxy-pyridine-2-carbonyl)amino]-6-methyl-4,9-dioxo-1,5-dioxonan-7-yl]2-methylpropanoate, [(3S,6S,7R,8R)-8-benzyl-3-[[3-(acetoxymethoxy)-4-methoxy-pyridine-2-carbonyl]amino]-6-methyl-4,9-dioxo-1,5-dioxonan-7-yl]2-methylpropanoate, [(3S,6S,7R,8R)-8-benzyl-3-[(3-isobutoxycarbonyloxy-4-methoxy-pyridine-2-carbonyl)amino]-6-methyl-4,9-dioxo-1,5-dioxonan-7-yl]2-methylpropanoate, [(3S,6S,7R,8R)-8-benzyl-3-[[3-(1,3-benzodioxol-5-ylmethoxy)-4-methoxy-pyridine-2-carbonyl]amino]-6-methyl-4,9-dioxo-1,5-dioxonan-7-yl]2-methylpropanoate, 3S,6S,7R,8R)-3-[[(3-hydroxy-4-methoxy-2-pyridinyl)carbonyl]amino]-6-methyl-4,9-dioxo-8-(phenylmethyl)-1,5-dioxonan-7-yl 2-methylpropanoate;
F.I-4) Other respiration inhibitors (complex I, uncouplers) diflumetorim; (5,8-difluoroquinazolin-4-yl)-{2-[2-fluoro-4-(4-trifluoromethylpyridin-2-yloxy)-phenyl]-ethyl}-amine; tecnazen; ametoctradin; silthiofam; nitrophenyl derivates: binapacryl, dinobuton, dinocap, fluazinam, ferimzone, nitrthalisopropyl,
and including organometal compounds: fentin salts, such as fentin-acetate, fentin chloride or fentin hydroxide;
F.II) Sterol biosynthesis inhibitors (SBI fungicides)
F.II-1) C14 demethylase inhibitors (DMI fungicides, e.g. triazoles, imidazoles) triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, paclobutrazole, penconazole, propiconazole, prothioconazole, simeconazole, tebuconazole, tetraconazole, triadimefon, triadimenol, triticonazole, uniconazole, 1-[rel-(2S;3R)-3-(2-chlorophenyl)-2-(2,4-difluorophenyl)-oxiranylmethyl]-5-thiocyanato-1H-[1,2,4]triazole, 2-[rel(2S;3R)-3-(2-chlorophenyl)-2-(2,4-difluorophenyl)-oxiranylmethyl]-2H-[1,2,4]triazole-3-thiol;
imidazoles: imazalil, pefurazoate, oxpoconazole, prochloraz, triflumizole;
pyrimidines, pyridines and piperazines: fenarimol, nuarimol, pyrifenox, triforine, 1-[rel-(2S,3R)-3-(2-chlorophenyl)-2-(2,4-difluorophenyl)-oxiranylmethyl]-5-thiocyanato-1H-[1,2,4]triazole, 2-[rel(2S;3R)-3-(2-chlorophenyl)-2-(2,4-difluorophenyl)-oxiranylmethyl]-2H-[1,2,4]triazole-3-thiol;
F.II-2) Delta14-reductase inhitors (Amines, e.g. morpholines, piperidines)
morpholines: aldimorph, dodemorph, dodemorph-acetate, fenpropimorph, tridemorph; piperidines: fenpropidin, piperalin; spiroketalamines: spiroxamine;
F.II-3) Inhibitors of 3-keto reductase: hydroxyanilides: fenhexamid;
F.III) Nucleic acid synthesis inhibitors
F.III-1) RNA, DNA synthesis
phenylamides or acyl amino acid fungicides: benalaxyl, benalaxyl-M, kiralaxyl, metalaxyl, metalaxyl-M (mefenoxam), ofurace, oxadixyl;
isoxazoles and iosothiazolones: hymexazole, octhilinone;
F.III-2) DNA topisomerase inhibitors: oxolinic acid;
F.III-3) Nucleotide metabolism (e.g. adenosin-deaminase), hydroxy(2-amino)-pyrimidines: bupirimate;
F.IV) Inhibitors of cell division and or cytoskeleton
F.IV-1) Tubulin inhibitors: benzimidazoles and thiophanates: benomyl, carbendazim, fuberidazole, thiabendazole, thiophanate-methyl;
triazolopyrimidines: 5-chloro-7 (4-methylpiperidin-1-yl)-6-(2,4,6-trifluorophenyl)[1,2,4]triazolo[1,5a]pyrimidine;
F.IV-2) Other cell division inhibitors
benzamides and phenyl acetamides: diethofencarb, ethaboxam, pencycuron, fluopicolide, zoxamide;
F.IV-3) Actin inhibitors: benzophenones: metrafenone, pyriofenone;
F.V) Inhibitors of amino acid and protein synthesis
F.V-1) Methionine synthesis inhibitors (anilino-pyrimidines)
anilino-pyrimidines: cyprodinil, mepanipyrim, nitrapyrin, pyrimethanil;
F.V-2) Protein synthesis inhibitors (anilino-pyrimidines)
antibiotics: blasticidin-S, kasugamycin, kasugamycin hydrochloride-hydrate, mildiomycin, streptomycin, oxytetracyclin, polyoxine, validamycin A;
F.VI) Signal transduction inhibitors
F.VI-1) MAP/Histidine kinase inhibitors (e.g. anilino-pyrimidines)
dicarboximides: fluoroimid, iprodione, procymidone, vinclozolin;
phenylpyrroles: fenpiclonil, fludioxonil;
F.VI-2) G protein inhibitors: quinolines: quinoxyfen;
F.VII) Lipid and membrane synthesis inhibitors
F.VII-1) Phospholipid biosynthesis inhibitors
organophosphorus compounds: edifenphos, iprobenfos, pyrazophos;
dithiolanes: isoprothiolane;
F.VII-2) Lipid peroxidation: aromatic hydrocarbons: dicloran, quintozene, tecnazene, tolclofosmethyl, biphenyl, chloroneb, etridiazole;
F.VII-3) Carboxyl acid amides (CAA fungicides)
cinnamic or mandelic acid amides: dimethomorph, flumorph, mandiproamid, pyrimorph; valinamide carbamates: benthiavalicarb, iprovalicarb, pyribencarb, valifenalate and N-(1-(1-(4-cyano-phenyl)ethanesulfonyl)-but-2-yl) carbamic acid-(4-fluorophenyl) ester;
F.VII-4) Compounds affecting cell membrane permeability and fatty acids: 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, carbamates: propamocarb, propamocarb-hydrochlorid,
F.VII-5) fatty acid amide hydrolase inhibitors: 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone;
F.VIII) Inhibitors with Multi Site Action
F.VIII-1) Inorganic active substances: Bordeaux mixture, copper acetate, copper hydroxide, copper oxychloride, basic copper sulfate, sulfur;
F.VIII-2) Thio- and dithiocarbamates: ferbam, mancozeb, maneb, metam, methasulphocarb, metiram, propineb, thiram, zineb, ziram;
F.VIII-3) Organochlorine compounds (e.g. phthalimides, sulfamides, chloronitriles): anilazine, chlorothalonil, captafol, captan, folpet, dichlofluanid, dichlorophen, flusulfamide, hexachlorobenzene, pentachlorphenole and its salts, phthalide, tolylfluanid, N-(4-chloro-2-nitrophenyl)-N-ethyl-4-methyl-benzenesulfonamide;
F.VIII-4) Guanidines and other: guanidine, dodine, dodine free base, guazatine, guazatineacetate, iminoctadine, iminoctadine-triacetate, iminoctadine-tris(albesilate), 2,6-dimethyl-1H,5H-[1,4]dithiino[2,3-c:5,6-c′]dipyrrole-1,3,5,7(2H,6H)-tetraone;
F.VIII-5) Ahtraquinones: dithianon;
F.IX) Cell wall synthesis inhibitors
F.IX-1) Inhibitors of glucan synthesis: validamycin, polyoxin B;
F.IX-2) Melanin synthesis inhibitors: pyroquilon, tricyclazole, carpropamide, dicyclomet, fenoxanil;
F.X) Plant defense inducers
F.X-1) Salicylic acid pathway: acibenzolar-S-methyl;
F.X-2) Others: probenazole, isotianil, tiadinil, prohexadione-calcium;
phosphonates: fosetyl, fosetyl-aluminum, phosphorous acid and its salts;
F.XI) Unknown mode of action:bronopol, chinomethionat, cyflufenamid, cymoxanil, dazomet, debacarb, diclomezine, difenzoquat, difenzoquat-methylsulfate, diphenylamin, fenpyrazamine, flumetover, flusulfamide, flutianil, methasulfocarb, nitrapyrin, nitrothal-isopropyl, oxathiapiprolin, oxin-copper, proquinazid, tebufloquin, tecloftalam, triazoxide, 2-butoxy-6-iodo-3-propylchromen-4-one, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N methyl formamidine, N′-(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2 methyl-4-(3-trimethylsilanyl-propoxy)-phenyl)-N-ethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl]-piperidin-4-yl}-thiazole-4-carboxylic acid methyl-(R)-1,2,3,4-tetrahydro-naphthalen-1-yl-amide, methoxy-acetic acid 6-tert-butyl-8-fluoro-2,3-dimethyl-quinolin-4-yl ester and N-Methyl-2-{1-[(5-methyl-3-trifluoromethyl-1H-pyrazol-1-yl)acetyl]-piperidin-4-yl}-N-[(1R)-1,2,3,4-tetrahydronaphthalen-1-yl]-4-thiazolecarboxamide, 3-[5-(4-chloro-phenyl)-2,3-dimethyl-isoxazolidin-3 yl]-pyridine, pyrisoxazole, 5-amino-2-isopropyl-3-oxo-4-ortho-tolyl-2,3-dihydro-pyrazole-1 carbothioic acid S-allyl ester, N-(6-methoxy-pyridin-3-yl)cyclopropanecarboxylic acid amide, 5-chloro-1 (4,6-dimethoxy-pyrimidin-2-yl)-2-methyl-1H-benzoimidazole, 2-(4-chloro-phenyl)-N-[4-(3,4-dimethoxy-phenyl)-isoxazol-5-yl]-2-prop-2-ynyloxy-acetamide.
F.XII) Growth regulators: abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N 6-benzyladenine, paclobutrazol, prohexadione (prohexadione-calcium), prohydrojasmon, thidiazuron, triapenthenol, tributyl phosphorotrithioate, 2,3,5 tri iodobenzoic acid, trinexapac-ethyl and uniconazole;
F.XIII) Biological control agents
Ampelomyces quisqualis (e.g. AQ 10® from Intrachem Bio GmbH & Co. KG, Germany), Aspergillus flavus (e.g. AFLAGUARD® from Syngenta, CH), Aureobasidium pullulans (e.g. BOTECTOR® from bio-ferm GmbH, Germany), Bacillus pumilus (e.g. NRRL Accession No. B-30087 in SONATA® and BALLAD® Plus from AgraQuest Inc., USA), Bacillus subtilis (e.g. isolate NRRL-Nr. B-21661 in RHAPSODY®, SERENADE® MAX and SERENADE® ASO from AgraQuest Inc., USA), Bacillus subtilis var. amyloliquefaciens FZB24 (e.g. TAEGRO® from Novozyme Biologicals, Inc., USA), Candida oleophila 1-82 (e.g. ASPIRE® from Ecogen Inc., USA), Candida saitoana (e.g. BIOCURE® (in mixture with lysozyme) and BIOCOAT® from Micro Flo Company, USA (BASF SE) and Arysta), Chitosan (e.g. ARMOUR-ZEN from BotriZen Ltd., NZ), Clonostachys rosea f. catenulata, also named Gliocladium catenulatum (e.g. isolate J1446: PRESTOP® from Verdera, Finland), Coniothyrium minitans (e.g. CONTANS® from Prophyta, Germany), Cryphonectria parasitica (e.g. Endothia parasitica from CNICM, France), Cryptococcus albidus (e.g. YIELD PLUS® from Anchor Bio-Technologies, South Africa), Fusarium oxysporum (e.g. BIOFOX® from S.I.A.P.A., Italy, FUSACLEAN® from Natural Plant Protection, France), Metschnikowia fructicola (e.g. SHEMER® from Agrogreen, Israel), Microdochium dimerum (e.g. ANTIBOT® from Agrauxine, France), Phiebiopsis gigantea (e.g. ROTSOP® from Verdera, Finland), Pseudozyma flocculosa (e.g. SPORODEX® from Plant Products Co. Ltd., Canada), Pythium oligandrum DV74 (e.g. POLYVERSUM® from Remeslo SSRO, Biopreparaty, Czech Rep.), Reynoutria sachlinensis (e.g. REGALIA® from Marrone Biolnnovations, USA), Talaromyces flavus V117b (e.g. PROTUS® from Prophyta, Germany), Trichoderma asperellum SKT-1 (e.g. ECO-HOPE® from Kumiai Chemical Industry Co., Ltd., Japan), T. atroviride LC52 (e.g. SENTINEL® from Agrimm Technologies Ltd, NZ), T. harzianum T-22 (e.g. PLANTSHIELD® der Firma BioWorks Inc., USA), T. harzianum TH 35 (e.g. ROOT PRO® from Mycontrol Ltd., Israel), T. harzianum T-39 (e.g. TRICHODEX® and TRICHODERMA 2000® from Mycontrol Ltd., Israel and Makhteshim Ltd., Israel), T. harzianum and T. viride (e.g. TRICHOPEL from Agrimm Technologies Ltd, NZ), T. harzianum ICC012 and T. viride ICC080 (e.g. REMEDIER® WP from Isagro Ricerca, Italy), T. polysporum and T. harzianum (e.g. BINAB® from BINAB Bio-Innovation AB, Sweden), T. stromaticum (e.g. TRICOVAB® from C.E.P.L.A.C., Brazil), T. virens GL-21 (e.g. SOILGARD® from Certis LLC, USA), T. viride (e.g. TRIECO® from Ecosense Labs. (India) Pvt. Ltd., Indien, BIO-CURE® F from T. Stanes & Co. Ltd., Indien), T. viride TV1 (e.g. T. viride TV1 from Agribiotec srl, Italy), Ulocladium oudemansii HRU3 (e.g. BOTRY-ZEN® from Botry-Zen Ltd, NZ).

The commercially available compounds II of the group F listed above may be found in The Pesticide Manual, 15th Edition, C. D. S. Tomlin, British Crop Protection Council (2011) among other publications. Their preparation and their activity against harmful fungi is known (cf.: http://www.alanwood.net/pesticides/); these substances are commercially available. The compounds described by IUPAC nomenclature, their preparation and their fungicidal activity are also known (cf. Can. J. Plant Sci. 48(6), 587-94, 1968; EP A 141 317; EP-A 152 031; EP-A 226 917; EP A 243 970; EP A 256 503; EP-A 428 941; EP-A 532 022; EP-A 1 028 125; EP-A 1 035 122; EP A 1 201 648; EP A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 11/028657).

The compounds of the invention may be mixed with soil, peat or other rooting media for the protection of plants against seed-borne, soil-borne or foliar fungal diseases.

Examples of suitable synergists for use in the compositions include piperonyl butoxide, sesamex, safroxan and dodecyl imidazole.

Suitable herbicides and plant-growth regulators for inclusion in the compositions will depend upon the intended target and the effect required.

An example of a rice selective herbicide which may be included is propanil. An example of a plant growth regulator for use in cotton is PIX™.

Some mixtures may comprise active ingredients which have significantly different physical, chemical or biological properties such that they do not easily lend themselves to the same

### Formulations

The invention also relates to agrochemical compositions comprising one or more auxiliary agents and at least one compound of formula (I) and/or one of its individual embodiments, which are applied in the methods of the present invention.

An agrochemical composition comprises a pesticidally effective amount of a compound of formula (I) and/or one of its individual embodiments. The term “effective amount” denotes an amount of the composition or of the compounds I, which is sufficient for controlling animal pests on cultivated plants or in the protection of materials and which does not result in a substantial damage to the treated plants. Such an amount can vary in a broad range and is dependent on various factors, such as the pest species to be controlled, the treated cultivated plant or material, the climatic conditions and the specific com-pound I used.

The compounds I, their N-oxides and salts can be converted into customary types of agrochemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Mono-graph No. 2, 6th Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e.g. toluene, paraffin, tetrahydronaphthalene, alkylated naphthalenes; alcohols, e.g. ethanol, propanol, butanol, benzylalcohol, cyclo-hexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emusifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl-naphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compound of formula (I) on the target. Examples are surfactants, mineral or vegetable oils, and other auxilaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anor-ganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

The agrochemical compositions generally comprise between 0.01 and 95%, preferably between 0.1 and 90%, and in particular between 0.5 and 75%, by weight of active substance. The active substances are employed in a purity of from 90% to 100%, preferably from 95% to 100% (according to NMR spectrum).

Solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds. The compositions in question give, after two-to-tenfold dilution, active substance concentrations of from 0.01 to 60% by weight, preferably from 0.1 to 40% by weight, in the ready-to-use preparations. Application can be carried out before or during sowing. Methods for applying compound of formula (I) and compositions thereof, respectively, on to plant propagation material, especially seeds include dressing, coating, pelleting, dusting, soaking and in-furrow application methods of the propagation material. Preferably, compound of formula (I) or the compositions thereof, respectively, are applied on to the plant propagation material by a method such that germination is not induced, e.g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials such as seeds, e.g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank and further auxiliaries may be added, if appropriate.

In a further embodiment, either individual components of the composition according to the invention or partially premixed components, e.g. components comprising compounds I (or one of its individual embodiments) and/or active substances from the groups M.1 to M.UN.X mentioned herein above, may be mixed by the user in a spray tank and further auxiliaries and additives may be added, if appropriate.

In a further embodiment, either individual components of the composition according to the invention or partially premixed components, e.g. components comprising compounds I (or one of its individual embodiments) and/or active substances from the groups M.1 to M.UN.X mentioned herein above, can be applied jointly (e.g. after tank mix) or consecutively.

### EXAMPLES

The present invention is now illustrated in further details by the following examples, without imposing any limitation thereto.

### C. Compounds

The Compounds for being applied in the methods of the present invention can be characterized e.g. by coupled High Performance Liquid Chromatography/mass spectrometry (HPLC/MS) or by ¹H-NMR.

### Analytical HPLC Column:

Analytical UPLC column: Phenomenex Kinetex 1.7 μm XB-C18 100A; 50×2.1 mm from Phenomenex, Germany. Elution: acetonitrile+0.1% trifluoroacetic acid (TFA)/water+0.1% trifluoroacetic acid (TFA) in a ratio from 5:95 to 100:0 in 1.5 min at 60° C. Flow: 0.8 mL/min to 1 mL/min in 1.5 min. MS-method: ESI positive.

¹H-NMR: The signals are characterized by chemical shift (ppm, δ [delta]) vs. tetramethylsilane, by their multiplicity and by their integral (relative number of hydrogen atoms given). The following abbreviations are used to characterize the multiplicity of the signals: m=multiplett, q=quartett, t=triplett, d=doublet and s=singulett.

Abbreviations used are: min for minute(s)

### C.1 Compound Examples 1

Compound examples 1-1 to 1-38 correspond to compounds of formula C.1:

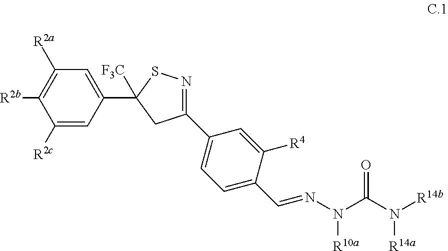

wherein R^{2a}, R^{2b}, R^{2c}, R⁴, R^{10a}, R^{14a} and R^{14b} of each synthesized compound is defined in one row of table C.1 below.

TABLE C.1

| | | | | | | HPLC-MS: Rₜ | |
|---|---|---|---|---|---|---|---|
| Ex. | R^{2a}, R^{2b}, R^{2c} | R⁴ | R^{10a} | R^{14a} | R^{14b} | (min) & [M + H]⁺ | |
| 1-1 | Cl, H, Cl | CH₃ | H | H | cyclopropylmethyl | 1.681 | 531.0 |
| 1-2 | Cl, H, Cl | CH₃ | H | H | CH₂CH₃ | 1.681 | 503.2 |
| 1-3 | Cl, H, Cl | Cl | H | H | CH₃ | 1.446 | 509.0 |
| 1-4 | Cl, H, Cl | Cl | H | H | CH₂CHF₂ | 1.475 | 560.9 |
| 1-5 | Cl, H, Cl | Cl | H | H | CH₂CF₃ | 1.502 | 578.9 |
| 1-6 | Cl, H, Cl | Cl | H | H | CH₂CH₃ | 1.319 | 523.0 |
| 1-7 | Cl, Cl, Cl | OCH₃ | H | H | CH₃ | 1.448 | 540.8 |
| 1-8 | Cl, Cl, Cl | OCH₃ | H | H | prop-2-ynyl | 1.459 | 564.8 |
| 1-9 | Cl, Cl, Cl | OCH₃ | H | H | CH₂CF₃ | 1.503 | 608.9 |
| 1-10 | Cl, Cl, Cl | SCH₃ | H | H | CH₂CF₃ | 1.546 | 625.1 |
| 1-11 | Cl, Cl, Cl | SCH₃ | H | H | CH₃ | 1.488 | 555.0 |
| 1-12 | Cl, Cl, Cl | SCH₃ | H | H | prop-2-ynyl | 1.497 | 580.8 |
| 1-13 | Cl, Cl, Cl | SCH₃ | H | H | CH₂CHF₂ | 1.518 | 607.1 |
| 1-14 | Cl, F, Cl | Cl | H | H | CH₂CF₃ | 1.501 | 597.1 |
| 1-15 | Cl, F, Cl | Cl | H | H | CH₃ | 1.455 | 529.1 |
| 1-16 | Cl, F, Cl | Cl | H | H | CH₂CHF₂ | 1.467 | 578.9 |
| 1-17 | Cl, Cl, Cl | S(O)CH₃ | H | H | CH₃ | 1.307 | 572.9 |
| 1-18 | Cl, Cl, Cl | S(O)CH₃ | H | H | CH₂CF₃ | 1.388 | 641.1 |
| 1-19 | Cl, Cl, Cl | CH₃ | H | H | CH₃ | 1.482 | 523.1 |
| 1-20 | Cl, Cl, Cl | CH₃ | H | H | CH₂CF₃ | 1.527 | 591.1 |
| 1-21 | Cl, Cl, Cl | CH₃ | H | H | OCH₃ | 1.445 | 541.1 |
| 1-22 | Cl, Cl, Cl | CH₃ | H | H | prop-2-ynyl | 1.473 | 547.2 |
| 1-23 | Cl, Cl, Cl | CH₃ | H | H | CH₂CHF₂ | 1.484 | 574.9 |
| 1-24 | Cl, F, Cl | CH₃ | H | H | CH₃ | 1.405 | 507.1 |
| 1-25 | Cl, F, Cl | CH₃ | H | H | CH₂CF₃ | 1.457 | 576.9 |
| 1-26 | Cl, F, Cl | CH₃ | H | H | CH₂CHF₂ | 1.436 | 557.1 |
| 1-27 | Cl, F, Cl | CH₃ | H | H | OCH₃ | 1.373 | 525.0 |
| 1-28 | Cl, F, Cl | CH₃ | H | H | prop-2-ynyl | 1.418 | 533.0 |
| 1-29 | Cl, Cl, Cl | Cl | H | H | OCH₃ | 1.460 | 560.9 |
| 1-30 | Cl, Cl, Cl | Cl | H | H | CH₂CF₃ | 1.530 | 612.9 |
| 1-31 | Cl, Cl, Cl | Cl | H | H | CH₂CHF₂ | 1.508 | 594.9 |
| 1-32 | Cl, Cl, Cl | Cl | H | H | prop-2-ynyl | 1.494 | 568.8 |
| 1-33 | Cl, Cl, Cl | Cl | H | H | CH₃ | 1.508 | 545.1 |
| 1-34 | Cl, H, Cl | Cl | CH₃ | H | phenyl | 1.457 | 585.1 |
| 1-35 | Cl, H, Cl | Cl | CH₃ | H | CH₃ | 1.362 | 524.8 |
| 1-36 | Cl, H, Cl | Cl | H | H | phenyl | 1.379 | 573.0 |
| 1-37 | Cl, Cl, Cl | SCH₃ | H | H | OCH₃ | 1.432 | 572.9 |
| 1-38 | Cl, Cl, Cl | F | H | H | CH₂CF₃ | 1.491 | 596.7 |

### C.2 Compound Examples 2

Compound examples 2-1 to 2-94 correspond to compounds of formula C.2:

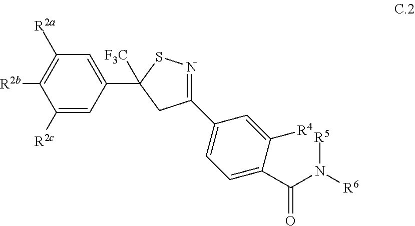

wherein R^{2a}, R^{2b}, R^{2c}, R⁴, R⁵, and R⁶ of each synthesized compound is defined in one row of table C.2 below.

TABLE C.2

| | | | | | HPLC-MS: Rₜ (min) | |
|---|---|---|---|---|---|---|
| Ex. | R^{2a}, R^{2b}, R^{2c} | R⁴ | R⁵ | R⁶ | & [M + H]⁺ or NMR | |
| 2-1 | Cl, H, Cl | CH₃ | H | cyclopropylmethyl | 1.491 | 487.1 |
| 2-2 | Cl, H, Cl | CH₃ | H | (3,3-difluorocyclobutyl)methyl | 1.466 | 537.3 |
| 2-3 | Cl, H, Cl | CH₃ | H | cyclobutylmethyl | 1.511 | 501.1 |
| 2-4 | Cl, H, Cl | CH₃ | H | cyclopentylmethyl | 1.546 | 516.8 |
| 2-5 | Cl, H, Cl | CH₃ | H | 1-cyanocyclopropyl | 1.391 | 498 |
| 2-6 | Cl, H, Cl | CH₃ | H | (1-cyanocyclopropyl)methyl | 1.392 | 512.1 |
| 2-7 | Cl, H, Cl | CH₃ | H | prop-2-ynyl | 1.396 | 471 |
| 2-8 | Cl, H, Cl | CH₃ | H | allyl | 1.423 | 473 |
| 2-9 | Cl, H, Cl | CH₃ | H | 3,4,4-trifluorobut-3-enyl | 1.477 | 541 |
| 2-10 | Cl, H, Cl | CH₃ | ═S(CH(CH₃)₂)₂ | | 1.355 | 549.3 |
| 2-11 | Cl, H, Cl | CH₃ | ═S(CH₂CH₃)₂ | | 1.308 | 521.3 |
| 2-12 | Cl, H, Cl | CH₃ | H | (2,2- | 1.51 | 557.2 |
| | | | | dichlorocyclopropyl)methyl | | |
| 2-13 | Cl, H, Cl | CH₃ | H | 2,2,2- | 1.317 | 573.3 |
| | | | | trifluoroethylcarbamoylamino | | |
| 2-14 | Cl, H, Cl | CH₃ | H | H | 1.310 | 433.2 |
| 2-15 | Cl, H, Cl | CH₃ | H | 2-oxo-2-(prop-2- | 1.319 | 528.37 |
| | | | | ynylamino)ethyl | | |
| 2-16 | Cl, H, Cl | CH₃ | H | 2,2- | 1.318 | 555.4 |
| | | | | difluoroethylcarbamoylamino | | |
| 2-17 | Cl, F, Cl | CH₃ | H | (3,3-difluorocyclobutyl)methyl | 1.463 | 555.2 |
| 2-18 | Cl, F, Cl | CH₃ | H | (1-cyanocyclopropyl)methyl | 1.379 | 530.1 |
| 2-19 | Cl, F, Cl | CH₃ | H | 2,2,2- | 1.321 | 591.1 |
| | | | | trifluoroethylcarbamoylamino | | |
| 2-20 | Cl, F, Cl | CH₃ | H | methylcarbamoylamino | 1.246 | 524.9 |
| 2-21 | Cl, F, Cl | CH₃ | H | 2,2-difluoroethyl- | ¹H NMR (400 MHz, | |
| | | | | carbamoylamino | CDCl₃): δ 8.8 (br. s, | |
| | | | | | 1H), 8.3 (br. s, 1H), 7.8- | |
| | | | | | 7.2 (m, 5H), 6.2 (br. s, | |
| | | | | | 1H), 5.8 (t, 1H), 4.2 (d, | |
| | | | | | 1H), 3.85 (d, 1H), 3.6- | |
| | | | | | 3.5 (m, 2H), 2.4 (s, 3H) | |
| 2-22 | Cl, F, Cl | Cl | H | (3,3-difluorocyclobutyl)methyl | 1.451 | 577.1 |
| 2-23 | Cl, F, Cl | Cl | H | (1-cyanocyclopropyl)methyl | 1.391 | 552.1 |
| 2-24 | Cl, F, Cl | Cl | H | 2,2- | 1.301 | 593 |
| | | | | difluoroethylcarbamoylamino | | |
| 2-25 | Cl, F, Cl | Cl | H | 2,2,2- | 1.312 | 611 |
| | | | | trifluoroethylcarbamoylamino | | |
| 2-26 | Cl, F, Cl | Cl | H | 1-cyanocyclopropyl | 1.405 | 538.1 |
| 2-27 | Cl, F, Cl | Cl | H | methylcarbamoylamino | 1.247 | 544.9 |
| 2-28 | Cl, F, Cl | CH₃ | H | 1-cyanocyclopropyl | 1.373 | 516.1 |
| 2-29 | Cl, F, Cl | CH₃ | H | cyclobutylmethyl | 1.475 | 519.1 |
| 2-30 | Cl, F, Cl | CH₃ | H | 2-oxo-2-(prop-2- | 1.308 | 546.1 |
| | | | | ynylamino)ethyl | | |
| 2-31 | Cl, F, Cl | CH₃ | H | (2,2- | 1.478 | 575 |
| | | | | dichlorocyclopropyl)methyl | | |
| 2-32 | Cl, F, Cl | CH₃ | H | cyclopropylmethyl | 1.427 | 505.1 |
| 2-33 | Cl, F, Cl | CH₃ | H | allyl | 1.398 | 491.1 |
| 2-34 | Cl, F, Cl | Cl | H | prop-2-ynyl | 1.381 | 509.73 |
| 2-35 | Cl, F, Cl | Cl | H | allyl | 1.414 | 513 |
| 2-36 | Cl, F, Cl | Cl | H | cyclobutylmethyl | 1.486 | 539.8 |
| 2-37 | Cl, F, Cl | Cl | H | (2,2- | 1.484 | 594.66 |
| | | | | dichlorocyclopropyl)methyl | | |
| 2-38 | Cl, F, Cl | Cl | H | cyclopentylmethyl | 1.545 | 555 |
| 2-39 | Cl, F, Cl | Cl | H | 3,4,4-trifluorobut-3-enyl | 1.474 | 581 |
| 2-40 | Cl, F, Cl | Cl | H | 2-oxo-2-(prop-2- | 1.32 | 566.78 |
| | | | | ynylamino)ethyl | | |
| 2-41 | Cl, F, Cl | Cl | H | cyclopropylmethyl | 1.437 | 527.1 |
| 2-42 | Cl, F, Cl | CH₃ | H | prop-2-ynyl | 1.39 | 489.1 |
| 2-43 | Cl, F, Cl | CH₃ | H | 3,4,4-trifluorobut-3-enyl | ¹H NMR (400 MHz, | |
| | | | | | CDCl₃): δ 7.6 (m, 2H), | |
| | | | | | 7.4 (m, 3H), 6.0 (br. s, | |
| | | | | | 1H), 4.2 (d, 1H), 3.85 | |
| | | | | | (d, 1H), 3.7-3.6 (m, | |
| | | | | | 2H), 2.8-2.6 (m, 2H), | |
| | | | | | 2.4 (s, 3H) | |
| 2-44 | Cl, H, Cl | CH₃ | H | 2-(cyclopropyl- | 1.376 | 580.9 |
| | | | | methylsulfonyl)ethyl | | |
| 2-45 | Cl, H, Cl | CH₃ | H | 2-(cyclopropylmethoxy)ethyl | 1.456 | 531.1 |
| 2-46 | Cl, H, Cl | CH₃ | H | (1-cyanocyclobutyl)methyl | 1.43 | 528.2 |
| 2-47 | Cl, H, Cl | CH₃ | H | 1-cyanocyclopentyl | 1.459 | 562.2 |
| 2-48 | Cl, H, Cl | CH₃ | H | 1-cyanocyclobutyl | 1.425 | 512.1 |
| 2-49 | Cl, H, Cl | CH₃ | H | (1-cyanocyclopentyl)methyl | 1.437 | 540.2 |
| 2-50 | Cl, H, Cl | CH₃ | H | (3,3-difluorocyclopentyl)methyl | 1.448 | 551.2 |
| 2-51 | Cl, H, Cl | CH₃ | H | 2-(cyclopropylmethylamino)-2- | 1.348 | 544.1 |
| | | | | oxo-ethyl | | |
| 2-52 | Cl, H, Cl | CH₃ | H | 2-(allylamino)-2-oxo-ethyl | 1.321 | 530.1 |
| 2-53 | Cl, H, Cl | CH₃ | H | 2-allyloxyethyl | 1.419 | 517.1 |
| 2-54 | Cl, H, Cl | CH₃ | H | 2-[(1-cyanocyclopropyl)amino]- | 1.289 | 555.1 |
| | | | | 2-oxo-ethyl | | |
| 2-55 | Cl, F, Cl | Cl | H | 2-(cyclopropylmethylamino)-2- | 1.343 | 584.0 |
| | | | | oxo-ethyl | | |
| 2-56 | Cl, F, Cl | CH₃ | H | 2-(cyclopropylmethylamino)-2- | 1.334 | 562.1 |
| | | | | oxo-ethyl | | |
| 2-57 | Cl, F, Cl | Cl | H | cyclohexylmethyl | 1.554 | 569.1 |
| 2-58 | Cl, F, Cl | CH₃ | H | cyclohexylmethyl | 1.544 | 547.2 |
| 2-59 | Cl, F, Cl | CH₃ | H | cyclopentylmethyl | 1.509 | 533.2 |
| 2-60 | Cl, F, Cl | CH₃ | ═S(CH₂CH₃)₂ | | 1.380 | 561.4 |
| 2-61 | Cl, F, Cl | CH₃ | H | 1-cyanocyclobutyl | 1.439 | 530.6 |
| 2-62 | Cl, F, Cl | Cl | H | 1-cyanocyclobutyl | 1.433 | 551.6 |
| 2-63 | Br, H, CF₃ | Cl | H | 1-CN-cyclopropyl | 1.402 | 597.6 |
| 2-64 | Br, H, CF₃ | Cl | H | allyl | 1.444 | 573.5 |
| 2-65 | Br, H, CF₃ | Cl | H | prop-2-ynyl | 1.415 | 569.6 |
| 2-66 | Br, H, CF₃ | Cl | H | cyclopropylmethyl | 1.466 | 584.7 |
| 2-67 | Cl, H, Cl | CH₃ | H | 2-allylsulfonylethyl | 1.359 | 564.8 |
| 2-68 | Cl, H, Cl | CH₃ | H | 2- | 1.419 | 594.6 |
| | | | | (cyclobutylmethylsulfonyl)ethyl | | |
| 2-69 | Cl, H, Cl | CH₃ | H | 2-prop-2-ynoxyethyl | 1.390 | 214.8 |
| 2-70 | Cl, H, Br | Cl | H | allyl | 1.443 | 539.5 |
| 2-71 | Cl, H, Br | Cl | H | prop-2-ynyl | 1.407 | 536.6 |
| 2-72 | Cl, H, Br | Cl | H | cyclopropylmethyl | 1.468 | 552.6 |
| 2-73 | Cl, H, Br | Cl | H | 1-CN-cyclopropyl | 1.400 | 563.6 |
| 2-74 | Cl, F, Cl | Cl | H | 1-cyanocyclopentyl | 1.454 | 563.8 |
| 2-75 | Cl, F, Cl | CH₃ | H | 1-cyanocyclopentyl | 1.442 | 543.8 |
| 2-76 | Cl, F, Cl | Cl | H | (2,2-difluorocyclopropyl)methyl | 1.451 | 563.6 |
| 2-77 | Cl, F, Cl | CH₃ | H | (2,2-difluorocyclopropyl)methyl | 1.431 | 542.5 |
| 2-78 | CF₃, H, CF₃ | Cl | H | cyclopropylmethyl | 1.440 | 574.8 |
| 2-79 | CF₃, H, CF₃ | Cl | H | 1-CN-cyclopropyl | 1.387 | 585.8 |
| 2-80 | CF₃, H, CF₃ | Cl | H | allyl | 1.414 | 560.8 |
| 2-81 | Br, F, Br | Cl | H | allyl | 1.441 | 600.5 |
| 2-82 | Br, F, Br | Cl | H | prop-2-ynyl | 1.411 | 598.6 |
| 2-83 | Br, F, Br | Cl | H | 1-CN-cyclopropyl | 1.405 | 625.6 |
| 2-84 | Br, F, Br | Cl | H | cyclopropylmethyl | 1.481 | 617.4 |
| 2-85 | Cl, H, Cl | CH₃ | H | 2-(cyclobutylmethoxy)ethyl | 1.544 | 544.8 |
| 2-86 | Cl, H, Cl | CH₃ | H | (2,2-difluorocyclopropyl)methyl | 1.443 | 524.6 |
| 2-87 | CF₃, H, CF₃ | Cl | H | prop-2-ynyl | 1.452 | 558.8 |
| 2-88 | Cl, F, Cl | Cl | H | 2-[(1-cyanocyclopropyl)- | 1.370 | 608.2 |
| | | | | methylamino]-2-oxo-ethyl | | |
| 2-89 | Cl, H, Cl | CH₃ | H | 2-[(1-cyanocyclopropyl)- | 1.349 | 568.4 |
| | | | | methylamino]-2-oxo-ethyl | | |
| 2-90 | Cl, F, Cl | Cl | H | 2-(allylamino)-2-oxo-ethyl | 1.341 | 569.6 |
| 2-91 | Cl, F, Cl | CH₃ | H | 2-(allylamino)-2-oxo-ethyl | 1.347 | 547.9 |
| 2-92 | Cl, F, Cl | NO₂ | H | cyclopropylmethyl | 1.411 | 535.8 |
| 2-93 | Cl, F, Cl | NO₂ | H | 1-CN-cyclopropyl | 1.356 | 546.8 |
| 2-94 | Cl, F, Cl | Cl | H | 2-[(1-cyanocyclopropyl)- | 1.321 | 586.9 |
| | | | | methylamino]-2-oxo-ethyl | | |

### C.3 Compound Examples 3

Compound examples 3-1 to 3-265 correspond to compounds of formula C.3:

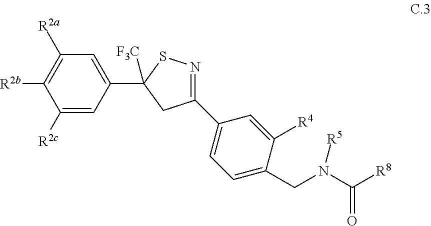

wherein R^{2a}, R^{2b}, R^{2c}, R⁴, R⁵ and R⁸ of each synthesized compound is defined in one row of table C.3 below.

TABLE C.3

| | | | | | HPLC-MS: Rₜ (min) | |
|---|---|---|---|---|---|---|
| Ex. | R^{2a}, R^{2b}, R^{2c} | R⁴ | R⁵ | R⁸ | & [M + H]⁺ or NMR | |
| 3-1 | Cl, H, Cl | CH₃ | CH₂CH₃ | methylsulfonylmethyl | ¹H NMR (400 MHz, | |
| | | | | | CDCl₃): δ 7.6-7.2 | |
| | | | | | (m, 6H), 4.7 (s, | |
| | | | | | 2H), 4.3-4.1 (m, | |
| | | | | | 3H), 3.85 (d, 1H), | |
| | | | | | 3.6-3.4 (m, 2H), 3.2 | |
| | | | | | (s, 3H), 2.35 (s, | |
| | | | | | 3H), 1.3-1.1 (m, | |
| | | | | | 2H) | |
| 3-2 | Cl, H, Cl | Cl | H | ethylsulfanylmethyl | 1.511 | 542.9 |
| 3-3 | Cl, H, Cl | Cl | H | cyclopropyl | 1.461 | 508.9 |
| 3-4 | Cl, H, Cl | Cl | CH₂CH₃ | CH₂CF₃ | 1.555 | 579.2 |
| 3-5 | Cl, H, Cl | Cl | CH₂CH₃ | CH₃ | 1.513 | 511.1 |
| 3-6 | Cl, H, Cl | Cl | CH₂CH₃ | CH₂CH₃ | 1.560 | 525.1 |
| 3-7 | Cl, H, Cl | Cl | CH₂CH₃ | CH₂S(O)₂CH₃ | 1.455 | 589.1 |
| 3-8 | Cl, H, Cl | Cl | CH₂CH₃ | ethylsulfanylmethyl | 1.596 | 571 |
| 3-9 | Cl, H, Cl | Cl | CH₂CH₃ | trifluoromethylsulfanyl- | 1.603 | 611 |
| | | | | methyl | | |
| 3-10 | Cl, H, Cl | Cl | H | CH₂CH₃ | 1.430 | 497.2 |
| 3-11 | Cl, H, Cl | Cl | H | CH₃ | 1.379 | 481.1 |
| 3-12 | Cl, H, Cl | Cl | H | CH₂CF₃ | 1.462 | 551.2 |
| 3-13 | Cl, H, Cl | Cl | CH₂CH₃ | 1,1-dioxothietan-3-yl | 1.495 | 601.2 |
| 3-14 | Cl, F, Cl | Cl | H | CH₃ | 1.397 | 501.0 |
| 3-15 | Cl, F, Cl | Cl | H | CH₂S(O)₂CH₃ | 1.379 | 578.9 |
| 3-16 | Cl, F, Cl | Cl | H | ethylsulfonylmethyl | 1.407 | 592.8 |
| 3-17 | Cl, F, Cl | Cl | H | cyclopropyl | 1.443 | 526.9 |
| 3-18 | Cl, F, Cl | Cl | H | CH₂CH₃ | 1.432 | 513.0 |
| 3-19 | Cl, F, Cl | Cl | H | CH₂CF₃ | 1.452 | 569.1 |
| 3-20 | Cl, F, Cl | Cl | H | 1,1-dioxothietan-3-yl | 1.373 | 591.0 |
| 3-21 | Cl, F, Cl | Cl | H | 2-pyridyl | 1.537 | 563.9 |
| 3-22 | Cl, F, Cl | Cl | H | 2,2- | 1.471 | 562.8 |
| | | | | difluorocyclopropyl | | |
| 3-23 | Cl, F, Cl | Cl | H | 2,2- | 1.506 | 595.0 |
| | | | | dichlorocyclopropyl | | |
| 3-24 | Cl, Cl, Cl | F | H | CH₃ | 1.400 | 499.0 |
| 3-25 | Cl, Cl, Cl | F | H | CH₂S(O)₂CH₃ | ¹H NMR (400 MHz, | |
| | | | | | CDCl₃): δ 7.6-7.4 | |
| | | | | | (m, 5H), 6.9 (br. s, | |
| | | | | | 1H), 4.6 (d, 2H), | |
| | | | | | 4.2 (d, 1H), 3.9 (s, | |
| | | | | | 2H), 3.8 (d, 1H), | |
| | | | | | 3.1 (s, 3H) | |
| 3-26 | Cl, Cl, Cl | F | H | ethylsulfonylmethyl | 1.379 | 578.9 |
| 3-27 | Cl, Cl, Cl | F | H | CH₂CH₃ | 1.459 | 515.0 |
| 3-28 | Cl, Cl, Cl | F | H | cyclopropyl | 1.455 | 525.1 |
| 3-29 | Cl, Cl, Cl | F | H | CH₂CF₃ | 1.462 | 569.0 |
| 3-30 | Cl, Cl, Cl | OCH₃ | H | CH₂CH₃ | 1.418 | 526.9 |
| 3-31 | Cl, Cl, Cl | OCH₃ | H | CH₂CF₃ | 1.447 | 581.2 |
| 3-32 | Cl, Cl, Cl | OCH₃ | H | CH₂S(O)₂CH₃ | 1.369 | 591.0 |
| 3-33 | Cl, Cl, Cl | OCH₃ | H | ethylsulfonylmethyl | 1.396 | 605.1 |
| 3-34 | Cl, Cl, Cl | SCH₃ | H | CH₂CF₃ | 1.466 | 597.0 |
| 3-35 | Cl, Cl, Cl | SCH₃ | H | CH₂S(O)₂CH₃ | 1.379 | 605.0 |
| 3-36 | Cl, Cl, Cl | SCH₃ | H | ethylsulfonylmethyl | 1.407 | 620.8 |
| 3-37 | Cl, Cl, Cl | SCH₃ | H | CH₂CH₃ | 1.442 | 542.9 |
| 3-38 | Cl, F, Cl | Cl | H | 1-fluorocyclopropyl | 1.518 | 543.6 |
| 3-39 | Cl, F, Cl | Cl | H | 2-chloro-2-fluoro- | 1.509 | 579.6 |
| | | | | cyclopropyl | | |
| 3-40 | Cl, F, Cl | Cl | H | 2,2- | 1.548 | 553.7 |
| | | | | dimethylcyclopropyl | | |
| 3-41 | Cl, F, Cl | Cl | H | 1-methylsulfonylethyl | 1.403 | 592.8 |
| 3-42 | Cl, F, Cl | Cl | H | 1-methyl-1- | 1.444 | 606.7 |
| | | | | methylsulfonyl-ethyl | | |
| 3-43 | Cl, F, Cl | Cl | H | 3,3,3-trifluoro-2- | 1.557 | 592.7 |
| | | | | methyl-prop-1-enyl | | |
| 3-44 | Cl, F, Cl | Cl | H | prop-1-enyl | 1.456 | 526.6 |
| 3-45 | Cl, F, Cl | Cl | H | 2-(2,2-difluoroethyl- | 1.401 | 606.7 |
| | | | | amino)-2-oxo-ethyl | | |
| 3-46 | Cl, F, Cl | Cl | H | 2-oxo-2-(prop-2- | 1.388 | 582.7 |
| | | | | ynylamino)ethyl | | |
| 3-47 | Cl, F, Cl | Cl | H | 1-methylcyclopropyl | 1.513 | 541.5 |
| 3-48 | Cl, F, Cl | Cl | H | 1-fluorocyclobutyl | 1.550 | 557.7 |
| 3-49 | Cl, F, Cl | Cl | H | tetrahydrofuran-2- | 1.467 | 569.7 |
| | | | | ylmethyl | | |
| 3-50 | Cl, F, Cl | Cl | H | tetrahydrofuran-3- | 1.426 | 571.5 |
| | | | | ylmethyl | | |
| 3-51 | Cl, F, Cl | Cl | H | 2-(cyclopropylamino)- | 1.381 | 581.8 |
| | | | | 2-oxo-ethyl | | |
| 3-52 | Cl, F, Cl | Cl | H | 3,3-dichlorocyclobutyl | 1.562 | 611.5 |
| 3-53 | Cl, H, Cl | Cl | CH₃ | CH₃ | 1.471 | 497.4 |
| 3-54 | Cl, H, Cl | Cl | cPr | CH₃ | 1.536 | 523.4 |
| 3-55 | Cl, H, Cl | Cl | cPr | CH₂CF₃ | 1.583 | 589.7 |
| 3-56 | Cl, H, Cl | Cl | CH₃ | CH₂CF₃ | 1.527 | 565.6 |
| 3-57 | Cl, H, Cl | Cl | CH₃ | CH₂S(O)₂CH₃ | 1.413 | 574.7 |
| 3-58 | Cl, H, Cl | Cl | cPr | CH₂S(O)₂CH₃ | 1.491 | 599.8 |
| 3-59 | Cl, H, Cl | Cl | Et | 2,2- | 1.643 | 605.6 |
| | | | | dichlorocyclopropyl | | |
| 3-60 | Cl, H, Cl | Cl | Et | ethylsulfonylmethyl | 1.485 | 602.6 |
| 3-61 | Cl, H, Cl | Cl | Et | 2,2- | 1.569 | 572.6 |
| | | | | difluorocyclopropyl | | |
| 3-62 | Cl, F, Cl | Cl | H | 1-cyanocyclobutyl | 1.509 | 563.8 |
| 3-63 | Cl, F, Cl | Cl | H | oxetan-2-yl | 1.435 | 542.6 |
| 3-64 | Cl, F, Cl | Cl | H | 2-(ethylamino)-2-oxo- | 1.388 | 569.8 |
| | | | | ethyl | | |
| 3-65 | Cl, F, Cl | Cl | H | methoxyiminomethyl | 1.488 | 543.6 |
| 3-66 | Cl, F, Cl | Cl | H | ethylcarbamoyl | ¹H NMR (400 MHz, | |
| | | | | | CDCl₃): δ 7.9 (br. s, | |
| | | | | | 1H), 7.8 (s, 1H), | |
| | | | | | 7.6 (m, 1H), 7.5-7.3 | |
| | | | | | (m, 3H), 4.6 (m, | |
| | | | | | 2H), 4.2 (d, 1H), | |
| | | | | | 3.8 (d, 1H), 3.4 (m, | |
| | | | | | 2H), 1.2 (t, 3H) | |
| 3-67 | Cl, F, Cl | Cl | H | prop-2-ynylcarbamoyl | 1.454 | 565.8 |
| 3-68 | Cl, F, Cl | Cl | H | 2,2,2-trifluoroethyl- | ¹H NMR (400 MHz, | |
| | | | | carbamoyl | CDCl₃): δ 7.9-7.3 | |
| | | | | | (m, 6H), 6.1-5.7 | |
| | | | | | (m, 1H), 4.6 (m, | |
| | | | | | 2H), 4.2 (d, 1H), | |
| | | | | | 3.85 (d, 1H), 3.8- | |
| | | | | | 3.7 (m, 2H) | |
| 3-69 | Cl, F, Cl | Cl | H | cyclobutyl | 1.517 | 539.6 |
| 3-70 | Cl, F, Cl | Cl | H | 2-oxo-2-(2,2,2- | 1.442 | 624.7 |
| | | | | trifluoro- | | |
| | | | | ethylamino)ethyl | | |
| 3-71 | Cl, F, Cl | Cl | H | cyclopropylcarbamoyl | 1.494 | 570.6 |
| 3-72 | Cl, F, Cl | Cl | H | 2,2- | ¹H NMR (400 MHz, | |
| | | | | difluoroethylcarbamoyl | CDCl₃): δ 7.9-7.3 | |
| | | | | | (m, 6H), 4.7-4.6 | |
| | | | | | (m, 2H), 4.2 (d, | |
| | | | | | 1H), 4.0-3.9 (m, | |
| | | | | | 2H), 3.8 (d, 1H) | |
| 3-73 | Br, H, CF₃ | Cl | H | CH₂CF₃ | 1.487 | 629.7 |
| 3-74 | Cl, F, Cl | Cl | H | n-propyl | 1.487 | 527.7 |
| 3-75 | Cl, F, Cl | Cl | H | 2,2-dimethylpropyl | 1.552 | 556.6 |
| 3-76 | Cl, F, Cl | Cl | H | 3,3,3-trifluoropropyl | 1.496 | 582.6 |
| 3-77 | Br, H, CF₃ | Br | H | CH₂S(O)₂CH₃ | 1.394 | 682.5 |
| 3-78 | Cl, F, Cl | Cl | H | sec-butyl | 1.537 | 541.7 |
| 3-79 | Cl, F, Cl | Cl | H | tert-Butyl | 1.528 | 542.6 |
| 3-80 | Br, H, CF₃ | Br | H | ethylsulfonylmethyl | 1.420 | 696.5 |
| 3-81 | Br, H, CF₃ | Br | H | tert-butoxy | ¹H NMR (400 MHz, | |
| | | | | | CDCl₃): δ 7.95 (s, | |
| | | | | | 1H), 7.8 (s, 1H), | |
| | | | | | 7.7 (s, 1H), 7.65 (d, | |
| | | | | | 1H), 7.6 (s, 1H), | |
| | | | | | 7.4 (d, 1H), 5.05 | |
| | | | | | (br. s, 1H), 4.4 (m, | |
| | | | | | 2H), 4.2 (d, 1H), | |
| | | | | | 3.8 (d, 1H), 1.4 (s, | |
| | | | | | 9H) | |
| 3-82 | Br, F, Br | Cl | H | tert-butoxy | ¹H NMR (400 MHz, | |
| | | | | | CDCl₃): δ 7.75 (s, | |
| | | | | | 1H), 7.6 (d, 1H), | |
| | | | | | 7.55 (m, 2H), 7.45 | |
| | | | | | (d, 1H), 5.0 (br. s, | |
| | | | | | 1H), 4.4 (m, 2H), | |
| | | | | | 4.2 (d, 1H), 3.8 (d, | |
| | | | | | 1H), 1.5 (s, 9H) | |
| 3-83 | Br, H, CF₃ | Br | H | CH₂CF₃ | 1.467 | 672.5 |
| 3-84 | Br, H, CF₃ | Br | H | CH₂CH₃ | 1.456 | 618.6 |
| 3-85 | Br, H, CF₃ | Br | H | CH₃ | 1.412 | 604.7 |
| 3-86 | Cl, F, Cl | Cl | H | isobutyl | 1.500 | 542.7 |
| 3-87 | Br, H, CF₃ | Br | H | cyclopropyl | 1.471 | 630.6 |
| 3-88 | Br, H, Cl | Cl | H | tert-butoxy | ¹H NMR (400 MHz, | |
| | | | | | CDCl₃): δ 7.8 (s, | |
| | | | | | 1H), 7.7-7.4 (m, | |
| | | | | | 4H), 7.3 (m, 1H), | |
| | | | | | 5.05 (br. s, 1H), 4.4 | |
| | | | | | (m, 2H), 4.2 (d, | |
| | | | | | 1H), 3.8 (d, 1H), | |
| | | | | | 1.4 (s, 9H) | |
| 3-89 | Cl, F, Cl | Cl | H | cyclopentyl | 1.537 | 554.6 |
| 3-90 | Cl, F, Cl | Cl | H | cyclohexyl | 1.566 | 566.8 |
| 3-91 | Cl, F, Cl | Cl | H | 2,2,3,3- | 1.518 | 612.6 |
| | | | | tetrafluorocyclobutyl | | |
| 3-92 | Cl, F, Cl | Cl | H | (2,2,3,3-tetrafluoro- | 1.514 | 626.6 |
| | | | | cyclobutyl)methyl | | |
| 3-93 | Cl, F, Cl | Cl | H | oxetan-3-yl | 1.379 | 542.6 |
| 3-94 | Cl, F, Cl | Cl | H | CH₂CN | 1.420 | 526.6 |
| 3-95 | Cl, F, Cl | Cl | H | 2,2- | 1.560 | 683.7 |
| | | | | dibromocyclopropyl | | |
| 3-96 | Br, H, CF₃ | Cl | H | CH₂S(O)₂CH₃ | ¹H NMR (400 MHz, | |
| | | | | | CDCl₃): δ 7.8 (m, | |
| | | | | | 2H), 7.75 (s, 1H), | |
| | | | | | 7.7-7.5 (m, 2H), 7.5 | |
| | | | | | (m, 1H), 6.9 (br. s, | |
| | | | | | 1H), 4.6 (m, 2H), | |
| | | | | | 4.2 (d, 1H), 3.9 (s, | |
| | | | | | 2H), 3.8 (d, 1H), | |
| | | | | | 3.1 (s, 3H) | |
| 3-97 | Br, H, CF₃ | Cl | H | ethylsulfonylmethyl | ¹H NMR (400 MHz, | |
| | | | | | CDCl₃): δ 7.8 (m, | |
| | | | | | 2H), 7.75 (s, 1H), | |
| | | | | | 7.7-7.5 (m, 2H), 7.5 | |
| | | | | | (m, 1H), 6.95 (br. s, | |
| | | | | | 1H), 4.6 (m, 2H), | |
| | | | | | 4.2 (d, 1H), 3.9 (s, | |
| | | | | | 2H), 3.85 (d, 1H), | |
| | | | | | 3.15 (q, 2H), 1.4 (t, | |
| | | | | | 3H) | |
| 3-98 | Br, H, CF₃ | Cl | H | CH₃ | 1.392 | 560.6 |
| 3-99 | Br, H, CF₃ | Cl | H | cyclopropyl | 1.452 | 586.6 |
| 3-100 | Br, H, CF₃ | Cl | H | n-propyl | 1.470 | 588.6 |
| 3-101 | Br, H, CF₃ | Cl | H | CH₂CH₃ | 1.432 | 574.6 |
| 3-102 | CF₃, H, CF₃ | Cl | H | CH₂CF₃ | 1.446 | 616.7 |
| 3-103 | CF₃, H, CF₃ | Cl | H | CH₂CH₃ | 1.415 | 562.8 |
| 3-104 | CF₃, H, CF₃ | Cl | H | CH₃ | 1.414 | 549.7 |
| 3-105 | CF₃, H, CF₃ | Cl | H | cyclopropyl | 1.470 | 575.7 |
| 3-106 | CF₃, H, CF₃ | Cl | H | n-propyl | 1.487 | 577.8 |
| 3-107 | CF₃, H, CF₃ | Cl | H | CH₂S(O)₂CH₃ | 1.361 | 626.7 |
| 3-108 | CF₃, H, CF₃ | Cl | H | ethylsulfonylmethyl | 1.388 | 640.8 |
| 3-109 | Br, F, Br | Cl | H | CH₂CF₃ | 1.502 | 657.6 |
| 3-110 | Br, F, Br | Cl | H | CH₂CH₃ | 1.473 | 603.6 |
| 3-111 | Cl, F, Cl | Cl | H | cyclobuten-1-yl | 1.480 | 538.6 |
| 3-112 | Cl, F, Cl | Cl | H | CH₂OCH₃ | 1.460 | 529.6 |
| 3-113 | Cl, F, Cl | Cl | H | dimethylaminomethyl | 1.184 | 544.6 |
| 3-114 | Cl, F, Cl | Cl | H | tetrahydrofuran-2-yl | 1.490 | 555.7 |
| 3-115 | Cl, F, Cl | Cl | H | 1,2,3,4,4,4- | ¹H NMR (400 MHz, | |
| | | | | hexafluoro-3- | CDCl₃): δ 7.8 (s, | |
| | | | | (trifluoromethyl)but-1- | 1H), 7.6 (m, 1H), | |
| | | | | enyl | 7.5 (m, 1H), 7.4 (m, | |
| | | | | | 2H), 6.8 (br. s, 1H), | |
| | | | | | 4.7 (m, 2H), 4.2 (d, | |
| | | | | | 1H), 3.85 (d, 1H) | |
| 3-116 | Cl, F, Cl | Cl | H | cyclopentylmethyl | 1.561 | 569.6 |
| 3-117 | Cl, F, Cl | Cl | H | 1-(trifluoromethyl)- | ¹H NMR (400 MHz, | |
| | | | | cyclopropyl | CDCl₃): δ 7.8 (s, | |
| | | | | | 1H), 7.6 (m, 1H), | |
| | | | | | 7.5-7.3 (m, 3H), 6.7 | |
| | | | | | (br. s, 1H), 4.6 (m, | |
| | | | | | 2H), 4.2 (d, 1H), | |
| | | | | | 3.8 (d, 1H), 1.45 | |
| | | | | | (m, 2H), 1.25 (m, | |
| | | | | | 2H) | |
| 3-118 | Cl, F, Cl | Cl | H | 2-(trifluoromethyl)- | ¹H NMR (400 MHz, | |
| | | | | cyclopropyl | CDCl₃): δ 7.8 (s, | |
| | | | | | 1H), 7.6 (m, 1H), | |
| | | | | | 7.5-7.25 (m, 3H), | |
| | | | | | 6.2 (br. s, 1H), 4.6 | |
| | | | | | (m, 2H), 4.2 (d, | |
| | | | | | 1H), 3.8 (d, 1H), | |
| | | | | | 2.0-1.8 (m, 2H), 1.6 | |
| | | | | | (m, 1H), 1.2 (m, | |
| | | | | | 1H) | |
| 3-119 | Cl, F, Cl | Cl | H | CH₂CH₂OCH₃ | 1.441 | 545.5 |
| 3-120 | Cl, F, Cl | Cl | H | cyclohexylmethyl | 1.609 | 583.7 |
| 3-121 | Cl, F, Cl | Cl | H | benzyl | 1.531 | 577.7 |
| 3-122 | Br, H, Cl | Cl | H | CH₂CF₃ | 1.463 | 594.6 |
| 3-123 | Br, H, Cl | Cl | H | CH₂CH₃ | 1.434 | 540.6 |
| 3-124 | Br, H, Cl | Cl | H | CH₃ | 1.389 | 526.6 |
| 3-125 | Br, H, Cl | Cl | H | CH₂S(O)₂CH₃ | ¹H NMR (400 MHz, | |
| | | | | | CDCl₃): δ 7.8 (s, | |
| | | | | | 1H), 7.6-7.5 (m, | |
| | | | | | 2H), 7.5-7.4 (m, | |
| | | | | | 2H), 7.3 (s, 1H), | |
| | | | | | 7.0 (br. s, 1H), 4.6 | |
| | | | | | (m, 2H), 4.2 (d, | |
| | | | | | 1H), 3.95 (s, 2H), | |
| | | | | | 3.8 (d, 1H), 3.1 (s, | |
| | | | | | 3H) | |
| 3-126 | Br, H, Cl | Cl | H | ethylsulfonylmethyl | ¹H NMR (400 MHz, | |
| | | | | | CDCl₃): δ 7.8 (s, | |
| | | | | | 1H), 7.6-7.5 (m, | |
| | | | | | 2H), 7.5-7.4 (m, | |
| | | | | | 2H), 7.3 (s, 1H), | |
| | | | | | 7.0 (br. s, 1H), 4.6 | |
| | | | | | (m, 2H), 4.2 (d, | |
| | | | | | 1H), 3.9 (s, 2H), | |
| | | | | | 3.8 (d, 1H), 3.2 (q, | |
| | | | | | 2H), 1.4 (t, 3H) | |
| 3-127 | Br, H, Cl | Cl | H | cyclopropyl | 1.488 | 553.7 |
| 3-128 | Br, H, Cl | Cl | H | n-propyl | 1.509 | 555.6 |
| 3-129 | Cl, F, Cl | Cl | H | isopropyl | 1.475 | 528.7 |
| 3-130 | Br, H, CF₃ | Br | H | n-propyl | 1.488 | 632.6 |
| 3-131 | Br, F, Br | Cl | H | CH₂S(O)₂CH₃ | 1.376 | 666.5 |
| 3-132 | Br, F, Br | Cl | H | ethylsulfonylmethyl | 1.405 | 682.4 |
| 3-133 | Br, F, Br | Cl | H | cyclopropyl | 1.461 | 614.6 |
| 3-134 | Br, F, Br | Cl | H | n-propyl | 1.477 | 616.6 |
| 3-135 | Br, F, Br | Cl | H | CH₃ | 1.396 | 588.6 |
| 3-136 | Cl, F, Cl | Cl | H | trifluoromethyl- | ¹H NMR (400 MHz, | |
| | | | | sulfanylmethyl | CDCl₃): δ 7.8 (s, | |
| | | | | | 1H), 7.6 (m, 1H), | |
| | | | | | 7.5 (m, 1H), 7.4 (m, | |
| | | | | | 2H), 6.9 (br. s, 1H), | |
| | | | | | 4.6 (m, 2H), 4.2 (d, | |
| | | | | | 1H), 3.8 (d, 1H), | |
| | | | | | 3.6 (s, 2H) | |
| 3-137 | Cl, F, Cl | Cl | H | tetrahydropyran-2- | 1.524 | 583.7 |
| | | | | ylmethyl | | |
| 3-138 | Cl, F, Cl | Cl | H | 2-thienylmethyl | 1.519 | 583.6 |
| 3-139 | Cl, F, Cl | Cl | H | pyrimidin-2-ylmethyl | 1.355 | 576.7 |
| 3-140 | Cl, F, Cl | Cl | H | oxetan-3-ylmethyl | 1.360 | 554.8 |
| 3-141 | Br, H, CF₃ | F | H | CH₂CF₃ | ¹H NMR (400 MHz, | |
| | | | | | CDCl₃): δ 7.8 (s, | |
| | | | | | 1H), 7.7 (s, 1H), | |
| | | | | | 7.6-7.4 (m, 4H), 6.2 | |
| | | | | | (br. s, 1H), 4.55 (m, | |
| | | | | | 2H), 4.2 (d, 1H), | |
| | | | | | 3.9 (d, 1H), 3.1 (m, | |
| | | | | | 2H) | |
| 3-142 | Br, H, CF₃ | F | H | CH₂CH₃ | 1.430 | 559.7 |
| 3-143 | Br, H, CF₃ | F | H | CH₃ | 1.383 | 545.6 |
| 3-144 | Br, H, CF₃ | F | H | CH₂S(O)₂CH₃ | ¹H NMR (400 MHz, | |
| | | | | | CDCl₃): δ 7.8 (s, | |
| | | | | | 1H), 7.7 (s, 1H), | |
| | | | | | 7.6-7.4 (m, 4H), 6.9 | |
| | | | | | (br. s, 1H), 4.55 (m, | |
| | | | | | 2H), 4.2 (d, 1H), | |
| | | | | | 3.9 (s, 2H), 3.85 (d, | |
| | | | | | 1H), 3.05 (s, 3H) | |
| 3-145 | Br, H, CF₃ | F | H | ethylsulfonylmethyl | ¹H NMR (400 MHz, | |
| | | | | | CDCl₃): δ 7.8 (s, | |
| | | | | | 1H), 7.7 (s, 1H), | |
| | | | | | 7.6-7.4 (m, 4H), 6.9 | |
| | | | | | (br. s, 1H), 4.6 (m, | |
| | | | | | 2H), 4.2 (d, 1H), | |
| | | | | | 3.9 (s, 2H), 3.85 (d, | |
| | | | | | 1H), 3.15 (q, 2H), | |
| | | | | | 1.4 (t, 3H) | |
| 3-146 | Br, H, CF₃ | F | H | cyclopropyl | 1.401 | 568.7 |
| 3-147 | Br, H, CF₃ | F | H | n-propyl | 1.418 | 572.6 |
| 3-148 | Cl, F, Cl | F | H | CH₂CF₃ | 1.412 | 550.8 |
| 3-149 | Cl, F, Cl | F | H | cyclopropyl | 1.392 | 510.5 |
| 3-150 | Cl, F, Cl | F | H | n-propyl | 1.411 | 510.8 |
| 3-151 | Cl, F, Cl | F | H | CH₂CH₃ | 1.402 | 498.5 |
| 3-152 | Cl, F, Cl | F | H | CH₃ | 1.371 | 482.8 |
| 3-153 | Cl, F, Cl | F | H | CH₂S(O)₂CH₃ | 1.382 | 560.7 |
| 3-154 | Cl, F, Cl | F | H | ethylsulfonylmethyl | 1.409 | 574.7 |
| 3-155 | Cl, F, Cl | Cl | H | C(O)NHCH₃ | ¹H NMR (400 MHz, | |
| | | | | | CDCl₃): δ 7.9 (br. s, | |
| | | | | | 1H), 7.8 (s, 1H), | |
| | | | | | 7.6 (m, 1H), 7.5-7.3 | |
| | | | | | (m, 3H), 4.6 (m, | |
| | | | | | 2H), 4.2 (d, 1H), | |
| | | | | | 3.8 (d, 1H), 2.8 (m, | |
| | | | | | 3H) | |
| 3-156 | Cl, F, Cl | Cl | H | thietan-3-yl | ¹H NMR (400 MHz, | |
| | | | | | CDCl₃): δ 7.8 (s, | |
| | | | | | 1H), 7.6 (m, 1H), | |
| | | | | | 7.5 (m, 1H), 7.4 (m, | |
| | | | | | 2H), 6.0 (br. s, 1H), | |
| | | | | | 4.6 (m, 2H), 4.2 (d, | |
| | | | | | 1H), 4.0 (m, 1H), | |
| | | | | | 3.8 (d, 1H), 3.6 (m, | |
| | | | | | 2H), 3.2 (m, 2H) | |
| 3-157 | Cl, F, Cl | Cl | H | 1,3-dioxolan-2- | 1.445 | 570.8 |
| | | | | ylmethyl | | |
| 3-158 | Cl, F, Cl | Cl | H | 1,3-dioxan-2-ylmethyl | 1.492 | 586.1 |
| 3-159 | Cl, H, CF₃ | Cl | H | CH₂CH₃ | 1.407 | 528.7 |
| 3-160 | Cl, H, CF₃ | Cl | H | n-propyl | 1.425 | 544.6 |
| 3-161 | Cl, H, CF₃ | Cl | H | cyclopropyl | 1.425 | 542.5 |
| 3-162 | Cl, H, CF₃ | Cl | H | CH₃ | 1.365 | 516.5 |
| 3-163 | Cl, H, CF₃ | Cl | H | CH₂S(O)₂CH₃ | 1.434 | 592.3 |
| 3-164 | Cl, H, CF₃ | Cl | H | ethylsulfonylmethyl | 1.463 | 606.4 |
| 3-165 | Cl, H, CF₃ | Cl | H | CH₂CF₃ | 1.551 | 582.3 |
| 3-166 | F, H, CF₃ | Cl | H | CH₂CF₃ | 1.503 | 566.4 |
| 3-167 | F, H, CF₃ | Cl | H | CH₂CH₃ | 1.385 | 512.8 |
| 3-168 | F, H, CF₃ | Cl | H | CH₃ | 1.342 | 498.7 |
| 3-169 | F, H, CF₃ | Cl | H | cyclopropyl | 1.401 | 524.8 |
| 3-170 | F, H, CF₃ | Cl | H | n-propyl | 1.419 | 526.8 |
| 3-171 | F, H, CF₃ | Cl | H | CH₂S(O)₂CH₃ | ¹H NMR (400 MHz, | |
| | | | | | CDCl₃): δ 7.8 (s, | |
| | | | | | 1H), 7.6 (d, 1H), | |
| | | | | | 7.5 (m, 2H), 7.4 (d, | |
| | | | | | 1H), 7.35 (d, 1H), | |
| | | | | | 7.0 (br. s, 1H), 4.6 | |
| | | | | | (m, 2H), 4.2 (d, | |
| | | | | | 1H), 3.95 (s, 2H), | |
| | | | | | 3.85 (d, 1H), 3.1 (s, | |
| | | | | | 3H) | |
| 3-172 | F, H, CF₃ | Cl | H | ethylsulfonylmethyl | ¹H NMR (400 MHz, | |
| | | | | | CDCl₃): δ 7.8 (s, | |
| | | | | | 1H), 7.6 (d, 1H), | |
| | | | | | 7.5 (m, 2H), 7.4 (d, | |
| | | | | | 1H), 7.35 (d, 1H), | |
| | | | | | 7.05 (br. s, 1H), 4.6 | |
| | | | | | (m, 2H), 4.2 (d, | |
| | | | | | 1H), 3.9 (s, 2H), | |
| | | | | | 3.85 (d, 1H), 3.15 | |
| | | | | | (q, 2H), 1.4 (t, 3H) | |
| 3-173 | Cl, F, Cl | Cl | H | 1-cyanocyclopropyl | 1.480 | 551.6 |
| 3-174 | Cl, F, Cl | Cl | H | 2-methoxycyclohexyl | 1.575 | 598.8 |
| 3-175 | Br, H, Br | Cl | H | CH₂CF₃ | 1.654 | 638.9 |
| 3-176 | Br, H, Br | Cl | H | cyclopropyl | 1.471 | 596.7 |
| 3-177 | Br, H, Br | Cl | H | CH₂CH₃ | 1.450 | 584.6 |
| 3-178 | Br, H, Br | Cl | H | CH₃ | 1.593 | 570.9 |
| 3-179 | Br, H, Br | Cl | H | CH₂S(O)₂CH₃ | 1.385 | 648.6 |
| 3-180 | Br, H, Br | Cl | H | ethylsulfonylmethyl | 1.414 | 662.6 |
| 3-181 | Cl, F, Cl | Cl | H | (1,1-dioxothiolan-3- | 1.363 | 618.7 |
| | | | | yl)methyl | | |
| 3-182 | Cl, F, Cl | Cl | H | 2,2,2-trifluoroethyl- | 1.493 | 612.7 |
| | | | | sulfanylmethyl | | |
| 3-183 | Cl, F, Cl | Cl | H | cyclobutylmethyl | 1.521 | 554.7 |
| 3-184 | Cl, F, Cl | Cl | H | (1,1-dioxothietan-3- | 1.370 | 602.8 |
| | | | | yl)methyl | | |
| 3-185 | Cl, F, Cl | Cl | H | 3,3-difluorocyclobutyl | 1.477 | 574.8 |
| 3-186 | Cl, F, Cl | Cl | H | vinyl | 1.472 | 510.7 |
| 3-187 | Br, H, Br | Cl | H | n-propyl | 1.525 | 599.0 |
| 3-188 | OCF₃, H, H | Cl | H | CH₂CH₃ | 1.383 | 510.8 |
| 3-189 | OCF₃, H, H | Cl | H | CH₂CF₃ | 1.415 | 564.8 |
| 3-190 | OCF₃, H, H | Cl | H | CH₃ | 1.341 | 496.8 |
| 3-191 | OCF₃, H, H | Cl | H | CH₂S(O)₂CH₃ | 1.328 | 575.2 |
| 3-192 | OCF₃, H, H | Cl | H | ethylsulfonylmethyl | 1.358 | 589.2 |
| 3-193 | OCF₃, H, H | Cl | H | n-propyl | 1.427 | 525.3 |
| 3-194 | OCF₃, H, H | Cl | H | cyclopropyl | 1.407 | 523.3 |
| 3-195 | Cl, F, Cl | Cl | H | 1,3-dioxolan-2-yl | 1.429 | 558.6 |
| 3-196 | Cl, F, Cl | Cl | H | 2,2,2-trifluoroethyl- | 1.469 | 644.7 |
| | | | | sulfonylmethyl | | |
| 3-197 | Cl, F, Cl | Cl | H | 2-furylmethyl | 1.481 | 566.6 |
| 3-198 | Cl, F, Cl | Cl | H | 1-chlorocyclopropyl | 1.573 | 560.7 |
| 3-199 | Cl, F, Cl | Cl | H | cyclopropylmethylcarbamoyl | 1.524 | 583.7 |
| 3-200 | Cl, F, Cl | Cl | H | C(CH₃)₂CN | 1.497 | 554.2 |
| 3-201 | Cl, F, Cl | Cl | H | cyclopropylmethyl | 1.488 | 540.6 |
| 3-202 | Br, H, CF₃ | NO₂ | H | CH₃ | 1.368 | 571.6 |
| 3-203 | Br, H, CF₃ | NO₂ | H | CH₂CH₃ | 1.409 | 585.7 |
| 3-204 | Br, H, CF₃ | NO₂ | H | n-propyl | 1.443 | 599.7 |
| 3-205 | Br, H, CF₃ | NO₂ | H | cyclopropyl | 1.426 | 597.7 |
| 3-206 | Br, H, CF₃ | NO₂ | H | CH₂CF₃ | 1.438 | 639.6 |
| 3-207 | Br, H, CF₃ | NO₂ | H | CH₂S(O)₂CH₃ | 1.350 | 647.7 |
| 3-208 | Br, H, CF₃ | NO₂ | H | ethylsulfonylmethyl | 1.377 | 663.6 |
| 3-209 | CF₃, F, H | Cl | H | CH₂CH₃ | 1.374 | 512.9 |
| 3-210 | CF₃, F, H | Cl | H | CH₂CF₃ | 1.406 | 566.8 |
| 3-211 | CF₃, F, H | Cl | H | CH₃ | 1.383 | 499.2 |
| 3-212 | CF₃, F, H | Cl | H | cyclopropyl | 1.392 | 524.8 |
| 3-213 | CF₃, F, H | Cl | H | CH₂S(O)₂CH₃ | 1.347 | 577.2 |
| 3-214 | CF₃, F, H | Cl | H | ethylsulfonylmethyl | 1.375 | 591.2 |
| 3-215 | CF₃, F, H | Cl | H | n-propyl | 1.410 | 526.9 |
| 3-216 | Br, H, CF₃ | CF₃ | H | CH₂CH₃ | 1.496 | 609.2 |
| 3-217 | Br, H, CF₃ | CF₃ | H | CH₂CF₃ | 1.518 | 663.1 |
| 3-218 | Br, H, CF₃ | CF₃ | H | CH₃ | 1.456 | 595 |
| 3-219 | Br, H, CF₃ | CF₃ | H | cyclopropyl | 1.511 | 621 |
| 3-220 | Br, H, CF₃ | CF₃ | H | CH₂S(O)₂CH₃ | 1.437 | 673.1 |
| 3-221 | Br, H, CF₃ | CF₃ | H | ethylsulfonylmethyl | 1.463 | 687.2 |
| 3-222 | Br, H, CF₃ | CF₃ | H | n-propyl | 1.529 | 623.1 |
| 3-223 | Cl, H, Cl | F | H | CH₂CH₃ | 1.393 | 478.8 |
| 3-224 | Cl, H, Cl | F | H | CH₂CF₃ | ¹H NMR (400 MHz, | |
| | | | | | CDCl₃): δ 7.7-7.3 | |
| | | | | | (m, 6H), 6.9 (br. s, | |
| | | | | | 1H), 4.5 (m, 2H), | |
| | | | | | 4.2 (d, 1H), 3.8 (d, | |
| | | | | | 1H), 3.2-3.0 (m, | |
| | | | | | 2H) | |
| 3-225 | Cl, H, Cl | F | H | cyclopropyl | 1.412 | 490.8 |
| 3-226 | Cl, H, Cl | F | H | CH₂S(O)₂CH₃ | ¹H NMR (400 MHz, | |
| | | | | | CDCl₃): δ 7.7-7.3 | |
| | | | | | (m, 6H), 7.2 (br. s, | |
| | | | | | 1H), 4.6 (m, 2H), | |
| | | | | | 4.2 (d, 1H), 3.95 (s, | |
| | | | | | 2H), 3.85 (d, 1H), | |
| | | | | | 3.1 (s, 3H) | |
| 3-227 | Cl, H, Cl | F | H | ethylsulfonylmethyl | 1.360 | 556.8 |
| 3-228 | Cl, H, Cl | F | H | n-propyl | 1.431 | 492.8 |
| 3-229 | Cl, H, Cl | F | H | CH₃ | 1.349 | 464.8 |
| 3-230 | I, H, CF₃ | Cl | H | CH₃ | 1.450 | 607.1 |
| 3-231 | I, H, CF₃ | Cl | H | CH₂CH₃ | 1.490 | 621.1 |
| 3-232 | I, H, CF₃ | Cl | H | n-propyl | 1.496 | 634.7 |
| 3-233 | I, H, CF₃ | Cl | H | cyclopropyl | 1.478 | 632.7 |
| 3-234 | I, H, CF₃ | Cl | H | CH₂S(O)₂CH₃ | 1.482 | 685.2 |
| 3-235 | I, H, CF₃ | Cl | H | ethylsulfonylmethyl | 1.455 | 699.1 |
| 3-236 | I, H, CF₃ | Cl | H | CH₂CF₃ | 1.517 | 675.1 |
| 3-237 | Cl, Cl, Cl | Cl | H | CH₂CF₃ | 1.546 | 585.1 |
| 3-238 | Cl, Cl, Cl | Cl | H | CH₃ | 1.484 | 517.2 |
| 3-239 | Cl, Cl, Cl | Cl | H | CH₂CH₃ | 1.492 | 530.7 |
| 3-240 | Cl, Cl, Cl | Cl | H | n-propyl | 1.527 | 544.7 |
| 3-241 | Cl, Cl, Cl | Cl | H | cyclopropyl | 1.509 | 542.7 |
| 3-242 | Cl, Cl, Cl | Cl | H | CH₂S(O)₂CH₃ | 1.428 | 594.7 |
| 3-243 | Cl, Cl, Cl | Cl | H | ethylsulfonylmethyl | 1.456 | 608.7 |
| 3-244 | Cl, H, Cl | Cl | H | tert-butoxy | 1.605 | 485.4 |
| 3-245 | OCF₃, H, Br | Cl | H | CH₂CF₃ | 1.485 | 644.6 |
| 3-246 | OCF₃, H, Br | Cl | H | CH₃ | 1.423 | 576.7 |
| 3-247 | OCF₃, H, Br | Cl | H | cyclopropyl | 1.510 | 603.2 |
| 3-248 | OCF₃, H, Br | Cl | H | CH₂S(O)₂CH₃ | 1.430 | 655.1 |
| 3-249 | OCF₃, H, Br | Cl | H | ethylsulfonylmethyl | 1.457 | 669.1 |
| 3-250 | OCF₃, H, Br | Cl | H | n-propyl | 1.495 | 604.7 |
| 3-251 | OCF₃, H, Br | Cl | H | CH₂CH₃ | 1.491 | 591.1 |
| 3-252 | OCF₃, H, Cl | Cl | H | CH₂CH₃ | 1.451 | 544.8 |
| 3-253 | OCF₃, H, Cl | Cl | H | CH₂CF₃ | 1.475 | 598.8 |
| 3-254 | OCF₃, H, Cl | Cl | H | CH₃ | 1.423 | 530.8 |
| 3-255 | OCF₃, H, Cl | Cl | H | cyclopropyl | 1.467 | 556.8 |
| 3-256 | OCF₃, H, Cl | Cl | H | CH₂S(O)₂CH₃ | 1.388 | 608.7 |
| 3-257 | OCF₃, H, Cl | Cl | H | ethylsulfonylmethyl | 1.414 | 622.8 |
| 3-258 | OCF₃, H, Cl | Cl | H | n-propyl | 1.484 | 558.8 |
| 3-259 | CF₃, H, Br | CH₃ | H | CH₂CF₃ | 1.441 | 608.6 |
| 3-260 | CF₃, H, Br | CH₃ | H | CH₂CH₃ | 1.412 | 554.7 |
| 3-261 | CF₃, H, Br | CH₃ | H | cyclopropyl | 1.429 | 566.6 |
| 3-262 | CF₃, H, Br | CH₃ | H | CH₂S(O)₂CH₃ | 1.350 | 618.6 |
| 3-263 | CF₃, H, Br | CH₃ | H | ethylsulfonylmethyl | 1.379 | 632.6 |
| 3-264 | CF₃, H, Br | CH₃ | H | n-propyl | 1.446 | 568.7 |
| 3-265 | CF₃, H, Br | CH₃ | H | CH₃ | 1.357 | 540.7 |

### C.4 Compound Examples 4

Compound examples 4-1 to 4-9 correspond to compounds of formula C.4:

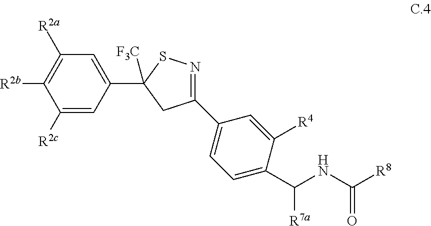

wherein R^{2a}, R^{2b}, R^{2c}, R⁴, R^{7a} and R⁸ of each synthesized compound is defined in one row of table C.4 below.

TABLE C.4

| | | | | | |
|---|---|---|---|---|---|
| | R^{2a}, R^{2b}, | | | | HPLC-MS: Rₜ (min) & |
| Ex. | R^{2c} | R⁴ | R^{7a} | R⁸ | [M + H]⁺ or NMR |

| 4-1 | Br, H, CF₃ | H | CH₃ | CH₃ | 1.375 | 540.6 |
|---|---|---|---|---|---|---|
| 4-2 | Br, H, CF₃ | H | CH₃ | CH₂CH₃ | 1.417 | 554.7 |
| 4-3 | Br, H, CF₃ | H | CH₃ | n-propyl | 1.453 | 568.7 |
| 4-4 | Br, H, CF₃ | H | CH₃ | CH₂CF₃ | 1.463 | 609.0 |
| 4-5 | Br, H, CF₃ | H | CH₃ | cyclopropyl | 1.435 | 566.6 |
| 4-6 | Br, H, CF₃ | H | CH₃ | CH₂S(O)₂CH₃ | 1.368 | 619.0 |
| 4-7 | Br, H, CF₃ | H | CH₃ | ethylsulfonyl- | 1.397 | 633.0 |
| | | | | methyl | | |
| 4-8 | Cl, Cl, Cl | F | CH₃ | CH₃ | ¹H NMR (500 MHz, | |
| | | | | | CDCl₃): δ 7.5 (s, 1H), | |
| | | | | | 7.5-7.4 (m, 3H), 7.4-7.3 | |
| | | | | | (m, 1H), 5.95 (br. s, 1H), | |
| | | | | | 5.25 (m, 1H), 4.15 (d, | |
| | | | | | 1H), 3.8 (d, 1H), 2.25 (q, | |
| | | | | | 2H), 1.5 (d, 3H), 1.15 | |
| | | | | | (t, 3H) | |
| 4-9 | Cl, Cl, Cl | F | CH₃ | CH₂CH₃ | 1.512 | 529.1 |

### B. Biology

The biological activity and effectivity of the compounds applied in the methods of the present invention can be evaluated e.g. in the following assay.

B.1 Green Soldier Stink Bug (Nezara viridula)

The active compound was dissolved at the desired concentration in a mixture of 1:1 (vol:vol) distilled water:acetone. Surfactant (Kinetic HV) was added at a rate of 0.01% (vol/vol). The test solution was prepared at the day of use.

Soybean pods were placed in glass Petri dishes lined with moist filter paper and inoculated with ten late 3rd instar N. viridula. Using a hand atomizer, approximately 2 ml solution is sprayed into each Petri dish. Assay arenas were kept at about 25° C. Percent mortality was recorded after 5 days.

In this test, compounds 1-1, 1-2, 1-3, 1-4, 1-5, 1-6, 1-7, 1-8, 1-9, 1-11, 1-14, 1-15, 1-16, 1-19, 1-20, 1-23, 1-24, 1-25, 1-26, 1-29, 1-30, 1-31, 1-33, 1-37, 2-13, 2-16, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-47, 2-48, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-69, 2-70, 2-71, 2-72, 2-73, 2-74, 2-75, 2-76, 2-77, 2-78, 2-80, 2-81, 2-82, 2-83, 2-84, 2-86, 2-87, 2-88, 2-89, 2-90, 3-1, 3-2, 3-3, 3-4, 3-5, 3-6, 3-7, 3-8, 3-9, 3-10, 3-11, 3-12, 3-13, 3-14, 3-15, 3-17, 3-18, 3-19, 3-20, 3-21, 3-22, 3-23, 3-24, 3-25, 3-27, 3-28, 3-29, 3-30, 3-31, 3-32, 3-34, 3-35, 3-37, 3-40, 3-44, 3-45, 3-46, 3-48, 3-49, 3-50, 3-51, 3-63, 3-64, 3-69, 3-70, 3-73, 3-74, 3-75, 3-76, 3-78, 3-83, 3-85, 3-86, 3-89, 3-94, 3-96, 3-98, 3-99, 3-100, 3-101, 3-102, 3-103, 3-104, 3-105, 3-106, 3-107, 3-109, 3-110, 3-111, 3-112, 3-114, 3-119, 3-122, 3-124, 3-125, 3-129, 3-130, 3-131, 3-133, 3-134, 3-135, 3-136, 3-139, 3-140, 3-141, 3-142, 3-143, 3-144, 3-146, 3-147, 3-148, 3-149, 3-150, 3-151, 3-152, 3-153, 3-156, 3-157, 3-158, 3-159, 3-160, 3-161, 3-162, 3-163, 3-165, 3-166, 3-167, 3-168, 3-169, 3-170, 3-171 and 3-172 at 500 ppm showed over 75% mortality in comparison with untreated controls.

Also in this test, compounds 1-7, 1-11, 1-15, 1-16, 1-29, 1-33, 1-37, 2-13, 2-16, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-47, 2-48, 2-50, 2-51, 2-52, 2-53, 2-54, 2-55, 2-56, 2-57, 2-59, 2-60, 2-61, 2-62, 2-63, 2-64, 2-65, 2-66, 2-72, 2-74, 2-75, 2-76, 2-77, 2-78, 2-80, 2-81, 2-82, 2-83, 2-84, 2-86, 2-87, 2-88, 2-89, 2-90, 3-2, 3-3, 3-4, 3-5, 3-6, 3-7, 3-10, 3-12, 3-13, 3-17, 3-18, 3-19, 3-24, 3-25, 3-27, 3-28, 3-29, 3-30, 3-31, 3-32, 3-34, 3-35, 3-37, 3-44, 3-49, 3-63, 3-64, 3-69, 3-73, 3-74, 3-78, 3-98, 3-99, 3-100, 3-102, 3-103, 3-104, 3-105, 3-106, 3-107, 3-109, 3-110, 3-112, 3-119, 3-122, 3-129, 3-130, 3-133, 3-134, 3-135, 3-139, 3-141, 3-142, 3-143, 3-144, 3-146, 3-147, 3-148, 3-149, 3-150, 3-151, 3-152, 3-156, 3-157, 3-158, 3-159, 3-160, 3-161, 3-162, 3-165, 3-167, 3-169 and 3-171 at 100 ppm showed over 75% mortality in comparison with untreated controls.

B.2 Neotropical Brown Stink Bug (Euschistus heros)

The active compound was dissolved at the desired concentration in a mixture of 1:1 (vol:vol) distilled water:acetone. Surfactant (Kinetic HV) was added at a rate of 0.01% (vol/vol). The test solution was prepared at the day of use.

Soybean pods were placed in microwavable plastic cups and inoculated with ten adult stage E. heros. Using a hand atomizer, approximately 1 ml solution is sprayed into each cup, insects and food present. A water source was provided (cotton wick with water). Each treatment was replicated 2-fold. Assay arenas were kept at about 25° C. Percent mortality was recorded after 5 days.

In this test, compounds 1-33, 2-13, 2-16, 2-18, 2-19, 2-20, 2-21, 2-22, 2-23, 2-24, 2-26, 2-27, 2-29, 2-30, 2-31, 2-32, 2-33, 2-48, 2-50, 2-54, 2-56, 2-61, 2-62, 2-66, 2-72, 2-74, 2-75, 2-76, 2-77, 2-78, 2-80, 2-82, 2-84, 2-86, 2-87, 3-19, 3-27, 3-28, 3-29, 3-31, 3-49, 3-69, 3-73, 3-74, 3-83, 3-102, 3-106, 3-107, 3-109, 3-110 and 3-135 at 100 ppm showed over 75% mortality in comparison with untreated controls.

B.3 Brown Marmorated Stink Bug (Halyomorpha halys)

The active compound was dissolved at the desired concentration in a mixture of 1:1 (vol:vol) distilled water:acetone. Surfactant (Kinetic HV) was added at a rate of 0.01% (vol/vol). The test solution was prepared at the day of use.

Row peanuts and soybean seeds were placed into microwavable plastic cups and inoculated with five adult stage H. halys. Using a hand atomizer, approximately 1 ml solution is sprayed into each cup, insects and food present. A water source was provided (cotton wick with water). Each treatment is replicated 4-fold. Assay arenas are kept at about 25° C. Percent mortality was recorded after 5 days.

In this test, compounds 2-13, 2-16, 2-18, 2-19, 2-20, 2-21, 2-24, 2-25, 2-27, 2-30, 2-32, 2-33, 2-34, 2-35, 2-38, 2-39, 2-40, 2-41, 2-42, 2-51, 2-52, 2-54, 2-55, 2-56, 2-57, 2-59, 2-66, 2-72, 2-76, 2-77, 2-80, 2-81, 2-82, 2-84, 2-87, 3-19, 3-24, 3-27, 3-28, 3-29, 3-98, 3-104, 3-110, 3-112, 3-119 and 3-135 at 100 ppm showed over 75% mortality in comparison with untreated controls.

1-36. (canceled)

37. A method for combating or controlling pests on soybean plants and/or on soybean crops comprising contacting the soybean plant, the crops, the pests, their food supply, habitat or breeding grounds with an isothiazoline compound of formula I or a composition comprising at least one compound of formula I: 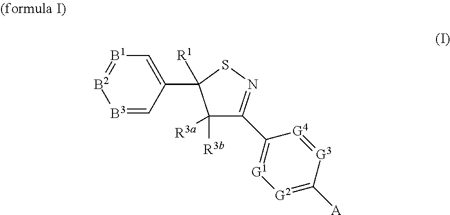
wherein
A is a group A¹, A² or A³;
wherein
A¹ is selected from the group consisting of —C(═NR⁶)R⁸, —S(O)ₙR⁹, and —N(R⁵)R⁶;
A² is a group of following formula: 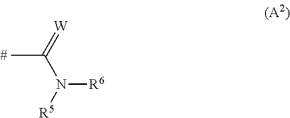
wherein
# denotes the bond to the remainder of the molecule;
W is selected from the group consisting of O and S;
A³ is a group of following formula: 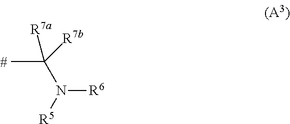
wherein
# denotes the bond to the aromatic ring of formula (I);
B¹, B² and B³ are each independently selected from the group consisting of N and CR², with the proviso that at most two of B¹, B² and B³ are N;
G¹, G², G³ and G⁴ are each independently selected from the group consisting of N and CR⁴, with the proviso that at most two of G¹, G², G³ and G⁴ are N;
R¹ is selected from the group consisting of C₁-C₄-alkyl, C₁-C₄-haloalkyl, C₁-C₄-alkoxy-C₁-C₄-alkyl, C₁-C₄-haloalkoxy-C₁-C₄-alkyl-, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl and —C(═O)OR¹⁵;
each R² is independently selected from the group consisting of hydrogen, halogen, cyano, azido, nitro, —SCN, —SF₅, C₁-C₆-alkyl, C₃-C₈-cycloalkyl, C₂-C₆-alkenyl, C₂-C₆-alkynyl, wherein the four last mentioned aliphatic and cycloaliphatic radicals may be partially or fully halogenated and/or may be substituted by one or more radicals R⁸, —Si(R¹²)₃, —OR⁹, —S(O)ₙR⁹, and —NR^{10a}R^{10b};
R^{3a}, R^{3b} are each independently selected from the group consisting of hydrogen, halogen, hydroxyl, —CO₂R^{3d}, C₁-C₃-alkyl, C₁-C₃-haloalkyl, C₂-C₃-alkenyl, C₂-C₃-alkynyl, C₁-C₃-alkoxy, C₁-C₃-haloalkoxy, C₁-C₃-alkylthio, C₁-C₃-haloalkylthio, C₁-C₃-alkylsulfonyl and C₁-C₃-haloalkylsulfonyl; or R^{3a} and R^{3b} together form a group ═O, ═C(R^{3c})₂, ═NOH or ═NOCH₃;
each R^{3c} is independently selected from the group consisting of hydrogen, halogen, CH₃ and CF₃;
R^{3d} is selected from the group consisting of hydrogen, C₁-C₆-alkyl and C₁-C₃-alkyloxy-C₁-C₃-alkyl-;
each R⁴ is independently selected from the group consisting of hydrogen, halogen, cyano, azido, nitro, —SCN, —SF₅, C₁-C₆-alkyl which may be partially or fully halogenated and/or may be substituted by one or more radicals R⁸, C₃-C₈-cycloalkyl which may be partially or fully halogenated and/or may be substituted by one or more radicals R⁸, C₂-C₆-alkenyl which may be partially or fully halogenated and/or may be substituted by one or more radicals R⁸, C₂-C₆-alkynyl which may be partially or fully halogenated and/or may be substituted by one or more radicals R⁸, —Si(R¹²)₃, —OR⁹, —S(O)ₙR⁹, —NR^{10a}R^{10b}, phenyl which may be substituted by 1, 2, 3, 4 or 5 radicals R¹¹, and a 3-, 4-, 5-, 6-7-, 8-, 9- or 10-membered saturated, partially unsaturated or maximally unsaturated heteromonocyclic or heterobicyclic ring containing 1, 2, 3 or 4 heteroatoms or heteroatom groups selected from the group consisting of N, O, S, NO, SO and SO₂, as ring members, where the heteromonocyclic or heterobicyclic ring may be substituted by one or more radicals R¹¹;
each R⁵ is independently selected from the group consisting of hydrogen, C₁-C₁₀-alkyl, C₃-C₈-cycloalkyl, C₂-C₁₀-alkenyl, and C₂-C₁₀-alkynyl, wherein the four last-mentioned aliphatic and cycloaliphatic radicals may be partially or fully halogenated and/or may be substituted with one or more substituents R⁸,
R⁶ in A² is selected from the group consisting of hydrogen, C₁-C₆-alkyl, C₁-C₆-haloalkyl, C₁-C₄-alkyl which carries one radical R⁸, C₂-C₆-alkenyl, C₂-C₆-haloalkenyl, C₂-C₆-alkynyl, C₃-C₆-cycloalkyl which may be substituted by 1 to 4 substituents selected from halogen and cyano; —N(R^{10a})R^{10b}, —CH═NOR⁹; phenyl which may be substituted with 1, 2, 3, 4, or 5 substituents R¹¹; and a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from the group consisting of N, O, S, NO, SO and SO₂, as ring members, wherein the heteromonocyclic or heterobicyclic ring may be substituted with one or more substituents R¹¹; or R⁵ and R⁶ form together a group ═S(O)ₘ(R⁹)₂;
each R⁶ in all other cases is independently selected from the group consisting of hydrogen, cyano, C₁-C₁₀-alkyl, C₃-C₈-cycloalkyl, C₂-C₁₀-alkenyl, and C₂-C₁₀-alkynyl, wherein the four last-mentioned aliphatic and cycloaliphatic radicals may be partially or fully halogenated and/or may be substituted by one or more substituents R⁸, —OR⁹, —NR^{10a}R^{10b}, —S(O)ₙR⁹, —C(═O)NR^{10a}N(R^{10a})R^{10b}), —Si(R¹²)₃, —C(═O)R⁸, phenyl which may be substituted with 1, 2, 3, 4, or 5 substituents R¹¹, and a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from the group consisting of N, O, S, NO, SO and SO₂, as ring members, wherein the heteromonocyclic or heterobicyclic ring may be substituted with one or more substituents R¹¹;
or
R⁵ and R⁶ or R⁵ and R^{6a}, together with the nitrogen atom to which they are bound, form a 3-, 4-, 5-, 6-, 7- or 8-membered saturated, partially unsaturated or maximally unsaturated heterocyclic ring, where the ring may further contain 1, 2, 3 or 4 heteroatoms or heteroatom-containing groups selected from the group consisting of O, S, N, SO, SO₂, C═O and C═S as ring members, wherein the heterocyclic ring may be substituted with 1, 2, 3, 4 or 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic or cycloaliphatic moieties in the twelve last-mentioned radicals may be substituted by one or more radicals R⁸, and phenyl which may be substituted with 1, 2, 3, 4 or 5 substituents R¹¹;
R^{7a}, R^{7b} are each independently selected from the group consisting of hydrogen, halogen, cyano, C₁-C₆-alkyl, C₃-C₈-cycloalkyl, C₂-C₆-alkenyl and C₂-C₆-alkynyl, wherein the four last-mentioned aliphatic and cycloaliphatic radicals may be partially or fully halogenated and/or may be substituted by one or more radicals R⁸;
each R⁸ is independently selected from the group consisting of cyano, azido, nitro, —SCN, —SF₅, C₃-C₈-cycloalkyl, C₃-C₈-halocycloalkyl, C₃-C₈-cycloalkenyl, C₃-C₈-halocycloalkenyl, wherein the cycloaliphatic moieties in the four last-mentioned radicals may be substituted by one or more radicals R¹³; —Si(R¹²)₃, —OR⁹, —OSO₂R⁹, —S(O)ₙR⁹, —N(R^{10a})R^{10b}, —C(═O)N(R^{10a})R^{10b}, —C(═S)N(R^{10a})R^{10b}, —C(═O)OR⁹, —CH═NOR⁹; phenyl, optionally substituted with 1, 2, 3, 4 or 5 substituents R¹⁶, and a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more substituents R¹⁶,
or
two R⁸ present on the same carbon atom of an alkyl, alkenyl, alkynyl or cycloalkyl group together form a group ═O, ═C(R¹³)₂; ═S; ═S(O)ₘ(R¹⁵)₂, ═S(O)ₘR¹⁵N(R^{14a})R^{14b}, ═NR^{10a}, ═NOR⁹; or ═NN(R^{10a})R^{10b};
or
two radicals R⁸, together with the carbon atoms of an alkyl, alkenyl, alkynyl or cycloalkyl group which they are bonded to, form a 3-, 4-, 5-, 6-, 7- or 8-membered saturated or partially unsaturated carbocyclic or heterocyclic ring, wherein the heterocyclic ring comprises 1, 2, 3 or 4 heteroatoms or heteroatom groups independently selected from the group consisting of N, O, S, NO, SO and SO₂, as ring members, and where the carbocyclic or heterocyclic ring is optionally substituted with one or more substituents R¹⁶; and
R⁸ as a substituent on a cycloalkyl ring is additionally selected from the group consisting of C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in these six radicals may be substituted by one or more radicals R¹³; and
R⁸ in the groups —C(═NR⁶)R⁸, and —C(═O)R⁸ is additionally selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl and C₂-C₆-haloalkynyl, wherein the aliphatic moieties in the six last-mentioned radicals may be substituted by one or more radicals R¹³;
each R⁹ is independently selected from the group consisting of hydrogen, cyano, C₁-C₆-alkyl, C₁-C₆-haloalkyl, C₃-C₈-cycloalkyl, C₃-C₈-cycloalkyl-C₁-C₄-alkyl-, C₃-C₈-halocycloalkyl, C₂-C₆-alkenyl, C₂-C₆-haloalkenyl, C₂-C₆-alkynyl, C₂-C₆-haloalkynyl, where the aliphatic and cycloaliphatic moieties in the nine last-mentioned radicals may be substituted by one or more radicals R¹³, —C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, —Si(R¹²)₃, —S(O)ₙR¹⁵, —S(O)ₙN(R^{14a})R^{14b}, —N(R^{10a})R^{10b}, —N═C(R¹³)₂, —C(═O)R¹³, —C(═O)N(R^{14a})R^{14b}, —C(═S)N(R^{14a})R^{14b}, —C(═O)OR¹⁵, phenyl, optionally substituted with one or more substituents R¹⁶; and a 3-, 4-, 5-, 6- or 7-membered saturated, partially unsaturated or maximally unsaturated heterocyclic ring comprising 1, 2 or 3 heteroatoms or heteroatom groups selected from the group consisting of N, O, S, NO, SO and SO₂, as ring members, wherein the heterocyclic ring is optionally substituted with one or more substituents R¹⁶; and R⁹ in the groups —S(O)ₙR⁹ and —OSO₂R⁹ is additionally selected from the group consisting of C₁-C₆-alkoxy and C₁-C₆-haloalkoxy;
R^{10a}, R^{10b} independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic and cycloaliphatic moieties in the eight last-mentioned radicals may be substituted by one or more radicals R¹³; —C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, C₁-C₆-alkoxy, C₁-C₆-haloalkoxy, C₁-C₆-alkylthio, C₁-C₆-haloalkylthio, —S(O)ₙR¹⁵, —S(O)ₙN(R^{14a})R^{14b}, —C(═O)R¹³, —C(═O)OR¹⁵, —C(═O)N(R^{14a})R^{14b}, —C(═S)R¹³, —C(═S)SR¹⁵, —C(═S)N(R^{14a})R^{14b}, —C(═NR¹⁴)R¹³; phenyl, optionally substituted with 1, 2, 3 or 4, substituents R¹⁶; and a 3-, 4-, 5-, 6- or 7-membered saturated, partially unsaturated or maximally unsaturated heterocyclic ring comprising 1, 2, 3 or 4 heteroatoms or heteroatom groups selected from the group consisting of N, O, S, NO, SO and SO₂, as ring members, where the heterocyclic ring is optionally substituted with one or more substituents R¹⁶;
or
R^{10a} and R^{10b} form together with the nitrogen atom they are bonded to a 3-, 4-, 5-, 6-, 7- or 8-membered saturated, partially unsaturated or maximally unsaturated heterocyclic ring, wherein the heterocyclic ring may additionally contain one or two heteroatoms or heteroatom groups selected from the group consisting of N, O, S, NO, SO and SO₂, as ring members, wherein the heterocyclic ring optionally carries one or more substituents selected from the group consisting of halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl, optionally substituted with 1, 2, 3, 4 or 5 substituents R¹⁶, and a 3-, 4-, 5-, 6-, or 7-membered saturated, partially unsaturated or maximally unsaturated heterocyclic ring comprising 1, 2 or 3 heteroatoms or heteroatom groups selected from the group consisting of N, O, S, NO, SO and SO₂, as ring members, wherein the heterocyclic ring optionally carries one or more substituents R¹⁶;
or R^{10a} and R^{10b} together form a group ═C(R¹³)₂, ═S(O)ₘ(R¹⁵)₂, ═S(O)ₘR¹⁵N(R^{14a})R^{14b}, ═NR¹⁴ or ═NOR¹⁵;
R¹¹ is independently selected from the group consisting of halogen, cyano, azido, nitro, —SCN, —SF₅, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted with one or more radicals R⁸, —OR⁹, —NR^{10a}R^{10b}, —S(O)ₙR⁹, —Si(R¹²)₃; phenyl, optionally substituted with 1, 2, 3, 4, or 5 substituents selected independently from R¹⁶; and a 3-, 4-, 5-, 6- or 7-membered saturated, partially unsaturated or maximally unsaturated aromatic heterocyclic ring comprising 1, 2, 3 or 4 heteroatoms or heteroatom groups selected from the group consisting of N, O, S, NO, SO and SO₂, as ring members, where the heterocyclic ring is optionally substituted with one or more substituents selected independently from R¹⁶;
or two R¹¹ present on the same ring carbon atom of an unsaturated or partially unsaturated heterocyclic ring may together form a group ═O, ═C(R¹³)₂; ═S; ═S(O)ₘ(R¹⁵)₂; ═S(O)ₘR¹⁵N(R^{14a})R^{14b}, ═NR¹⁴, ═NOR¹⁵, or ═NN(R^{14a})R^{14b};
or two R¹¹ bound on adjacent ring atoms form together with the ring atoms to which they are bound a saturated 3-, 4-, 5-, 6-, 7-, 8- or 9-membered ring, wherein the ring may contain 1 or 2 heteroatoms or heteroatom groups selected from the group consisting of O, S, N, NR¹⁴, NO, SO and SO₂ and/or 1 or 2 groups selected from C═O, C═S and C═NR¹⁴ as ring members, and wherein the ring may be substituted by one or more radicals selected from the group consisting of halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁶, and a 3-, 4-, 5-, 6- or 7-membered saturated, partially unsaturated or maximally unsaturated heterocyclic ring containing 1, 2 or 3 heteroatoms or heteroatom groups selected from the group consisting of N, O, S, NO, SO and SO₂, as ring members, wherein the heterocyclic ring may be substituted by one or more radicals R¹⁶;
each R¹² is independently selected from the group consisting of hydrogen, halogen, C₁-C₆-alkyl, C₁-C₆-haloalkyl, C₁-C₆-alkoxy, C₁-C₆-haloalkoxy, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, and phenyl, optionally substituted with 1, 2, 3, 4, or 5 substituents R¹⁶;
each R¹³ is independently selected from the group consisting of cyano, nitro, —OH, —SH, —SCN, —SF₅, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —NR^{14a}R^{14b}, —C(═O)NR^{14a}R^{14b}, trimethylsilyl, triethylsilyl, tert-butyldimethylsilyl, C₃-C₈-cycloalkyl which may be unsubstituted, partially or fully halogenated and/or may carry 1 or 2 radicals selected from cyano, C₁-C₄-alkyl, C₃-C₄-cycloalkyl, C₁-C₄-alkoxy, C₁-C₄-haloalkoxy and oxo; phenyl, benzyl, phenoxy, wherein the phenyl moiety in the three last-mentioned radicals may be unsubstituted or carry 1, 2, 3, 4 or 5 substituents R¹⁶; and a 3-, 4-, 5-, 6- or 7-membered saturated, partially unsaturated or maximally unsaturated heterocyclic ring containing 1, 2 or 3 heteroatoms or heteroatom groups selected from the group consisting of N, O, S, NO, SO and SO₂, as ring members, where the heterocyclic ring may be substituted by 1, 2 or 3 substituents R¹⁶;
or
two R¹³ present on the same carbon atom of an alkyl, alkenyl, alkynyl or cycloalkyl group may together be ═O, ═CH(C₁-C₄-alkyl), ═C(C₁-C₄-alkyl)C₁-C₄-alkyl, ═NR¹⁷ or ═NOR¹⁷;
and
R¹³ as a substituent on a cycloalkyl ring is additionally selected from the group consisting of C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 substituents selected from the group consisting of CN, C₃-C₄-cycloalkyl, C₁-C₄-alkoxy, C₁-C₄-haloalkoxy and oxo;
and
R¹³ in the groups ═C(R¹³)₂, —N═C(R¹³)₂, —C(═O)R¹³, —C(═S)R¹³ and —C(═NR¹⁴)R¹³ is additionally selected from the group consisting of hydrogen, halogen, C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 radicals selected from the group consisting of CN, C₃-C₄-cycloalkyl, C₁-C₄-alkoxy, C₁-C₄-haloalkoxy and oxo;
each R¹⁴ is independently selected from the group consisting of hydrogen, cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —C(═O)NR^{18a}R^{18b}, trimethylsilyl, triethylsilyl, tert-butyldimethylsilyl, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of CN, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl which may be substituted by 1 or 2 substituents selected from halogen and cyano; and oxo; C₃-C₈-cycloalkyl which may be unsubstituted, partially or fully halogenated and/or may carry 1 or 2 radicals selected from cyano, C₁-C₄-alkyl, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl, C₃-C₄-cycloalkyl-C₁-C₄-alkyl-, wherein the cycloalkyl moiety in the two last-mentioned radicals may be substituted by 1 or 2 substituents selected from the group consisting of halogen and cyano; and oxo; phenyl, benzyl, pyridyl, phenoxy, wherein the cyclic moieties in the four last-mentioned radicals may be unsubstituted and/or carry 1, 2 or 3 substituents selected from the group consisting of halogen, C₁-C₆-alkyl, C₁-C₆-haloalkyl, C₁-C₆-alkoxy, C₁-C₆-haloalkoxy and (C₁-C₆-alkoxy)carbonyl; and a 3-, 4-, 5- or 6-membered saturated, partially unsaturated or maximally unsaturated heterocyclic ring comprising 1 or 2 heteroatoms or heteroatom groups selected from the group consisting of N, O, S, NO, SO and SO₂, as ring members, where the heterocyclic ring is optionally substituted with one or more substituents R¹⁶;
R^{14a} and R^{14b}, independently of each other, have one of the meanings given for R¹⁴;
or
R^{14a} and R^{14b}, together with the nitrogen atom to which they are bound, form a 3-, 4-, 5-, 6- or 7-membered saturated, partially unsaturated or maximally unsaturated heterocyclic ring, wherein the heterocyclic ring may additionally contain 1 or 2 heteroatoms or heteroatom groups selected from the group consisting of N, O, S, NO, SO and SO₂, as ring members, wherein the heterocyclic ring optionally carries one or more substituents selected from the group consisting of halogen, C₁-C₄-alkyl, C₁-C₄-haloalkyl, C₁-C₄-alkoxy and C₁-C₄-haloalkoxy;
or
R^{14a} and R¹⁴ or R^{14b} and R¹⁴, together with the nitrogen atoms to which they are bound in the group —C(═NR¹⁴)N(R^{14a})R^{14b}, form a 3-, 4-, 5-, 6- or 7-membered partially unsaturated or maximally unsaturated heterocyclic ring, wherein the heterocyclic ring may additionally contain 1 or 2 heteroatoms or heteroatom groups selected from the group consisting of N, O, S, NO, SO and SO₂, as ring members, wherein the heterocyclic ring optionally carries one or more substituents selected from the group consisting of halogen, C₁-C₄-haloalkyl, C₁-C₄-alkoxy and C₁-C₄-haloalkoxy;
each R¹⁵ is independently selected from the group consisting of hydrogen, cyano, trimethylsilyl, triethylsilyl, tert-butyldimethylsilyl, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo; C₃-C₈-cycloalkyl which may be unsubstituted, partially or fully halogenated and/or may carry 1 or 2 radicals selected from the group consisting of C₁-C₄-alkyl, C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo; phenyl, benzyl, pyridyl and phenoxy, wherein the four last-mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆-haloalkoxy and (C₁-C₆-alkoxy)carbonyl;
each R¹⁶ is independently selected from the group consisting of halogen, nitro, cyano, —OH, —SH, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl; C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of C₃-C₄-cycloalkyl, C₁-C₄-alkoxy, C₁-C₄-haloalkoxy and oxo; C₃-C₈-cycloalkyl which may be unsubstituted, partially or fully halogenated and/or may carry 1 or 2 radicals selected from the group consisting of C₁-C₄-alkyl, C₃-C₄-cycloalkyl, C₁-C₄-alkoxy, C₁-C₄-haloalkoxy and oxo; phenyl, benzyl, pyridyl and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆-haloalkoxy and (C₁-C₆-alkoxy)carbonyl;
or
two R¹⁶ present together on the same atom of an unsaturated or partially unsaturated ring may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
or
two R¹⁶ on two adjacent carbon atoms form together with the carbon atoms they are bonded to a 4-, 5-, 6-, 7- or 8-membered saturated, partially unsaturated or maximally unsaturated ring, wherein the ring may contain 1 or 2 heteroatoms or heteroatom groups selected from the group consisting of N, O, S, NO, SO and SO₂, as ring members, and wherein the ring optionally carries one or more substituents selected from halogen, C₁-C₄-haloalkyl, C₁-C₄-alkoxy and C₁-C₄-haloalkoxy;
R¹⁷, R^{18a} and R^{18b}, independently of each other and independently of each occurrence, are selected from the group consisting of hydrogen, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of CN, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl which may be substituted by 1 or 2 substituents selected from halogen and cyano; and oxo; C₃-C₈-cycloalkyl which may be unsubstituted, partially or fully halogenated and/or may carry 1 or 2 radicals selected from the group consisting of cyano, C₁-C₄-alkyl and C₁-C₄-haloalkyl; phenyl and benzyl;
each n is independently 0, 1 or 2; and
each m is independently 0 or 1;
or a stereoisomer, enantiomer, diastereomer, N-oxide, or an agriculturally or veterinary acceptable salt thereof.

38. The method according to claim 37, wherein
R⁸ is not —CH═NOR⁹; and
A² is a group of following formula: 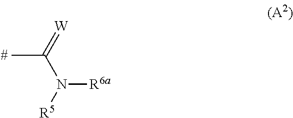
wherein
# denotes the bond to the remainder of the molecule;
W is selected from O and S;
R⁵ is selected from the group consisting of hydrogen, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted with one or more substituents R⁸, and
R^{6a} is selected from the group consisting of —X—R^{6b} and —N(R^{5a})R^{6c}; wherein
X is selected from the group consisting of —C(R^{a})₂—, —C(R^{a})₂—C(R^{a})₂—, —C(R^{a})₂—C(═O)—NR^{10a}—C(R^{a})₂—, —C(R^{a})₂—C(R^{a})₂—C(═O)—NR^{10a}—C(R^{a})₂—, —C(R^{a})₂S(O)ₙ—C(R^{a})₂—, —C(R^{a})₂—C(R^{a})₂—S(O)ₙ—C(R^{a})₂—, —C(R^{a})₂—O—C(R^{a})₂—, and —C(R^{a})₂—C(R^{a})₂—O—C(R^{a})₂—, wherein
each R^{a} is independently selected from the group consisting of hydrogen, halogen, C₁-C₃-alkyl, and C₁-C₃-haloalkyl;
R^{5a} has independently one of the meanings given for R⁵;
R^{6b} is selected from the group consisting of C₃-C₈-cycloalkyl, C₂-C₈-alkenyl, C₂-C₈-alkynyl, wherein the three last-mentioned radicals may be partially or fully halogenated and/or may be substituted by one or more substituents R⁸; and
R^{6c} is selected from the group consisting of hydrogen, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic and cycloaliphatic moieties in the eight last-mentioned radicals may be substituted by one or more radicals R¹³; —C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, C₁-C₆-alkoxy, C₁-C₆-haloalkoxy, C₁-C₆-alkylthio, C₁-C₆-haloalkylthio, —S(O)ₙR¹⁵, —S(O)ₙN(R^{14a})R^{14b}, —C(═O)R¹³, —C(═O)OR¹⁵, —C(═O)N(R^{14a})R^{14b}, —C(═S)R¹³, —C(═S)SR¹⁵, —C(═S)N(R^{14a})R^{14b}, and —C(═NR¹⁴)R¹³;
wherein if R⁵ is hydrogen, R^{6a} is further selected from the group consisting of hydrogen, 1-cyanocyclopropyl, 1-cyanocyclobutyl and 1-cyanopentyl; or
R⁵ and R^{6a} form together a group ═S(O)ₘ(R⁹)₂;
wherein R⁸, R⁹ and m are as defined in claim 37, except for R⁸ being —CH═NOR⁹;
or a stereoisomer, enantiomer, diastereomer, N-oxide, or an agriculturally or veterinary acceptable salt thereof.

39. A method for treating, preventing and protecting soybean crops and growing soybean plants from attack or infestation by pests comprising contacting a plant, or soil or water in which the plant is growing, with an isothiazoline compound of formula I or a composition comprising at least one compound of formula I 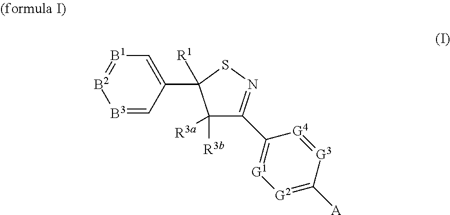
wherein
A is a group A¹, A² or A³;
wherein
A¹ is selected from the group consisting of —C(═NR⁶)R⁸, —S(O)ₙR⁹, and —N(R⁵)R⁶;
A² is a group of following formula: 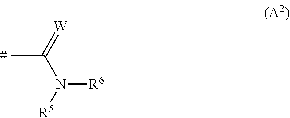
wherein
# denotes the bond to the remainder of the molecule;
W is selected from the group consisting of O and S;
A³ is a group of following formula: 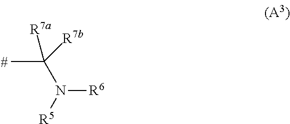
wherein
# denotes the bond to the aromatic ring of formula (I);
B¹, B² and B³ are each independently selected from the group consisting of N and CR², with the proviso that at most two of B¹, B² and B³ are N;
G¹, G², G³ and G⁴ are each independently selected from the group consisting of N and CR⁴, with the proviso that at most two of G¹, G², G³ and G⁴ are N;
R¹ is selected from the group consisting of C₁-C₄-alkyl, C₁-C₄-haloalkyl, C₁-C₄-alkoxy-C₁-C₄-alkyl, C₁-C₄-haloalkoxy-C₁-C₄-alkyl-, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl and —C(═O)OR¹⁵;
each R² is independently selected from the group consisting of hydrogen, halogen, cyano, azido, nitro, —SCN, —SF₅, C₁-C₆-alkyl, C₃-C₈-cycloalkyl, C₂-C₆-alkenyl, C₂-C₆-alkynyl, wherein the four last mentioned aliphatic and cycloaliphatic radicals may be partially or fully halogenated and/or may be substituted by one or more radicals R⁸, —Si(R¹²)₃, —OR⁹, —S(O)ₙR⁹, and —NR^{10a}R^{10b};
R^{3a}, R^{3b} are each independently selected from the group consisting of hydrogen, halogen, hydroxyl, —CO₂R^{3d}, C₁-C₃-alkyl, C₁-C₃-haloalkyl, C₂-C₃-alkenyl, C₂-C₃-alkynyl, C₁-C₃-alkoxy, C₁-C₃-haloalkoxy, C₁-C₃-alkylthio, C₁-C₃-haloalkylthio, C₁-C₃-alkylsulfonyl and C₁-C₃-haloalkylsulfonyl; or R^{3a} and R^{3b} together form a group ═O, ═C(R³c)₂, ═NOH or ═NOCH₃;
each R^{3c} is independently selected from the group consisting of hydrogen, halogen, CH₃ and CF₃;
R^{3d} is selected from the group consisting of hydrogen, C₁-C₆-alkyl and C₁-C₃-alkyloxy-C₁-C₃-alkyl-;
each R⁴ is independently selected from the group consisting of hydrogen, halogen, cyano, azido, nitro, —SCN, —SF₅, C₁-C₆-alkyl which may be partially or fully halogenated and/or may be substituted by one or more radicals R⁸, C₃-C₈-cycloalkyl which may be partially or fully halogenated and/or may be substituted by one or more radicals R⁸, C₂-C₆-alkenyl which may be partially or fully halogenated and/or may be substituted by one or more radicals R⁸, C₂-C₆-alkynyl which may be partially or fully halogenated and/or may be substituted by one or more radicals R⁸, —Si(R¹²)₃, —OR⁹, —S(O)ₙR⁹, —NR^{10a}R^{10b}, phenyl which may be substituted by 1, 2, 3, 4 or 5 radicals R¹¹, and a 3-, 4-, 5-, 6-7-, 8-, 9- or 10-membered saturated, partially unsaturated or maximally unsaturated heteromonocyclic or heterobicyclic ring containing 1, 2, 3 or 4 heteroatoms or heteroatom groups selected from the group consisting of N, O, S, NO, SO and SO₂, as ring members, where the heteromonocyclic or heterobicyclic ring may be substituted by one or more radicals R¹¹;
each R⁵ is independently selected from the group consisting of hydrogen, C₁-C₁₀-alkyl, C₃-C₈-cycloalkyl, C₂-C₁₀-alkenyl, and C₂-C₁₀-alkynyl, wherein the four last-mentioned aliphatic and cycloaliphatic radicals may be partially or fully halogenated and/or may be substituted with one or more substituents R⁸,
R⁶ in A² is selected from the group consisting of hydrogen, C₁-C₆-alkyl, C₁-C₆-haloalkyl, C₁-C₄-alkyl which carries one radical R⁸, C₂-C₆-alkenyl, C₂-C₆-haloalkenyl, C₂-C₆-alkynyl, C₃-C₆-cycloalkyl which may be substituted by 1 to 4 substituents selected from halogen and cyano; —N(R^{10a})R^{10b}, —CH═NOR⁹; phenyl which may be substituted with 1, 2, 3, 4, or 5 substituents R¹¹; and a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from the group consisting of N, O, S, NO, SO and SO₂, as ring members, wherein the heteromonocyclic or heterobicyclic ring may be substituted with one or more substituents R¹¹; or R⁵ and R⁶ form together a group ═S(O)ₘ(R⁹)₂;
each R⁶ in all other cases is independently selected from the group consisting of hydrogen, cyano, C₁-C₁₀-alkyl, C₃-C₈-cycloalkyl, C₂-C₁₀-alkenyl, and C₂-C₁₀-alkynyl, wherein the four last-mentioned aliphatic and cycloaliphatic radicals may be partially or fully halogenated and/or may be substituted by one or more substituents R⁸, —OR⁹, —NR^{10a}R^{10b}, —S(O)ₙR⁹, —C(═O)NR^{10a}N(R^{10a})R^{10b}), —Si(R¹²)₃, —C(═O)R⁸, phenyl which may be substituted with 1, 2, 3, 4, or 5 substituents R¹¹, and a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from the group consisting of N, O, S, NO, SO and SO₂, as ring members, wherein the heteromonocyclic or heterobicyclic ring may be substituted with one or more substituents R¹¹;
or
R⁵ and R⁶ or R⁵ and R^{6a}, together with the nitrogen atom to which they are bound, form a 3-, 4-, 5-, 6-, 7- or 8-membered saturated, partially unsaturated or maximally unsaturated heterocyclic ring, where the ring may further contain 1, 2, 3 or 4 heteroatoms or heteroatom-containing groups selected from the group consisting of O, S, N, SO, SO₂, C═O and C═S as ring members, wherein the heterocyclic ring may be substituted with 1, 2, 3, 4 or 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic or cycloaliphatic moieties in the twelve last-mentioned radicals may be substituted by one or more radicals R⁸, and phenyl which may be substituted with 1, 2, 3, 4 or 5 substituents R¹¹;
R^{7a}, R^{7b} are each independently selected from the group consisting of hydrogen, halogen, cyano, C₁-C₆-alkyl, C₃-C₈-cycloalkyl, C₂-C₆-alkenyl and C₂-C₆-alkynyl, wherein the four last-mentioned aliphatic and cycloaliphatic radicals may be partially or fully halogenated and/or may be substituted by one or more radicals R⁸;
each R⁸ is independently selected from the group consisting of cyano, azido, nitro, —SCN, —SF₅, C₃-C₈-cycloalkyl, C₃-C₈-halocycloalkyl, C₃-C₈-cycloalkenyl, C₃-C₈-halocycloalkenyl, wherein the cycloaliphatic moieties in the four last-mentioned radicals may be substituted by one or more radicals R¹³; —Si(R¹²)₃, —OR⁹, —OSO₂R⁹, —S(O)ₙR⁹, —N(R^{10a})R^{10b}, —C(═O)N(R^{10a})R^{10b}, —C(═S)N(R^{10a})R^{10b}, —C(═O)OR⁹, —CH═NOR⁹; phenyl, optionally substituted with 1, 2, 3, 4 or 5 substituents R¹⁶, and a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more substituents R¹⁶,
or
two R⁸ present on the same carbon atom of an alkyl, alkenyl, alkynyl or cycloalkyl group together form a group ═O, ═C(R¹³)₂; ═S; ═S(O)ₘ(R¹⁵)₂, ═S(O)ₘR¹⁵N(R^{14a})R^{14b}, ═NR^{10a}, ═NOR⁹; or ═NN(R^{10a})R^{10b};
or
two radicals R⁸, together with the carbon atoms of an alkyl, alkenyl, alkynyl or cycloalkyl group which they are bonded to, form a 3-, 4-, 5-, 6-, 7- or 8-membered saturated or partially unsaturated carbocyclic or heterocyclic ring, wherein the heterocyclic ring comprises 1, 2, 3 or 4 heteroatoms or heteroatom groups independently selected from the group consisting of N, O, S, NO, SO and SO₂, as ring members, and where the carbocyclic or heterocyclic ring is optionally substituted with one or more substituents R¹⁶; and
R⁸ as a substituent on a cycloalkyl ring is additionally selected from the group consisting of C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in these six radicals may be substituted by one or more radicals R¹³; and
R⁸ in the groups —C(═NR⁶)R⁸, and —C(═O)R⁸ is additionally selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl and C₂-C₆-haloalkynyl, wherein the aliphatic moieties in the six last-mentioned radicals may be substituted by one or more radicals R¹³;
each R⁹ is independently selected from the group consisting of hydrogen, cyano, C₁-C₆-alkyl, C₁-C₆-haloalkyl, C₃-C₈-cycloalkyl, C₃-C₈-cycloalkyl-C₁-C₄-alkyl-, C₃-C₈-halocycloalkyl, C₂-C₆-alkenyl, C₂-C₆-haloalkenyl, C₂-C₆-alkynyl, C₂-C₆-haloalkynyl, where the aliphatic and cycloaliphatic moieties in the nine last-mentioned radicals may be substituted by one or more radicals R¹³, —C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, —Si(R¹²)₃, —S(O)ₙR¹⁵, —S(O)ₙN(R^{14a})R^{14b}, —N(R^{10a})R^{10b}, —N═C(R¹³)₂, —C(═O)R¹³, —C(═O)N(R^{14a})R^{14b}, —C(═S)N(R^{14a})R^{14b}, —C(═O)OR¹⁵, phenyl, optionally substituted with one or more substituents R¹⁶; and a 3-, 4-, 5-, 6- or 7-membered saturated, partially unsaturated or maximally unsaturated heterocyclic ring comprising 1, 2 or 3 heteroatoms or heteroatom groups selected from the group consisting of N, O, S, NO, SO and SO₂, as ring members, wherein the heterocyclic ring is optionally substituted with one or more substituents R¹⁶; and R⁹ in the groups —S(O)ₙR⁹ and —OSO₂R⁹ is additionally selected from the group consisting of C₁-C₆-alkoxy and C₁-C₆-haloalkoxy;
R^{10a}, R^{10b} independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic and cycloaliphatic moieties in the eight last-mentioned radicals may be substituted by one or more radicals R¹³; —C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, C₁-C₆-alkoxy, C₁-C₆-haloalkoxy, C₁-C₆-alkylthio, C₁-C₆-haloalkylthio, —S(O)ₙR¹⁵, —S(O)ₙN(R^{14a})R^{14b}, —C(═O)R¹³, —C(═O)OR¹⁵, —C(═O)N(R^{14a})R^{14b}, —C(═S)R¹³, —C(═S)SR¹⁵, —C(═S)N(R^{14a})R^{14b}, —C(═NR¹⁴)R¹³; phenyl, optionally substituted with 1, 2, 3 or 4, substituents R¹⁶; and a 3-, 4-, 5-, 6- or 7-membered saturated, partially unsaturated or maximally unsaturated heterocyclic ring comprising 1, 2, 3 or 4 heteroatoms or heteroatom groups selected from the group consisting of N, O, S, NO, SO and SO₂, as ring members, where the heterocyclic ring is optionally substituted with one or more substituents R¹⁶;
or
R^{10a} and R^{10b} form together with the nitrogen atom they are bonded to a 3-, 4-, 5-, 6-, 7- or 8-membered saturated, partially unsaturated or maximally unsaturated heterocyclic ring, wherein the heterocyclic ring may additionally contain one or two heteroatoms or heteroatom groups selected from the group consisting of N, O, S, NO, SO and SO₂, as ring members, wherein the heterocyclic ring optionally carries one or more substituents selected from the group consisting of halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl, optionally substituted with 1, 2, 3, 4 or 5 substituents R¹⁶, and a 3-, 4-, 5-, 6-, or 7-membered saturated, partially unsaturated or maximally unsaturated heterocyclic ring comprising 1, 2 or 3 heteroatoms or heteroatom groups selected from the group consisting of N, O, S, NO, SO and SO₂, as ring members, wherein the heterocyclic ring optionally carries one or more substituents R¹⁶;
or R^{10a} and R^{10b} together form a group ═C(R¹³)₂, ═S(O)ₘ(R¹⁵)₂, ═S(O)ₘR¹⁵N(R^{14a})R^{14b}, ═NR¹⁴ or ═NOR¹⁵;
R¹¹ is independently selected from the group consisting of halogen, cyano, azido, nitro, —SCN, —SF₅, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted with one or more radicals R⁸, —OR⁹, —NR^{10a}R^{10b}, —S(O)ₙR⁹, —Si(R¹²)₃; phenyl, optionally substituted with 1, 2, 3, 4, or 5 substituents selected independently from R¹⁶; and a 3-, 4-, 5-, 6- or 7-membered saturated, partially unsaturated or maximally unsaturated aromatic heterocyclic ring comprising 1, 2, 3 or 4 heteroatoms or heteroatom groups selected from the group consisting of N, O, S, NO, SO and SO₂, as ring members, where the heterocyclic ring is optionally substituted with one or more substituents selected independently from R¹⁶;
or two R¹¹ present on the same ring carbon atom of an unsaturated or partially unsaturated heterocyclic ring may together form a group ═O, ═C(R¹³)₂; ═S; ═S(O)ₘ(R¹⁵)₂; ═S(O)ₘR¹⁵N(R^{14a})R^{14b}, ═NR¹⁴, ═NOR¹⁵, or ═NN(R^{14a})R^{14b};
or two R¹¹ bound on adjacent ring atoms form together with the ring atoms to which they are bound a saturated 3-, 4-, 5-, 6-, 7-, 8- or 9-membered ring, wherein the ring may contain 1 or 2 heteroatoms or heteroatom groups selected from the group consisting of O, S, N, NR¹⁴, NO, SO and SO₂ and/or 1 or 2 groups selected from C═O, C═S and C═NR¹⁴ as ring members, and wherein the ring may be substituted by one or more radicals selected from the group consisting of halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁶, and a 3-, 4-, 5-, 6- or 7-membered saturated, partially unsaturated or maximally unsaturated heterocyclic ring containing 1, 2 or 3 heteroatoms or heteroatom groups selected from the group consisting of N, O, S, NO, SO and SO₂, as ring members, wherein the heterocyclic ring may be substituted by one or more radicals R¹⁶;
each R¹² is independently selected from the group consisting of hydrogen, halogen, C₁-C₆-alkyl, C₁-C₆-haloalkyl, C₁-C₆-alkoxy, C₁-C₆-haloalkoxy, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, and phenyl, optionally substituted with 1, 2, 3, 4, or 5 substituents R¹⁶;
each R¹³ is independently selected from the group consisting of cyano, nitro, —OH, —SH, —SCN, —SF₅, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —NR^{14a}R^{14b}, —C(═O)NR^{14a}R^{14b}, trimethylsilyl, triethylsilyl, tert-butyldimethylsilyl, C₃-C₈-cycloalkyl which may be unsubstituted, partially or fully halogenated and/or may carry 1 or 2 radicals selected from cyano, C₁-C₄-alkyl, C₃-C₄-cycloalkyl, C₁-C₄-alkoxy, C₁-C₄-haloalkoxy and oxo; phenyl, benzyl, phenoxy, wherein the phenyl moiety in the three last-mentioned radicals may be unsubstituted or carry 1, 2, 3, 4 or 5 substituents R¹⁶; and a 3-, 4-, 5-, 6- or 7-membered saturated, partially unsaturated or maximally unsaturated heterocyclic ring containing 1, 2 or 3 heteroatoms or heteroatom groups selected from the group consisting of N, O, S, NO, SO and SO₂, as ring members, where the heterocyclic ring may be substituted by 1, 2 or 3 substituents R¹⁶;
or
two R¹³ present on the same carbon atom of an alkyl, alkenyl, alkynyl or cycloalkyl group may together be ═O, ═CH(C₁-C₄-alkyl), ═C(C₁-C₄-alkyl)C₁-C₄-alkyl, ═NR¹⁷ or ═NOR¹⁷;
and
R¹³ as a substituent on a cycloalkyl ring is additionally selected from the group consisting of C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 substituents selected from the group consisting of CN, C₃-C₄-cycloalkyl, C₁-C₄-alkoxy, C₁-C₄-haloalkoxy and oxo;
and
R¹³ in the groups ═C(R¹³)₂, —N═C(R¹³)₂, —C(═O)R¹³, —C(═S)R¹³ and —C(═NR¹⁴)R¹³ is additionally selected from the group consisting of hydrogen, halogen, C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 radicals selected from the group consisting of CN, C₃-C₄-cycloalkyl, C₁-C₄-alkoxy, C₁-C₄-haloalkoxy and oxo;
each R¹⁴ is independently selected from the group consisting of hydrogen, cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —C(═O)NR^{18a}R^{18b}, trimethylsilyl, triethylsilyl, tert-butyldimethylsilyl, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of CN, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl which may be substituted by 1 or 2 substituents selected from halogen and cyano; and oxo; C₃-C₈-cycloalkyl which may be unsubstituted, partially or fully halogenated and/or may carry 1 or 2 radicals selected from cyano, C₁-C₄-alkyl, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl, C₃-C₄-cycloalkyl-C₁-C₄-alkyl-, wherein the cycloalkyl moiety in the two last-mentioned radicals may be substituted by 1 or 2 substituents selected from the group consisting of halogen and cyano; and oxo; phenyl, benzyl, pyridyl, phenoxy, wherein the cyclic moieties in the four last-mentioned radicals may be unsubstituted and/or carry 1, 2 or 3 substituents selected from the group consisting of halogen, C₁-C₆-alkyl, C₁-C₆-haloalkyl, C₁-C₆-alkoxy, C₁-C₆-haloalkoxy and (C₁-C₆-alkoxy)carbonyl; and a 3-, 4-, 5- or 6-membered saturated, partially unsaturated or maximally unsaturated heterocyclic ring comprising 1 or 2 heteroatoms or heteroatom groups selected from the group consisting of N, O, S, NO, SO and SO₂, as ring members, where the heterocyclic ring is optionally substituted with one or more substituents R¹⁶;
R^{14a} and R^{14b}, independently of each other, have one of the meanings given for R¹⁴;
or
R^{14a} and R^{14b}, together with the nitrogen atom to which they are bound, form a 3-, 4-, 5-, 6- or 7-membered saturated, partially unsaturated or maximally unsaturated heterocyclic ring, wherein the heterocyclic ring may additionally contain 1 or 2 heteroatoms or heteroatom groups selected from the group consisting of N, O, S, NO, SO and SO₂, as ring members, wherein the heterocyclic ring optionally carries one or more substituents selected from the group consisting of halogen, C₁-C₄-alkyl, C₁-C₄-haloalkyl, C₁-C₄-alkoxy and C₁-C₄-haloalkoxy;
or
R^{14a} and R¹⁴ or R^{14b} and R¹⁴, together with the nitrogen atoms to which they are bound in the group —C(═NR¹⁴)N(R^{14a})R^{14b}, form a 3-, 4-, 5-, 6- or 7-membered partially unsaturated or maximally unsaturated heterocyclic ring, wherein the heterocyclic ring may additionally contain 1 or 2 heteroatoms or heteroatom groups selected from the group consisting of N, O, S, NO, SO and SO₂, as ring members, wherein the heterocyclic ring optionally carries one or more substituents selected from the group consisting of halogen, C₁-C₄-haloalkyl, C₁-C₄-alkoxy and C₁-C₄-haloalkoxy;
each R¹⁵ is independently selected from the group consisting of hydrogen, cyano, trimethylsilyl, triethylsilyl, tert-butyldimethylsilyl, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo; C₃-C₈-cycloalkyl which may be unsubstituted, partially or fully halogenated and/or may carry 1 or 2 radicals selected from the group consisting of C₁-C₄-alkyl, C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo; phenyl, benzyl, pyridyl and phenoxy, wherein the four last-mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆-haloalkoxy and (C₁-C₆-alkoxy)carbonyl;
each R¹⁶ is independently selected from the group consisting of halogen, nitro, cyano, —OH, —SH, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl; C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of C₃-C₄-cycloalkyl, C₁-C₄-alkoxy, C₁-C₄-haloalkoxy and oxo; C₃-C₈-cycloalkyl which may be unsubstituted, partially or fully halogenated and/or may carry 1 or 2 radicals selected from the group consisting of C₁-C₄-alkyl, C₃-C₄-cycloalkyl, C₁-C₄-alkoxy, C₁-C₄-haloalkoxy and oxo; phenyl, benzyl, pyridyl and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆-haloalkoxy and (C₁-C₆-alkoxy)carbonyl;
or
two R¹⁶ present together on the same atom of an unsaturated or partially unsaturated ring may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
or
two R¹⁶ on two adjacent carbon atoms form together with the carbon atoms they are bonded to a 4-, 5-, 6-, 7- or 8-membered saturated, partially unsaturated or maximally unsaturated ring, wherein the ring may contain 1 or 2 heteroatoms or heteroatom groups selected from the group consisting of N, O, S, NO, SO and SO₂, as ring members, and wherein the ring optionally carries one or more substituents selected from halogen, C₁-C₄-haloalkyl, C₁-C₄-alkoxy and C₁-C₄-haloalkoxy;
R¹⁷, R^{18a} and R^{18b}, independently of each other and independently of each occurrence, are selected from the group consisting of hydrogen, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of CN, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl which may be substituted by 1 or 2 substituents selected from halogen and cyano; and oxo; C₃-C₈-cycloalkyl which may be unsubstituted, partially or fully halogenated and/or may carry 1 or 2 radicals selected from the group consisting of cyano, C₁-C₄-alkyl and C₁-C₄-haloalkyl; phenyl and benzyl;
each n is independently 0, 1 or 2; and
each m is independently 0 or 1;
or a stereoisomer, enantiomer, diastereomer, N-oxide, or an agriculturally or veterinary acceptable salt thereof.

40. The method of claim 37, wherein the pests are from the family of Pentatomidae (stink bugs).

41. The method of claim 40, wherein the pests are Acrosternum spp., Euschistus spp., Nezara spp., Piezodrus spp., Dichelops spp., Halyomorpha halys, Thyanta accerra, Podisus maculiventris, and/or Megacopta cribraria.

42. The method of claim 41, wherein the pests are Acrosternum spp., Euschistus spp., Nezara spp. and/or Piezodrus spp.

43. The method of claim 42, wherein the pests are Acrosternum hilare, Euschistus heros, Nezara viridula and/or Piezodrus guildinii.

44. The method of claim 41, wherein the pest is Halyomorpha halys.

45. The method of claim 37, wherein the compound of formula I is applied by foliar application.

46. A method for controlling and protecting soybean plant propagation material from infestation by pests from the family of Pentatomidae (stink bugs) comprising contacting the plant propagation material, especially the seeds, before sowing and/or after pregermination with an isothiazoline compound of formula I or a composition comprising at least one compound of formula I: 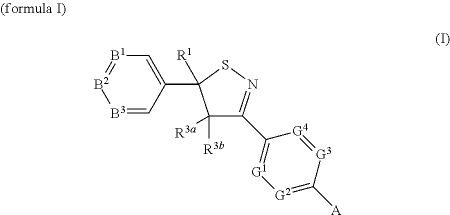
wherein
A is a group A¹, A² or A³;
wherein
A¹ is selected from the group consisting of —C(═NR⁶)R⁸, —S(O)ₙR⁹, and —N(R⁵)R⁶;
A² is a group of following formula: 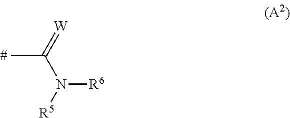
wherein
# denotes the bond to the remainder of the molecule;
W is selected from the group consisting of O and S;
A³ is a group of following formula: 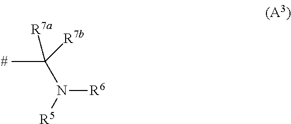
wherein
# denotes the bond to the aromatic ring of formula (I);
B¹, B² and B³ are each independently selected from the group consisting of N and CR², with the proviso that at most two of B¹, B² and B³ are N;
G¹, G², G³ and G⁴ are each independently selected from the group consisting of N and CR⁴, with the proviso that at most two of G¹, G², G³ and G⁴ are N;
R¹ is selected from the group consisting of C₁-C₄-alkyl, C₁-C₄-haloalkyl, C₁-C₄-alkoxy-C₁-C₄-alkyl, C₁-C₄-haloalkoxy-C₁-C₄-alkyl-, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl and —C(═O)OR¹⁵;
each R² is independently selected from the group consisting of hydrogen, halogen, cyano, azido, nitro, —SCN, —SF₅, C₁-C₆-alkyl, C₃-C₈-cycloalkyl, C₂-C₆-alkenyl, C₂-C₆-alkynyl, wherein the four last mentioned aliphatic and cycloaliphatic radicals may be partially or fully halogenated and/or may be substituted by one or more radicals R⁸, —Si(R¹²)₃, —OR⁹, —S(O)ₙR⁹, and —NR^{10a}R^{10b};
R^{3a}, R^{3b} are each independently selected from the group consisting of hydrogen, halogen, hydroxyl, —CO₂R^{3d}, C₁-C₃-alkyl, C₁-C₃-haloalkyl, C₂-C₃-alkenyl, C₂-C₃-alkynyl, C₁-C₃-alkoxy, C₁-C₃-haloalkoxy, C₁-C₃-alkylthio, C₁-C₃-haloalkylthio, C₁-C₃-alkylsulfonyl and C₁-C₃-haloalkylsulfonyl; or R^{3a} and R^{3b} together form a group ═O, ═C(R^{3c})₂, ═NOH or ═NOCH₃;
each R^{3c} is independently selected from the group consisting of hydrogen, halogen, CH₃ and CF₃;
R^{3d} is selected from the group consisting of hydrogen, C₁-C₆-alkyl and C₁-C₃-alkyloxy-C₁-C₃-alkyl-;
each R⁴ is independently selected from the group consisting of hydrogen, halogen, cyano, azido, nitro, —SCN, —SF₅, C₁-C₆-alkyl which may be partially or fully halogenated and/or may be substituted by one or more radicals R⁸, C₃-C₈-cycloalkyl which may be partially or fully halogenated and/or may be substituted by one or more radicals R⁸, C₂-C₆-alkenyl which may be partially or fully halogenated and/or may be substituted by one or more radicals R⁸, C₂-C₆-alkynyl which may be partially or fully halogenated and/or may be substituted by one or more radicals R⁸, —Si(R¹²)₃, —OR⁹, —S(O)ₙR⁹, —NR^{10a}R^{10b}, phenyl which may be substituted by 1, 2, 3, 4 or 5 radicals R¹¹, and a 3-, 4-, 5-, 6-7-, 8-, 9- or 10-membered saturated, partially unsaturated or maximally unsaturated heteromonocyclic or heterobicyclic ring containing 1, 2, 3 or 4 heteroatoms or heteroatom groups selected from the group consisting of N, O, S, NO, SO and SO₂, as ring members, where the heteromonocyclic or heterobicyclic ring may be substituted by one or more radicals R¹¹;
each R⁵ is independently selected from the group consisting of hydrogen, C₁-C₁₀-alkyl, C₃-C₈-cycloalkyl, C₂-C₁₀-alkenyl, and C₂-C₁₀-alkynyl, wherein the four last-mentioned aliphatic and cycloaliphatic radicals may be partially or fully halogenated and/or may be substituted with one or more substituents R⁸,
R⁶ in A² is selected from the group consisting of hydrogen, C₁-C₆-alkyl, C₁-C₆-haloalkyl, C₁-C₄-alkyl which carries one radical R⁸, C₂-C₆-alkenyl, C₂-C₆-haloalkenyl, C₂-C₆-alkynyl, C₃-C₆-cycloalkyl which may be substituted by 1 to 4 substituents selected from halogen and cyano; —N(R^{10a})R^{10b}, —CH═NOR⁹; phenyl which may be substituted with 1, 2, 3, 4, or 5 substituents R¹¹; and a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from the group consisting of N, O, S, NO, SO and SO₂, as ring members, wherein the heteromonocyclic or heterobicyclic ring may be substituted with one or more substituents R¹¹; or R⁵ and R⁶ form together a group ═S(O)ₘ(R⁹)₂;
each R⁶ in all other cases is independently selected from the group consisting of hydrogen, cyano, C₁-C₁₀-alkyl, C₃-C₈-cycloalkyl, C₂-C₁₀-alkenyl, and C₂-C₁₀-alkynyl, wherein the four last-mentioned aliphatic and cycloaliphatic radicals may be partially or fully halogenated and/or may be substituted by one or more substituents R⁸, —OR⁹, —NR^{10a}R^{10b}, —S(O)ₙR⁹, —C(═O)NR^{10a}N(R^{10a})R^{10b}), —Si(R¹²)₃, —C(═O)R⁸, phenyl which may be substituted with 1, 2, 3, 4, or 5 substituents R¹¹, and a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from the group consisting of N, O, S, NO, SO and SO₂, as ring members, wherein the heteromonocyclic or heterobicyclic ring may be substituted with one or more substituents R¹¹;
or
R⁵ and R⁶ or R⁵ and R^{6a}, together with the nitrogen atom to which they are bound, form a 3-, 4-, 5-, 6-, 7- or 8-membered saturated, partially unsaturated or maximally unsaturated heterocyclic ring, where the ring may further contain 1, 2, 3 or 4 heteroatoms or heteroatom-containing groups selected from the group consisting of O, S, N, SO, SO₂, C═O and C═S as ring members, wherein the heterocyclic ring may be substituted with 1, 2, 3, 4 or 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic or cycloaliphatic moieties in the twelve last-mentioned radicals may be substituted by one or more radicals R⁸, and phenyl which may be substituted with 1, 2, 3, 4 or 5 substituents R¹¹;
R^{7a}, R^{7b} are each independently selected from the group consisting of hydrogen, halogen, cyano, C₁-C₆-alkyl, C₃-C₈-cycloalkyl, C₂-C₆-alkenyl and C₂-C₆-alkynyl, wherein the four last-mentioned aliphatic and cycloaliphatic radicals may be partially or fully halogenated and/or may be substituted by one or more radicals R⁸;
each R⁸ is independently selected from the group consisting of cyano, azido, nitro, —SCN, —SF₅, C₃-C₈-cycloalkyl, C₃-C₈-halocycloalkyl, C₃-C₈-cycloalkenyl, C₃-C₈-halocycloalkenyl, wherein the cycloaliphatic moieties in the four last-mentioned radicals may be substituted by one or more radicals R¹³; —Si(R¹²)₃, —OR⁹, —OSO₂R⁹, —S(O)ₙR⁹, —N(R^{10a})R^{10b}, —C(═O)N(R^{10a})R^{10b}, —C(═S)N(R^{10a})R^{10b}, —C(═O)OR⁹, —CH═NOR⁹; phenyl, optionally substituted with 1, 2, 3, 4 or 5 substituents R¹⁶, and a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more substituents R¹⁶,
or
two R⁸ present on the same carbon atom of an alkyl, alkenyl, alkynyl or cycloalkyl group together form a group ═O, ═C(R¹³)₂; ═S; ═S(O)ₘ(R¹⁵)₂, ═S(O)ₘR¹⁵N(R^{14a})R^{14b}, ═NR^{10a}, ═NOR⁹; or ═NN(R^{10a})R^{10b};
or
two radicals R⁸, together with the carbon atoms of an alkyl, alkenyl, alkynyl or cycloalkyl group which they are bonded to, form a 3-, 4-, 5-, 6-, 7- or 8-membered saturated or partially unsaturated carbocyclic or heterocyclic ring, wherein the heterocyclic ring comprises 1, 2, 3 or 4 heteroatoms or heteroatom groups independently selected from the group consisting of N, O, S, NO, SO and SO₂, as ring members, and where the carbocyclic or heterocyclic ring is optionally substituted with one or more substituents R¹⁶; and
R⁸ as a substituent on a cycloalkyl ring is additionally selected from the group consisting of C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in these six radicals may be substituted by one or more radicals R¹³; and
R⁸ in the groups —C(═NR⁶)R⁸, and —C(═O)R⁸ is additionally selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl and C₂-C₆-haloalkynyl, wherein the aliphatic moieties in the six last-mentioned radicals may be substituted by one or more radicals R¹³;
each R⁹ is independently selected from the group consisting of hydrogen, cyano, C₁-C₆-alkyl, C₁-C₆-haloalkyl, C₃-C₈-cycloalkyl, C₃-C₈-cycloalkyl-C₁-C₄-alkyl-, C₃-C₈-halocycloalkyl, C₂-C₆-alkenyl, C₂-C₆-haloalkenyl, C₂-C₆-alkynyl, C₂-C₆-haloalkynyl, where the aliphatic and cycloaliphatic moieties in the nine last-mentioned radicals may be substituted by one or more radicals R¹³, —C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, —Si(R¹²)₃, —S(O)ₙR¹⁵, —S(O)ₙN(R^{14a})R^{14b}, —N(R^{10a})R^{10b}, —N═C(R¹³)₂, —C(═O)R¹³, —C(═O)N(R^{14a})R^{14b}, —C(═S)N(R^{14a})R^{14b}, —C(═O)OR¹⁵, phenyl, optionally substituted with one or more substituents R¹⁶; and a 3-, 4-, 5-, 6- or 7-membered saturated, partially unsaturated or maximally unsaturated heterocyclic ring comprising 1, 2 or 3 heteroatoms or heteroatom groups selected from the group consisting of N, O, S, NO, SO and SO₂, as ring members, wherein the heterocyclic ring is optionally substituted with one or more substituents R¹⁶; and R⁹ in the groups —S(O)ₙR⁹ and —OSO₂R⁹ is additionally selected from the group consisting of C₁-C₆-alkoxy and C₁-C₆-haloalkoxy;
R^{10a}, R^{10b} independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic and cycloaliphatic moieties in the eight last-mentioned radicals may be substituted by one or more radicals R¹³; —C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, C₁-C₆-alkoxy, C₁-C₆-haloalkoxy, C₁-C₆-alkylthio, C₁-C₆-haloalkylthio, —S(O)ₙR¹⁵, —S(O)ₙN(R^{14a})R^{14b}, —C(═O)R¹³, —C(═O)OR¹⁵, —C(═O)N(R^{14a})R^{14b}, —C(═S)R¹³, —C(═S)SR¹⁵, —C(═S)N(R^{14a})R^{14b}, —C(═NR¹⁴)R¹³; phenyl, optionally substituted with 1, 2, 3 or 4, substituents R¹⁶; and a 3-, 4-, 5-, 6- or 7-membered saturated, partially unsaturated or maximally unsaturated heterocyclic ring comprising 1, 2, 3 or 4 heteroatoms or heteroatom groups selected from the group consisting of N, O, S, NO, SO and SO₂, as ring members, where the heterocyclic ring is optionally substituted with one or more substituents R¹⁶;
or
R^{10a} and R^{10b} form together with the nitrogen atom they are bonded to a 3-, 4-, 5-, 6-, 7- or 8-membered saturated, partially unsaturated or maximally unsaturated heterocyclic ring, wherein the heterocyclic ring may additionally contain one or two heteroatoms or heteroatom groups selected from the group consisting of N, O, S, NO, SO and SO₂, as ring members, wherein the heterocyclic ring optionally carries one or more substituents selected from the group consisting of halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl, optionally substituted with 1, 2, 3, 4 or 5 substituents R¹⁶ and a 3-, 4-, 5-, 6-, or 7-membered saturated, partially unsaturated or maximally unsaturated heterocyclic ring comprising 1, 2 or 3 heteroatoms or heteroatom groups selected from the group consisting of N, O, S, NO, SO and SO₂, as ring members, wherein the heterocyclic ring optionally carries one or more substituents R¹⁶;
or R^{10a} and R^{10b} together form a group ═C(R¹³)₂, ═S(O)ₘ(R¹⁵)₂, ═S(O)ₘR¹⁵N(R^{14a})R^{14b}, ═NR¹⁴ or ═NOR¹⁵;
R¹¹ is independently selected from the group consisting of halogen, cyano, azido, nitro, —SCN, —SF₅, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted with one or more radicals R⁸, —OR⁹, —NR^{10a}R^{10b}, —S(O)ₙR⁹, —Si(R¹²)₃; phenyl, optionally substituted with 1, 2, 3, 4, or 5 substituents selected independently from R¹⁶; and a 3-, 4-, 5-, 6- or 7-membered saturated, partially unsaturated or maximally unsaturated aromatic heterocyclic ring comprising 1, 2, 3 or 4 heteroatoms or heteroatom groups selected from the group consisting of N, O, S, NO, SO and SO₂, as ring members, where the heterocyclic ring is optionally substituted with one or more substituents selected independently from R¹⁶;
or two R¹¹ present on the same ring carbon atom of an unsaturated or partially unsaturated heterocyclic ring may together form a group ═O, ═C(R¹³)₂; ═S; ═S(O)ₘ(R¹⁵)₂; ═S(O)ₘR¹⁵N(R^{14a})R^{14b}, ═NR¹⁴, ═NOR¹⁵, or ═NN(R^{14a})R^{14b}; or two R¹¹ bound on adjacent ring atoms form together with the ring atoms to which they are bound a saturated 3-, 4-, 5-, 6-, 7-, 8- or 9-membered ring, wherein the ring may contain 1 or 2 heteroatoms or heteroatom groups selected from the group consisting of O, S, N, NR¹⁴, NO, SO and SO₂ and/or 1 or 2 groups selected from C═O, C═S and C═NR¹⁴ as ring members, and wherein the ring may be substituted by one or more radicals selected from the group consisting of halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁶, and a 3-, 4-, 5-, 6- or 7-membered saturated, partially unsaturated or maximally unsaturated heterocyclic ring containing 1, 2 or 3 heteroatoms or heteroatom groups selected from the group consisting of N, O, S, NO, SO and SO₂, as ring members, wherein the heterocyclic ring may be substituted by one or more radicals R¹⁶;
each R¹² is independently selected from the group consisting of hydrogen, halogen, C₁-C₆-alkyl, C₁-C₆-haloalkyl, C₁-C₆-alkoxy, C₁-C₆-haloalkoxy, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, and phenyl, optionally substituted with 1, 2, 3, 4, or 5 substituents R¹⁶;
each R¹³ is independently selected from the group consisting of cyano, nitro, —OH, —SH, —SCN, —SF₅, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —NR^{14a}R^{14b}, —C(═O)NR^{14a}R^{14b}, trimethylsilyl, triethylsilyl, tert-butyldimethylsilyl, C₃-C₈-cycloalkyl which may be unsubstituted, partially or fully halogenated and/or may carry 1 or 2 radicals selected from cyano, C₁-C₄-alkyl, C₃-C₄-cycloalkyl, C₁-C₄-alkoxy, C₁-C₄-haloalkoxy and oxo; phenyl, benzyl, phenoxy, wherein the phenyl moiety in the three last-mentioned radicals may be unsubstituted or carry 1, 2, 3, 4 or 5 substituents R¹⁶; and a 3-, 4-, 5-, 6- or 7-membered saturated, partially unsaturated or maximally unsaturated heterocyclic ring containing 1, 2 or 3 heteroatoms or heteroatom groups selected from the group consisting of N, O, S, NO, SO and SO₂, as ring members, where the heterocyclic ring may be substituted by 1, 2 or 3 substituents R¹⁶;
or
two R¹³ present on the same carbon atom of an alkyl, alkenyl, alkynyl or cycloalkyl group may together be ═O, ═CH(C₁-C₄-alkyl), ═C(C₁-C₄-alkyl)C₁-C₄-alkyl, ═NR¹⁷ or ═NOR¹⁷;
and
R¹³ as a substituent on a cycloalkyl ring is additionally selected from the group consisting of C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 substituents selected from the group consisting of CN, C₃-C₄-cycloalkyl, C₁-C₄-alkoxy, C₁-C₄-haloalkoxy and oxo;
and
R¹³ in the groups ═C(R¹³)₂, —N═C(R¹³)₂, —C(═O)R¹³, —C(═S)R¹³ and —C(═NR¹⁴)R¹³ is additionally selected from the group consisting of hydrogen, halogen, C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 radicals selected from the group consisting of CN, C₃-C₄-cycloalkyl, C₁-C₄-alkoxy, C₁-C₄-haloalkoxy and oxo;
each R¹⁴ is independently selected from the group consisting of hydrogen, cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —C(═O)NR^{18a}R^{18b}, trimethylsilyl, triethylsilyl, tert-butyldimethylsilyl, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of CN, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl which may be substituted by 1 or 2 substituents selected from halogen and cyano; and oxo; C₃-C₈-cycloalkyl which may be unsubstituted, partially or fully halogenated and/or may carry 1 or 2 radicals selected from cyano, C₁-C₄-alkyl, C₁-C₄-alkoxy, C₁-C₄-haloalkoxy, C₁i-C₄-alkylthio, C₁-C₄-alkylsulfinyl, C₁-C₄-alkylsulfonyl, C₃-C₄-cycloalkyl, C₃-C₄-cycloalkyl-C₁-C₄-alkyl-, wherein the cycloalkyl moiety in the two last-mentioned radicals may be substituted by 1 or 2 substituents selected from the group consisting of halogen and cyano; and oxo; phenyl, benzyl, pyridyl, phenoxy, wherein the cyclic moieties in the four last-mentioned radicals may be unsubstituted and/or carry 1, 2 or 3 substituents selected from the group consisting of halogen, C₁-C₆-alkyl, C₁-C₆-haloalkyl, C₁-C₆-alkoxy, C₁-C₆-haloalkoxy and (C₁-C₆-alkoxy)carbonyl; and a 3-, 4-, 5- or 6-membered saturated, partially unsaturated or maximally unsaturated heterocyclic ring comprising 1 or 2 heteroatoms or heteroatom groups selected from the group consisting of N, O, S, NO, SO and SO₂, as ring members, where the heterocyclic ring is optionally substituted with one or more substituents R¹⁶;
R^{14a} and R^{14b}, independently of each other, have one of the meanings given for R¹⁴;
or
R^{14a} and R^{14b}, together with the nitrogen atom to which they are bound, form a 3-, 4-, 5-, 6- or 7-membered saturated, partially unsaturated or maximally unsaturated heterocyclic ring, wherein the heterocyclic ring may additionally contain 1 or 2 heteroatoms or heteroatom groups selected from the group consisting of N, O, S, NO, SO and SO₂, as ring members, wherein the heterocyclic ring optionally carries one or more substituents selected from the group consisting of halogen, C₁-C₄-alkyl, C₁-C₄-haloalkyl, C₁-C₄-alkoxy and C₁-C₄-haloalkoxy;
or
R^{14a} and R¹⁴ or R^{14b} and R¹⁴, together with the nitrogen atoms to which they are bound in the group —C(═NR¹⁴)N(R^{14a})R^{14b}, form a 3-, 4-, 5-, 6- or 7-membered partially unsaturated or maximally unsaturated heterocyclic ring, wherein the heterocyclic ring may additionally contain 1 or 2 heteroatoms or heteroatom groups selected from the group consisting of N, O, S, NO, SO and SO₂, as ring members, wherein the heterocyclic ring optionally carries one or more substituents selected from the group consisting of halogen, C₁-C₄-haloalkyl, C₁-C₄-alkoxy and C₁-C₄-haloalkoxy;
each R¹⁵ is independently selected from the group consisting of hydrogen, cyano, trimethylsilyl, triethylsilyl, tert-butyldimethylsilyl, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo; C₃-C₈-cycloalkyl which may be unsubstituted, partially or fully halogenated and/or may carry 1 or 2 radicals selected from the group consisting of C₁-C₄-alkyl, C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo; phenyl, benzyl, pyridyl and phenoxy, wherein the four last-mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆-haloalkoxy and (C₁-C₆-alkoxy)carbonyl;
each R¹⁶ is independently selected from the group consisting of halogen, nitro, cyano, —OH, —SH, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl; C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of C₃-C₄-cycloalkyl, C₁-C₄-alkoxy, C₁-C₄-haloalkoxy and oxo; C₃-C₈-cycloalkyl which may be unsubstituted, partially or fully halogenated and/or may carry 1 or 2 radicals selected from the group consisting of C₁-C₄-alkyl, C₃-C₄-cycloalkyl, C₁-C₄-alkoxy, C₁-C₄-haloalkoxy and oxo; phenyl, benzyl, pyridyl and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆-haloalkoxy and (C₁-C₆-alkoxy)carbonyl;
or
two R¹⁶ present together on the same atom of an unsaturated or partially unsaturated ring may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
or
two R¹⁶ on two adjacent carbon atoms form together with the carbon atoms they are bonded to a 4-, 5-, 6-, 7- or 8-membered saturated, partially unsaturated or maximally unsaturated ring, wherein the ring may contain 1 or 2 heteroatoms or heteroatom groups selected from the group consisting of N, O, S, NO, SO and SO₂, as ring members, and wherein the ring optionally carries one or more substituents selected from halogen, C₁-C₄-haloalkyl, C₁-C₄-alkoxy and C₁-C₄-haloalkoxy;
R¹⁷, R^{18a} and R^{18b}, independently of each other and independently of each occurrence, are selected from the group consisting of hydrogen, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of CN, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl which may be substituted by 1 or 2 substituents selected from halogen and cyano; and oxo; C₃-C₈-cycloalkyl which may be unsubstituted, partially or fully halogenated and/or may carry 1 or 2 radicals selected from the group consisting of cyano, C₁-C₄-alkyl and C₁-C₄-haloalkyl; phenyl and benzyl;
each n is independently 0, 1 or 2; and
each m is independently 0 or 1;
or a stereoisomer, enantiomer, diastereomer, N-oxide, or an agriculturally or veterinary acceptable salt thereof.

47. A method for combating or controlling pests from the family of Pentatomidae (stink bugs) comprising contacting the stink bugs, their food supply, habitat or breeding grounds, the plant or the crop with an isothiazoline compound of formula I or a composition comprising at least one compound of formula I: 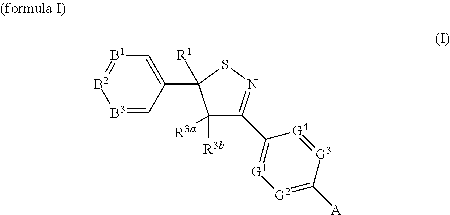
wherein
A is a group A¹, A² or A³;
wherein
A¹ is selected from the group consisting of —C(═NR⁶)R⁸, —S(O)ₙR⁹, and —N(R⁵)R⁶;
A² is a group of following formula: 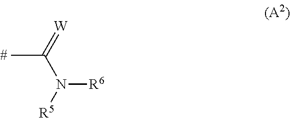
wherein
# denotes the bond to the remainder of the molecule;
W is selected from the group consisting of O and S;
A³ is a group of following formula: 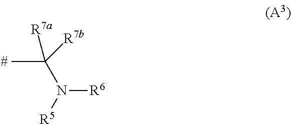
wherein
# denotes the bond to the aromatic ring of formula (I);
B¹, B² and B³ are each independently selected from the group consisting of N and CR², with the proviso that at most two of B¹, B² and B³ are N;
G¹, G², G³ and G⁴ are each independently selected from the group consisting of N and CR⁴, with the proviso that at most two of G¹, G², G³ and G⁴ are N;
R¹ is selected from the group consisting of C₁-C₄-alkyl, C₁-C₄-haloalkyl, C₁-C₄-alkoxy-C₁-C₄-alkyl, C₁-C₄-haloalkoxy-C₁-C₄-alkyl-, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl and —C(═O)OR¹⁵;
each R² is independently selected from the group consisting of hydrogen, halogen, cyano, azido, nitro, —SCN, —SF₅, C₁-C₆-alkyl, C₃-C₈-cycloalkyl, C₂-C₆-alkenyl, C₂-C₆-alkynyl, wherein the four last mentioned aliphatic and cycloaliphatic radicals may be partially or fully halogenated and/or may be substituted by one or more radicals R⁸, —Si(R¹²)₃, —OR⁹, —S(O)ₙR⁹, and —NR^{10a}R^{10b};
R^{3a}, R^{3b} are each independently selected from the group consisting of hydrogen, halogen, hydroxyl, —CO₂R^{3d}, C₁-C₃-alkyl, C₁-C₃-haloalkyl, C₂-C₃-alkenyl, C₂-C₃-alkynyl, C₁-C₃-alkoxy, C₁-C₃-haloalkoxy, C₁-C₃-alkylthio, C₁-C₃-haloalkylthio, C₁-C₃-alkylsulfonyl and C₁-C₃-haloalkylsulfonyl; or R^{3a} and R^{3b} together form a group ═O, ═C(R^{3c})₂, ═NOH or ═NOCH₃;
each R^{3c} is independently selected from the group consisting of hydrogen, halogen, CH₃ and CF₃;
R^{3d} is selected from the group consisting of hydrogen, C₁-C₆-alkyl and C₁-C₃-alkyloxy-C₁-C₃-alkyl-;
each R⁴ is independently selected from the group consisting of hydrogen, halogen, cyano, azido, nitro, —SCN, —SF₅, C₁-C₆-alkyl which may be partially or fully halogenated and/or may be substituted by one or more radicals R⁸, C₃-C₈-cycloalkyl which may be partially or fully halogenated and/or may be substituted by one or more radicals R⁸, C₂-C₆-alkenyl which may be partially or fully halogenated and/or may be substituted by one or more radicals R⁸, C₂-C₆-alkynyl which may be partially or fully halogenated and/or may be substituted by one or more radicals R⁸, —Si(R¹²)₃, —OR⁹, —S(O)ₙR⁹, —NR^{10a}R^{10b}, phenyl which may be substituted by 1, 2, 3, 4 or 5 radicals R¹¹, and a 3-, 4-, 5-, 6-7-, 8-, 9- or 10-membered saturated, partially unsaturated or maximally unsaturated heteromonocyclic or heterobicyclic ring containing 1, 2, 3 or 4 heteroatoms or heteroatom groups selected from the group consisting of N, O, S, NO, SO and SO₂, as ring members, where the heteromonocyclic or heterobicyclic ring may be substituted by one or more radicals R¹¹;
each R⁵ is independently selected from the group consisting of hydrogen, C₁-C₁₀-alkyl, C₃-C₈-cycloalkyl, C₂-C₁₀-alkenyl, and C₂-C₁₀-alkynyl, wherein the four last-mentioned aliphatic and cycloaliphatic radicals may be partially or fully halogenated and/or may be substituted with one or more substituents R⁸,
R⁶ in A² is selected from the group consisting of hydrogen, C₁-C₆-alkyl, C₁-C₆-haloalkyl, C₁-C₄-alkyl which carries one radical R⁸, C₂-C₆-alkenyl, C₂-C₆-haloalkenyl, C₂-C₆-alkynyl, C₃-C₆-cycloalkyl which may be substituted by 1 to 4 substituents selected from halogen and cyano; —N(R^{10a})R^{10b}, —CH═NOR⁹; phenyl which may be substituted with 1, 2, 3, 4, or 5 substituents R¹¹; and a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from the group consisting of N, O, S, NO, SO and SO₂, as ring members, wherein the heteromonocyclic or heterobicyclic ring may be substituted with one or more substituents R¹¹; or R⁵ and R⁶ form together a group ═S(O)ₘ(R⁹)₂;
each R⁶ in all other cases is independently selected from the group consisting of hydrogen, cyano, C₁-C₁₀-alkyl, C₃-C₈-cycloalkyl, C₂-C₁₀-alkenyl, and C₂-C₁₀-alkynyl, wherein the four last-mentioned aliphatic and cycloaliphatic radicals may be partially or fully halogenated and/or may be substituted by one or more substituents R⁸, —OR⁹, —NR^{10a}R^{10b}, —S(O)ₙR⁹, —C(═O)NR^{10a}N(R^{10a})R^{10b}), —Si(R¹²)₃, —C(═O)R⁸, phenyl which may be substituted with 1, 2, 3, 4, or 5 substituents R¹¹, and a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from the group consisting of N, O, S, NO, SO and SO₂, as ring members, wherein the heteromonocyclic or heterobicyclic ring may be substituted with one or more substituents R¹¹;
or
R⁵ and R⁶ or R⁵ and R^{6a}, together with the nitrogen atom to which they are bound, form a 3-, 4-, 5-, 6-, 7- or 8-membered saturated, partially unsaturated or maximally unsaturated heterocyclic ring, where the ring may further contain 1, 2, 3 or 4 heteroatoms or heteroatom-containing groups selected from the group consisting of O, S, N, SO, SO₂, C═O and C═S as ring members, wherein the heterocyclic ring may be substituted with 1, 2, 3, 4 or 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic or cycloaliphatic moieties in the twelve last-mentioned radicals may be substituted by one or more radicals R⁸, and phenyl which may be substituted with 1, 2, 3, 4 or 5 substituents R¹¹;
R^{7a}, R^{7b} are each independently selected from the group consisting of hydrogen, halogen, cyano, C₁-C₆-alkyl, C₃-C₈-cycloalkyl, C₂-C₆-alkenyl and C₂-C₆-alkynyl, wherein the four last-mentioned aliphatic and cycloaliphatic radicals may be partially or fully halogenated and/or may be substituted by one or more radicals R⁸;
each R⁸ is independently selected from the group consisting of cyano, azido, nitro, —SCN, —SF₅, C₃-C₈-cycloalkyl, C₃-C₈-halocycloalkyl, C₃-C₈-cycloalkenyl, C₃-C₈-halocycloalkenyl, wherein the cycloaliphatic moieties in the four last-mentioned radicals may be substituted by one or more radicals R¹³; —Si(R¹²)₃, —OR⁹, —OSO₂R⁹, —S(O)ₙR⁹, —N(R^{10a})R^{10b}, —C(═O)N(R^{10a})R^{10b}, —C(═S)N(R^{10a})R^{10b}, —C(═O)OR⁹, —CH═NOR⁹; phenyl, optionally substituted with 1, 2, 3, 4 or 5 substituents R¹⁶, and a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more substituents R¹⁶,
or
two R⁸ present on the same carbon atom of an alkyl, alkenyl, alkynyl or cycloalkyl group together form a group ═O, ═C(R¹³)₂; ═S; ═S(O)ₘ(R¹⁵)₂, ═S(O)ₘR¹⁵N(R^{14a})R^{14b}, ═NR^{10a}, ═NOR⁹; or ═NN(R^{10a})R^{10b};
or
two radicals R⁸, together with the carbon atoms of an alkyl, alkenyl, alkynyl or cycloalkyl group which they are bonded to, form a 3-, 4-, 5-, 6-, 7- or 8-membered saturated or partially unsaturated carbocyclic or heterocyclic ring, wherein the heterocyclic ring comprises 1, 2, 3 or 4 heteroatoms or heteroatom groups independently selected from the group consisting of N, O, S, NO, SO and SO₂, as ring members, and where the carbocyclic or heterocyclic ring is optionally substituted with one or more substituents R¹⁶; and
R⁸ as a substituent on a cycloalkyl ring is additionally selected from the group consisting of C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in these six radicals may be substituted by one or more radicals R¹³; and
R⁸ in the groups —C(═NR⁶)R⁸, and —C(═O)R⁸ is additionally selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl and C₂-C₆-haloalkynyl, wherein the aliphatic moieties in the six last-mentioned radicals may be substituted by one or more radicals R¹³;
each R⁹ is independently selected from the group consisting of hydrogen, cyano, C₁-C₆-alkyl, C₁-C₆-haloalkyl, C₃-C₈-cycloalkyl, C₃-C₈-cycloalkyl-C₁-C₄-alkyl-, C₃-C₈-halocycloalkyl, C₂-C₆-alkenyl, C₂-C₆-haloalkenyl, C₂-C₆-alkynyl, C₂-C₆-haloalkynyl, where the aliphatic and cycloaliphatic moieties in the nine last-mentioned radicals may be substituted by one or more radicals R¹³, —C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, —Si(R¹²)₃, —S(O)ₙR¹⁵, —S(O)ₙN(R^{14a})R^{14b}, —N(R^{10a})R^{10b}, —N═C(R¹³)₂, —C(═O)R¹³, —C(═O)N(R^{14a})R^{14b}, —C(═S)N(R^{14a})R^{14b}, —C(═O)OR¹⁵, phenyl, optionally substituted with one or more substituents R¹⁶; and a 3-, 4-, 5-, 6- or 7-membered saturated, partially unsaturated or maximally unsaturated heterocyclic ring comprising 1, 2 or 3 heteroatoms or heteroatom groups selected from the group consisting of N, O, S, NO, SO and SO₂, as ring members, wherein the heterocyclic ring is optionally substituted with one or more substituents R¹⁶; and R⁹ in the groups —S(O)ₙR⁹ and —OSO₂R⁹ is additionally selected from the group consisting of C₁-C₆-alkoxy and C₁-C₆-haloalkoxy;
R^{10a}, R^{10b} independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic and cycloaliphatic moieties in the eight last-mentioned radicals may be substituted by one or more radicals R¹³; —C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, C₁-C₆-alkoxy, C₁-C₆-haloalkoxy, C₁-C₆-alkylthio, C₁-C₆-haloalkylthio, —S(O)ₙR¹⁵, —S(O)ₙN(R^{14a})R^{14b}, —C(═O)R¹³, —C(═O)OR¹⁵, —C(═O)N(R^{14a})R^{14b}, —C(═S)R¹³, —C(═S)SR¹⁵, —C(═S)N(R^{14a})R^{14b}, —C(═NR¹⁴)R¹³; phenyl, optionally substituted with 1, 2, 3 or 4, substituents R¹⁶; and a 3-, 4-, 5-, 6- or 7-membered saturated, partially unsaturated or maximally unsaturated heterocyclic ring comprising 1, 2, 3 or 4 heteroatoms or heteroatom groups selected from the group consisting of N, O, S, NO, SO and SO₂, as ring members, where the heterocyclic ring is optionally substituted with one or more substituents R¹⁶;
or
R^{10a} and R^{10b} form together with the nitrogen atom they are bonded to a 3-, 4-, 5-, 6-, 7- or 8-membered saturated, partially unsaturated or maximally unsaturated heterocyclic ring, wherein the heterocyclic ring may additionally contain one or two heteroatoms or heteroatom groups selected from the group consisting of N, O, S, NO, SO and SO₂, as ring members, wherein the heterocyclic ring optionally carries one or more substituents selected from the group consisting of halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl, optionally substituted with 1, 2, 3, 4 or 5 substituents R¹⁶, and a 3-, 4-, 5-, 6-, or 7-membered saturated, partially unsaturated or maximally unsaturated heterocyclic ring comprising 1, 2 or 3 heteroatoms or heteroatom groups selected from the group consisting of N, O, S, NO, SO and SO₂, as ring members, wherein the heterocyclic ring optionally carries one or more substituents R¹⁶;
or R^{10a} and R^{10b} together form a group ═C(R¹³)₂, ═S(O)ₘ(R¹⁵)₂, ═S(O)ₘR¹⁵N(R^{14a})R^{14b}, ═NR¹⁴ or ═NOR¹⁵;
R¹¹ is independently selected from the group consisting of halogen, cyano, azido, nitro, —SCN, —SF₅, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted with one or more radicals R⁸, —OR⁹, —NR^{10a}R^{10b}, —S(O)ₙR⁹, —Si(R¹²)₃; phenyl, optionally substituted with 1, 2, 3, 4, or 5 substituents selected independently from R¹⁶; and a 3-, 4-, 5-, 6- or 7-membered saturated, partially unsaturated or maximally unsaturated aromatic heterocyclic ring comprising 1, 2, 3 or 4 heteroatoms or heteroatom groups selected from the group consisting of N, O, S, NO, SO and SO₂, as ring members, where the heterocyclic ring is optionally substituted with one or more substituents selected independently from R¹⁶;
or two R¹¹ present on the same ring carbon atom of an unsaturated or partially unsaturated heterocyclic ring may together form a group ═O, ═C(R¹³)₂; ═S; ═S(O)ₘ(R¹⁵)₂; ═S(O)ₘR¹⁵N(R^{14a})R^{14b}, ═NR¹⁴, ═NOR¹⁵, or ═NN(R^{14a})R^{14b}; or two R¹¹ bound on adjacent ring atoms form together with the ring atoms to which they are bound a saturated 3-, 4-, 5-, 6-, 7-, 8- or 9-membered ring, wherein the ring may contain 1 or 2 heteroatoms or heteroatom groups selected from the group consisting of O, S, N, NR¹⁴, NO, SO and SO₂ and/or 1 or 2 groups selected from C═O, C═S and C═NR¹⁴ as ring members, and wherein the ring may be substituted by one or more radicals selected from the group consisting of halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁶, and a 3-, 4-, 5-, 6- or 7-membered saturated, partially unsaturated or maximally unsaturated heterocyclic ring containing 1, 2 or 3 heteroatoms or heteroatom groups selected from the group consisting of N, O, S, NO, SO and SO₂, as ring members, wherein the heterocyclic ring may be substituted by one or more radicals R¹⁶;
each R¹² is independently selected from the group consisting of hydrogen, halogen, C₁-C₆-alkyl, C₁-C₆-haloalkyl, C₁-C₆-alkoxy, C₁-C₆-haloalkoxy, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, and phenyl, optionally substituted with 1, 2, 3, 4, or 5 substituents R¹⁶;
each R¹³ is independently selected from the group consisting of cyano, nitro, —OH, —SH, —SCN, —SF₅, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —NR^{14a}R^{14b}, —C(═O)NR^{14a}R^{14b}, trimethylsilyl, triethylsilyl, tert-butyldimethylsilyl, C₃-C₈-cycloalkyl which may be unsubstituted, partially or fully halogenated and/or may carry 1 or 2 radicals selected from cyano, C₁-C₄-alkyl, C₃-C₄-cycloalkyl, C₁-C₄-alkoxy, C₁-C₄-haloalkoxy and oxo; phenyl, benzyl, phenoxy, wherein the phenyl moiety in the three last-mentioned radicals may be unsubstituted or carry 1, 2, 3, 4 or 5 substituents R¹⁶; and a 3-, 4-, 5-, 6- or 7-membered saturated, partially unsaturated or maximally unsaturated heterocyclic ring containing 1, 2 or 3 heteroatoms or heteroatom groups selected from the group consisting of N, O, S, NO, SO and SO₂, as ring members, where the heterocyclic ring may be substituted by 1, 2 or 3 substituents R¹⁶;
or
two R¹³ present on the same carbon atom of an alkyl, alkenyl, alkynyl or cycloalkyl group may together be ═O, ═CH(C₁-C₄-alkyl), ═C(C₁-C₄-alkyl)C₁-C₄-alkyl, ═NR¹⁷ or ═NOR¹⁷;
and
R¹³ as a substituent on a cycloalkyl ring is additionally selected from the group consisting of C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 substituents selected from the group consisting of CN, C₃-C₄-cycloalkyl, C₁-C₄-alkoxy, C₁-C₄-haloalkoxy and oxo;
and
R¹³ in the groups ═C(R¹³)₂, —N═C(R¹³)₂, —C(═O)R¹³, —C(═S)R¹³ and —C(═NR¹⁴)R¹³ is additionally selected from the group consisting of hydrogen, halogen, C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 radicals selected from the group consisting of CN, C₃-C₄-cycloalkyl, C₁-C₄-alkoxy, C₁-C₄-haloalkoxy and oxo;
each R¹⁴ is independently selected from the group consisting of hydrogen, cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —C(═O)NR^{18a}R^{18b}, trimethylsilyl, triethylsilyl, tert-butyldimethylsilyl, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of CN, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl which may be substituted by 1 or 2 substituents selected from halogen and cyano; and oxo; C₃-C₈-cycloalkyl which may be unsubstituted, partially or fully halogenated and/or may carry 1 or 2 radicals selected from cyano, C₁-C₄-alkyl, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl, C₃-C₄-cycloalkyl-C₁-C₄-alkyl-, wherein the cycloalkyl moiety in the two last-mentioned radicals may be substituted by 1 or 2 substituents selected from the group consisting of halogen and cyano; and oxo; phenyl, benzyl, pyridyl, phenoxy, wherein the cyclic moieties in the four last-mentioned radicals may be unsubstituted and/or carry 1, 2 or 3 substituents selected from the group consisting of halogen, C₁-C₆-alkyl, C₁-C₆-haloalkyl, C₁-C₆-alkoxy, C₁-C₆-haloalkoxy and (C₁-C₆-alkoxy)carbonyl; and a 3-, 4-, 5- or 6-membered saturated, partially unsaturated or maximally unsaturated heterocyclic ring comprising 1 or 2 heteroatoms or heteroatom groups selected from the group consisting of N, O, S, NO, SO and SO₂, as ring members, where the heterocyclic ring is optionally substituted with one or more substituents R¹⁶;
R^{14a} and R^{14b}, independently of each other, have one of the meanings given for R¹⁴;
or
R^{14a} and R^{14b}, together with the nitrogen atom to which they are bound, form a 3-, 4-, 5-, 6- or 7-membered saturated, partially unsaturated or maximally unsaturated heterocyclic ring, wherein the heterocyclic ring may additionally contain 1 or 2 heteroatoms or heteroatom groups selected from the group consisting of N, O, S, NO, SO and SO₂, as ring members, wherein the heterocyclic ring optionally carries one or more substituents selected from the group consisting of halogen, C₁-C₄-alkyl, C₁-C₄-haloalkyl, C₁-C₄-alkoxy and C₁-C₄-haloalkoxy;
or
R^{14a} and R¹⁴ or R^{14b} and R¹⁴, together with the nitrogen atoms to which they are bound in the group —C(═NR¹⁴)N(R^{14a})R^{14b}, form a 3-, 4-, 5-, 6- or 7-membered partially unsaturated or maximally unsaturated heterocyclic ring, wherein the heterocyclic ring may additionally contain 1 or 2 heteroatoms or heteroatom groups selected from the group consisting of N, O, S, NO, SO and SO₂, as ring members, wherein the heterocyclic ring optionally carries one or more substituents selected from the group consisting of halogen, C₁-C₄-haloalkyl, C₁-C₄-alkoxy and C₁-C₄-haloalkoxy;
each R¹⁵ is independently selected from the group consisting of hydrogen, cyano, trimethylsilyl, triethylsilyl, tert-butyldimethylsilyl, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo; C₃-C₈-cycloalkyl which may be unsubstituted, partially or fully halogenated and/or may carry 1 or 2 radicals selected from the group consisting of C₁-C₄-alkyl, C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo; phenyl, benzyl, pyridyl and phenoxy, wherein the four last-mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆-haloalkoxy and (C₁-C₆-alkoxy)carbonyl;
each R¹⁶ is independently selected from the group consisting of halogen, nitro, cyano, —OH, —SH, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl; C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of C₃-C₄-cycloalkyl, C₁-C₄-alkoxy, C₁-C₄-haloalkoxy and oxo; C₃-C₈-cycloalkyl which may be unsubstituted, partially or fully halogenated and/or may carry 1 or 2 radicals selected from the group consisting of C₁-C₄-alkyl, C₃-C₄-cycloalkyl, C₁-C₄-alkoxy, C₁-C₄-haloalkoxy and oxo; phenyl, benzyl, pyridyl and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆-haloalkoxy and (C₁-C₆-alkoxy)carbonyl;
or
two R¹⁶ present together on the same atom of an unsaturated or partially unsaturated ring may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
or
two R¹⁶ on two adjacent carbon atoms form together with the carbon atoms they are bonded to a 4-, 5-, 6-, 7- or 8-membered saturated, partially unsaturated or maximally unsaturated ring, wherein the ring may contain 1 or 2 heteroatoms or heteroatom groups selected from the group consisting of N, O, S, NO, SO and SO₂, as ring members, and wherein the ring optionally carries one or more substituents selected from halogen, C₁-C₄-haloalkyl, C₁-C₄-alkoxy and C₁-C₄-haloalkoxy;
R¹⁷, R^{18a} and R^{18b}, independently of each other and independently of each occurrence, are selected from the group consisting of hydrogen, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of CN, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl which may be substituted by 1 or 2 substituents selected from halogen and cyano; and oxo; C₃-C₈-cycloalkyl which may be unsubstituted, partially or fully halogenated and/or may carry 1 or 2 radicals selected from the group consisting of cyano, C₁-C₄-alkyl and C₁-C₄-haloalkyl; phenyl and benzyl;
each n is independently 0, 1 or 2; and
each m is independently 0 or 1;
or a stereoisomer, enantiomer, diastereomer, N-oxide, or an agriculturally or veterinary acceptable salt thereof.

48. The method of claim 46, wherein the stink bugs are Acrosternum spp., Euschistus spp., Nezara spp., Piezodrus spp., Dichelops spp., Halyomorpha halys, Thyanta accerra, Podisus maculiventris and/or Megacopta cribraria.

49. The method of claim 48, wherein the stink bugs are Acrosternum spp., Euschistus spp., Nezara spp. and/or Piezodrus spp.

50. The method of claim 49, wherein the stink bugs are Acrosternum hilare, Euschistus heros, Nezara viridula and/or Piezodrus guildinii.

51. The method of claim 48, wherein the stink bug is Halyomorpha halys.

52. A method for combating or controlling pests that resistant to one or more insecticides comprising contacting the pest, their food supply, habitat or breeding grounds, the plant or the crop with an isothiazoline compound of formula I or a composition comprising at least one compound of formula I: 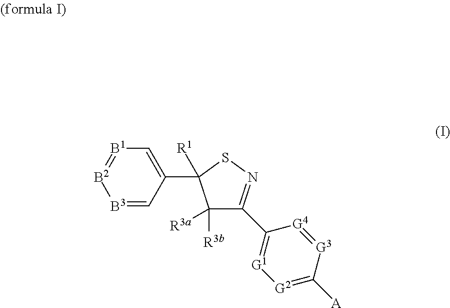
wherein
A is a group A¹, A² or A³;
wherein
A¹ is selected from the group consisting of —C(═NR⁶)R⁸, —S(O)ₙR⁹, and —N(R⁵)R⁶;
A² is a group of following formula: 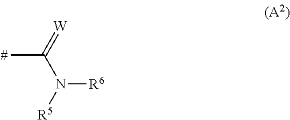
wherein
# denotes the bond to the remainder of the molecule;
W is selected from the group consisting of O and S;
A³ is a group of following formula: 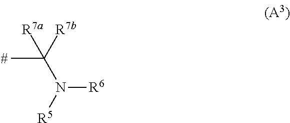
wherein
# denotes the bond to the aromatic ring of formula (I);
B¹, B² and B³ are each independently selected from the group consisting of N and CR², with the proviso that at most two of B¹, B² and B³ are N;
G¹, G², G³ and G⁴ are each independently selected from the group consisting of N and CR⁴, with the proviso that at most two of G¹, G², G³ and G⁴ are N;
R¹ is selected from the group consisting of C₁-C₄-alkyl, C₁-C₄-haloalkyl, C₁-C₄-alkoxy-C₁-C₄-alkyl, C₁-C₄-haloalkoxy-C₁-C₄-alkyl-, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl and —C(═O)OR¹⁵;
each R² is independently selected from the group consisting of hydrogen, halogen, cyano, azido, nitro, —SCN, —SF₅, C₁-C₆-alkyl, C₃-C₈-cycloalkyl, C₂-C₆-alkenyl, C₂-C₆-alkynyl, wherein the four last mentioned aliphatic and cycloaliphatic radicals may be partially or fully halogenated and/or may be substituted by one or more radicals R⁸, —Si(R¹²)₃, —OR⁹, —S(O)ₙR⁹, and —NR^{10a}R^{10b};
R^{3a}, R^{3b} are each independently selected from the group consisting of hydrogen, halogen, hydroxyl, —CO₂R^{3d}, C₁-C₃-alkyl, C₁-C₃-haloalkyl, C₂-C₃-alkenyl, C₂-C₃-alkynyl, C₁-C₃-alkoxy, C₁-C₃-haloalkoxy, C₁-C₃-alkylthio, C₁-C₃-haloalkylthio, C₁-C₃-alkylsulfonyl and C₁-C₃-haloalkylsulfonyl; or R^{3a} and R^{3b} together form a group ═O, ═C(R^{3c})₂, ═NOH or ═NOCH₃;
each R^{3c} is independently selected from the group consisting of hydrogen, halogen, CH₃ and CF₃;
R^{3d} is selected from the group consisting of hydrogen, C₁-C₆-alkyl and C₁-C₃-alkyloxy-C₁-C₃-alkyl-;
each R⁴ is independently selected from the group consisting of hydrogen, halogen, cyano, azido, nitro, —SCN, —SF₅, C₁-C₆-alkyl which may be partially or fully halogenated and/or may be substituted by one or more radicals R⁸, C₃-C₈-cycloalkyl which may be partially or fully halogenated and/or may be substituted by one or more radicals R⁸, C₂-C₆-alkenyl which may be partially or fully halogenated and/or may be substituted by one or more radicals R⁸, C₂-C₆-alkynyl which may be partially or fully halogenated and/or may be substituted by one or more radicals R⁸, —Si(R¹²)₃, —OR⁹, —S(O)ₙR⁹, —NR^{10a}R^{10b}, phenyl which may be substituted by 1, 2, 3, 4 or 5 radicals R¹¹, and a 3-, 4-, 5-, 6-7-, 8-, 9- or 10-membered saturated, partially unsaturated or maximally unsaturated heteromonocyclic or heterobicyclic ring containing 1, 2, 3 or 4 heteroatoms or heteroatom groups selected from the group consisting of N, O, S, NO, SO and SO₂, as ring members, where the heteromonocyclic or heterobicyclic ring may be substituted by one or more radicals R¹¹;
each R⁵ is independently selected from the group consisting of hydrogen, C₁-C₁₀-alkyl, C₃-C₈-cycloalkyl, C₂-C₁₀-alkenyl, and C₂-C₁₀-alkynyl, wherein the four last-mentioned aliphatic and cycloaliphatic radicals may be partially or fully halogenated and/or may be substituted with one or more substituents R⁸,
R⁶ in A² is selected from the group consisting of hydrogen, C₁-C₆-alkyl, C₁-C₆-haloalkyl, C₁-C₄-alkyl which carries one radical R⁸, C₂-C₆-alkenyl, C₂-C₆-haloalkenyl, C₂-C₆-alkynyl, C₃-C₆-cycloalkyl which may be substituted by 1 to 4 substituents selected from halogen and cyano; —N(R^{10a})R^{10b}, —CH═NOR⁹; phenyl which may be substituted with 1, 2, 3, 4, or 5 substituents R¹¹; and a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from the group consisting of N, O, S, NO, SO and SO₂, as ring members, wherein the heteromonocyclic or heterobicyclic ring may be substituted with one or more substituents R¹¹; or R⁵ and R⁶ form together a group ═S(O)ₘ(R⁹)₂;
each R⁶ in all other cases is independently selected from the group consisting of hydrogen, cyano, C₁-C₁₀-alkyl, C₃-C₈-cycloalkyl, C₂-C₁₀-alkenyl, and C₂-C₁₀-alkynyl, wherein the four last-mentioned aliphatic and cycloaliphatic radicals may be partially or fully halogenated and/or may be substituted by one or more substituents R⁸, —OR⁹, —NR^{10a}R^{10b}, —S(O)ₙR⁹, —C(═O)NR^{10a}N(R^{10a})R^{10b}), —Si(R¹²)₃, —C(═O)R⁸, phenyl which may be substituted with 1, 2, 3, 4, or 5 substituents R¹¹, and a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from the group consisting of N, O, S, NO, SO and SO₂, as ring members, wherein the heteromonocyclic or heterobicyclic ring may be substituted with one or more substituents R¹¹;
or
R⁵ and R⁶ or R⁵ and R^{6a}, together with the nitrogen atom to which they are bound, form a 3-, 4-, 5-, 6-, 7- or 8-membered saturated, partially unsaturated or maximally unsaturated heterocyclic ring, where the ring may further contain 1, 2, 3 or 4 heteroatoms or heteroatom-containing groups selected from the group consisting of O, S, N, SO, SO₂, C═O and C═S as ring members, wherein the heterocyclic ring may be substituted with 1, 2, 3, 4 or 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic or cycloaliphatic moieties in the twelve last-mentioned radicals may be substituted by one or more radicals R⁸, and phenyl which may be substituted with 1, 2, 3, 4 or 5 substituents R¹¹;
R^{7a}, R^{7b} are each independently selected from the group consisting of hydrogen, halogen, cyano, C₁-C₆-alkyl, C₃-C₈-cycloalkyl, C₂-C₆-alkenyl and C₂-C₆-alkynyl, wherein the four last-mentioned aliphatic and cycloaliphatic radicals may be partially or fully halogenated and/or may be substituted by one or more radicals R⁸;
each R⁸ is independently selected from the group consisting of cyano, azido, nitro, —SCN, —SF₅, C₃-C₈-cycloalkyl, C₃-C₈-halocycloalkyl, C₃-C₈-cycloalkenyl, C₃-C₈-halocycloalkenyl, wherein the cycloaliphatic moieties in the four last-mentioned radicals may be substituted by one or more radicals R¹³; —Si(R¹²)₃, —OR⁹, —OSO₂R⁹, —S(O)ₙR⁹, —N(R^{10a})R^{10b}, —C(═O)N(R^{10a})R^{10b}, —C(═S)N(R^{10a})R^{10b}, —C(═O)OR⁹, —CH═NOR⁹; phenyl, optionally substituted with 1, 2, 3, 4 or 5 substituents R¹⁶, and a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more substituents R¹⁶,
or
two R⁸ present on the same carbon atom of an alkyl, alkenyl, alkynyl or cycloalkyl group together form a group ═O, ═C(R¹³)₂; ═S; ═S(O)ₘ(R¹⁵)₂, ═S(O)ₘR¹⁵N(R^{14a})R^{14b}, ═NR^{10a}, ═NOR⁹; or ═NN(R^{10a})R^{10b};
or
two radicals R⁸, together with the carbon atoms of an alkyl, alkenyl, alkynyl or cycloalkyl group which they are bonded to, form a 3-, 4-, 5-, 6-, 7- or 8-membered saturated or partially unsaturated carbocyclic or heterocyclic ring, wherein the heterocyclic ring comprises 1, 2, 3 or 4 heteroatoms or heteroatom groups independently selected from the group consisting of N, O, S, NO, SO and SO₂, as ring members, and where the carbocyclic or heterocyclic ring is optionally substituted with one or more substituents R¹⁶; and
R⁸ as a substituent on a cycloalkyl ring is additionally selected from the group consisting of C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in these six radicals may be substituted by one or more radicals R¹³; and
R⁸ in the groups —C(═NR⁶)R⁸, and —C(═O)R⁸ is additionally selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl and C₂-C₆-haloalkynyl, wherein the aliphatic moieties in the six last-mentioned radicals may be substituted by one or more radicals R¹³;
each R⁹ is independently selected from the group consisting of hydrogen, cyano, C₁-C₆-alkyl, C₁-C₆-haloalkyl, C₃-C₈-cycloalkyl, C₃-C₈-cycloalkyl-C₁-C₄-alkyl-, C₃-C₈-halocycloalkyl, C₂-C₆-alkenyl, C₂-C₆-haloalkenyl, C₂-C₆-alkynyl, C₂-C₆-haloalkynyl, where the aliphatic and cycloaliphatic moieties in the nine last-mentioned radicals may be substituted by one or more radicals R¹³, —C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, —Si(R¹²)₃, —S(O)ₙR¹⁵, —S(O)ₙN(R^{14a})R^{14b}, —N(R^{10a})R^{10b}, —N═C(R¹³)₂, —C(═O)R¹³, —C(═O)N(R^{14a})R^{14b}, —C(═S)N(R^{14a})R^{14b}, —C(═O)OR¹⁵, phenyl, optionally substituted with one or more substituents R¹⁶; and a 3-, 4-, 5-, 6- or 7-membered saturated, partially unsaturated or maximally unsaturated heterocyclic ring comprising 1, 2 or 3 heteroatoms or heteroatom groups selected from the group consisting of N, O, S, NO, SO and SO₂, as ring members, wherein the heterocyclic ring is optionally substituted with one or more substituents R¹⁶; and R⁹ in the groups —S(O)ₙR⁹ and —OSO₂R⁹ is additionally selected from the group consisting of C₁-C₆-alkoxy and C₁-C₆-haloalkoxy;
R^{10a}, R^{10b} independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic and cycloaliphatic moieties in the eight last-mentioned radicals may be substituted by one or more radicals R¹³; —C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, C₁-C₆-alkoxy, C₁-C₆-haloalkoxy, C₁-C₆-alkylthio, C₁-C₆-haloalkylthio, —S(O)ₙR¹⁵, —S(O)ₙN(R^{14a})R^{14b}, —C(═O)R¹³, —C(═O)OR¹⁵, —C(═O)N(R^{14a})R^{14b}, —C(═S)R¹³, —C(═S)SR¹⁵, —C(═S)N(R^{14a})R^{14b}, —C(═NR¹⁴)R¹³; phenyl, optionally substituted with 1, 2, 3 or 4, substituents R¹⁶; and a 3-, 4-, 5-, 6- or 7-membered saturated, partially unsaturated or maximally unsaturated heterocyclic ring comprising 1, 2, 3 or 4 heteroatoms or heteroatom groups selected from the group consisting of N, O, S, NO, SO and SO₂, as ring members, where the heterocyclic ring is optionally substituted with one or more substituents R¹⁶;
or
R^{10a} and R^{10b} form together with the nitrogen atom they are bonded to a 3-, 4-, 5-, 6-, 7- or 8-membered saturated, partially unsaturated or maximally unsaturated heterocyclic ring, wherein the heterocyclic ring may additionally contain one or two heteroatoms or heteroatom groups selected from the group consisting of N, O, S, NO, SO and SO₂, as ring members, wherein the heterocyclic ring optionally carries one or more substituents selected from the group consisting of halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl, optionally substituted with 1, 2, 3, 4 or 5 substituents R¹⁶, and a 3-, 4-, 5-, 6-, or 7-membered saturated, partially unsaturated or maximally unsaturated heterocyclic ring comprising 1, 2 or 3 heteroatoms or heteroatom groups selected from the group consisting of N, O, S, NO, SO and SO₂, as ring members, wherein the heterocyclic ring optionally carries one or more substituents R¹⁶;
or R^{10a} and R^{10b} together form a group ═C(R¹³)₂, ═S(O)ₘ(R¹⁵)₂, ═S(O)ₘR¹⁵N(R^{14a})R^{14b}, ═NR¹⁴ or ═NOR¹⁵;
R¹¹ is independently selected from the group consisting of halogen, cyano, azido, nitro, —SCN, —SF₅, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted with one or more radicals R⁸, —OR⁹, —NR^{10a}R^{10b}, —S(O)ₙR⁹, —Si(R¹²)₃; phenyl, optionally substituted with 1, 2, 3, 4, or 5 substituents selected independently from R¹⁶; and a 3-, 4-, 5-, 6- or 7-membered saturated, partially unsaturated or maximally unsaturated aromatic heterocyclic ring comprising 1, 2, 3 or 4 heteroatoms or heteroatom groups selected from the group consisting of N, O, S, NO, SO and SO₂, as ring members, where the heterocyclic ring is optionally substituted with one or more substituents selected independently from R¹⁶;
or two R¹¹ present on the same ring carbon atom of an unsaturated or partially unsaturated heterocyclic ring may together form a group ═O, ═C(R¹³)₂; ═S; ═S(O)ₘ(R¹⁵)₂; ═S(O)ₘR¹⁵N(R^{14a})R^{14b}, ═NR¹⁴, ═NOR¹⁵, or ═NN(R^{14a})R^{14b};
or two R¹¹ bound on adjacent ring atoms form together with the ring atoms to which they are bound a saturated 3-, 4-, 5-, 6-, 7-, 8- or 9-membered ring, wherein the ring may contain 1 or 2 heteroatoms or heteroatom groups selected from the group consisting of O, S, N, NR¹⁴, NO, SO and SO₂ and/or 1 or 2 groups selected from C═O, C═S and C═NR¹⁴ as ring members, and wherein the ring may be substituted by one or more radicals selected from the group consisting of halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁶, and a 3-, 4-, 5-, 6- or 7-membered saturated, partially unsaturated or maximally unsaturated heterocyclic ring containing 1, 2 or 3 heteroatoms or heteroatom groups selected from the group consisting of N, O, S, NO, SO and SO₂, as ring members, wherein the heterocyclic ring may be substituted by one or more radicals R¹⁶;
each R¹² is independently selected from the group consisting of hydrogen, halogen, C₁-C₆-alkyl, C₁-C₆-haloalkyl, C₁-C₆-alkoxy, C₁-C₆-haloalkoxy, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, and phenyl, optionally substituted with 1, 2, 3, 4, or 5 substituents R¹⁶;
each R¹³ is independently selected from the group consisting of cyano, nitro, —OH, —SH, —SCN, —SF₅, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —NR^{14a}R^{14b}, —C(═O)NR^{14a}R^{14b}, trimethylsilyl, triethylsilyl, tert-butyldimethylsilyl, C₃-C₈-cycloalkyl which may be unsubstituted, partially or fully halogenated and/or may carry 1 or 2 radicals selected from cyano, C₁-C₄-alkyl, C₃-C₄-cycloalkyl, C₁-C₄-alkoxy, C₁-C₄-haloalkoxy and oxo; phenyl, benzyl, phenoxy, wherein the phenyl moiety in the three last-mentioned radicals may be unsubstituted or carry 1, 2, 3, 4 or 5 substituents R¹⁶; and a 3-, 4-, 5-, 6- or 7-membered saturated, partially unsaturated or maximally unsaturated heterocyclic ring containing 1, 2 or 3 heteroatoms or heteroatom groups selected from the group consisting of N, O, S, NO, SO and SO₂, as ring members, where the heterocyclic ring may be substituted by 1, 2 or 3 substituents R¹⁶;
or
two R¹³ present on the same carbon atom of an alkyl, alkenyl, alkynyl or cycloalkyl group may together be ═O, ═CH(C₁-C₄-alkyl), ═C(C₁-C₄-alkyl)C₁-C₄-alkyl, ═NR¹⁷ or ═NOR¹⁷;
and
R¹³ as a substituent on a cycloalkyl ring is additionally selected from the group consisting of C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 substituents selected from the group consisting of CN, C₃-C₄-cycloalkyl, C₁-C₄-alkoxy, C₁-C₄-haloalkoxy and oxo;
and
R¹³ in the groups ═C(R¹³)₂, —N═C(R¹³)₂, —C(═O)R¹³, —C(═S)R¹³ and —C(═NR¹⁴)R¹³ is additionally selected from the group consisting of hydrogen, halogen, C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 radicals selected from the group consisting of CN, C₃-C₄-cycloalkyl, C₁-C₄-alkoxy, C₁-C₄-haloalkoxy and oxo;
each R¹⁴ is independently selected from the group consisting of hydrogen, cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —C(═O)NR^{18a}R^{18b}, trimethylsilyl, triethylsilyl, tert-butyldimethylsilyl, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of CN, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl which may be substituted by 1 or 2 substituents selected from halogen and cyano; and oxo; C₃-C₈-cycloalkyl which may be unsubstituted, partially or fully halogenated and/or may carry 1 or 2 radicals selected from cyano, C₁-C₄-alkyl, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl, C₃-C₄-cycloalkyl-C₁-C₄-alkyl-, wherein the cycloalkyl moiety in the two last-mentioned radicals may be substituted by 1 or 2 substituents selected from the group consisting of halogen and cyano; and oxo; phenyl, benzyl, pyridyl, phenoxy, wherein the cyclic moieties in the four last-mentioned radicals may be unsubstituted and/or carry 1, 2 or 3 substituents selected from the group consisting of halogen, C₁-C₆-alkyl, C₁-C₆-haloalkyl, C₁-C₆-alkoxy, C₁-C₆-haloalkoxy and (C₁-C₆-alkoxy)carbonyl; and a 3-, 4-, 5- or 6-membered saturated, partially unsaturated or maximally unsaturated heterocyclic ring comprising 1 or 2 heteroatoms or heteroatom groups selected from the group consisting of N, O, S, NO, SO and SO₂, as ring members, where the heterocyclic ring is optionally substituted with one or more substituents R¹⁶;
R^{14a} and R^{14b}, independently of each other, have one of the meanings given for R¹⁴;
or
R^{14a} and R^{14b}, together with the nitrogen atom to which they are bound, form a 3-, 4-, 5-, 6- or 7-membered saturated, partially unsaturated or maximally unsaturated heterocyclic ring, wherein the heterocyclic ring may additionally contain 1 or 2 heteroatoms or heteroatom groups selected from the group consisting of N, O, S, NO, SO and SO₂, as ring members, wherein the heterocyclic ring optionally carries one or more substituents selected from the group consisting of halogen, C₁-C₄-alkyl, C₁-C₄-haloalkyl, C₁-C₄-alkoxy and C₁-C₄-haloalkoxy;
or
R^{14a} and R¹⁴ or R^{14b} and R¹⁴, together with the nitrogen atoms to which they are bound in the group —C(═NR¹⁴)N(R^{14a})R^{14b}, form a 3-, 4-, 5-, 6- or 7-membered partially unsaturated or maximally unsaturated heterocyclic ring, wherein the heterocyclic ring may additionally contain 1 or 2 heteroatoms or heteroatom groups selected from the group consisting of N, O, S, NO, SO and SO₂, as ring members, wherein the heterocyclic ring optionally carries one or more substituents selected from the group consisting of halogen, C₁-C₄-haloalkyl, C₁-C₄-alkoxy and C₁-C₄-haloalkoxy;
each R¹⁵ is independently selected from the group consisting of hydrogen, cyano, trimethylsilyl, triethylsilyl, tert-butyldimethylsilyl, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo; C₃-C₈-cycloalkyl which may be unsubstituted, partially or fully halogenated and/or may carry 1 or 2 radicals selected from the group consisting of C₁-C₄-alkyl, C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo; phenyl, benzyl, pyridyl and phenoxy, wherein the four last-mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆-haloalkoxy and (C₁-C₆-alkoxy)carbonyl;
each R¹⁶ is independently selected from the group consisting of halogen, nitro, cyano, —OH, —SH, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl; C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of C₃-C₄-cycloalkyl, C₁-C₄-alkoxy, C₁-C₄-haloalkoxy and oxo; C₃-C₈-cycloalkyl which may be unsubstituted, partially or fully halogenated and/or may carry 1 or 2 radicals selected from the group consisting of C₁-C₄-alkyl, C₃-C₄-cycloalkyl, C₁-C₄-alkoxy, C₁-C₄-haloalkoxy and oxo; phenyl, benzyl, pyridyl and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆-haloalkoxy and (C₁-C₆-alkoxy)carbonyl;
or
two R¹⁶ present together on the same atom of an unsaturated or partially unsaturated ring may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
or
two R¹⁶ on two adjacent carbon atoms form together with the carbon atoms they are bonded to a 4-, 5-, 6-, 7- or 8-membered saturated, partially unsaturated or maximally unsaturated ring, wherein the ring may contain 1 or 2 heteroatoms or heteroatom groups selected from the group consisting of N, O, S, NO, SO and SO₂, as ring members, and wherein the ring optionally carries one or more substituents selected from halogen, C₁-C₄-haloalkyl, C₁-C₄-alkoxy and C₁-C₄-haloalkoxy;
R¹⁷, R^{18a} and R^{18b}, independently of each other and independently of each occurrence, are selected from the group consisting of hydrogen, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from the group consisting of CN, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl which may be substituted by 1 or 2 substituents selected from halogen and cyano; and oxo; C₃-C₈-cycloalkyl which may be unsubstituted, partially or fully halogenated and/or may carry 1 or 2 radicals selected from the group consisting of cyano, C₁-C₄-alkyl and C₁-C₄-haloalkyl; phenyl and benzyl;
each n is independently 0, 1 or 2; and
each m is independently 0 or 1;
or a stereoisomer, enantiomer, diastereomer, N-oxides, or agriculturally or veterinary acceptable salt thereof.

53. The method of claim 52, wherein the pests are from the family of Pentatomidae (stink bugs).

54. The method of claim 53, wherein the pests are Acrosternum spp., Euschistus spp., Nezara spp., Piezodrus spp., Dichelops spp., Halyomorpha halys, Thyanta accerra, Podisus maculiventris and/or Megacopta cribraria.

55. The method claim 54, wherein the pests are Acrosternum spp., Euschistus spp., Nezara spp. and/or Piezodrus spp.

56. The method of claim 55, wherein the pests are Acrosternum hilare, Euschistus heros, Nezara viridula and/or Piezodrus guildinii.

57. The method claim 54, wherein the pest is Halyomorpha halys.

58. The method of claim 52, wherein the pests are resistant to pyrethroids, neonicotionoids and/or organophosphates.

59. The method of claim 37, wherein the isothiazoline compound applied in the method is of formula I.1, 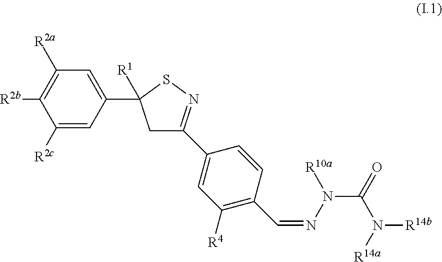
wherein
R¹ is C₁-C₄-haloalkyl and is in particular CF₃;
R^{2a}, R^{2b} and R^{2c} are selected independently from one another from the group consisting of hydrogen, halogen and C₁-C₂-haloalkyl;
R⁴ is selected from the group consisting of hydrogen, halogen, cyano, C₁-C₄-alkyl, C₁-C₄-haloalkyl, C₁-C₄-alkoxy, C₁-C₄-haloalkoxy, C₁-C₄-alkylthio and C₁-C₄-haloalkylthio;
R^{10a} and R^{14a} independently from each other are selected from the group consisting of hydrogen, C₁-C₆-alkyl, C₁-C₆-haloalkyl;
R^{14b} is selected from the group consisting of hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkyl-C₁-C₄-alkyl-, wherein the cycloalkyl moieties in the three last-mentioned groups may be substituted by a cyano group; C₁-C₆-alkyl substituted with a cyano group, C₁-C₆-alkoxy, C₁-C₆-haloalkoxy, phenyl which is optionally substituted with 1, 2, 3 or 4, substituents each independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; and a heteromonocyclic ring selected from rings of formulae D-1 to D-181 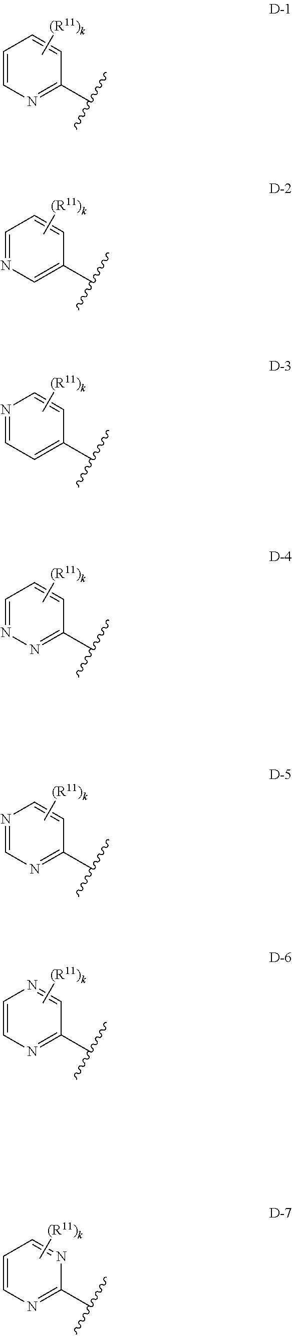 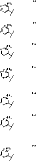 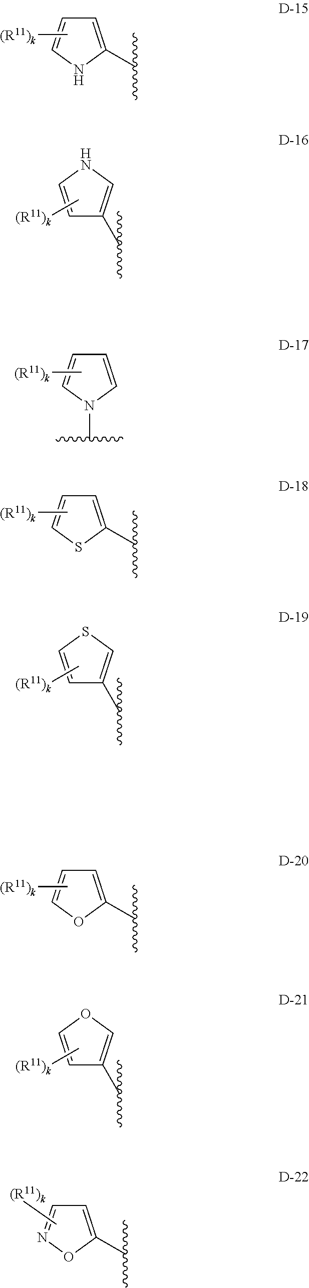 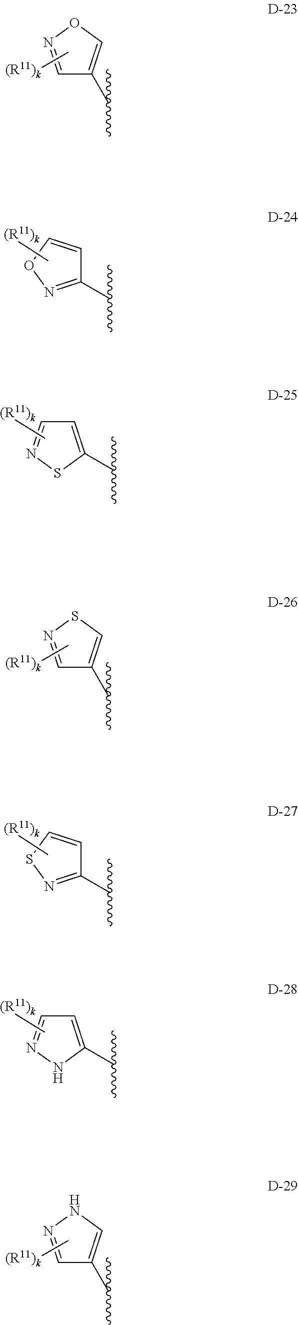 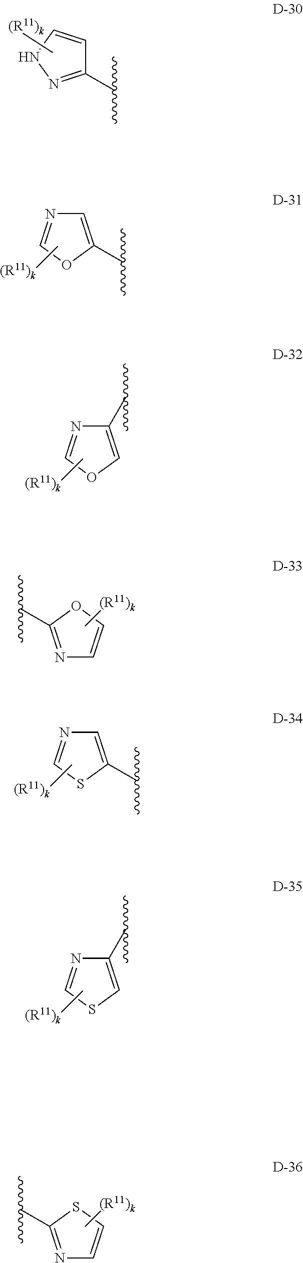 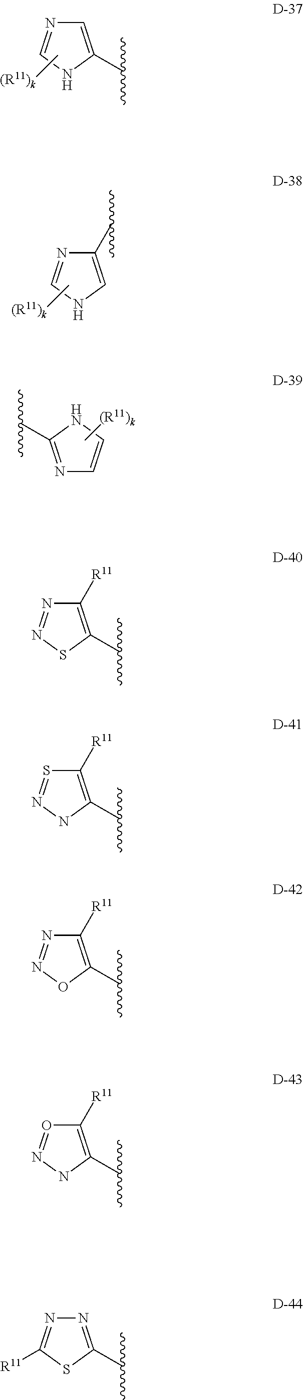 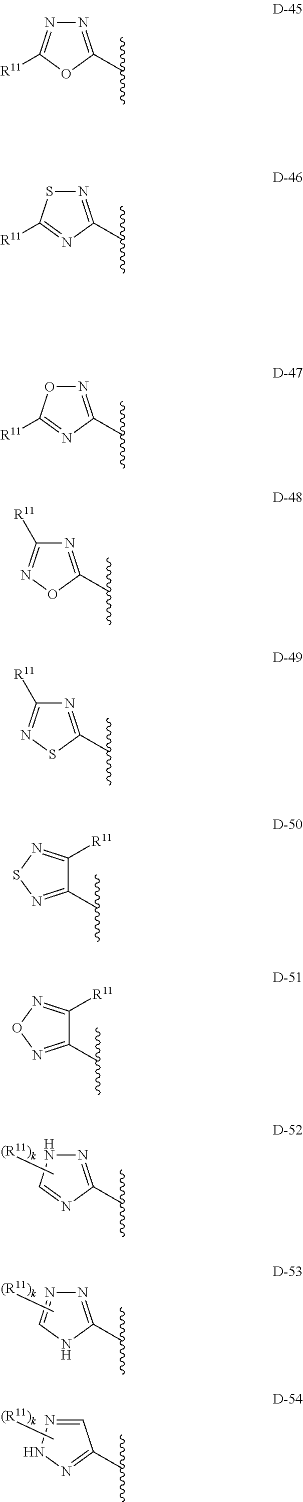 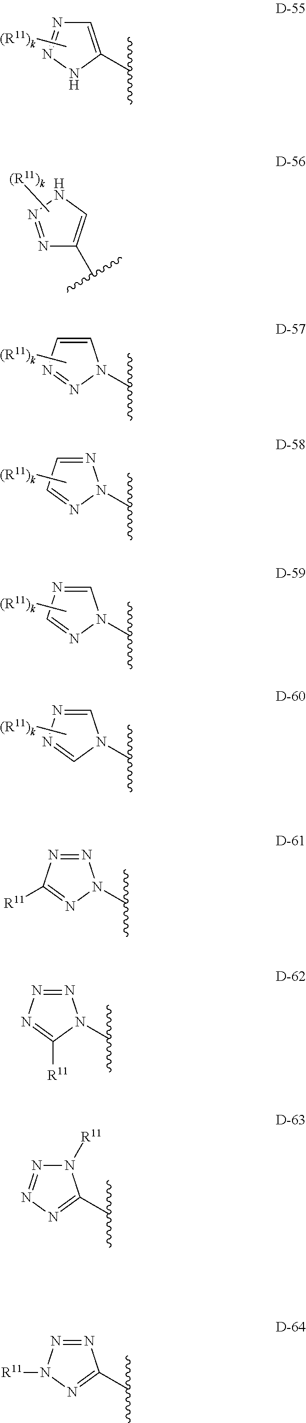 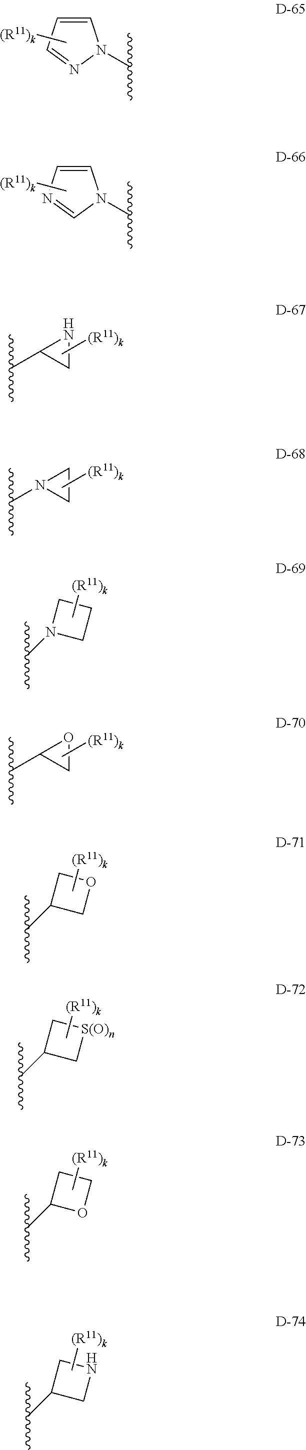 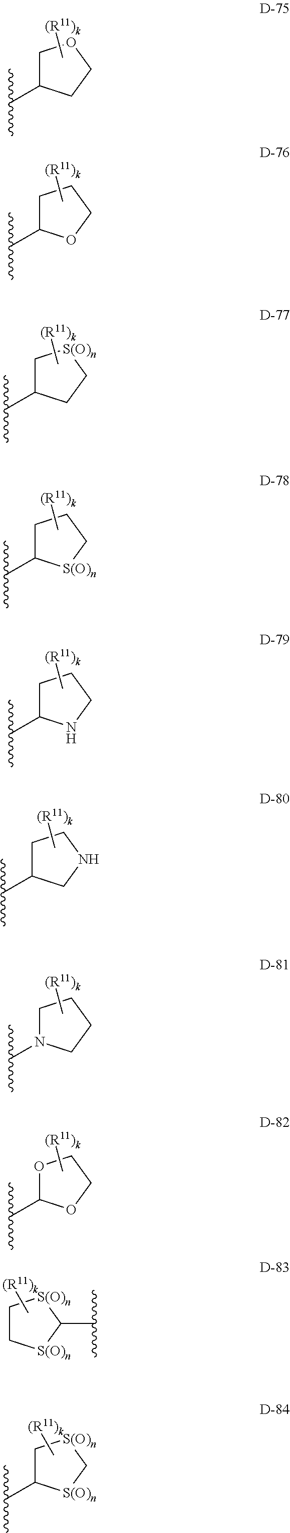 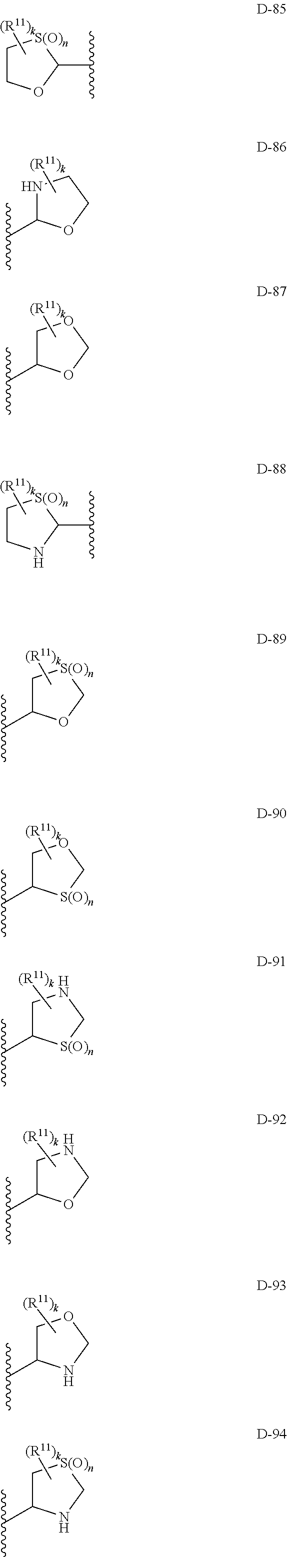 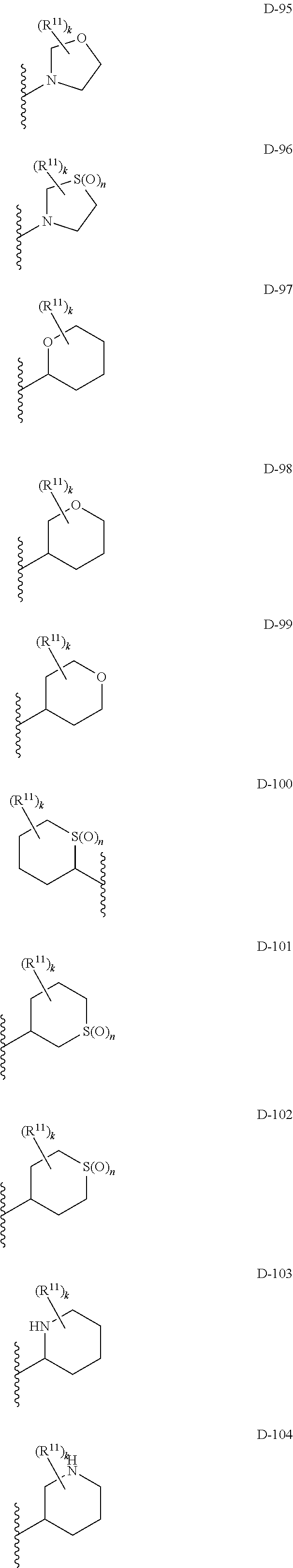 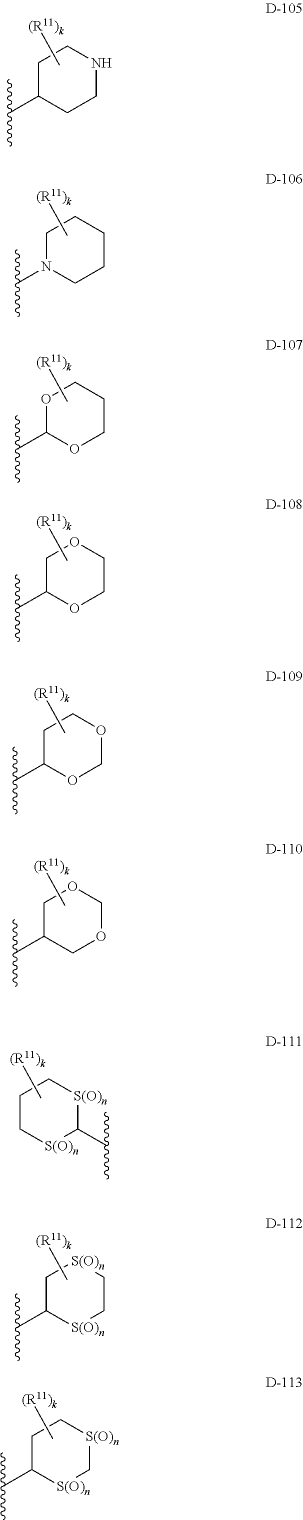 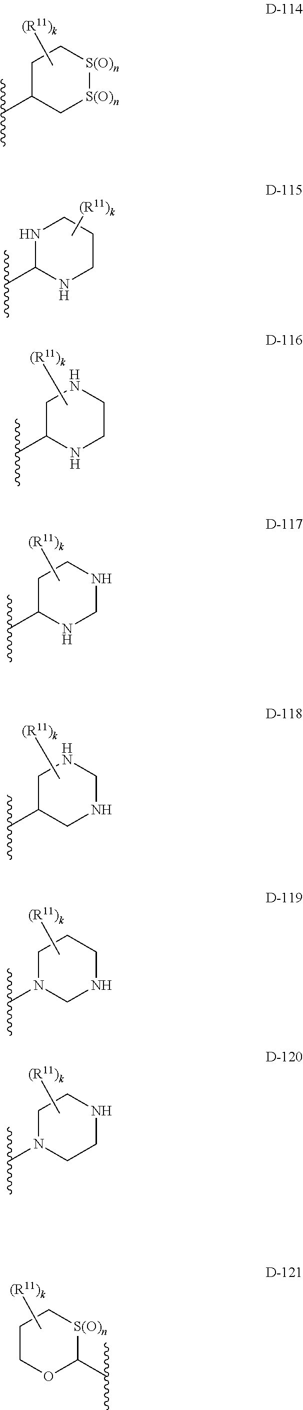 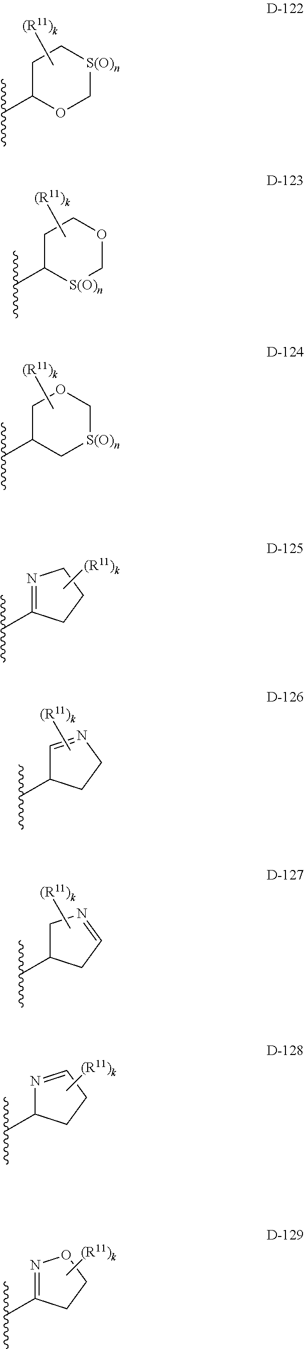 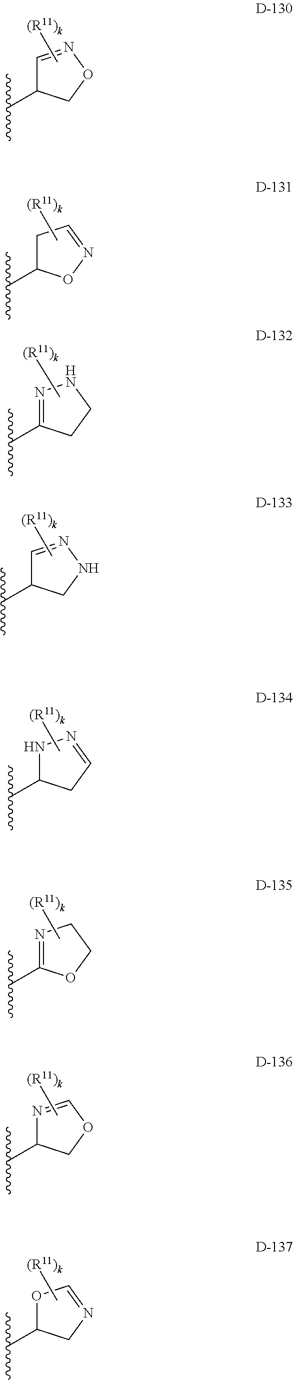 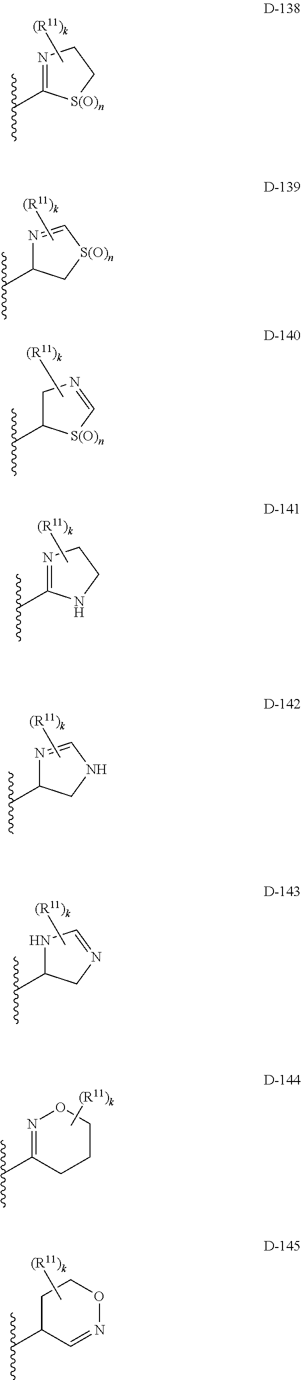 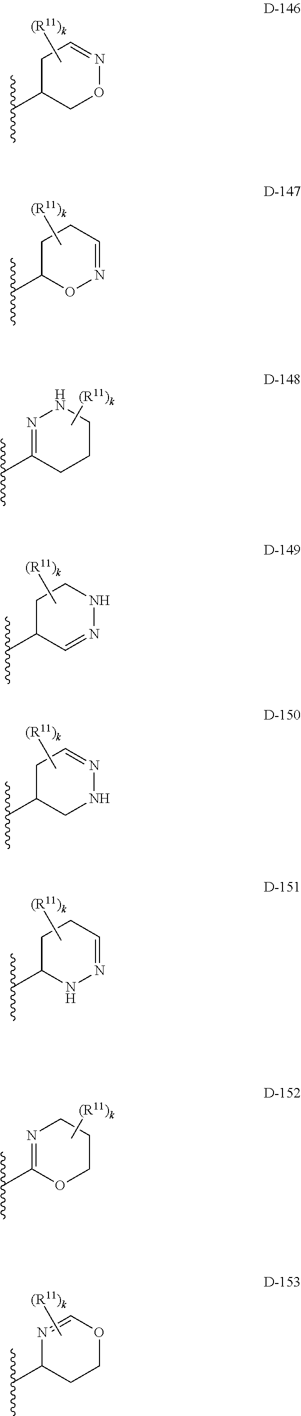 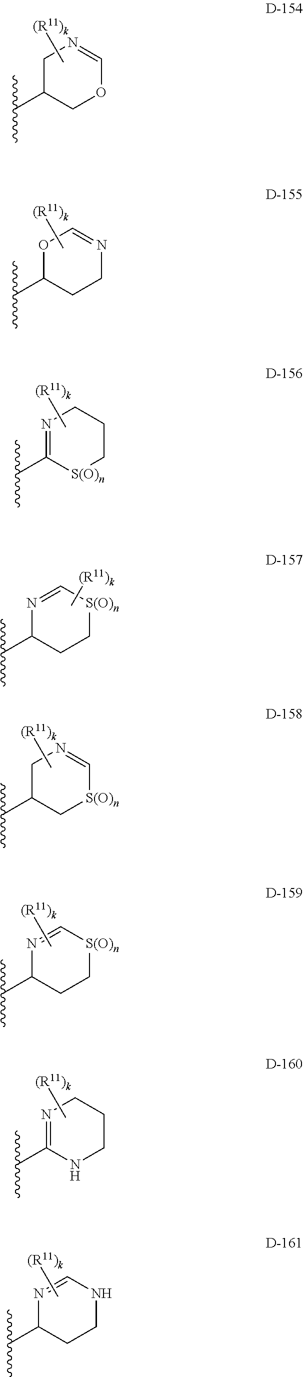 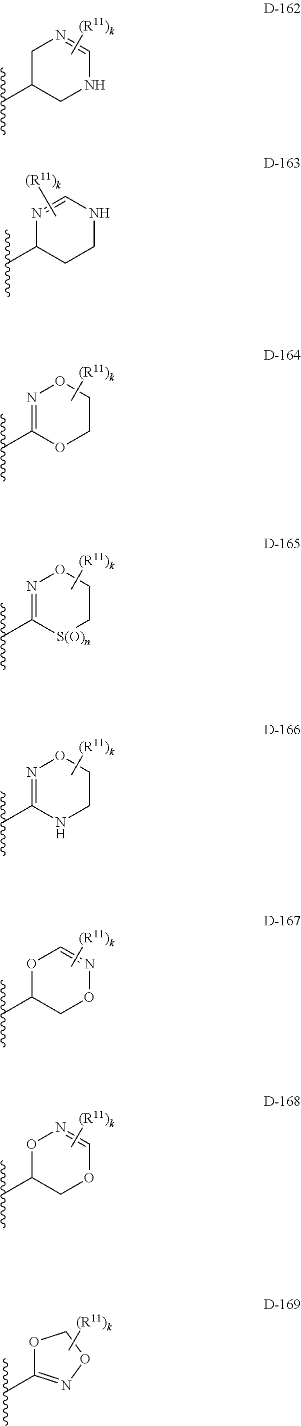 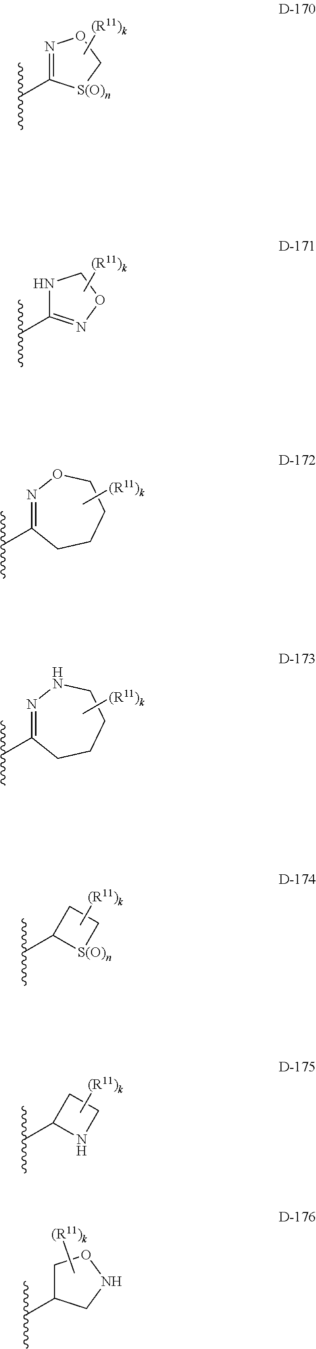 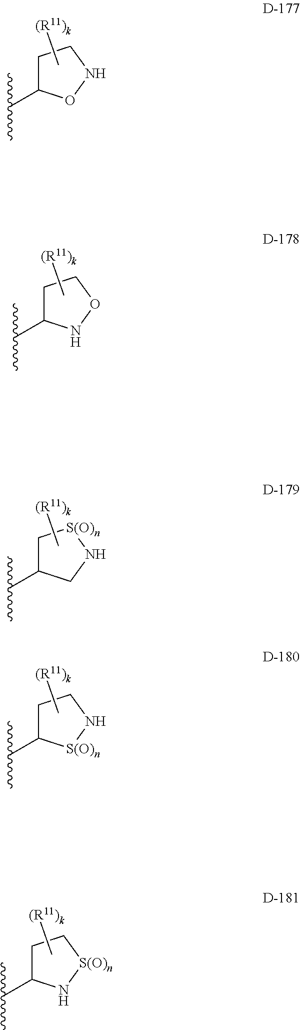
wherein
k is 0, 1, 2 or 3;
n is 0, 1 or 2; and
each R¹¹ is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl, C₁-C₄-alkylcarbonyl, C₁-C₄-alkylaminocarbonyl, or
two R¹¹ present on the same carbon atom of a saturated heterocyclic ring may form together ═O or ═S.

60. The method of claim 37, wherein the isothiazoline compound applied in the method is of formula I.2: 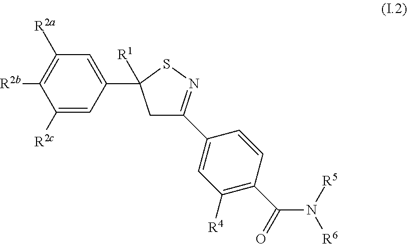
wherein
R¹ is C₁-C₄-haloalkyl and is in particular CF₃;
R^{2a}, R^{2b} and R^{2c} are selected independently from one another from the group consisting of hydrogen, halogen and C₁-C₂-haloalkyl;
R⁴ is selected from the group consisting of hydrogen, halogen, cyano, C₁-C₄-alkyl, C₁-C₄-haloalkyl, C₁-C₄-alkoxy, C₁-C₄-haloalkoxy, C₁-C₄-alkylthio and C₁-C₄-haloalkylthio;
R⁵ is selected from the group consisting of hydrogen, C₁-C₆-alkyl, C₂-C₃-alkenyl, C₂-C₃-alkynyl, —CH₂—CN and C₁-C₆-alkoxy-methyl-; and
R⁶ is selected from the group consisting of hydrogen, C₁-C₆-alkyl, C₁-C₆-haloalkyl, C₁-C₄-alkyl which carries one radical R⁸, C₂-C₆-alkenyl, C₂-C₆-haloalkenyl, C₂-C₆-alkynyl, C₃-C₆-cycloalkyl which may be substituted by 1 to 4 substituents selected from halogen, cyano; N(R^{10a})R^{10b}, —CH═NOR⁹; phenyl which may be substituted with 1, 2, 3, 4, or 5 substituents R¹¹; or a heteromonocyclic ring selected from rings of formulae D-1 to D-181 with its respective substituents R¹¹ wherein R¹¹ is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl, C₁-C₄-alkylcarbonyl, C₁-C₄-alkylaminocarbonyl, or
two R¹¹ present on the same carbon atom of a saturated heterocyclic ring may form together ═O or ═S;
R⁸ is selected from the group consisting of hydrogen, OH, CN, C₃-C₈-cycloalkyl, C₂-C₆-alkenyl, C₂-C₆-alkynyl, wherein the three last-mentioned radicals may be partially or fully halogenated and/or may be substituted by one or more cyano group; C₁-C₆-alkoxy, C₁-C₆-alkylthio, C₁-C₆-alkylsulfinyl, C₁-C₆-alkylsulfonyl, wherein the four last-mentioned radicals may be partially or fully halogenated and/or may be substituted by one or more substituents selected from cyano; C₃-C₆-cycloalkyl, C₂-C₆-alkenyl, C₂-C₆-alkynyl, —C(═O)N(R^{14a})R^{14b}; phenyl, optionally substituted with 1, 2, 3, 4 or 5 substituents R¹¹; or a heteromonocyclic ring selected from rings of formulae D-1 to D-181 with its respective substituents R¹¹ wherein R¹¹ is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl, C₁-C₄-alkylcarbonyl, C₁-C₄-alkylaminocarbonyl, or
two R¹¹ present on the same carbon atom of a saturated heterocyclic ring may form together ═O or ═S;
R⁹ is selected from the group consisting of hydrogen, C₁-C₆-alkyl, C₃-C₆-cycloalkyl, C₃-C₆-cycloalkylmethyl, wherein the three last-mentioned radicals may be partially or fully halogenated and/or may be substituted by one or more CN;
R^{10a} is selected from the group consisting of hydrogen and C₁-C₆-alkyl;
R^{10b} is selected from the group consisting of hydrogen, C(═O)N(R^{14a})R^{14b}; phenyl, optionally substituted with 1, 2, 3, 4 or 5 substituents R¹¹; and a heteromonocyclic ring selected from rings of formulae D-1 to D-181 with its respective substituents R¹¹ wherein R¹¹ is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl, C₁-C₄-alkylcarbonyl, C₁-C₄-alkylaminocarbonyl, or
two R¹¹ present on the same carbon atom of a saturated heterocyclic ring may form together ═O or ═S;
each R¹¹ is independently selected from the group consisting of hydrogen, halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl, C₁-C₄-alkylcarbonyl, C₁-C₄-alkylaminocarbonyl;
each R^{14a} is selected from the group consisting of hydrogen and C₁-C₆-alkyl;
each R^{14b} is selected from the group consisting of hydrogen, C₁-C₆-alkyl, C₃-C₆-cycloalkyl, C₃-C₆-cycloalkylmethyl, C₂-C₄-alkynyl, C₂-C₄-alkenyl, C₁-C₄-alkoxy, wherein the six last-mentioned radicals may be partially or fully halogenated and/or may be substituted with one cyano group.

61. The method of claim 37, wherein the isothiazoline compound applied in the method is of formula I.2a 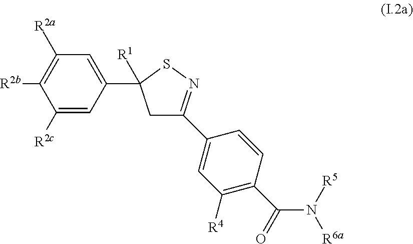
wherein
R¹ is C₁-C₄-haloalkyl and is in particular CF₃;
R^{2a}, R^{2b} and R^{2c} are selected independently from one another from the group consisting of hydrogen, halogen and C₁-C₂-haloalkyl;
R⁴ is selected from the group consisting of hydrogen, halogen, cyano, C₁-C₄-alkyl, C₁-C₄-haloalkyl, C₁-C₄-alkoxy, C₁-C₄-haloalkoxy, C₁-C₄-alkylthio and C₁-C₄-haloalkylthio;
R⁵ is selected from the group consisting of hydrogen, C₁-C₆-alkyl, C₂-C₃-alkenyl, C₂-C₃-alkynyl, CH₂—CN and C₁-C₆-alkoxy-methyl-; and
R^{6a} is selected from the group consisting of —X—R^{6b} and —N(R^{5a})R^{6c}; wherein
X is selected from the group consisting of —C(R^{a})₂—, —C(R^{a})₂—C(R^{a})₂—, —C(R^{a})₂—C(═O)—NR^{10a}—C(R^{a})₂—, —C(R^{a})₂S(O)ₙ—C(R^{a})₂—, —C(R^{a})₂—C(R^{a})₂—S(O)ₙ—C(R^{a})₂—, —C(R^{a})₂—O—C(R^{a})₂—, and —C(R^{a})₂—C(R^{a})₂—O—C(R^{a})₂—, wherein
n is 0, 1 or 2, and
each R^{a} is independently selected from the group consisting of hydrogen, halogen, C₁-C₃-alkyl, and C₁-C₃-haloalkyl;
R^{5a} is selected from the group consisting of hydrogen and C₁-C₆-alkyl;
R^{6b} is selected from the group consisting of C₃-C₆-cycloalkyl, C₂-C₆-alkenyl, C₂-C₆-alkynyl, wherein the three last-mentioned radicals may be partially or fully halogenated and/or may be substituted by one or more substituents R⁸;
R^{6c} is —C(═O)N(R^{14a})R^{14b}
R⁸ is selected from the group consisting of cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —C(═O)N(R^{14a})R^{14b};
each R^{14a} is selected from the group consisting of hydrogen and C₁-C₆-alkyl;
each R^{14b} is selected from the group consisting of hydrogen, C₁-C₆-alkyl, C₃-C₆-cycloalkyl, C₃-C₆-cycloalkylmethyl, C₂-C₄-alkynyl, C₂-C₄-alkenyl, C₁-C₄-alkoxy, wherein the six last-mentioned radicals may be partially or fully halogenated and/or may be substituted with one cyano group;
wherein if R⁵ is hydrogen, R^{6a} is further selected from the group consisting of
hydrogen, 1-cyanocyclopropyl, 1-cyanocyclobutyl and 1-cyanocyclopentyl.

62. The method of claim 37, wherein the isothiazoline compound applied in the method is of formula I.3 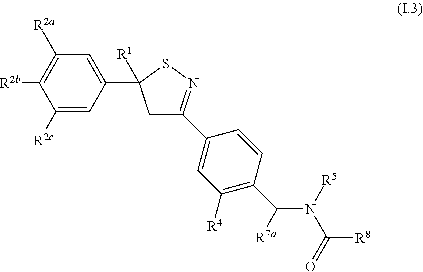
wherein
R¹ is C₁-C₄-haloalkyl and is in particular CF₃;
R^{2a}, R^{2b} and R^{2c} are selected independently from one another from the group consisting of hydrogen, halogen, C₁-C₂-haloalkyl and C₁-C₂-haloalkoxy;
R⁴ is selected from the group consisting of hydrogen, halogen, cyano, nitro, C₁-C₄-alkyl, C₁-C₄-haloalkyl, C₁-C₄-alkoxy, C₁-C₄-haloalkoxy, C₁-C₄-alkylthio and C₁-C₄-haloalkylthio;
R⁵ is selected from the group consisting of hydrogen, C₁-C₄-alkyl, C₂-C₃-alkynyl, —CH₂—CN, C₁-C₆-alkoxy-methyl- and C₃-C₆-cycloalkyl;
R^{7a} is hydrogen or methyl;
R⁸ is selected from the group consisting of hydrogen, C₁-C₆-alkyl, C₃-C₆-cycloalkyl, C₃-C₆-cycloalkenyl, C₂-C₆-alkenyl, wherein the three last-mentioned radicals may be partially or fully halogenated and/or may be substituted by one or more substituents R¹³; C₃-C₈-alkynyl, —N(R^{10a})R^{10b}, —C(═O)N(R^{14a})R^{14b}, —OR⁹, —CH═NOR⁹, phenyl, optionally substituted with 1, 2, 3, 4 or 5 substituents R¹¹; and a heterocyclic ring selected from rings of formulae D-1 to D-181 with its respective substituents R¹¹ wherein R¹¹ is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl, C₁-C₄-alkylcarbonyl, C₁-C₄-alkylaminocarbonyl, or
two R¹¹ present on the same carbon atom of a saturated heterocyclic ring may form together ═O or ═S;
R⁹ is selected from the group consisting of hydrogen, C₁-C₆-alkyl, C₃-C₆-cycloalkyl, C₃-C₆-cycloalkylmethyl, wherein the three last-mentioned radicals may be partially or fully halogenated and/or may be substituted by one or more cyano group;
R^{10a} is selected from the group consisting of hydrogen and C₁-C₆-alkyl;
R^{10b} is selected from the group consisting of hydrogen, C₁-C₆-alkyl, C₁-C₆-cycloalkyl, wherein the two last-mentioned radicals may be partially or fully halogenated and/or are optionally substituted by one cyano group; —C(═O)N(R^{14a})R^{14b}; phenyl, optionally substituted with 1, 2, 3, 4 or 5 substituents R¹¹; and a heteromonocyclic ring selected from rings of formulae D-1 to D-181 with its respective substituents R¹¹ as defined herein above;
each R¹¹ is independently selected from the group consisting of hydrogen, halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl, C₁-C₄-alkylcarbonyl, C₁-C₄-alkylaminocarbonyl; or
two R¹¹ present on the same carbon atom of a saturated heterocyclic ring may form together ═O or ═S;
R¹³ is selected from the group consisting of cyano, C₃-C₆-cycloalkyl, C₃-C₆-halocycloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —N(R^{14a})R^{14b}, —C(═O)N(R^{14a})R^{14b}, phenyl, optionally substituted with 1, 2, 3, 4 or 5 substituents R¹¹, and a heterocyclic ring selected from rings of formulae D-1 to D-181 with its respective substituents R¹¹ as defined herein above;
and as a substituent on a cycloalkyl or cycloalkenyl ring is additionally selected from the group consisting of C₁-C₄-alkyl and C₁-C₄-haloalkyl;
each R^{14a} is selected from the group consisting of hydrogen and C₁-C₆-alkyl; and
each R^{14b} is selected from the group consisting of hydrogen, C₁-C₆-alkyl, C₂-C₄-alkynyl, C₂-C₄-alkenyl, —CH₂—CN, C₁-C₆-haloalkyl, C₃-C₆-cycloalkyl, C₃-C₆-halocycloalkyl, C₁-C₄-alkoxy, C₁-C₄-haloalkoxy and C₃-C₆-cycloalkylmethyl;

63. The method of claim 37, wherein the isothiazoline compound applied in the method is of formula I.3 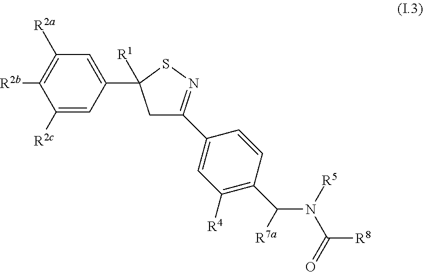
wherein
R¹ is C₁-C₄-haloalkyl and is in particular CF₃;
R^{2a}, R^{2b} and R^{2c} are selected independently from one another from the group consisting of hydrogen, halogen and C₁-C₂-haloalkyl;
R⁴ is selected from the group consisting of hydrogen, halogen, cyano, C₁-C₄-alkyl, C₁-C₄-haloalkyl, C₁-C₄-alkoxy, C₁-C₄-haloalkoxy, C₁-C₄-alkylthio and C₁-C₄-haloalkylthio;
R⁵ is selected from the group consisting of hydrogen, C₁-C₄-alkyl, C₂-C₃-alkynyl, —CH₂—CN and C₁-C₆-alkoxy-methyl-;
R^{7a} is hydrogen or methyl;
R⁸ is selected from the group consisting of hydrogen, C₁-C₆-alkyl, C₃-C₆-cycloalkyl, C₃-C₆-alkenyl, wherein the three last-mentioned radicals may be partially or fully halogenated and/or may be substituted by one or more substituents R¹³; C₃-C₈-alkynyl, —N(R^{10a})R^{10b}, —C(═O)N(R^{14a})R^{14b}, —CH═NOR⁹; phenyl, optionally substituted with 1, 2, 3, 4 or 5 substituents R¹¹; and a heterocyclic ring selected from rings of formulae D-1 to D-181 with its respective substituents R¹¹ wherein R¹¹ is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl, C₁-C₄-alkylcarbonyl, C₁-C₄-alkylaminocarbonyl, or
two R¹¹ present on the same carbon atom of a saturated heterocyclic ring may form together ═O or ═S;
R⁹ is selected from the group consisting of hydrogen, C₁-C₆-alkyl, C₃-C₆-cycloalkyl, C₃-C₆-cycloalkylmethyl, wherein the three last-mentioned radicals may be partially or fully halogenated and/or may be substituted by one or more cyano group;
R^{10a} is selected from the group consisting of hydrogen and C₁-C₆-alkyl;
R^{10b} is selected from the group consisting of hydrogen, C₁-C₆-alkyl, C₁-C₆-cycloalkyl, wherein the two last-mentioned radicals may be partially or fully halogenated and/or are optionally substituted by one cyano group; —C(═O)N(R^{14a})R^{14b}; phenyl, optionally substituted with 1, 2, 3, 4 or 5 substituents R¹¹; and a heteromonocyclic ring selected from rings of formulae D-1 to D-181 with its respective substituents R¹¹ wherein R¹¹ is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl, C₁-C₄-alkylcarbonyl, C₁-C₄-alkylaminocarbonyl, or
two R¹¹ present on the same carbon atom of a saturated heterocyclic ring may form together ═O or ═S;
each R¹¹ is independently selected from the group consisting of hydrogen, halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl, C₁-C₄-alkylcarbonyl, C₁-C₄-alkylaminocarbonyl;
R¹³ is selected from the group consisting of cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —C(═O)N(R^{14a})R^{14b}; phenyl, optionally substituted with 1, 2, 3, 4 or 5 substituents R¹¹; or a heterocyclic ring selected from rings of formulae D-1 to D-181 with its respective substituents R¹¹ wherein R¹¹ is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl, C₁-C₄-alkylcarbonyl, C₁-C₄-alkylaminocarbonyl, or
two R¹¹ present on the same carbon atom of a saturated heterocyclic ring may form together ═O or ═S;
each R^{14a} is selected from the group consisting of hydrogen and C₁-C₆-alkyl; and
each R^{14b} is selected from the group consisting of hydrogen, C₁-C₆-alkyl, C₂-C₄-alkynyl, C₂-C₄-alkenyl, —CH₂—CN, C₁-C₆-haloalkyl, C₃-C₆-cycloalkyl, C₃-C₆-halocycloalkyl, C₁-C₄-alkoxy and C₁-C₄-haloalkoxy, and C₃-C₆-cycloalkylmethyl.

64. The method of claim 37, wherein the isothiazoline compound of formula I is applied in combination with one or more active ingredient selected from the group consisting of organophosphates, fiproles, pyrethroids and neonicotinoids.

65. The method of claim 64, wherein the isothiazoline compound applied in combination with one or more active ingredient is of formula I.1, I.2, I.2a or I.3.
